(19) United States
(12) Patent Application Publication
(10) Pub. No.: US 2012/0195900 A1
(43) Pub. Date: Aug. 2, 2012

(54) TRI-VARIABLE DOMAIN BINDING PROTEINS AND USES THEREOF

(75) Inventors: TARIQ GHAYUR, HOLLISTON, MA (US); CHENGBIN WU, SHANGHAI (CN); HUA YING, SHANGHAI (CN); CARRIE L. GOODREAU, LUDLOW, MA (US); PHILIP BARDWELL, BOSTON, MA (US)

(73) Assignee: ABBOTT LABORATORIES, ABBOTT PARK, IL (US)

(21) Appl. No.: 13/333,362

(22) Filed: Dec. 21, 2011

Related U.S. Application Data
(60) Provisional application No. 61/426,133, filed on Dec. 22, 2010.

(51) Int. Cl. A61K 39/395; C12N 15/13; C12N 15/63; A61P 37/04; C12N 5/10; C12N 1/19; C12P 21/00; G01N 33/53; C07K 16/24; C12N 1/21

(52) U.S. Cl. 424/136.1; 530/387.3; 530/391.1; 530/391.7; 424/178.1; 536/23.53; 435/320.1; 435/252.33; 435/325; 435/419; 435/254.11; 435/349; 435/348; 435/358; 435/365; 435/254.21; 435/69.6; 436/501; 435/7.92

(57) ABSTRACT

The present invention provides engineered multivalent and multispecific binding proteins, as well as methods of making them. Methods for using the multivalent and multispecific binding proteins of the invention in the prevention, diagnosis, and/or treatment of disease are also provided.

### RELATED APPLICATIONS

This application claims priority to U.S. Provisional Patent Application Ser. No. 61/426,133, filed on Dec. 22, 2010, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 14, 2011, is named 117813US.txt and is 278,971 bytes in size.

### BACKGROUND OF THE INVENTION

Engineered proteins, such as multispecific antibodies that can bind to two or more antigens are known in the art. Such multispecific binding proteins can be generated using cell fusion, chemical conjugation, or recombinant DNA techniques.

Bispecific antibodies have been produced using quadroma technology (see Milstein, C. and Cuello, A. C. (1983) Nature 305(5934): 537-40) based on the somatic fusion of two different hybridoma cell lines expressing murine monoclonal antibodies with the desired specificities of the bispecific antibody. Because of the random pairing of two different immunoglobulin (Ig) heavy and light chains within the resulting hybrid-hybridoma (or quadroma) cell line, up to ten different Ig species are generated, of which only one is the functional bispecific antibody. The presence of mis-paired by-products, and significantly reduced production yields, means sophisticated purification procedures are required.

Bispecific antibodies can also be produced by chemical conjugation of two different monoclonal antibodies (see Staerz, U. D. et al. (1985) Nature 314(6012): 628-31). This approach, however, does not yield a homogeneous preparation. Other approaches have used chemical conjugation of two different monoclonal antibodies or smaller antibody fragments (see Brennan, M. et al. (1985) Science 229(4708): 81-3).

Another method used to produce bispecific antibodies is the coupling of two parental antibodies with a hetero-bifunctional crosslinker, but the resulting bispecific antibodies suffer from significant molecular heterogeneity because reaction of the crosslinker with the parental antibodies is not site-directed. To obtain more homogeneous preparations of bispecific antibodies two different Fab fragments have been chemically crosslinked at their hinge cysteine residues in a site-directed manner (see Glennie, M. J. et al. (1987) J. Immunol. 139(7): 2367-75). But this method results in Fab′2 fragments, not a full IgG molecule.

A wide variety of other recombinant bispecific antibody formats have been developed (see Kriangkum, J. et al. (2001) Biomol. Engin. 18(2): 31-40). Amongst them tandem single-chain Fv molecules and diabodies, and various derivatives thereof, are the most widely used. Routinely, construction of these molecules starts from two single-chain Fv (scFv) fragments that recognize different antigens (see Economides, A. N. et al. (2003) Nat. Med. 9(1): 47-52). Tandem scFv molecules (taFv) represent a straightforward format simply connecting the two scFv molecules with an additional peptide linker. The two scFv fragments present in these tandem scFv molecules form separate folding entities. Various linkers can be used to connect the two scFv fragments and linkers with a length of up to 63 residues (see Nakanishi, K. et al. (2001) Ann. Rev. Immunol. 19: 423-74). Although the parental scFv fragments can normally be expressed in soluble form in bacteria, it is, however, often observed that tandem scFv molecules form insoluble aggregates in bacteria. Hence, refolding protocols or the use of mammalian expression systems are routinely applied to produce soluble tandem scFv molecules. In a recent study, in vivo expression by transgenic rabbits and cattle of a tandem scFv directed against CD28 and a melanoma-associated proteoglycan was reported (see Gracie, J. A. et al. (1999) J. Clin. Invest. 104(10): 1393-401). In this construct, the two scFv molecules were connected by a CH1 linker and serum concentrations of up to 100 mg/L of the bispecific antibody were found. Various strategies including variations of the domain order or using middle linkers with varying length or flexibility were employed to allow soluble expression in bacteria. A few studies have now reported expression of soluble tandem scFv molecules in bacteria (see Leung, B. P. et al. (2000) J. Immunol. 164(12): 6495-502; Ito, A. et al. (2003) J. Immunol. 170(9): 4802-9; Karni, A. et al. (2002) J. Neuroimmunol. 125(1-2): 134-40) using either a very short Ala3 linker or long glycine/serine-rich linkers. In a recent study, phage display of a tandem scFv repertoire containing randomized middle linkers with a length of 3 or 6 residues was employed to enrich for those molecules that are produced in soluble and active form in bacteria. This approach resulted in the isolation of a tandem scFv molecule with a 6 amino acid residue linker (see Arndt, M. and Krauss, J. (2003) Methods Mol. Biol. 207: 305-21). It is unclear whether this linker sequence represents a general solution to the soluble expression of tandem scFv molecules. Nevertheless, this study demonstrated that phage display of tandem scFv molecules in combination with directed mutagenesis is a powerful tool to enrich for these molecules, which can be expressed in bacteria in an active form.

Bispecific diabodies (Db) utilize the diabody format for expression. Diabodies are produced from scFv fragments by reducing the length of the linker connecting the VH and VL domain to approximately 5 residues (see Peipp, M. and Valerius, T. (2002) Biochem. Soc. Trans. 30(4): 507-11). This reduction of linker size facilitates dimerization of two polypeptide chains by crossover pairing of the VH and VL domains. Bispecific diabodies are produced by expressing, two polypeptide chains with, either the structure VHA-VLB and VHB-VLA (VH-VL configuration), or VLA-VHB and VLB-VHA (VL-VH configuration) within the same cell. A large variety of different bispecific diabodies have been produced in the past and most of them can be expressed in soluble form in bacteria. A recent comparative study demonstrates that the orientation of the variable domains can influence expression and formation of active binding sites (see Mack, M. et al. (1995) Proc. Natl. Acad. Sci. USA 92(15): 7021-5). Nevertheless, soluble expression in bacteria represents an important advantage over tandem scFv molecules. However, since two different polypeptide chains are expressed within a single cell, inactive homodimers can be produced together with active heterodimers. This necessitates the implementation of additional purification steps in order to obtain homogenous preparations of bispecific diabodies. One approach to force the generation of bispecific diabodies is the production of knob-into-hole diabodies (see Holliger, P., et al. (1993) Proc. Natl. Acad. Sci. USA 90(14): 6444-8.18). This was demonstrated for a bispecific diabody directed against HER2 and CD3. A large knob was introduced in the VH domain by exchanging Val37 with Phe and Leu45 with Trp and a complementary hole was produced in the VL domain by mutating Phe98 to Met and Tyr87 to Ala, either in the anti-HER2 or the anti-CD3 variable domains. By using this approach the production of bispecific diabodies could be increased from 72% by the parental diabody to over 90% by the knob-into-hole diabody. Importantly, production yields did only slightly decrease as a result of these mutations. However, a reduction in antigen-binding activity was observed for several analyzed constructs. Thus, this rather elaborate approach requires the analysis of various constructs in order to identify those mutations that produce heterodimeric molecule with unaltered binding activity. In addition, such approach requires mutational modification of the immunoglobulin sequence at the constant region, thus creating non-native and non-natural form of the antibody sequence, which may result in increased immunogenicity, poor in vivo stability, as well as undesirable pharmacokinetics.

Single-chain diabodies (scDb) represent an alternative strategy to improve the formation of bispecific diabody-like molecules (see Holliger, P. and Winter, G. (1997) Cancer Immunol. Immunother. 45(3-4): 128-30; Wu, A. M. et al. (1996) Immunotechnology 2(1): p. 21-36). Bispecific single-chain diabodies are produced by connecting the two diabody-forming polypeptide chains with an additional middle linker with a length of approximately 15 amino acid residues. Consequently, all molecules with a molecular weight corresponding to monomeric single-chain diabodies (50-60 kDa) are bispecific. Several studies have demonstrated that bispecific single chain diabodies are expressed in bacteria in soluble and active form with the majority of purified molecules present as monomers (see Holliger, P. and Winter, G. (1997) Cancer Immunol. Immunother. 45(3-4): 128-30; Wu, A. M. et al. (1996) Immunotechnol. 2(1): 21-36; Pluckthun, A. and Pack, P. (1997) Immunotechnol. 3(2): 83-105; Ridgway, J. B. et al. (1996) Protein Engin. 9(7): 617-21). Thus, single-chain diabodies combine the advantages of tandem scFvs (all monomers are bispecific) and diabodies (soluble expression in bacteria).

More recently diabodies have been fused to Fc to generate more Ig-like molecules, named di-diabodies (see Lu, D. et al. (2004) J. Biol. Chem. 279(4): 2856-65). In addition, multivalent antibody construct comprising two Fab repeats in the heavy chain of an IgG and that can bind to four antigen molecules has been described (see PCT Publication No. WO 0177342A1, and Miller, K. et al. (2003) J. Immunol. 170(9): 4854-61).

There is a need in the art for improved multivalent binding proteins that can bind two or more antigens. U.S. Pat. No. 7,612,181 provides a novel family of binding proteins, which can bind two or more antigens with high affinity and which are called dual variable domain immunoglobulins (DVD-Ig™) (the entire contents of which are incorporated herein by reference).

### SUMMARY OF THE INVENTION

The present invention provides a novel family of binding proteins that can bind to three or more antigens with high affinity.

In one aspect, the present invention provides binding proteins comprising a polypeptide chain, wherein the polypeptide chain comprises VD1-(X1)n-VD2-(X2)n-VD3-C-(X3)n, wherein; VD1 is a first heavy chain variable domain; VD2 is a second heavy chain variable domain; VD3 is a third heavy chain variable domain; C is a heavy chain constant domain; X1 is a first linker; X2 is a second linker; X3 is an Fc region; and n is 0 or 1; wherein the binding protein is capable of binding one to three target antigens.

In another aspect, the present invention provides binding proteins comprising a polypeptide chain, wherein said polypeptide chain comprises VD1-(X1)n-VD2-(X2)n-VD3-C-(X3)n, wherein VD1 is a first light heavy chain variable domain, VD2 is a second light heavy chain variable domain, VD3 is a third light chain variable domain, C is a light chain constant domain, X1 is a first linker, X1 is a second linker, X3 does not comprise an Fc region, and n is 0 or 1, wherein the binding protein is capable of binding one to three target antigens.

In another aspect, the present invention provides binding proteins comprising a first and a second polypeptide chain, wherein said first polypeptide chain comprises a first VD1-(X1)n-VD2-(X2)n-VD3-C-(X3)n, wherein VD1 is a first heavy chain variable domain, VD2 is a second heavy chain variable domain, VD3 is a third heavy chain variable domain, C is a heavy chain constant domain, X1 is a first linker, X2 is a second linker, and X3 is an Fc region, and wherein said second polypeptide chain comprises a second VD1-(X1)n-VD2-(X2)n-VD3-C-(X3)n, wherein VD1 is a first light chain variable domain, VD2 is a second light chain variable domain, VD3 is a third light chain variable domain, C is a light chain constant domain, X1 is a first linker, X2 is a second linker, and X3 does not comprise an Fc region, and n is 0 or 1, wherein the binding protein is capable of binding one to three target antigens.

In yet another aspect, the present invention provides binding proteins comprising four polypeptide chains, wherein each of the first and third polypeptide chains independently comprise VD1-(X1)n-VD2-(X2)n-VD3-C-(X3)n, wherein VD1 is a first heavy chain variable domain, VD2 is a second heavy chain variable domain, VD3 is a third heavy chain variable domain, C is a heavy chain constant domain, X1 is a first linker, X2 is a second linker, X3 is an Fc region, and wherein each of the second and fourth polypeptide chains independently comprise VD1-(X1)n-VD2-(X2)n-VD3-C-(X3)n, wherein VD1 is a first light chain variable domain, VD2 is a second light chain variable domain, VD3 is a third light chain variable domain, C is a light chain constant domain, X1 is a linker, X2 is a second linker, X3 does not comprise an Fc region, and n is 0 or 1, wherein the binding protein is capable of binding one to six target antigens.

In one embodiment, the Fc region is selected from the group consisting of native sequence Fc region and a variant sequence Fc region. In another embodiment, the Fc region is selected from the group consisting of an Fc region from an IgG1, IgG2, IgG3, IgG4, IgA, IgM, IgE, and IgD.

In one embodiment, two or more of VD1, VD2, and VD3 are independently obtained from a same parent binding protein, e.g., antibody, or antigen-binding portion thereof. In another embodiment, each of VD1, VD2, and VD3 are independently obtained from a same parent binding protein, e.g., antibody, or antigen-binding portion thereof. In another embodiment, two or more of VD1, VD2, and VD3 are independently obtained from a different parent binding protein, e.g., antibody, or antigen-binding portion thereof. In another embodiment, each of VD1, VD2, and VD3 are independently obtained from a different parent binding protein, e.g., antibody, or antigen-binding portion thereof. In one embodiment, the different parent binding proteins, e.g., antibodies, or antigen-binding portion thereof, bind the same epitope on a target antigen. In another embodiment, the different parent binding proteins, e.g., antibodies, or antigen-binding portion thereof, bind different epitopes on a target antigen. In one embodiment, the different parent binding proteins, e.g., antibodies, or antigen-binding portion thereof, bind their respective target antigens with a different potency. In one embodiment, the different parent binding proteins, e.g., antibodies, or antigen-binding portion thereof, bind their respective targets with a different affinity.

In one embodiment, the different parent binding proteins, e.g., antibodies, or antigen-binding portion thereof, are independently selected from the group consisting of a human antibody, a CDR grafted antibody, and a humanized antibody. In another embodiment, the different parent binding proteins, e.g., antibodies, or antigen-binding portion thereof, are independently selected from the group consisting of a Fab fragment; a F(ab′)₂ fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment; an isolated complementarity determining region (CDR); a single chain antibody; a receptor-antibody (Rab); and a diabody.

In one embodiment, the same parent binding protein, e.g., antibody, or antigen-binding portion thereof, is selected from the group consisting of a human antibody, a CDR grafted antibody, and a humanized antibody. In another embodiment, the same parent binding protein, e.g., antibody, or antigen-binding portion thereof, is selected from the group consisting of a Fab fragment; a F(ab′)₂ fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment; an isolated complementarity determining region (CDR); a single chain antibody; and a diabody.

In one embodiment, the binding protein possesses at least one desired property exhibited by the parent binding protein, e.g., antibody, or antigen-binding portion thereof. In one embodiment, the desired property is selected from one or more binding protein, e.g., antibody, parameters. In one embodiment, the binding protein parameters are selected from the group consisting of antigen specificity, affinity to antigen, potency, biological function, epitope recognition, stability, solubility, production efficiency, immunogenicity, pharmacokinetics, bioavailability, tissue cross reactivity, and orthologous antigen-binding.

In one embodiment, the one or more of the target antigens is selected from the group consisting of ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; C1q; C1r; C1; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); Factor VII; Factor IX; Factor V; Factor VIIa; Factor Factor X; Factor XII; Factor XIII; FADD; FasL; FASN; Fc gamma receptor; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; glycoprotein (GP) IIb/IIIa; HAVCR2; HDAC4; HDAC5; HDAC7A; HDAC9; Her2; HGF; HIF1A; HIP1; histamine and histamine receptors; HLA-A; HLA-DRA; HM74; HMGB1; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL-1α; IL-1β; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGS1; RGS13; RGS3; RNF110 (ZNF144); ROBO2; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STAT6; STEAP; STEAP2; substamce P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPRS/CCXCR1); YY1; and ZFPM2.

In one embodiment, the binding protein is capable of binding three target antigens selected from the group consisting of prostaglandin E2 (PGE2), interleukin 13 (IL-13), and interleukin 18 (IL-18) and/or Tumor Necrosis factor alpha (TNFα), interleukin 13 (IL-13), and interleukin 18 (IL-18), and/or interleukin 12 (IL-12), interleukin 23 (IL-23), and Tumor Necrosis factor alpha (TNFα).

In another embodiment, the binding protein is capable of binding three target antigens selected from the group consisting of prostaglandin E2 (PGE2), interleukin 13 (IL-13), interleukin 18 (IL-18), Tumor Necrosis factor alpha (TNFα), interleukin 23 (IL-23), IL-12, HMGB1, VEGF, RAGE, NGF, IL-1α, IL-1β, E-selectin, L-selectin, glycoprotein (GP) thrombomodulin, thrombin, CGRP, TREM, PAI-I, αVβ3, uPA, Her2, IGF1R, EGFR, CD3, Fc gamma receptor, NKG2D, substance P, Protein C, Factor VII, Factor IX, plasminogen activator, Factor V, Factor VIIa, Factor Factor X, Factor XII, Factor XIII, C1q, C1r C1s, C4a, C4b, C2a, C2b, C, C3a and C3b.

In one embodiment, the binding protein is capable of modulating a biological function of the one or more target antigens.

In one embodiment, the binding protein is capable of neutralizing a biological function of the one or more of the target antigens.

In one embodiment, the one or more target antigens is selected from the group consisting of cytokine, chemokine, cell surface protein, enzyme and receptor.

In one embodiment, the cytokine is selected from the group consisting of lymphokines, monokines, and polypeptide hormones. In another embodiment, the cytokine is selected from the group consisting of growth hormone; parathyroid hormone; thyroxine; insulin; proinsulin; relaxin; prorelaxin; glycoprotein hormones; hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); IL-1, IL-2, IL-3, IL-4, IL-5, IL-6, IL-7, IL-8, IL-9, IL-10, IL-11, IL-12, IL-13, IL-15, IL-18, IL-21, IL-22, IL-23, and IL-33; a tumor necrosis factor, LIF and kit ligand (KL).

In one embodiment, the binding protein comprises a triple variable domain immunoglobulin (TVD-Ig) heavy chain amino acid sequence selected from the group consisting of SEQ ID NOs: 45 and 55; and a triple variable domain immunoglobulin (TVD-Ig) light chain amino acid sequence selected from the group consisting of SEQ ID NOs: 50 and 58. In another embodiment, the binding protein comprises a triple variable domain immunoglobulin (TVD-Ig) heavy chain amino acid sequence set forth in SEQ ID NO:69; and a triple variable domain immunoglobulin (TVD-Ig) light chain amino acid sequence set forth in SEQ ID NO:72. In another embodiment, the binding protein comprises a triple variable domain immunoglobulin (TVD-Ig) heavy chain amino acid sequence set forth in SEQ ID NOs: 185 and 187; and a triple variable domain immunoglobulin (TVD-Ig) light chain amino acid sequence set forth in SEQ ID NOs: 186 and 188. In another embodiment, the binding protein comprises a triple variable domain immunoglobulin (TVD-Ig) heavy chain amino acid sequence set forth in SEQ ID NOs: 193 and 195; and a triple variable domain immunoglobulin (TVD-Ig) light chain amino acid sequence set forth in SEQ ID NOs: 194 and 196. In another embodiment, the binding protein comprises a triple variable domain immunoglobulin (TVD-Ig) heavy chain amino acid sequence set forth in SEQ ID NOs: 201 and 203; and a triple variable domain immunoglobulin (TVD-Ig) light chain amino acid sequence set forth in SEQ ID NOs: 202 and 204.

In one embodiment, the chemokine is selected from the group consisting of CCR2, CCR5 and CXCL-13

In one embodiment, the cell surface protein is selected from the group consisting of CTLA4 and TNFRSF1B.

In one embodiment, the enzyme is selected from the group consisting of kinases and proteases.

In one embodiment, the receptor is selected from the group consisting of lymphokine receptor, monokine receptor, and polypeptide hormone receptor.

In one embodiment, the first and second linker comprise an amino acid sequence independently selected from the group consisting of AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9), SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); GHEAAAVMQVQYPAS (SEQ ID NO: 26); TVAAPSVFIFPPTVAAPSVFIFPP (SEQ ID NO: 27); and ASTKGPSVFPLAPASTKGPSVFPLAP (SEQ ID NO: 28).

In one aspect, the present invention provides a binding protein conjugate comprising a binding protein of the invention and an agent selected from the group consisting of an immunoadhension molecule, an imaging agent, a therapeutic agent, and a cytotoxic agent. In one embodiment, the agent is an imaging agent selected from the group consisting of a radiolabel, an enzyme, a fluorescent label, a luminescent label, a bioluminescent label, a magnetic label, and biotin. In one embodiment, the imaging agent is a radiolabel selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm. In one embodiment, the agent is a therapeutic or cytotoxic agent selected from the group consisting of an anti-metabolite, an alkylating agent, an antibiotic, a growth factor, a cytokine, an anti-angiogenic agent, an anti-mitotic agent, an anthracycline, toxin, and an apoptotic agent.

In one embodiment, the binding protein is a crystallized binding protein. In one embodiment, the crystallized binding protein is a carrier-free pharmaceutical controlled release crystal. In one embodiment, the crystallized binding protein has a greater half life in vivo than the soluble counterpart of the binding protein. In another embodiment, the crystallized binding protein retains biological activity.

In one embodiment, the binding protein is produced according to a method comprising, culturing a host cell in culture medium under conditions sufficient to produce the binding protein, wherein the host cell comprises a vector, the vector comprising a nucleic acid encoding the binding protein.

In another aspect, the invention provides a pharmaceutical composition comprising a binding protein of the invention and a pharmaceutically acceptable carrier. In one embodiment, the pharmaceutical composition further comprises at least one additional agent. In one embodiment, the additional agent is a therapeutic or imaging agent. In one embodiment, the additional agent is selected from the group consisting of: Therapeutic agent, imaging agent, cytotoxic agent, angiogenesis inhibitors; kinase inhibitors; co-stimulation molecule blockers; adhesion molecule blockers; anti-cytokine antibody or functional fragment thereof; methotrexate; cyclosporin; rapamycin; FK506; detectable label or reporter; a TNF antagonist; an antirheumatic; a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist.

In yet another aspect, the present invention provides a pharmaceutical composition comprising a binding protein conjugate of the invention and a pharmaceutically acceptable carrier. In one embodiment, the binding protein conjugate comprises an imaging agent selected from the group consisting of a radiolabel, an enzyme, a fluorescent label, a luminescent label, a bioluminescent label, a magnetic label, and biotin. In one embodiment, the imaging agent is a radiolabel selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm. In another embodiment, the binding protein conjugate comprises a therapeutic or cytotoxic agent selected from the group consisting of an anti-metabolite, an alkylating agent, an antibiotic, a growth factor, a cytokine, an anti-angiogenic agent, an anti-mitotic agent, an anthracycline, a toxin, and an apoptotic agent.

In one embodiment, the pharmaceutical composition of the invention further comprises a second agent. In one embodiment, the second agent is a therapeutic or imaging agent. In one embodiment, the therapeutic or imaging agent is selected from the group: cytotoxic agent, angiogenesis inhibitors, kinase inhibitors; co-stimulation molecule blockers; adhesion molecule blockers; anti-cytokine antibody or functional fragment thereof; methotrexate; cyclosporin; rapamycin; FK506; detectable label or reportor; a TNF antagonist; an antiheumatic; a muscle relaxant, a narcotic, anon-steroid anti-inflammatory dug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppresive, a growth hormone, a hormone replacement drug, a radiopharmaceutical, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm, a fluorescent label, a luminescent label, a bioluminescent label, a magnetic label, biotin, an antidepressant, an antipsychotic, a stimulant, an asthma medication, a beta agonist, an inhaled steroid, an epinephrine or analog, a cytokine, and a cytokine antagonist.

In one embodiment, the binding protein has an on rate constant (Kₒₙ) to said one or more targets selected from the group consisting of: at least about 10²M⁻¹s⁻¹; at least about 10³M⁻¹s⁻¹; at least about 10⁴M⁻¹s⁻¹; at least about 10⁵M⁻¹s⁻¹; and at least about 10⁶M⁻¹s⁻¹, as measured by surface plasmon resonance. In another embodiment, the binding protein has an off rate constant (K_{off}) to said one or more targets selected from the group consisting of: at most about 10⁻³s⁻¹; at most about 10⁻⁴s⁻¹; at most about 10⁻⁵s⁻¹; and at most about 10⁻⁶s⁻¹, as measured by surface plasmon resonance. In yet another embodiment, the binding protein has a dissociation constant (K_{D}) to said one or more targets selected from the group consisting of: at most about 10⁻⁷ M; at most about 10⁻⁸ M; at most about 10⁻⁹ M; at most about 10⁻¹⁰ M; at most about 10⁻¹¹ M; at most about 10⁻¹² M; and at most 10⁻¹³M.

In one aspect, the present invention also provides an isolated nucleic acid molecule comprising a nucleotide sequence encoding a binding protein of the invention.

In another aspect, the present invention provides a vector comprising the isolated nucleic acid molecules of the invention. In one embodiment, the vector is selected from the group consisting of pcDNA, pTT, pTT3, pEFBOS, pBV, pJV, pcDNA3.1 TOPO, pEF6 TOPO, and pBJ.

In another aspect, the present invention provides a host cell comprising a vector of the invention. In one embodiment, the host cell is a prokaryotic cell, such as E. coli. In another embodiment, the host cell is a eukaryotic cell. In one embodiment, the eukaryotic cell is selected from the group consisting of protist cell, animal cell, plant cell and fungal cell. In another embodiment, the eukaryotic cell is an animal cell selected from the group consisting of; a mammalian cell, an avian cell, and an insect cell. In one embodiment, the host cell is a CHO cell. In another embodiment, the host cell is a COS cell. In one embodiment, the host cell is a yeast cell, such as Saccharomyces cerevisiae. In one embodiment, the host cell is an insect Sf9 cell.

The present invention also provides methods of producing a binding protein of the invention, comprising culturing the host cell of the invention in culture medium under conditions sufficient to produce the binding protein. In one embodiment, 50%-75% of the binding protein produced is a multi-specific, e.g., triple specific, multi-valent, e.g., sextavalent, binding protein. In another embodiment, 75%-90% of the binding protein produced is a multi-specific, e.g., triple specific, multi-valent, e.g., sextavalent binding protein. In one embodiment, 90%-95% of the binding protein produced is a multi-specific, e.g., triple specific, multi-valent, e.g., sextavalent binding protein.

The present invention also provides proteins produced according to the methods of the invention.

In one aspect, the present invention provides a method for treating a subject for a disease or a disorder, comprising administering to the subject a therapeutically effective amount of the binding protein of the invention, thereby treating the disease or disorder. In one embodiment, the disorder is selected from the group consisting of rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma) Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), H is bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, alopecia areata, anaphylaxis, anti-phospholipid antibody syndrome, aplastic anemia, arteriosclerosis, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, conjunctivitis, childhood onset psychiatric disorder, chronic obstructive pulmonary disease (COPD), dacryocystitis, dermatomyositis, diabetic retinopathy, diabetes mellitus, disk herniation, disk prolaps, drug induced immune hemolytic anemia, endocarditis, endometriosis, endophthalmitis, episcleritis, erythema multiforme, erythema multiforme major, gestational pemphigoid, Guillain-Barré syndrome (GBS), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, ovarian cancer, pauciarticular JRA, peripheral artery occlusive disease (PAOD), peripheral vascular disease (PVD), peripheral artery, disease (PAD), phlebitis, polyarteritis nodosa (or periarteritis nodosa), polychondritis, polymyalgia rheumatica, poliosis, polyarticular JRA, polyendocrine deficiency syndrome, polymyositis, polymyalgia rheumatica (PMR), post-pump syndrome, primary Parkinsonism, prostate and rectal cancer and hematopoietic malignancies (leukemia and lymphoma), prostatitis, pure red cell aplasia, primary adrenal insufficiency, recurrent neuromyelitis optica, restenosis, rheumatic heart disease, sapho (synovitis, acne, pustulosis, hyperostosis, and osteitis), scleroderma, secondary amyloidosis, shock lung, scleritis, sciatica, secondary adrenal insufficiency, silicone associated connective tissue disease, sneddon-wilkinson dermatosis, spondilitis ankylosans, Stevens-Johnson syndrome (SJS), systemic inflammatory response syndrome, temporal arteritis, toxoplasmic retinitis, toxic epidermal necrolysis, transverse myelitis, TRAPS (tumor necrosis factor receptor, type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, wound healing, yersinia and salmonella associated arthropathy.

In one embodiment, the administering to the subject is by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

In another aspect, the present invention provides a method for generating a Tri-Variable Domain Immunoglobulin (TVD-Ig) capable of binding three antigens, comprising obtaining a first parent binding protein, e.g., antibody, or antigen-binding portion thereof, capable of binding a first target antigen, obtaining a second parent binding protein, e.g., antibody, or antigen-binding portion thereof, capable of binding a second target antigen, obtaining a third parent binding protein, e.g., antibody, or antigen-binding portion thereof, capable of binding a third target antigen, constructing first and third polypeptide chains comprising VD1-(X1)n-VD2-(X2)n-VD3-C-(X3)n, wherein VD1 is a first heavy chain variable domain obtained from the first parent binding protein, e.g., antibody, or antigen-binding portion thereof, VD2 is a second heavy chain variable domain obtained from the second parent binding protein, e.g., antibody, or antigen-binding portion thereof, VD3 is a third heavy chain variable domain obtained from the third parent binding protein, e.g., antibody, or antigen-binding portion thereof, C is a heavy chain constant domain, X2 is a first linker, X1 is a second linker, X3 is an Fc region, and n is 0 or 1, and constructing second and fourth polypeptide chains comprising VD1-(X1)n-VD2-(X2)n-VD3-C-(X3)n, wherein VD1 is a first light chain variable domain obtained from the first parent binding protein, e.g., antibody, or antigen-binding portion thereof, VD2 is a second light chain variable domain obtained from the second parent binding protein, e.g., antibody, or antigen-binding portion thereof, VD3 is a second light chain variable domain obtained from the third parent binding protein, e.g., antibody, or antigen-binding portion thereof, C is a light chain constant domain, X1 is a first linker, X2 is a second linker, X3 does not comprise an Fc region, and n is 0 or 1, and expressing the first, second, third and fourth polypeptide chains, such that a Tri-Variable Domain Immunoglobulin capable of binding the first, second, and third target antigens is generated.

In one embodiment, the VD1, VD2, and VD3 heavy chain variable domains comprise an amino acid sequence selected from the group consisting of SEQ ID NOs: 46, 47, 48, 70, 71, 163, 165, 167, 169, and 171 wherein the VD1, VD2, and VD3 light chain variable domains comprise an amino acid sequence selected from the group consisting of SEQ ID NOs: 51, 52, 53, 73, 74, 164, 166, 168, 170, and 172.

In one embodiment, the first, second, and third parent binding protein, e.g., antibody, or antigen-binding portion thereof, are independently selected from the group consisting of a human antibody, a CDR grafted antibody, and a humanized antibody. In another embodiment, the first, second, and third parent binding protein, e.g., antibody, or antigen-binding portion thereof, and are independently selected from the group consisting of a Fab fragment, a F(ab′)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody, a dAb fragment, an isolated complementarity determining region (CDR), a single chain antibody, a receptor-antibody (Rab) and diabodies.

In one embodiment, the first parent binding protein, e.g., antibody, or antigen-binding portion thereof, possesses at least one desired property exhibited by the Tri-Variable Domain Immunoglobulin. In one embodiment, the second parent binding protein, e.g., antibody, or antigen-binding portion thereof possesses at least one desired property exhibited by the Tri-Variable Domain Immunoglobulin. In one embodiment, the third parent binding protein, e.g., antibody, or antigen-binding portion thereof possesses at least one desired property exhibited by the Tri-Variable Domain Immunoglobulin.

In one embodiment, the Fc region is selected from the group consisting of a native sequence Fc region and a variant sequence Fc region. In another embodiment, the Fc region is selected from the group consisting of an Fc region from an IgG1, IgG2, IgG3, IgG4, IgA, IgM, IgE, and IgD.

In one embodiment, the desired property is selected from one or more binding protein, e.g., antibody, parameters. In one embodiment, the binding protein, e.g., antibody, parameter is selected from the group consisting of antigen specificity, affinity to antigen, potency, biological function, epitope recognition, stability, solubility, production efficiency, immunogenicity, pharmacokinetics, bioavailability, tissue cross reactivity, and orthologous antigen-binding.

In one embodiment, the first parent binding protein, e.g., antibody, or antigen-binding portion thereof, binds the first antigen with a different affinity than the affinity with which the second parent binding protein, e.g., antibody, or antigen-binding portion thereof, binds the second antigen or with which the third parent binding protein, e.g., antibody, or antigen-binding portion thereof, binds the third antigen. In another embodiment, the first parent binding protein, e.g., antibody, or antigen-binding portion thereof, binds the first antigen with a different potency than the potency with which the second parent binding protein, e.g., antibody, or antigen-binding portion thereof, binds the second antigen or with which the third parent binding protein, e.g., antibody, or antigen-binding portion thereof, binds the third antigen.

In one aspect of the invention, a method of determining the presence, amount or concentration of an antigen, or fragment thereof, in a test sample, wherein the antigen, or fragment thereof, is selected from the group consisting of prostaglandin E2 (PGE2), interleukin 13 (IL-13), Tumor Necrosis factor alpha (TNFα), interleukin 13 (IL-13), and interleukin 18 (IL-18) is provided. The methods include, assaying the test sample for the antigen, or fragment thereof, by an immunoassay, wherein the immunoassay employs at least one binding protein and at least one detectable label and comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or a fragment thereof, in a control or a calibrator, wherein the calibrator is optionally part of a series of calibrators in which each of the calibrators differs from the other calibrators in the series by the concentration of the antigen, or fragment thereof, and wherein one of the at least one binding protein comprises one or more polypeptide chains comprising VD1-(X1)n-VD2-(X2)n-VD3-C-(X3)n, wherein VD1 is a first heavy chain variable domain obtained from a first parent binding protein, e.g., antibody, or antigen-binding portion thereof, VD2 is a second heavy chain variable domain obtained from a second parent binding protein, e.g., antibody, or antigen-binding portion thereof, VD3 is a third heavy chain variable domain obtained from a third parent binding protein, e.g., antibody, or antigen-binding portion thereof, C is a heavy chain constant domain, X1 is a first linker, X2 is a second linker, X3 is an Fc region, and n is 0 or 1, and can bind a triplet of antigens selected from the group consisting of prostaglandin E2 (PGE2), interleukin 13 (IL-13), and interleukin 18 (IL-18); and Tumor Necrosis factor alpha (TNFα), interleukin 13 (IL-13), and interleukin 18 (IL-18), whereupon the presence, amount or concentration of an antigen, or a fragment thereof, in the test sample is determined.

In another aspect, the present invention provides a method of determining the presence, amount or concentration of an antigen, or fragment thereof, in a test sample, wherein the antigen, or fragment thereof, is selected from the group consisting of prostaglandin E2 (PGE2), interleukin 13 (IL-13), Tumor Necrosis factor alpha (TNFα), interleukin 13 (IL-13), and interleukin 18 (IL-18). The method includes assaying the test sample for the antigen, or fragment thereof, by an immunoassay, wherein the immunoassay employs at least one binding protein and at least one detectable label and comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or a fragment thereof, in a control or a calibrator, wherein the calibrator is optionally part of a series of calibrators in which each of the calibrators differs from the other calibrators in the series by the concentration of the antigen, or fragment thereof, and wherein one of the at least one binding protein comprises one or more polypeptide chains comprising VD1-(X1)n-VD2-(X2)n-VD3-C-(X3)n, wherein, VD1 is a first heavy chain variable domain obtained from a first parent binding protein, e.g., antibody, or antigen-binding portion thereof, VD2 is a second heavy chain variable domain obtained from a second parent binding protein, e.g., antibody, or antigen-binding portion thereof, VD3 is a third heavy chain variable domain obtained from a third parent binding protein, e.g., antibody, or antigen-binding portion thereof, C is a heavy chain constant domain, X1 is a first linker, X2 is a second linker, X3 is an Fc region, and n is 0 or 1, and can bind a triplet of antigens selected from the group consisting of prostaglandin E2 (PGE2), interleukin 13 (IL-13), and interleukin 18 (IL-18); and Tumor Necrosis factor alpha (TNFα), interleukin 13 (IL-13), and interleukin 18 (IL-18), whereupon the presence, amount or concentration of an antigen, or a fragment thereof, in the test sample is determined.

In one embodiment, the method includes contacting the test sample with at least one capture agent, which binds to an epitope on the antigen, or fragment thereof, so as to form a capture agent/antigen, or fragment thereof, complex, contacting the capture agent/antigen, or fragment thereof, complex with at least one detection agent, which comprises a detectable label and binds to an epitope on the antigen, or fragment thereof, that is not bound by the capture agent, to form a capture agent/antigen, or fragment thereof/detection agent complex, and determining the presence, amount or concentration of the antigen, or fragment thereof, in the test sample based on the signal generated by the detectable label in the capture agent/antigen, or a fragment thereof/detection agent complex formed, whereupon the presence, amount or concentration of the antigen, or a fragment thereof, in the test sample is determined wherein at least one capture agent and/or at least one detection agent is the at least one binding protein.

In another embodiment, the methods include contacting the test sample with at least one capture agent, which binds to an epitope on the antigen, or fragment thereof, so as to form a capture agent/antigen, or fragment thereof, complex, and simultaneously or sequentially, in either order, contacting the test sample with detectably labeled antigen, or fragment thereof, which can compete with any antigen, or fragment thereof, in the test sample for binding to the at least one capture agent, wherein any antigen (or fragment thereof) present in the test sample and the detectably labeled antigen compete with each other to form a capture agent/antigen, or fragment thereof, complex and a capture agent/detectably labeled antigen, or fragment thereof, complex, respectively, and determining the presence, amount or concentration of the antigen, or fragment thereof, in the test sample based on the signal generated by the detectable label in the capture agent/detectably labeled antigen, or fragment thereof, complex formed wherein at least one capture agent is the at least one binding protein, wherein the signal generated by the detectable label in the capture agent/detectably labeled antigen, or fragment thereof, complex is inversely proportional to the amount or concentration of antigen, or fragment thereof, in the test sample, whereupon the presence, amount or concentration of antigen, or fragment thereof, in the test sample is determined.

In one embodiment, the test sample is from a patient and the methods further comprise diagnosing, prognosticating, or assessing the efficacy of therapeutic/prophylactic treatment of the patient, wherein, if the method further comprises assessing the efficacy of therapeutic/prophylactic treatment of the patient, the method optionally further comprises modifying the therapeutic/prophylactic treatment of the patient as needed to improve efficacy.

In one embodiment, the methods are adapted for use in an automated system or a semi-automated system.

In one aspect, the present invention provides a kit for assaying a test sample for an antigen, fragment thereof. The kit includes at least one component for assaying the test sample for an antigen, or fragment thereof, and instructions for assaying the test sample for an antigen, or fragment thereof, wherein the at least one component includes at least one composition comprising a binding protein, which comprises one or more polypeptide chains comprising VD1-(X1)n-VD2-(X2)n-VD3-C-(X3)n, wherein, VD1 is a first heavy chain variable domain obtained from a first parent binding protein, e.g., antibody, or antigen-binding portion thereof, VD2 is a second heavy chain variable domain obtained from a second parent binding protein, e.g., antibody, or antigen-binding portion thereof, VD3 is a third heavy chain variable domain obtained from a third parent binding protein, e.g., antibody, or antigen-binding portion thereof, C is a heavy chain constant domain, X1 is a first linker, X2 is a second linker, X3 is an Fc region, and n is 0 or 1, and can bind a triplet of antigens selected from the group consisting of prostaglandin E2 (PGE2), interleukin 13 (IL-13), and interleukin 18 (IL-18); and Tumor Necrosis factor alpha (TNFα), interleukin 13 (IL-13), and interleukin 18 (IL-18), wherein the binding protein is optionally detectably labeled.

In another aspect, the present invention provides a kit for assaying a test sample for an antigen, or fragment thereof. The lit includes at least one component for assaying the test sample for an antigen, or fragment thereof, and instructions for assaying the test sample for an antigen, or fragment thereof, wherein the at least one component includes at least one composition comprising a binding protein, which comprises one or more polypeptide chains comprising VD1-(X1)n-VD2-(X2)n-VD3-C-(X3)n, wherein, VD1 is a first heavy chain variable domain obtained from a first parent binding protein, e.g., antibody, or antigen-binding portion thereof, VD2 is a second heavy chain variable domain obtained from a second parent binding protein, e.g., antibody, or antigen-binding portion thereof, VD3 is a third heavy chain variable domain obtained from a third parent binding protein, e.g., antibody, or antigen-binding portion thereof, C is a heavy chain constant domain, X1 is a first linker, X2 is a second linker, X3 is an Fc region, and n is 0 or 1, and can bind a triplet of antigens selected from the group consisting of prostaglandin E2 (PGE2), interleukin 13 (IL-13), and interleukin 18 (IL-18); and Tumor Necrosis factor alpha (TNFα), interleukin 13 (IL-13), and interleukin 18 (IL-18), wherein the binding protein is optionally detectably labeled.

### BRIEF DESCRIPTION OF THE DRAWINGS

FIG. 1 is a schematic representation of Tri-Variable Domain (TVD)-Ig constructs and shows the strategy for generation of a TVD-Ig protein from parent binding proteins, e.g., antibodies.

### DETAILED DESCRIPTION OF THE INVENTION

This present disclosure pertains to multivalent and/or multispecific binding proteins that can bind to three or more antigens. Specifically, the present disclosure relates to triple or tri-variable (TVD) domain binding proteins, and pharmaceutical compositions thereof, as well as nucleic acids, recombinant expression vectors and host cells for making such TVD binding proteins. Methods of using the TVD binding proteins of the present disclosure to detect specific antigens, either in vitro or in vivo are also encompassed by the present disclosure.

Unless otherwise defined herein, scientific and technical terms used in connection with the present disclosure shall have the meanings that are commonly understood by those of ordinary skill in the art. The meaning and scope of the terms should be clear; however, in the event of any latent ambiguity, definitions provided herein take precedent over any dictionary or extrinsic definition. Further, unless otherwise required by context, singular terms shall include pluralities and plural terms shall include the singular. In this application, the use of the term “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise. Unless otherwise clear from context, all values herein can be understood to be modified by the term “about”. The amount of variation tolerated will depend on the specific value, but is typically considered to be within two standard deviations of the mean. “About” can be understood to be a variation of up to 10%, 9%, 8%, 7%, 6%, 5%, 4%, 3%, 2%, 1%, 0.1%, or 0.01%. Ranges provided herein are understood to include all of the values within the range, or any subset of ranges or values within the range. For example, 1-10 is understood to include 1, 2, 3, 4, 5, 6, 7, 8, 9, and 10, or any range or subset of those values, and fractional values when appropriate. Similarly, ranges provided as “up to” a certain value are understood to include values from zero to the top end of the range; and “less than” is understood to include values from that number to zero.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the present disclosure may be more readily understood, select terms are defined below.

The term, ‘binding protein” or “binding molecule” as used herein includes molecules that contain at least one antigen binding site that specifically binds to a molecule of interest. A binding protein may be an antibody or any other polypeptide, e.g., a receptor-antibody (Rab) protein.

The terms “tri-variable binding protein”, “triple variable binding protein”, and “TVD binding protein”, as used herein include molecules that contain three or six antigen binding sites, each of which independently and specifically binds a target antigen. In one embodiment, a TVD binding protein is a TVD-Immunoglobulin (TVD-Ig) binding protein.

The terms “specific binding” or “specifically binding,” as used herein, in reference to the interaction of a binding protein with a second chemical species, such as a protein or polypeptide, mean that the interaction is dependent upon the presence of a particular structure (e.g., an antigenic determinant or epitope) on the chemical species; for example, a binding protein recognizes and binds to a specific protein structure, rather than to proteins generally. If a binding protein is specific for epitope “A,” the presence of a molecule containing epitope A (or free, unlabeled A) in a reaction containing labeled “A” and the binding protein will reduce the amount of labeled A bound to the antibody. It should be noted that a binding protein that specifically binds a target antigen(s) may, however, have cross-reactivity to target antigen(s) from other species.

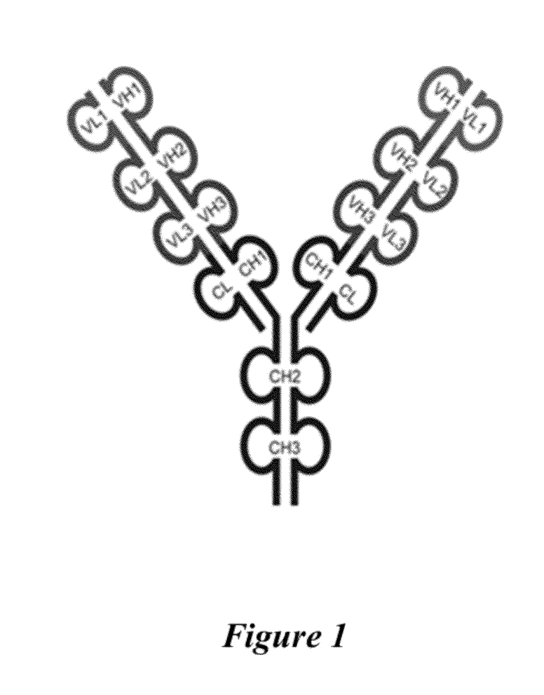

In general, the TVD binding proteins of the invention comprise a polypeptide chain comprising VD1-(X1)n-VD2-(X2)n-VD3-C-(X3)n, wherein VD1 is a first variable domain, VD2 is a second variable domain, VD3 is a third variable domain, C is a constant domain, X1 is a first linker, X2 is a second linker, X3 is an Fc region and n is 0 or 1, and are capable of binding three target antigens. FIG. 1 depicts the structure of an exemplary TVD binding protein of the invention.

In exemplary embodiments, each of “VD1”, “VD2”, and “VD3” is independently a heavy chain variable domain or a light chain variable domain.

As used herein, the phrases a “heavy chain variable domain” or a “heavy chain antigen binding domain” (referred to herein as VD or VD_{H}) are intended to include a heavy chain variable domain of a dual heavy chain variable domain, a triple heavy chain variable domain, a domain antibody, an ScFv, a receptor, and a scaffold antigen binding protein. It is understood that the heavy chain antigen binding domain may or may not bind an antigen independently of a paired light chain variable domain present on a second polypeptide of the binding proteins of the invention. For example, if a heavy chain variable domain is derived from a domain antibody, an scFv, or a receptor, it would be expected to bind a target independent of any amino acid sequences on a second polypeptide claim. As the binding proteins of the invention form functional antigen binding sites, if the heavy chain antigen binding domain cannot specifically bind a target antigen independently (i.e., does not alone provide a functional antibody binding site), a second polypeptide should be present to provide a complementary light chain variable domain to provide a functional antibody binding site.

As used herein, the phrases a “light chain variable domain” or a “light chain antigen binding domain” (referred to herein as VD or VD_{L}) are intended to include a light chain variable domain of a dual light chain variable domain, a triple light chain variable domain, a domain antibody, an ScFv, a receptor, and a scaffold antigen binding protein. It is understood that the light chain antigen binding domain may or may not bind an antigen independently of a paired heavy chain variable domain present on another polypeptide of the binding proteins of the invention. For example, if a light chain variable domain is derived from a domain antibody, an scFv, or a receptor, it would be expected to bind a target independent of any amino acid sequences on a second polypeptide claim.

As used herein, “VD” alone is to be understood to be either a heavy chain antigen binding domain or a light chain antigen binding unless otherwise clear from context.

In one embodiment, VD1, VD2, and VD3 are each a heavy chain variable domain. In another embodiment, VD1, VD2, and VD3 are each a light chain variable domain.

In exemplary embodiments, each of “X1” and “X2” is a linker and each of “n” is independently 0 or 1.

The term “linker” refers to polypeptides comprising two or more amino acid residues joined by peptide bonds used to link one or more antigen-binding portions or domains. Such linker polypeptides are well known in the art (see, e.g., Holliger, P. et al. (1993) Proc. Natl. Acad. Sci. USA 90: 6444-6448; Poljak, R. J. et al. (1994) Structure 2:_{—}1121-1123). Exemplary linkers include, but are not limited to, AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9), SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); GHEAAAVMQVQYPAS (SEQ ID NO: 26); TVAAPSVFIFPPTVAAPSVFIFPP (SEQ ID NO: 27); and ASTKGPSVFPLAPASTKGPSVFPLAP (SEQ ID NO: 28).

In one embodiment, (X1)n is 0. In another embodiment, (X1)n is 1. In yet another embodiment, (X2)n is 0. In a further embodiment, (X2)n is 1. In another embodiment, (X2)n is not CH1 and may be either 0 or 1.

In exemplary embodiments, “C” is heavy chain or light chain constant domain.

As used herein, a “light chain constant domain” (also referred to herein as “C_{L}”) refers to a domain derived from the constant domain of the light chain of an immunoglobulin molecule. As used herein a “light chain constant domain” may be a lambda light chain constant region or a kappa light chain constant region, unless specified. Human light chain constant domain amino acid sequences are known in the art.

As used herein, a “heavy chain constant domain” (also referred to herein as “C_{H}”) refers to a domain derived from the constant domain of the heavy chain of an immunoglobulin molecule. The heavy chain constant region is comprised of three domains, CH1, CH2 and CH3, and optionally a fourth domain, CH4. Human heavy chain constant domain amino acid sequences are known in the art.

It is understood that, as used herein, “C” alone can be understood to be either a heavy chain constant domain or a light chain constant unless otherwise clear from context.

In one embodiment, C is a light chain constant domain. In another embodiment, C is a heavy chain constant domain. In yet another embodiment, C is a heavy chain CH1 domain. In one embodiment, C is a heavy chain CH2 domain. In another embodiment, C is a heavy chain CH3 domain. In yet another embodiment, C is a heavy chain CH4 domain. In one embodiment, C is not a heavy chain CH1 domain. In another embodiment, C is not a heavy chain CH2 domain. In one embodiment, C is not a heavy chain CH3 domain. In another embodiment, C is not a heavy chain CH4 domain.

In exemplary embodiments, “(X3)n” is an Fc region and “n” is 0 or 1. In one embodiment, n is 0. In another embodiment, n is 1.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, e.g., cytokine induction, ADCC, phagocytosis, complement dependent cytotoxicity (CDC), and half-life/clearance rate of antibody and antigen-antibody complexes. In some cases these effector functions are desirable for a therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the binding protein, e.g., antibody, for example the Fc region of the antibody, such that effector functions of the binding protein, e.g., antibody are altered. The dimerization of two identical heavy chains of an immunoglobulin is mediated by the dimerization of CH3 domains and is stabilized by the disulfide bonds within the hinge region (Huber et al. (1976) Nature 264: 415-20; Thies et al. (1999) J. Mol. Biol. 293: 67-79).

TVD binding proteins comprising two heavy chain TVD polypeptides and two light chain TVD polypeptides and are referred to herein as “TVD-Ig proteins” or “TVD-Ig binding proteins”. Each half of a TVD-Ig protein comprises a heavy chain TVD polypeptide, and a light chain TVD polypeptide, and three antigen-binding sites. Each binding site comprises a heavy chain variable domain and a light chain variable domain with a total of six CDRs involved in antigen binding per antigen-binding site.

The term “polypeptide or “polypeptide chain”, as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric. Use of “polypeptide” herein is intended to encompass polypeptides, and fragments and variants (including fragments of variants) thereof, unless otherwise stated. For an antigenic polypeptide, a fragment of polypeptide optionally contains at least one contiguous or nonlinear epitope of polypeptide. The precise boundaries of the at least one epitope fragment can be confirmed using ordinary skill in the art. The fragment comprises at least about 5 contiguous amino acids, such as at least about 10 contiguous amino acids, at least about 15 contiguous amino acids, or at least about 20 contiguous amino acids. A variant of polypeptide is as described herein.

The binding proteins of the invention may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. Binding proteins may have both a heavy and a light chain. As used herein, the term binding protein also includes, antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, so long as they exhibit the desired activity, e.g., binding to a target antigen(s).

The term “antibody,” as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art, and nonlimiting examples thereof are discussed herein below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG 3, IgG4, IgA1 and IgA2), or subclass.

The term “antigen-binding portion” of a binding protein, e.g., an antibody (or simply “antigen-binding fragments thereof”), as used herein, refers to one or more fragments of an binding protein, e.g., antibody, that retain the ability to bind specifically to an antigen. It has been shown that the antigen-binding function of a binding protein, e.g., an antibody, can be performed by fragments of a full-length binding protein, e.g., antibody. Such binding protein, e.g., antibody, embodiments may also be bispecific, dual specific, or multi-specific formats (specifically binding to two or more different antigens). Examples of binding fragments encompassed within the term “antigen-binding portion” of a binding protein, e.g., an antibody, include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward (1989) Nature 341: 544-546; and PCT Publication No. WO 90/05144 A1), which comprises a single variable domain; (vi) receptor-antibody (Rab) fragments, and (vii) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see, e.g., Holliger, P. et al. (1993) Proc. Natl. Acad. Sci. USA 90: 6444-6448; Poljak, R. J. et al. (1994) Structure 2:_{—}1121-1123). Such binding portions are known in the art (Kontermann and Dubel eds., Antibody Engineering (2001) Springer-Verlag. New York. pp. 790 (ISBN 3-540-41354-5)). In addition single chain antibodies also include “linear antibodies” comprising a pair of tandem Fv segments (VH-CH1-VH-CH1) that, together with complementary light chain polypeptides, form a pair of antigen-binding regions (Zapata et al. (1995) Protein Eng. 8(10): 1057-1062 and U.S. Pat. No. 5,641,870).

The term “multivalent binding protein” is used throughout this specification to denote a binding protein comprising two or more antigen-binding sites. In one embodiment, the multivalent binding protein is engineered to have three or more antigen-binding sites and is generally not a naturally occurring antibody, e.g., is an isolated an/or recombinant antibody. In one embodiment, the multivalent binding protein is engineered to have three antigen-binding sites. In another embodiment, the multivalent binding protein is engineered to have six antigen-binding sites.

The term “multispecific binding protein” refers to a binding protein that can bind two or more related or unrelated targets. The binding proteins of the present invention comprise three or six antigen-binding sites and are trivalent or sextavalant multivalent binding proteins. The binding proteins of the present invention may be monospecific, i.e., capable of binding one target, or multispecific, e.g. capable of binding two or more targets, i.e., two, three, four, five, or six targets.

As used herein, “Dual Variable Domain Immunoglobulin” or “DVD-Ig™” and the like are understood to include immunoglobulin molecules having the structure schematically represented in FIG. 1A and provided in US Patent Publications 20100260668 and 20090304693 both of which are incorporated herein by reference including sequence listings. A DVD-Ig™ comprises a paired heavy chain DVD polypeptide and a light chain DVD polypeptide with each paired heavy and light chain providing two antigen binding sites. Each binding site includes a total of 6 CDRs involved in antigen binding per antigen binding site. A DVD-Ig™ is typically has two arms (is divalent), with each arm of the DVD being dual-specific, providing an immunoglobulin with four binding sites.

The term “bispecific antibody,” as used herein, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al. (1985) Nature 314(6012): 628-631), or by knob-into-hole or similar approaches, which introduce mutations in the Fc region (see Holliger, P. et al. (1993) Proc. Natl. Acad. Sci. USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen it binds to. In one embodiment of the invention, a TVD-Ig protein is bispecific in that the three variable domains on a first arm each independently bind to the same antigen and the three variable domains on the other arm each independently bind to the same antigen which is different from the antigen bound by the first arm.

The term “dual-specific binding-protein,” as used herein, refers to full-length antibodies that can bind two different antigens (or epitopes) in each of its two binding arms (a pair of HC/LC) (see PCT Publication No. WO 02/02773). Accordingly a dual-specific binding protein has two identical antigen binding arms, with identical specificity and identical CDR sequences, and is bivalent for each antigen to which it binds. In one embodiment of the invention, a TVD-Ig protein is dual-specific in that both of the arms of the TVD-Ig protein are identical in that two of the three variable domains on each binding arm each independently bind a first antigen and the third variable domain on each binding arm binds a a different second antigen.

As used herein, “Receptor-Antibody Immunoglobulin” or “RAb-Ig” and the like are understood to include immunoglobulin molecules provided in US Patent Application 2002/0127231, which is incorporated herein by reference including sequence listings. RAb-Ig comprises a heavy chain RAb polypeptide, and a light chain RAb polypeptide, which together form three antigen binding sites in total. One antigen binding site is formed by the pairing of the heavy and light antibody variable domains present in each of the heavy chain RAb polypeptide and the light chain RAb polypeptide to form a single binding site with a total of 6 CDRs providing a first antigen binding site. Each of the heavy chain RAb polypeptide and the light chain RAb polypeptide include a receptor sequence that independently binds a ligand providing the second and third “antigen” binding sites.

A “functional antigen-binding site” of a binding protein is one that that can bind to a target antigen. The antigen-binding affinity of the antigen-binding site is not necessarily as strong as the parent binding protein, e.g., antibody, from which the antigen-binding site is derived, but the ability to bind antigen must be measurable using any one of a variety of methods known for evaluating antibody binding to an antigen. Moreover, the antigen-binding affinity of each of the antigen-binding sites of a multivalent binding protein, e.g., antibody herein need not be quantitatively the same.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering,” as used herein, refers to the process of rendering a chemical species, such as a polypeptide, substantially free of naturally associated components by isolation, e.g., using protein purification techniques well known in the art.

“Biological activity,” as used herein, refers to any one or more inherent biological properties of a molecule (whether present naturally as found in vivo, or provided or enabled by recombinant means). Biological properties include but are not limited to binding a receptor, inducing cell proliferation, inhibiting cell growth, inducing other cytokines, inducing apoptosis, and enzymatic activity. Biological activity also includes activity of an Ig molecule.

The term “cytokine” is a generic term for proteins released by one cell population, which act on another cell population as intercellular mediators. Examples of such cytokines are lymphokines, monokines, and traditional polypeptide hormones. Included among the cytokines are growth hormone, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors, such as NGF-alpha; platelet-growth factor; placental growth factor, transforming growth factors (TGFs), such as TGF-alpha and TGF-beta; insulin-like growth factor-1 and -11; erythropoietin (EPO); osteoinductive factors; interferons, such as interferon-alpha, -beta and -gamma; colony stimulating factors (CSFs), such as macrophage-CSF (M-CSF), granulocyte macrophage-CSF (GM-CSF), and granulocyte-CSF (G-CSF); interleukins (ILs), such as IL-1, IL-2, IL-3, IL-4, IL-5, IL-6, IL-7, IL-8, IL-9, IL-10, IL-11, IL-12, IL-13, IL-15, IL-18, IL-21, IL-22, IL-23, and IL-33; a tumor necrosis factor, such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “monoclonal antibody” or “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method.

The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35: 425-445; Gavilondo, J. V. and Larrick, J. W. (2002) BioTechniques 29: 128-145; Hoogenboom, H. and Chames, P. (2000) Immunol. Today 21:_{—}371-378), antibodies isolated from an animal (e.g., a mouse) that is transgenic for human immunoglobulin genes (see, Taylor, L. D. et al. (1992) Nucl. Acids Res. 20: 6287-6295; Kellermann, S-A. and Green, L. L. (2002) Cur. Opin. in Biotechnol. 13: 593-597; Little, M. et al. (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies are sequences that, while derived from and related to human germline VH and VL sequences, may not naturally exist within the human antibody germline repertoire in vivo.

An “affinity matured” antibody is an antibody with one or more alterations in one or more CDRs thereof, which result an improvement in the affinity of the antibody for antigen compared to a parent antibody, which does not possess those alteration(s). Exemplary affinity matured antibodies will have nanomolar or even picomolar affinities for the target antigen. Affinity matured antibodies are produced by procedures known in the art. Marks et al. (1992) Bio/Technology 10: 779-783 describes affinity maturation by VH and VL domain shuffling. Random mutagenesis of CDR and/or framework residues is described by Barbas, et al. (1994) Proc Nat. Acad. Sci. USA 91: 3809-3813; Schier et al. (1995) Gene 169: 147-155; Yelton et al., (1995) J. Immunol. 155: 1994-2004; Jackson et al. (1995) J. Immunol. 154(7): 3310-9; and Hawkins et al. (1992) J. Mol. Biol. 226: 889-896; and selective mutation at selective mutagenesis positions, contact or hypermutation positions with an activity enhancing amino acid residue is described in U.S. Pat. No. 6,914,128.

The term “chimeric antibody” refers to antibodies, which comprise heavy and light chain variable region sequences from one species and constant region sequences from another species, such as antibodies having murine heavy and light chain variable regions linked to human constant regions.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the FR regions are those of a human immunoglobulin consensus sequence. In one embodiment, a humanized antibody also comprises at least a portion of an immunoglobulin Fc region, typically that of a human immunoglobulin. In some, embodiments a humanized antibody contains the light chain, as well as at least the variable domain of a heavy chain. The antibody also may include the CH1 hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The terms “Kabat numbering,” “Kabat definitions,” and “Kabat labeling” are used interchangeably herein. These terms, which are recognized in the art, refer to a system of numbering amino acid residues, which are more variable (i.e., hypervariable) than other amino acid residues in the heavy and light chain variable regions of an antibody, or an antigen-binding portion thereof (Kabat et al. (1971) Ann. NY Acad, Sci. 190: 382-391; and, Kabat, E. A. et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within binding protein, e.g., antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al. (1987; 1991) Sequences of Proteins of Immunological Interest (National Institutes of Health, Bethesda, Md.) not only provides an unambiguous residue numbering system applicable to any variable region of a binding protein, e.g., an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2 and L3 or H1, H2 and H3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen-binding. The methods used herein may utilize CDRs defined according to any of these systems, although certain embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al. (2002) Crit. Rev. Immunol. 22(3): 183-200; Marchalonis et al. (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen when a binding protein specifically binds to the antigen. In one embodiment, the neutralizing binding protein binds to the cytokine and reduces its biologically activity by at least about 20%, 40%, 60%, 80%, 85% or more.

The term “activity” includes activities such as the binding specificity and affinity of a TVD binding protein for two or more antigens.

The term “epitope” includes any polypeptide determinant that can specifically bind to an immunoglobulin or T-cell receptor. In certain embodiments, epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope is a region of an antigen that is bound by a binding protein, e.g., an antibody. An epitope thus consists of the amino acid residues of a region of an antigen (or fragment thereof) known to bind to the complementary site on the specific binding partner. An antigenic fragment can contain more than one epitope. In certain embodiments, a binding protein, e.g., an antibody, is said to specifically bind an antigen when it recognizes its target antigen in a complex mixture of proteins and/or macromolecules. Binding proteins, e.g., antibodies are said to “bind to the same epitope” if the binding proteins, e.g., antibodies, cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “surface plasmon resonance,” as used herein, refers to an optical phenomenon that allows for the analysis of real-time bio specific interactions by detection of alterations in protein concentrations within a biosensor matrix, for example, using the BIAcore® system (BIAcore International AB, a GE Healthcare company, Uppsala, Sweden and Piscataway, N.J.). For further descriptions, see Jönsson, U. et al. (1993) Ann. Biol. Clin. 51: 19-26; Jönsson, U. et al. (1991) Biotechniques 11: 620-627; Johnsson, B. et al. (1995) J. Mol. Recognit. 8: 125-131; and Johnnson, B. et al. (1991) Anal. Biochem. 198: 268-277.

The term “Kₒₙ,” as used herein, is intended to refer to the on rate constant for association of a binding protein (e.g., an antibody) to the antigen to form the, e.g., antibody/antigen complex as is known in the art. The “Kₒₙ” also is known by the terms “association rate constant,” or “kₐ,” as used interchangeably herein. This value indicating the binding rate of an antibody to its target antigen or the rate of complex formation between an antibody and antigen also is shown by the equation: Antibody (“Ab”)+Antigen (“Ag”)→Ab−Ag.

The term “K_{off},” as used herein, is intended to refer to the off rate constant for dissociation of a binding protein (e.g., an antibody) from the, e.g., antibody/antigen complex as is known in the art. The “K_{off}” also is known by the terms “dissociation rate constant” or “k_{d}” as used interchangeably herein. This value indicates the dissociation rate of an antibody from its target antigen or separation of Ab-Ag complex over time into free antibody and antigen as shown by the equation: Ab+Ag←Ab−Ag.

The terms “equilibrium dissociation constant” or “K_{D},” as used interchangeably herein, refer to the value obtained in a titration measurement at equilibrium, or by dividing the dissociation rate constant (k_{off}) by the association rate constant (kₒₙ). The association rate constant, the dissociation rate constant, and the equilibrium dissociation constant are used to represent the binding affinity of a binding protein, e.g., an antibody, to an antigen. Methods for determining association and dissociation rate constants are well known in the art. Using fluorescence—based techniques offers high sensitivity and the ability to examine samples in physiological buffers at equilibrium. Other experimental approaches and instruments, such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

“Label” and “detectable label” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Thus, the term “labeled binding protein” as used herein, refers to a protein with a label incorporated that provides for the identification of the binding protein. In one embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm); chromogens; fluorescent labels (e.g., FITC, rhodamine, and lanthanide phosphors); enzymatic labels (e.g., horseradish peroxidase, luciferase, and alkaline phosphatase); chemiluminescent markers; biotinyl groups; predetermined polypeptide epitopes recognized by a secondary reporter (e.g., leucine zipper pair sequences, binding sites for secondary antibodies, metal binding domains, and epitope tags); and magnetic agents, such as gadolinium chelates. Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of “detectably labeled” is intended to encompass the latter type of detectable labeling.

The term “conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. In one embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody is a detectably labeled antibody used as the detection antibody.

The terms “crystal” and “crystallized” as used herein, refer to a binding protein (e.g., an antibody), or antigen-binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 201-16, Oxford University Press, New York, N.Y., (1999).

The term “polynucleotide” means a polymeric form of two or more nucleotides, either ribonucleotides or deoxynucleotides or a modified form of either type of nucleotide. The term includes single and double stranded forms of DNA.

The term “isolated polynucleotide” shall mean a polynucleotide (e.g., of genomic, cDNA, or synthetic origin, or some combination thereof) that, by virtue of its origin, the “isolated polynucleotide” is not associated with all or a portion of a polynucleotide with which the “isolated polynucleotide” is found in nature; is operably linked to a polynucleotide that it is not linked to in nature; or does not occur in nature as part of a larger sequence.

The term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest.

The term “expression control sequence” as used herein refers to polynucleotide sequences, which are necessary to effect the expression and processing of coding sequences to which they are ligated. Expression control sequences include appropriate transcription initiation, termination, promoter and enhancer sequences; efficient RNA processing signals, such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs, depending upon the host organism; in prokaryotes, such control sequences generally include a promoter, a ribosomal binding site, and a transcription termination sequence; in eukaryotes, generally, such control sequences include a promoter and a transcription termination sequence. The term “control sequences” is intended to include components whose presence is essential for expression and processing, and can also include additional components whose presence is advantageous, for example, leader sequences and fusion partner sequences.

“Transformation” refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication, either as an autonomously replicating plasmid or as part of the host chromosome. They also include cells, which transiently express the inserted DNA or RNA for limited periods of time.

The term “recombinant host cell” (or simply “host cell”) is intended to refer to a cell into which exogenous DNA has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein. In one embodiment, host cells include prokaryotic and eukaryotic cells selected from any of the Kingdoms of life. In another embodiment, eukaryotic cells include protist, fungal, plant and animal cells. In another embodiment, host cells include, but are not limited to, the prokaryotic cell line E. coli; mammalian cell lines CHO, HEK 293, COS, NS0, SP2 and PER.C6; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. (1989) Molecular Cloning: A Laboratory Manual (2d ed., Cold Spring Harbor Laboratory Press, Cold Spring Harbor, N.Y.).

“Transgenic organism,” as known in the art, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of a cytokine). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator” is a compound capable of changing or altering an activity or function of a molecule of interest (e.g., the biological activity of a cytokine). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in PCT Publication No. WO 01/83525.

The term “agonist” refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, polypeptides, nucleic acids, carbohydrates, and any other molecules that bind to the antigen.

The term “antagonist” or “inhibitor” refers to a modulator that, when contacted with a molecule of interest, causes a decrease in the magnitude of a certain activity or function of the molecule compared to the magnitude of the activity or function observed in the absence of the antagonist. Particular antagonists of interest include those that block or modulate the biological or immunological activity of the antigen. Antagonists and inhibitors of antigens may include, but are not limited to, proteins, nucleic acids, carbohydrates, and any other molecules, which bind to the antigen.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent).

“Patient” and “subject” may be used interchangeably herein to refer to an animal, such as a mammal, including a primate (for example, a human, a monkey, and a chimpanzee), a non-primate (for example, a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. Preferably, the patient or subject is a human, such as a human being treated or assessed for a disease, disorder or condition, a human at risk for a disease, disorder or condition, a human having a disease, disorder or condition, and/or human being treated for a disease, disorder or condition.

The term “sample,” as used herein, is used in its broadest sense. A “biological sample,” as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, (e.g., whole blood), plasma, serum, urine, amniotic fluid, synovial fluid, endothelial cells, leukocytes, monocytes, other cells, organs, tissues, bone marrow, lymph nodes and spleen.

“Component,” “components,” and “at least one component,” refer generally to a capture binding protein, e.g., antibody, a detection or conjugate binding protein, e.g., antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Thus, in the context of the present disclosure, “at least one component,” “component,” and “components” can include a polypeptide or other analyte as above, such as a composition comprising an analyte such as polypeptide, which is optionally immobilized on a solid support, such as by binding to an anti-analyte (e.g., anti-polypeptide) antibody. Some components can be in solution or lyophilized for reconstitution for use in an assay.

“Control” refers to a composition known to not contain analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, it is well-known that cutoff values may vary depending on the nature of the assay, such as an immunoassay (e.g., antibodies employed, etc.). It further is well within the ordinary skill of one in the art to adapt the disclosure herein for other assays, e.g., immunoassays, to obtain immunoassay-specific cutoff values for those other immunoassays based on this disclosure. Whereas the precise value of the predetermined cutoff/level may vary between assays, correlations as described herein (if any) should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., polypeptide of interest) may entail release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay.

“Quality control reagents” in the context of assays, e.g., immunoassays, and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Risk” refers to the possibility or probability of a particular event occurring either presently or at some point in the future. “Risk stratification” refers to an array of known clinical risk factors that allows physicians to classify patients into a low, moderate, high or highest risk of developing a particular disease, disorder or condition.

“Specific” and “specificity” in the context of an interaction between members of a specific binding pair (e.g., an antigen (or fragment thereof) and a binding protein, e.g., an antibody, (or antigenically reactive fragment thereof)) refer to the selective reactivity of the interaction. The phrase “specifically binds to” and analogous phrases refer to the ability of, e.g., antibodies (or antigenically reactive fragments thereof) to bind specifically to analyte (or a fragment thereof) and not bind specifically to other entities. Specific binding is understood as a preference for binding a certain antigen, epitope, receptor ligand, or binding partner with at least a 10³, 10⁴, 10⁵, 10⁶, 10⁷, 10⁸, 10⁹-fold preference over a control non-specific antigen, epitope, receptor ligand, or binding partner. Methods of selecting appropriate non-specific controls is within the ability of those of skill in the art.

“Specific binding partner” is a member of a specific binding pair. A specific binding pair comprises two different molecules, which specifically bind to each other through chemical or physical means. Therefore, in addition to, e.g., antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced.

“Variant” as used herein means a polypeptide that differs from a given polypeptide (e.g., TNFα, PGE2, IL-12, IL-13, IL-18, HMGB1, VEGF, RAGE, NGF, IL-1α, IL-1β, E-selectin, L-selectin, glycoprotein (GP) IIb/IIIa, thrombomodulin, thrombin, TREM, PAI-I, αVβ3, uPA, Her2, IGF1R, EGFR, CD3, Fc gamma receptor, NKG2D, substance P, CGRP, Protein C, Factor VII, Factor IX, plasminogen activator, Factor V, Factor VIIa, Factor Factor X, Factor XII, Factor XIII, C1q, C1r C1s, C4a, C4b, C2a, C2b, C, C3a and C3b polypeptide or anti-polypeptide antibody) in amino acid sequence by the addition (e.g., insertion), deletion, or conservative substitution of amino acids, but that retains the biological activity of the given polypeptide (e.g., a variant IL-18 can compete with anti-IL-18 antibody for binding to IL-18). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). The hydropathic index of an amino acid is based on a consideration of its hydrophobicity and charge. It is known in the art that amino acids of similar hydropathic indexes can be substituted and still and the polypeptide will retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to describe a polypeptide or fragment thereof that has been differentially processed, such as by proteolysis, phosphorylation, or other post-translational modification, yet retains its biological activity or antigen reactivity, e.g., the ability to bind to IL-18. Use of “variant” herein is intended to encompass fragments of a variant unless otherwise contradicted by context.

### I. Generation of TVD Binding Proteins

The present invention pertains to Tri-Variable Domain (TVD) binding proteins comprising three or six antigen-binding sites that can bind one or more targets and methods of making the same. FIG. 1 provides a schematic of the structure of an exemplary TVD binding protein of the invention.

In general, the binding proteins of the invention comprise a polypeptide chain, wherein the polypeptide chain comprises VD1-(X1)n-VD2-(X2)n-VD3-C-(X3)n, wherein VD1 is a first variable domain, VD2 is a second variable domain, VD3 is a third variable domain, C is a constant domain, X1 is a first linker, X2 is a second linker, X3 is an Fc region and n is 0 or 1.

In exemplary embodiments, each of “VD1”, “VD2”, and “VD3” is independently a heavy chain variable domain or a light chain variable domain. As used herein, “VD” alone is to be understood to be either a heavy chain antigen biding domain or a light chain antigen binding unless otherwise clear from context.

In one embodiment, VD1 is a heavy chain variable domain. In another embodiment, VD2 is a heavy chain variable domain. In yet another embodiment, VD3 is a heavy chain variable domain. In one embodiment, VD1 is a light chain variable domain. In another embodiment, VD2 is a light chain variable domain. In yet another embodiment, VD3 is a light chain variable domain.

In exemplary embodiments, each of “X1” and “X2” is a linker and each of “n” is independently 0 or 1.

In one embodiment, (X1)n is 0. In another embodiment, (X1)n is 1. In yet another embodiment, (X2)n is 0. In a further embodiment, (X2)n is 1. In another embodiment, (X2)n is not CH1 and may be either 0 or 1.

In exemplary embodiments, “C” is heavy chain or light chain constant domain. It is understood that, as used herein, “C” alone can be understood to be either a heavy chain constant domain or a light chain constant unless otherwise clear from context.

In one embodiment, C is a light chain constant domain. In another embodiment, C is a heavy chain constant domain. In yet another embodiment, C is a heavy chain CH1 domain. In a further embodiment, C is a heavy chain CH2 domain. In another embodiment, C is a heavy chain CH3 domain. In yet another embodiment, C is a heavy chain CH4 domain. In one embodiment, C is not a heavy chain CH1 domain. In another embodiment, C is not a heavy chain CH2 domain. In yet another embodiment, C is not a heavy chain CH3 domain. In one embodiment, C is not a heavy chain CH4 domain.

In exemplary embodiments, “(X3)n” is an Fc region and “n” is 0 or 1. In one embodiment, (X3)n is 0. In another embodiment, (X3)n is 1.

TVD binding proteins comprising two heavy chain TVD polypeptides and two light chain TVD polypeptides and are referred to herein as “TVD-Ig proteins” or “TVD-Ig binding proteins”. Each half of a TVD-Ig protein comprises a heavy chain TVD polypeptide, and a light chain TVD polypeptide, and three antigen-binding sites. Each binding site comprises a heavy chain variable domain and a light chain variable domain with a total of six CDRs involved in antigen binding per antigen-binding site.

In one embodiment of the invention, a TVD binding protein comprises a polypeptide chain comprising VD1-(X1)n-VD2-(X2)n-VD3-C-(X3)n wherein VD1 is a first heavy chain variable domain, VD2 is a second heavy chain variable domain, VD3 is a third heavy chain variable domain, C is a heavy chain constant domain, X1 is a first linker, X2 is a second linker, X3 is an Fc region, and n is 0 or 1, wherein the binding protein is capable of binding one to three target antigens.

In another embodiment of the invention, a TVD binding protein comprises a polypeptide chain comprising VD1-(X1)n-VD2-(X2)n-VD3-C-(X3)n, wherein, VD1 is a first light heavy chain variable domain, VD2 is a second light heavy chain variable domain, VD3 is a third light chain variable domain, C is a light chain constant domain, X1 is a first linker, X1 is a second linker, X3 does not comprise an Fc region, and n is 0 or 1; wherein the binding protein is capable of binding one to three target antigens.

In one embodiment, a binding protein of the invention comprises a first and a second polypeptide chain. In this embodiment of the invention, the first polypeptide chain comprises a first VD1-(X1)n-VD2-(X2)n-VD3-C-(X3)n, wherein VD1 is a first heavy chain variable domain, VD2 is a second heavy chain variable domain, VD3 is a third heavy chain variable domain, C is a heavy chain constant domain, X1 is a first linker, X2 is a second linker, and X3 is an Fc region. The second polypeptide chain comprises a second VD1-(X1)n-VD2-(X2)n-VD3-C-(X3)n, wherein VD1 is a first light chain variable domain, VD2 is a second light chain variable domain, VD3 is a third light chain variable domain, C is a light chain constant domain, X1 is a first linker, X2 is a second linker, and X3 does not comprise an Fc region and n is 0 or 1, wherein the binding protein is capable of binding one to six target antigens.

In another embodiment, a binding protein of the invention comprises four polypeptide chains. In this embodiment of the invention, each of the first and third polypeptide chains independently comprise VD1-(X1)n-VD2-(X2)n-VD3-C-(X3)n, wherein VD1 is a first heavy chain variable domain, VD2 is a second heavy chain variable domain, VD3 is a third heavy chain variable domain, C is a heavy chain constant domain, X1 is a first linker, X2 is a second linker, X3 is an Fc region, and n is 0 or 1. Each of the second and fourth polypeptide chains independently comprise VD1-(X1)n-VD2-(X2)n-VD3-C-(X3)n, wherein D1 is a first light heavy chain variable domain, VD2 is a second light heavy chain variable domain, VD3 is a third light chain variable domain, C is a light chain constant domain, X1 is a first linker, X1 is a second linker, X3 does not comprise an Fc region, and n is 0 or 1; wherein the binding protein is capable of binding one to six target antigens.

The variable domains for use in the binding proteins of the present invention may be derived from or obtained from any suitable or desired binding protein, such as a polypeptide encoding a receptor of interest and/or a parent binding protein, e.g., antibody that binds a target antigen of interest. Parent binding proteins, e.g., antibodies may be any suitable binding proteins, e.g., antibodies, including, but not limited to, chimeric, polyclonal, and monoclonal antibodies that bind target antigen(s) of interest. These antibodies may be naturally occurring or may be generated by recombinant technology.

The variable domains for use in the binding proteins of the invention may be obtained from the same or different parent binding proteins, e.g., parent antibodies. In one embodiment, two or more of VD1, VD2, and VD3 are independently obtained from a same parent binding protein, e.g., antibody, or antigen-binding portion thereof. In another embodiment, each of VD1, VD2, and VD3 are independently obtained from a same parent binding protein, e.g., antibody, or antigen-binding portion thereof. In one embodiment, two or more of VD1, VD2, and VD3 are independently obtained from a different parent binding protein, e.g., antibody, or antigen-binding portion thereof. In another embodiment, each of VD1, VD2, and VD3 are independently obtained from a different parent binding protein, e.g., antibody, or antigen-binding portion thereof.

The same or different parent binding proteins, e.g., parent antibodies, or antigen-binding portions thereof, may be independently selected from the group consisting of a human antibody, a CDR grafted antibody, and a humanized antibody. In another embodiment, the same or different parent antibody, or antigen-binding portion thereof, are independently selected from the group consisting of a Fab fragment; a F(ab′)₂ fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment; an isolated complementarity determining region (CDR); a single chain antibody; a Rab; and a diabody.

In embodiments of the invention in which different parent binding proteins, e.g., antibodies, are used to derive a variable domain of interest, the different parent binding proteins, e.g., antibodies, or antigen-binding fragments thereof, may bind the same epitope or different epitopes on a target antigen. In embodiments of the invention in which different parent binding proteins, e.g., antibodies are used to derive a variable domain of interest, the different parent binding proteins, e.g., antibodies, or antigen-binding fragments thereof, may bind their respective target antigens with a different potency and/or a different affinity.

The parent binding proteins, e.g., parent antibodies, for use in the binding proteins of the present invention can be generated using various techniques. The present disclosure provides expression vectors, host cells, and methods of generating the binding proteins.

### A. Generation of Parent Binding Proteins

### A1. Monoclonal Antibodies

The variable domains of the TVD binding proteins can be obtained from parent binding proteins, e.g., antibodies, including polyclonal and monoclonal antibodies that can bind antigens of interest. These antibodies may be naturally occurring or may be generated by recombinant technology.

For example, monoclonal antibodies for use on the binding protein of the invention may be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al. (1988) Antibodies: A Laboratory Manual, (Cold Spring Harbor Laboratory Press, 2nd ed.); Hammerling, et al. (1981) in: Monoclonal Antibodies and T-Cell Hybridomas 563-681 (Elsevier, N.Y.). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Hybridomas are selected, cloned and further screened for desirable characteristics, including robust hybridoma growth, high antibody production and desirable antibody characteristics, as discussed in Example 1 below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art. In a particular embodiment, the hybridomas are mouse hybridomas. In another embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an antibody that can bind a specific antigen. Recombinant monoclonal antibodies are also generated from single, isolated lymphocytes using a procedure referred to in the art as the selected lymphocyte antibody method (SLAM), as described in U.S. Pat. No. 5,627,052; PCT Publication No. WO 92/02551, and Babcock, J. S. et al. (1996) Proc. Natl. Acad. Sci. USA 93: 7843-7848. In this method, single cells secreting antibodies of interest, e.g., lymphocytes derived from an immunized animal, are identified, and heavy- and light-chain variable region cDNAs are rescued from the cells by reverse transcriptase-PCR. These variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to the antigen of interest. The amplified immunoglobulin sequences further can be manipulated in vitro, such as by in vitro affinity maturation methods, such as those described in PCT Publication Nos. WO 97/29131 and WO 00/56772.

Monoclonal antibodies are also produced by immunizing a non-human animal comprising some, or all, of the human immunoglobulin locus with an antigen of interest. In one embodiment, the non-human animal is a XENOMOUSE transgenic mouse, an engineered mouse strain that comprises large fragments of the human immunoglobulin loci and is deficient in mouse antibody production. See, e.g., Green et al. (1994) Nature Genet. 7: 13-21 and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/037504. The XENOMOUSE transgenic mouse produces an adult-like human repertoire of fully human antibodies, and generates antigen-specific human monoclonal antibodies. The XENOMOUSE transgenic mouse contains approximately 80% of the human antibody repertoire through introduction of megabase sized, germline configuration YAC fragments of the human heavy chain loci and x light chain loci. See Mendez et al. (1997) Nature Genet. 15: 146-156; Green and Jakobovits (1998) J. Exp. Med. 188: 483-495.

In vitro methods also can be used to make the parent antibodies, wherein an antibody library is screened to identify an antibody having the desired binding specificity. Methods for such screening of recombinant antibody libraries are well known in the art and include methods described in, for example, Ladner et al., U.S. Pat. No. 5,223,409; PCT Publication Nos. WO 92/18619; WO 91/17271; WO 92/20791; WO 92/15679; WO 93/01288; WO 92/01047; WO 92/09690 and WO 97/29131; Fuchs et al. (1991) Bio/Technology 9: 1370-1372; Hay et al. (1992) Hum. Antibod. Hybridomas 3: 81-85; Huse et al. (1989) Science 246: 1275-1281; McCafferty et al. (1990) Nature 348: 552-554; Griffiths et al. (1993) EMBO J. 12: 725-734; Hawkins et al. (1992) J. Mol. Biol. 226: 889-896; Clackson et al. (1991) Nature 352: 624-628; Gram et al. (1992) Proc. Natl. Acad. Sci. USA 89: 3576-3580; Garrad et al. (1991) Bio/Technology 9: 1373-1377; Hoogenboom et al. (1991) Nucl. Acid Res. 19: 4133-4137; and Barbas et al. (1991) Proc. Natl. Acad. Sci. USA 88: 7978-7982, and U.S. Patent Publication No. 2003/0186374.

A2. scFv and In Vitro Generated Antibodies

Parent binding proteins of the present disclosure can also be generated using various phage display methods known in the art. In phage display methods, functional antibody domains are displayed on the surface of phage particles that carry the polynucleotide sequences encoding them. In a particular, such phage can be utilized to display antigen-binding domains expressed from a repertoire or combinatorial antibody library (e.g., human or murine). Phage expressing an antigen-binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present disclosure include those disclosed in Brinkman et al. (1995) J. Immunol. Methods 182: 41-50; Ames et al. (1995) J. Immunol. Methods 184: 177-186; Kettleborough et al. (1994) Eur. J. Immunol. 24: 952-958; Persic et al. (1997) Gene 187: 9-18; Burton et al. (1994) Advances in Immunol. 57: 191-280; PCT Application No. PCT/GB91/01134; PCT Publication Nos. WO 90/02809; WO 91/10737; WO 92/01047; WO 92/18619; WO 93/11236; WO 95/15982; and WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the herein references, after phage selection, the antibody coding regions from the phage can be isolated and used to generate whole antibodies including human antibodies or any other desired antigen-binding fragment, and expressed in any desired host, including mammalian cells, insect cells, plant cells, yeast, and bacteria, e.g., as described in detail below. For example, techniques to produce recombinantly Fab, Fab′ and F(ab′)2 fragments can also be employed using methods known in the art such as those disclosed in PCT Publication No. WO 92/22324; Mullinax et al. (1992) BioTechniques 12(6): 864-869; Sawai et al. (1995) AJRI 34: 26-34; and Better et al. (1988) Science 240: 1041-1043. Examples of techniques, which can be used to produce single-chain Fvs and antibodies, include those described in U.S. Pat. Nos. 4,946,778 and 5,258,498; Huston et al. (1991), Methods Enzymol. 203:46-88; Shu et al. (1993) Proc. Natl. Acad. Sci. USA 90: 7995-7999; and Skerra et al. (1988) Science 240: 1038-1040.

Alternative to screening of recombinant antibody libraries by phage display, other methodologies known in the art for screening large combinatorial libraries can be applied to the identification of parent antibodies. One type of alternative expression system is one in which the recombinant antibody library is expressed as RNA-protein fusions, as described in PCT Publication No. WO 98/31700, and in Roberts, R. W. and Szostak, J. W. (1997) Proc. Natl. Acad. Sci. USA 94: 12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described herein (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described herein.

In another approach the parent binding proteins can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the parent antibodies include those disclosed in U.S. Pat. No. 6,699,658.

### A3. Humanized and Engineered Antibodies

Parent binding proteins of the present disclosure can also be modified to generate CDR grafted and humanized parent antibodies. CDR-grafted parent antibodies comprise heavy and light chain variable region sequences from a human antibody wherein one or more of the CDR regions of V_{H} and/or V_{L} are replaced with CDR sequences of murine antibodies that can bind antigen of interest. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, In one embodiment, the human variable framework that is chosen to replace the murine variable framework apart from the CDRs have at least a 65% sequence identity with the murine antibody variable region framework. In one embodiment, the human and murine variable regions apart from the CDRs have at least 70% sequence identify. In a particular embodiment, that the human and murine variable regions apart from the CDRs have at least 75% sequence identity. In another embodiment, the human and murine variable regions apart from the CDRs have at least 80% sequence identity. Methods for producing such antibodies are known in the art (see EP 239,400; PCT Publication No. WO 91/09967; and U.S. Pat. Nos. 5,225,539; 5,530,101; and 5,585,089), veneering or resurfacing (EP 592,106; EP 519,596; Padlan (1991) Mol. Immunol. 28(4/5): 489-498; Studnicka et al. (1994) Prot. Engineer. 7(6): 805-814; and Roguska et al. (1994) Proc. Acad. Sci. USA 91: 969-973), chain shuffling (U.S. Pat. No. 5,565,352), and anti-idiotypic antibodies.

Humanized antibodies are antibody molecules from non-human species that bind the desired antigen and have one or more CDRs from the non-human species and framework regions from a human immunoglobulin molecule. Known human Ig sequences are disclosed, e.g., www.ncbi.nlm.nih.gov/entrez-/query.fcgi; www.atcc.org/phage/hdb.html; www.sciquest.com; www.abcam.com; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kuby05.htm; www.library.thinkquest.org/12429/Immune/Antibody.html; www.hhmi.org/grants/lectures/1996/vlab; www.path.cam.ac.uk/.about.mrc7/m-ikeimages.html; www.antibodyresource.com; mcb.harvard.edu/BioLinks/Immuno-logy.html.; www.immunologylink coin; pathbox.wustl.edu/.about.hcenter/index.-html; www.biotech.ufl.edu/.about.hcl; www.pebio.com/pa/340913/340913.html-; www.nal.usda.gov/awic/pubs/antibody; www.m.ehime-u.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.uni-marburg.de/.about.rek/AEP-Start.html; baserv.uci.kun.nl/.about.jraats/linksl.html; www.recab.uni-hd.de/immuno.bme.nwu.edu/; www.mrc-cpe.cam.ac.uk/imt-doc/pu-blic/INTRO.html; www.ibt.unam.mx/vir/V_mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOsem-inar/Slide01.html; www.cryst.bbk.ac.uld.aboutubcg07s/; www.nimr.mrc.ac.uk/CC/ccaewg/ccaewg.htm; www.path.cam.ac.uk/.about.mrc7/h-umanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Web-pages/Pept/spottech.html; www.jerini.de/fr roducts.htm; www.patents.ibm.com/ibm.html; and Kabat et al., Sequences of Proteins of Immunological Interest, U.S. Dept. Health (1983). Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, e.g., improve, antigen-binding. These framework substitutions are identified by methods well known in the art, e.g., by modeling of the interactions of the CDR and framework residues to identify framework residues important for antigen-binding and sequence comparison to identify unusual framework residues at particular positions (See, e.g., U.S. Pat. No. 5,585,089; Riechmann et al. (1988) Nature 332: 323). Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen-binding. Antibodies can be humanized using a variety of techniques known in the art, such as, but not limited to, those described in Jones et al. (1986) Nature 321: 522; Verhoeyen et al. (1988) Science 239: 1534; Sims et al. (1993) J. Immunol. 151: 2296; Chothia and Lesk (1987) J. Mol. Biol. 196: 901; Carter et al. (1992) Proc. Natl. Acad. Sci. USA 89: 4285; Presta et al. (1993) J. Immunol. 151: 2623; Padlan (1991) Mol. Immunol. 28(4/5): 489-498; Studnicka et al. (1994) Prot. Engineer. 7(6): 805-814; Roguska et al., (1994) Proc. Natl. Acad. Sci. USA 91: 969-973; PCT Publication No. WO 91/09967: 0598/16280; 0596/18978; 0591/09630; 0591/05939; 0594/01234; GB89/01334; GB91/01134; GB92/01755; WO90/14443; WO90/14424; and WO90/14430; European Patent Publication Nos. EP 229246; EP 592,106; EP 519,596; and EP 239,400; and U.S. Pat. Nos. 5,565,332; 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

### A4. Exemplary Single Variable Domains

Exemplary single variable domains for use in the TVD binding proteins of the instant invention include the following variable domain sequences.

TABLE 1
List of Amino Acid Sequences of VH and VL
regions of Binding Proteins for Generating
TVD-Binding Proteins

| | | |
|---|---|---|
| 1D4.1 VH | SEQ ID NO: 46 | EVTLRESGPALVKPTQTLTL |
| | | TCTFSGFSLSKSVMGVSWIR |
| | | QPPGKALEWLAHIYWDDDKY |
| | | YNPSLKSRLTISKDTSKNQV |
| | | VLTMTNMDPVDTATYYCARR |
| | | GIRSAMDYWGQGTTVTVSS |
| mAB 2.5 VH | SEQ ID NO: 47 | EVQLVQSGTEVKKPGESLKI |
| | | SCKGSGYTVTSYWIGWVRQM |
| | | PGKGLEWMGFIYPGDSETRY |
| | | SPTFQGQVTISADKSFNTAF |
| | | LQWSSLKASDTAMYYCARVG |
| | | SGWYPYTFDIWGQGTMVTVS |
| | | S |
| 2B5 VH | SEQ ID NO: 48 | EVQLVQSGAEVKKPGASVKV |
| | | SCKASGYTFTKYWLGWVRQA |
| | | PGQGLEWMGDIYPGYDYTHY |
| | | NEKFKDRVTLTTDTSTSTAY |
| | | MELRSLRSDDTAVYYCARSD |
| | | GSSTYWGQGTLVTVSS |
| 1D4.1 VL | SEQ ID NO: 51 | DIVMTQSPDSLAVSLGERAT |
| | | INCKASQSVSNDVAWYQQKP |
| | | GQPPKLLIYYASNRYTGVPD |
| | | RFSGSGSGTDFTLTISSLQA |
| | | EDVAVYYCQQDYNSPWTFGG |
| | | GTKVEIKR |
| mAB 2.5 VL | SEQ ID NO: 52 | EIVMTQSPATLSVSPGERAT |
| | | LSCRASESISSNLAWYQQKP |
| | | GQAPRLFIYTASTRATDIPA |
| | | RFSGSGSGTEFTLTISSLQS |
| | | EDFAVYYCQQYNNWPSITFG |
| | | QGTRLEIKR |
| 2B5 | SEQ ID NO: 53 | DVLMTQTPLSLPVTPGEPAS |
| | | ISCTSSQNIVHSNGNTYLEW |
| | | YLQKPGQSPQLLIYKVSNRF |
| | | SGVPDRFSGSGSGTDFTLKI |
| | | SRVEAEDVGVYYCFQVSHVP |
| | | YTFGGGTKVEIKR |
| D2E7 VH | SEQ ID NO: 70 | EVQLVESGGGLVQPGRSLRL |
| | | SCAASGFTFDDYAMHWVRQA |
| | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | S |
| 13C5.5L3F VH | SEQ ID NO: 71 | EVTLRESGPGLVKPTQTLTL |
| | | TCTLYGFSLSTSDMGVDWIR |
| | | QPPGKGLEWLAHIWWDDVKR |
| | | YNPALKSRLTISKDTSKNQV |
| | | VLKLTSVDPVDTATYYCART |
| | | VSSGYIYYAMDYWGQGTLVT |
| | | VSS |
| D2E7 VL | SEQ ID NO: 73 | DIQMTQSPSSLSASVGDRVT |
| | | ITCRASQGIRNYLAWYQQKP |
| | | GKAPKLLIYAASTLQSGVPS |
| | | RFSGSGSGTDFTLTISSLQP |
| | | EDVATYYCQRYNRAPYTFGQ |
| | | GTKVEIKR |
| 13C5.5L3F VL | SEQ ID NO: 74 | DIQMTQSPSSLSASVGDRVT |
| | | ISCRASQDIRNYLNWYQQKP |
| | | GKAPKLLIFYTSKLHSGVPS |
| | | RFSGSGSGTDYTLTISSLQP |
| | | EDIATYYCQQGLTPPLTFGG |
| | | GTKVEIKR |
| VH HIV | SEQ ID NO: 75 | QVQLQQSGAELMKPGASVKI |
| (seq. 1) | | SCKASGYTFTSYWIEWIKQR |
| | | PGHGLEWIGEILPGTGSLNN |
| | | NEKFRDKATFTADTSSNTAY |
| | | MQLSSLTSEDSAVYYCARGY |
| | | RYDGWFAYWGQGTLVTVSA |
| VL HIV | SEQ ID NO: 76 | DIQMTQSPASLSASVGETVT |
| (seq. 1) | | ITCRTSENIYSYLAWYQQKP |
| | | GKSPHLLVYNTKTLAEGVPS |
| | | RFSGSGSGTQFSLKINSLQP |
| | | EDFGSYYCQHHYDSPLTFGS |
| | | GTKLELKR |
| VH NGAL | SEQ ID NO: 77 | EVQLVESGGGLVQPGGSLKL |
| (seq. 1) | | SCAASGFTFNNYYMSW |
| | | VRQTPERRLEWVAYISSSGG |
| | | STYYSDSVRGRFTISRDTAR |
| | | NTLYLQMTSLKSEDTAMYYC |
| | | ARHFGDYSYFDYWGQGTTLT |
| | | VSS |
| VL NGAL | SEQ ID NO: 78 | DIQMTQSPASLSASVGETVT |
| (seq. 1) | | ITCRASENFYSYLAWYQQKQ |
| | | GKSPQLLVYNAKTLAEGVPS |
| | | RFSGSGSGTQFSLKINSLQP |
| | | EDFGTYYCQHHYDIPLTFGA |
| | | GTKLELKR |
| VH NGAL | SEQ ID NO: 79 | KIQLVQSGPELKKPGETVKI |
| (seq. 2) | | SCKASGYTFTNYGMNWVKQA |
| | | PGKGLKWMGWININTGEPTY |
| | | AEEFKGRFAFSLETSATTAF |
| | | LQINNLKNEDTATYLCARDS |
| | | YSGGFDYWGQGTIVTVSS |
| VL NGAL | SEQ ID NO: 80 | DIVMTQSPSSLSVSAGEKVT |
| (seq. 2) | | LSCKSSQSLLISGDQKNYLA |
| | | WYQQKPGQPPKLLIYGASTR |
| | | DSGVPDRFTGSGSGADFTLT |
| | | ISSVQAEDLAVYYCQNDHSF |
| | | PPTFGAGTKLELKR |
| VH HIV | SEQ ID NO: 81 | QIQLVQSGPELKKPGETVKI |
| (seq. 2) | | SCKASGYTFTDYSMHWVKQA |
| | | PGKGLKWMGWIHTETGEPRY |
| | | VDDFKGRFAFSLETSASTAY |
| | | LQINNLKNEDTATYFCARDS |
| | | YYFGSSYYFDYWGQGTTLTV |
| | | SS |
| VL HIV | SEQ ID NO: 82 | DTVMTQSHKFMSTSVGDRVS |
| (seq. 2) | | ITCKASQDVSSAVAWYQQKP |
| | | GQSPKLLIYSASYRYTGVPD |
| | | RFTGSGSGMDFTFTISSVQA |
| | | EDLAVYYCQQHYSTPLTFGA |
| | | GTKLELER |
| VH HIV | SEQ ID NO: 83 | EVQLQQSGPELVKPGASMKI |
| (seq. 3) | | SCKASDYSFTAYTIHWMKQS |
| | | HGKNLEWIGLINPYNGGTSY |
| | | NQKFQGRATLTVDKSSSIAY |
| | | MELLSLTSEDSAVYYCARRG |
| | | YDREGHYYAMDYWGQGTSVT |
| | | VSS |
| VL HIV | SEQ ID NO: 84 | DIQMTQSPASLAASVGETVT |
| (seq. 3) | | ITCRASENIYTFLAWYQQKQ |
| | | GKSPQLLVYTTKTLAEGVPS |
| | | RFSGSGSGTQFSLKIKSLQP |
| | | EDFGSYYCQHHYGLPLTFGA |
| | | GTKLELKR |
| VH HIV | SEQ ID NO: 85 | EVQLQQSGPELVQPGASMKI |
| (seq. 4) | | SCKASGYSFTDYTMNWVKQS |
| | | HGKNLEWIGLINPYNGGSRY |
| | | NQKFMAKATLTVDKSSNTAY |
| | | MELLSVTSEDSAVYYCARDA |
| | | GYFGSGFYFDYWGQGTTLTV |
| | | SS |
| VL HIV | SEQ ID NO: 86 | DIVMTQSHKFMSTSVGDRVS |
| (seq. 4) | | ITCKASQDVSTAVAWYQQKP |
| | | GQSPKLLIYSASYRSTGVPD |
| | | RFTGSGSGTDFTFTISSVQA |
| | | EDLAVYYCQQHYSTPTFGAG |
| | | TKLELKR |
| VH IL-18 | SEQ ID NO: 87 | QVQLQQPGSELVRPGASVKL |
| | | SCKASGYTFTSYWMHWVKQR |
| | | PGQGLEWIGNIYPGTVNTNY |
| | | DEKFKNKATLTVDTSSSTAY |
| | | MLLSSLTSEDSAVYYCTRDY |
| | | YGGGLNYWGQGTTLTVSS |
| VL IL-18 | SEQ ID NO: 88 | SIVMTQTPKFLLVSAGDRVT |
| | | ITCKASQSVSNDVAWFQQKP |
| | | GQSPKLLIYYASNRYAGVPD |
| | | RFTGSGFGTDFTFTISTVQA |
| | | EDLAVYFCHQDYSSPRTFGG |
| | | GTKLEIKR |
| VH BNP | SEQ ID NO: 89 | QIQLVQSGPELRKPGETVKI |
| (seq. 1) | | SCKGSGYTFTHYGINWVKQT |
| | | PRKDLKWMGWINTHTGEAYY |
| | | ADDFKGRFAFSLETSANTAY |
| | | LQINNLNNGDMGTYFCTRSH |
| | | RFGLDYWGQGTSVTVSS |
| VL BNP | SEQ ID NO: 90 | DNVLTQSPPSLAVSLGQRAT |
| (seq. 1) | | ISCKANWPVDYNGDSYLNWY |
| | | QQKPGQPPKFLIYAASNLES |
| | | GIPARFSGSGSGTDFNLNIH |
| | | PVEEEDAATYYCQQSNEDPF |
| | | TFGSGTKLEIKR |
| VH BNP | SEQ ID NO: 91 | QVQLQQPGAELVRPGASVKL |
| (seq. 2) | | SCKASGYTFTSYWMNWVKQR |
| | | PEQGLEWIGRIDPYDSETHY |
| | | NQKFKDKAILTVDKSSSTAF |
| | | VQLTSLTSEDSAVYYCVSDG |
| | | YWGAGTTVTVSS |
| VL BNP | SEQ ID NO: 92 | DVVMTQTPLTLSVTTGQPAS |
| (seq. 2) | | ISCKSSQSLLDSDGKTYLNW |
| | | LFQRPGESPKLLIYVVSKLE |
| | | SGVPDRFTGSGSGTDFTLKI |
| | | SRVEAEDLGVYYCLQATHFP |
| | | WTFGGGTKLEIKR |
| VH BNP | SEQ ID NO: 93 | QVQLQQPGAELVRPGASVKL |
| (seq. 4) | | SCKASGYTFTSYWMNWVKQR |
| | | PEQGLEWIGRIDPYDSETHY |
| | | NQKFKDKAILTVDKSSSTAF |
| | | VQLTSLTSEDSAVYYCVSDG |
| | | YWGAGTTVTVSS |
| VL BNP | SEQ ID NO: 94 | DVVMTQTPLTLSVTTGQPAS |
| (seq. 4) | | ISCKSSQSLLDSDGKTYLNW |
| | | LFQRPGESPKLLIYVTDILE |
| | | SGVPDRFTGSGSGTDFTLKI |
| | | SRVEAEDLGVYYCLQATHFP |
| | | WTFGGGTKLEIKR |
| VH TnI | SEQ ID NO: 95 | EVQLQQSGPDLVKPGASVRI |
| | | SCKASGYTFTDYNLHWVKQS |
| | | HGKSLEWIGYIYPYNGITGY |
| | | NQKFKSKATLTVDSSSNTAY |
| | | MDLRSLTSEDSAVYFCARDA |
| | | YDYDYLTDWGQGTLVTVSA |
| VL TnI | SEQ ID NO: 96 | DILLTQSPVILSVSPGERVS |
| | | FSCRTSKNVGTNIHWYQQRT |
| | | NGSPRLLIKYASERLPGIPS |
| | | RFSGSGSGTDFTLSINSVES |
| | | EDIADYYCQQSNNWPYTFGG |
| | | GTKLEIKR |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| IGF1R VH | SEQ ID NO: 167 | EVQLLESGGGLVQPGGSLRL |
| | | SCTASGFTFSSYAMNWVRQA |
| | | PGKGLEWVSAISGSGGTTFY |
| | | ADSVKGRFTISRDNSRTTLY |
| | | LQMNSLRAEDTAVYYCAKDL |
| | | GWSDSYYYYYGMDVWGQGTT |
| | | VTVSS |
| IGF1R VL | SEQ ID NO: 168 | DIQMTQFPSSLSASVGDRVT |
| | | ITCRASQGIRNDLGWYQQKP |
| | | GKAPKRLIYAASRLHRGVPS |
| | | RFSGSGSGTEFTLTISSLQP |
| | | EDFATYYCLQHNSYPCSFGQ |
| | | GTKLEIKR |
| CD20 VH | SEQ ID NO: 169 | QVQLQQPGAELVKPGASVKM |
| | | SCKASGYTFTSYNMHWVKQT |
| | | PGRGLEWIGAIYPGNGDTSY |
| | | NQKFKGKATLTADKSSSTAY |
| | | MQLSSLTSEDSAVYYCARST |
| | | YYGGDWYFNVWGAGTTVTVS |
| | | A |
| CD20 VL | SEQ ID NO: 170 | QIVLSQSPAILSPSPGEKVT |
| | | MTCRASSSVSYIHWFQQKPG |
| | | SSPKPWIYATSNLASGVPVR |
| | | FSGSGSGTSYSLTISRVEAE |
| | | DAATYYCQQWTSNPPTFGGG |
| | | TKLEIKR |
| HER2 VH | SEQ ID NO: 171 | EVQLVESGGGLVQPGGSLRL |
| | | SCAASGFNIKDTYIHWVRQA |
| | | PGKGLEWVARIYPTNGYTRY |
| | | ADSVKGRFTISADTSKNTAY |
| | | LQMNSLRAEDTAVYYCSRWG |
| | | GDGFYAMDYWGQGTLVTVSS |
| HER2 VL | SEQ ID NO: 172 | DIQMTQSPSSLSASVGDRVT |
| | | ITCRASQDVNTAVAWYQQKP |
| | | GKAPKLLIYSASFLYSGVPS |
| | | RFSGSRSGTDFTLTISSLQP |
| | | EDFATYYCQQHYTTPPTFGQ |
| | | GTKVEIKR |

Further single variable domain sequences are provided in the Examples below.

### A5. Dual Variable Domain Binding Proteins

Variable domains of interest (heavy and/or light) for use in the TVD binding proteins of the present invention can be derived from the sequences provided in US Patent Publications 20100260668 and 20090304693. It is understood that the single variable domains can be selected from the dual variable domain binding proteins disclosed therein for use in the TVD binding proteins of the present invention. Sequences can also be selected from the following tables.

Exemplary dual variable domains for use in the binding proteins of the instant invention include the following dual variable domain sequences for binding the indicated proteins.

TABLE 2
Dual Variable Domain sequences for binding HIV

| | DVD | Outer | | Inner | |
|---|---|---|---|---|---|
| SEQ | Variable | Variable | | Variable | |
| ID | Domain | Domain | | Domain | Sequence |
| NO: | Name | Name | Linker | Name | 123456789012345678901234567890123456 |
| 97 | DVD715H | AB081VH | HG- | AB081VH | QVQLQQSGAELMKPGASVKISCKASGYTFTSYWIEW |
| | | | short | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVTVSAASTKGPQVQLQQSGAELMKPGASVK |
| | | | | | ISCKASGYTFTSYWIEWIKQRPGHGLEWIGEILPGT |
| | | | | | GSLNNNEKFRDKATFTADTSSNTAYMQLSSLTSEDS |
| | | | | | AVYYCARGYRYDGWFAYWGQGTLVTVSA |
| 98 | DVD715L | AB081VL | LK- | AB081VL | DIQMTQSPASLSASVGETVTITCRTSENIYSYLAWY |
| | | | short | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPDIQMTQSPASLSASVGETVTITCRTSENIYS |
| | | | | | YLAWYQQKPGKSPHLLVYNTKTLAEGVPSRFSGSGS |
| | | | | | GTQFSLKINSLQPEDFGSYYCQHHYDSPLTFGSGTK |
| | | | | | LELKR |
| 99 | DVD716H | AB081VH | HG- | AB081VH | QVQLQQSGAELMKPGASVKISCKASGYTFTSYWIEW |
| | | | long | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVTVSAASTKGPSVFPLAPQVQLQQSGAELM |
| | | | | | KPGASVKISCKASGYTFTSYWIEWIKQRPGHGLEWI |
| | | | | | GEILPGTGSLNNNEKFRDKATFTADTSSNTAYMQLS |
| | | | | | SLTSEDSAVYYCARGYRYDGWFAYWGQGTLVTVSA |
| 100 | DVD716L | AB081VL | LK- | AB081VL | DIQMTQSPASLSASVGETVTITCRTSENIYSYLAWY |
| | | | long | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPDIQMTQSPASLSASVGETVTITCR |
| | | | | | TSENIYSYLAWYQQKPGKSPHLLVYNTKTLAEGVPS |
| | | | | | RFSGSGSGTQFSLKINSLQPEDFGSYYCQHHYDSPL |
| | | | | | TFGSGTKLELKR |
| 101 | DVD717H | AB081VH | HG- | AB081VH | QVQLQQSGAELMKPGASVKISCKASGYTFTSYWIEW |
| | | | longX2 | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVIVSAASTKGPSVFPLAPASTKGPSVFPLA |
| | | | | | PQVQLQQSGAELMKPGASVKISCKASGYTFTSYWIE |
| | | | | | WIKQRPGHGLEWIGEILPGTGSLNNNEKFRDKATFT |
| | | | | | ADTSSNTAYMQLSSLTSEDSAVYYCARGYRYDGWFA |
| | | | | | YWGQGTLVTVSA |
| 102 | DVD717L | AB081VL | LK- | AB081VL | DIQMTQSPASLSASVGETVTITCRTSENIYSYLAWY |
| | | | longX2 | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPTVAAPSVFIFPPDIQMTQSPASLS |
| | | | | | ASVGETVTITCRTSENIYSYLAWYQQKPGKSPHLLV |
| | | | | | YNTKTLAEGVPSRFSGSGSGTQFSLKINSLQPEDFG |
| | | | | | SYYCQHHYDSPLTFGSGTKLELKR |
| 103 | DVD746H | AB081VH | HG- | AB085VH | QVQLQQSGAELMKPGASVKISCKASGYTFTSYWIEW |
| | | | Long | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVTVSAASTKGPSVFPLAPEVQLQQSGPELV |
| | | | | | KPGASMKISCKASDYSFTAYTIHWMKQSHGKNLEWI |
| | | | | | GLINPYNGGTSYNQKFQGRATLTVDKSSSIAYMELL |
| | | | | | SLTSEDSAVYYCARRGYDREGHYYAMDYWGQGTSVT |
| | | | | | VSS |
| 104 | DVD746L | AB081VL | LK- | AB085VL | DIQMTQSPASLSASVGETVTITCRTSENIYSYLAWY |
| | | | long | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPDIQMTQSPASLAASVGETVTITCR |
| | | | | | ASENIYTFLAWYQQKQGKSPQLLVYTTKTLAEGVPS |
| | | | | | RFSGSGSGTQFSLKIKSLQPEDFGSYYCQHHYGLPL |
| | | | | | TFGAGTKLELKR |
| 105 | DVD747H | AB081VH | HG- | AB085VH | QVQLQQSGAELMKPGASVKISCKASGYTFTSYWIEW |
| | | | longX2 | | IKQRPGHGLEWIGEILPGTGSLNNNEKFRDKATFTA |
| | | | | | DTSSNTAYMQLSSLTSEDSAVYYCARGYRYDGWFAY |
| | | | | | WGQGTLVTVSAASTKGPSVFPLAPASTKGPSVFPLA |
| | | | | | PEVQLQQSGPELVKPGASMKISCKASDYSFTAYTIH |
| | | | | | WMKQSHGKNLEWIGLINPYNGGTSYNQKFQGRATLT |
| | | | | | VDKSSSIAYMELLSLTSEDSAVYYCARRGYDREGHY |
| | | | | | YAMDYWGQGTSVTVSS |
| 106 | DVD747L | AB081VL | LK- | AB085VL | DIQMTQSPASLSASVGETVTITCRTSENIYSYLAWY |
| | | | longX2 | | QQKPGKSPHLLVYNTKTLAEGVPSRFSGSGSGTQFS |
| | | | | | LKINSLQPEDFGSYYCQHHYDSPLTFGSGTKLELKR |
| | | | | | TVAAPSVFIFPPTVAAPSVFIFPPDIQMTQSPASLA |
| | | | | | ASVGETVTITCRASENIYTFLAWYQQKQGKSPQLLV |
| | | | | | YTTKTLAEGVPSRFSGSGSGTQFSLKIKSLQPEDFG |
| | | | | | SYYCQHHYGLPLTFGAGTKLELKR |
| 107 | DVD748H | AB085VH | HG- | AB081VH | EVQLQQSGPELVKPGASMKISCKASDYSFFAYTIHW |
| | | | Long | | MKQSHGKNLEWIGLINPYNGGTSYNQKFQGRATLTV |
| | | | | | DKSSSIAYMELLSLTSEDSAVYYCARRGYDREGHYY |
| | | | | | AMDYWGQGTSVTVSSASTKGPSVFPLAPQVQLQQSG |
| | | | | | AELMKPGASVKISCKASGYTFTSYWIEWIKQRPGHG |
| | | | | | LEWIGEILPGTGSLNNNEKFRDKATFTADTSSNTAY |
| | | | | | MQLSSLTSEDSAVYYCARGYRYDGWFAYWGQGTLVT |
| | | | | | VSA |
| 108 | DVD748L | AB085VL | LK- | AB081VL | DIQMTQSPASLAASVGETVTITCRASENIYTFLAWY |
| | | | long | | QQKQGKSPQLLVYTTKTLAEGVPSRFSGSGSGTQFS |
| | | | | | LKIKSLQPEDFGSYYCQHHYGLPLTFGAGTKLELKR |
| | | | | | TVAAPSVFIFPPDIQMTQSPASLSASVGETVTITCR |
| | | | | | TSENIYSYLAWYQQKPGKSPHLLVYNTKTLAEGVPS |
| | | | | | RFSGSGSGTQFSLKINSLQPEDFGSYYCQHHYDSPL |
| | | | | | TFGSGTKLELKR |
| 109 | DVD749H | AB085VH | HG- | AB081VH | EVQLQQSGPELVKPGASMKISCKASDYSFTAYTIHW |
| | | | longX2 | | MKQSHGKNLEWIGLINPYNGGTSYNQKFQGRATLTV |
| | | | | | DKSSSIAYMELLSLTSEDSAVYYCARRGYDREGHYY |
| | | | | | AMDYWGQGTSVTVSSASTKGPSVFPLAPASTKGPSV |
| | | | | | FPLAPQVQLQQSGAELMKPGASVKISCKASGYTFTS |
| | | | | | YWIEWIKQRPGHGLEWIGEILPGTGSLNNNEKFRDK |
| | | | | | ATFTADTSSNTAYMQLSSLTSEDSAVYYCARGYRYD |
| | | | | | GWFAYWGQGTLVTVSA |
| 110 | DVD749L | AB085VL | LK- | AB081VL | DIQMTQSPASLAASVGETVTITCRASENIYTFLAWY |
| | | | longX2 | | QQKQGKSPQLLVYTTKTLAEGVPSRFSGSGSGTQFS |
| | | | | | LKIKSLQPEDFGSYYCQHHYGLPLTFGAGTKLELKR |
| | | | | | TVAAPSVFIFPPTVAAPSVFIFPPDIQMTQSPASLS |
| | | | | | ASVGETVTITCRTSENIYSYLAWYQQKPGKSPHLLV |
| | | | | | YNTKTLAEGVPSRFSGSGSGTQFSLKINSLQPEDFG |
| | | | | | SYYCQHHYDSPLTFGSGTKLELKR |
| 111 | DVD744H | AB084VH | HG- | AB084VH | QIQLVQSGPELKKPGETVKISCKASGYTFTDYSMHW |
| | | | long | | VKQAPGKGLKWMGWIHTETGEPRYVDDFKGRFAFSL |
| | | | | | ETSASTAYLQINNLKNEDTATYFCARDSYYFGSSYY |
| | | | | | FDYWGQGTTLTVSSASTKGPSVFPLAPQIQLVQSGP |
| | | | | | ELKKPGETVKISCKASGYTFTDYSMHWVKQAPGKGL |
| | | | | | KWMGWIHTETGEPRYVDDFKGRFAFSLETSASTAYL |
| | | | | | QINNLKNEDTATYFCARDSYYFGSSYYFDYWGQGTT |
| | | | | | LTVSS |
| 112 | DVD744L | AB084VL | LK- | AB084VL | DTVMTQSHKFMSTSVGDRVSITCKASQDVSSAVAWY |
| | | | long | | QQKPGQSPKLLIYSASYRYTGVPDRFTGSGSGMDFT |
| | | | | | FTISSVQAEDLAVYYCQQHYSTPLTFGAGTKLELER |
| | | | | | TVAAPSVFIFPPDTVMTQSHKFMSTSVGDRVSITCK |
| | | | | | ASQDVSSAVAWYQQKPGQSPKLLIYSASYRYTGVPD |
| | | | | | RFTGSGSGMDFTFTISSVQAEDLAVYYCQQHYSTPL |
| | | | | | TFGAGTKLELER |
| 113 | DVD745H | AB084VH | HG- | AB084VH | QIQLVQSGPELKKPGETVKISCKASGYTFTDYSMHW |
| | | | short | | VKQAPGKGLKWMGWIHTETGEPRYVDDFKGRFAFSL |
| | | | | | ETSASTAYLQINNLKNEDTATYFCARDSYYFGSSYY |
| | | | | | FDYWGQGTTLTVSSASTKGPQIQLVQSGPELKKPGE |
| | | | | | TVKISCKASGYTFTDYSMHWVKQAPGKGLKWMGWIH |
| | | | | | TETGEPRYVDDFKGRFAFSLETSASTAYLQINNLKN |
| | | | | | EDTATYFCARDSYYFGSSYYFDYWGQGTTLTVSS |
| 114 | DVD745L | AB084VL | LK- | AB084VL | DTVMTQSHKFMSTSVGDRVSITCKASQDVSSAVAWY |
| | | | short | | QQKPGQSPKLLIYSASYRYTGVPDRFTGSGSGMDFT |
| | | | | | FTISSVQAEDLAVYYCQQHYSTPLTFGAGTKLELER |
| | | | | | TVAAPDTVMTQSHKFMSTSVGDRVSITCKASQDVSS |
| | | | | | AVAWYQQKPGQSPKLLIYSASYRYTGVPDRFTGSGS |
| | | | | | GMDFTFTISSVQAEDLAVYYCQQHYSTPLTFGAGTK |
| | | | | | LELER |
| 115 | DVD750H | AB086VH | HG- | AB086VH | EVQLQQSGPELVQPGASMKISCKASGYSFTDYTMNW |
| | | | long | | VKQSHGKNLEWIGLINPYNGGSRYNQKFMAKATLTV |
| | | | | | DKSSNTAYMELLSVTSEDSAVYYCARDAGYFGSGFY |
| | | | | | FDYWGQGTTLTVSSASTKGPSVFPLAPEVQLQQSGP |
| | | | | | ELVQPGASMKISCKASGYSFTDYTMNWVKQSHGKNL |
| | | | | | EWIGLINPYNGGSRYNQKFMAKATLTVDKSSNTAYM |
| | | | | | ELLSVTSEDSAVYYCARDAGYFGSGFYFDYWGQGTT |
| | | | | | LTVSS |
| 116 | DVD750L | AB086VL | LH- | AB086VL | DIVMTQSHKFMSTSVGDRVSITCKASQDVSTAVAWY |
| | | | long | | QQKPGQSPKLLIYSASYRSTGVPDRFTGSGSGTDFT |
| | | | | | FTISSVQAEDLAVYYCQQHYSTPTFGAGTKLELKRT |
| | | | | | VAAPSVFIFPPDIVMTQSHKFMSTSVGDRVSITCKA |
| | | | | | SQDVSTAVAWYQQKPGQSPKLLIYSASYRSTGVPDR |
| | | | | | FTGSGSGTDFTFTISSVQAEDLAVYYCQQHYSTPTF |
| | | | | | GAGTKLELKR |

TABLE 3
Dual Variable Domain sequences for binding NGAL

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 117 | DVD719H | AB082VH | HG- | AB082VH | EVQLVESGGGLVQPGGSLKLSCAASGFTFNNYYMSW |
| | | | long | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPEVQLVESGGGLV |
| | | | | | QPGGSLKLSCAASGFTFNNYYMSWVRQTPERRLEWV |
| | | | | | AYISSSGGSTYYSDSVRGRFTISRDTARNTLYLQMT |
| | | | | | SLKSEDTAMYYCARHFGDYSYFDYWGQGTTLTVSS |
| 118 | DVD719L | AB082VL | LK- | AB082VL | DIQMTQSPASLSASVGETVTITCRASENFYSYLAWY |
| | | | long | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPDIQMTQSPASLSASVGETVTITCR |
| | | | | | ASENFYSYLAWYQQKQGKSPQLLVYNAKTLAEGVPS |
| | | | | | RFSGSGSGTQFSLKINSLQPEDFGTYYCQHHYDIPL |
| | | | | | TFGAGTKLELKR |
| 119 | DVD720H | AB082VH | HG- | AB082VH | EVQLVESGGGLVQPGGSLKLSCAASGFTFNNYYMSW |
| | | | short | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPEVQLVESGGGLVQPGGSLK |
| | | | | | LSCAASGFTFNNYYMSWVRQTPERRLEWVAYISSSG |
| | | | | | GSTYYSDSVRGRFTISRDTARNTLYLQMTSLKSEDT |
| | | | | | AMYYCARHFGDYSYFDYWGQGTTLTVSS |
| 120 | DVD720L | AB082VL | LK- | AB082VL | DIQMTQSPASLSASVGETVTITCRASENFYSYLAWY |
| | | | short | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPDIQMTQSPASLSASVGETVTITCRASENFYS |
| | | | | | YLAWYQQKQGKSPQLLVYNAKTLAEGVPSRFSGSGS |
| | | | | | GTQFSLKINSLQPEDFGTYYCQHHYDIPLTFGAGTK |
| | | | | | LELKR |
| 121 | DVD721H | AB083VH | HG- | AB083VH | KIQLVQSGPELKKPGETVKISCKASGYTFTNYGMNW |
| | | | long | | VKQAPGKGLKWMGWININTGEPTYAEEFKGRFAFSL |
| | | | | | ETSATTAFLQINNLKNEDTATYLCARDSYSGGFDYW |
| | | | | | GQGTIVTVSSASTKGPSVFPLAPKIQLVQSGPELKK |
| | | | | | PGETVKISCKASGYTFTNYGMNWVKQAPGKGLKWMG |
| | | | | | WININTGEPTYAEEFKGRFAFSLETSATTAFLQINN |
| | | | | | LKNEDTATYLCARDSYSGGFDYWGQGTIVTVSS |
| 122 | DVD721L | AB083VL | LK- | AB083VL | DIVMTQSPSSLSVSAGEKVTLSCKSSQSLLISGDQK |
| | | | long | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPSVFIFPPDIVMTQSPSSLSVSAGEK |
| | | | | | VTLSCKSSQSLLISGDQKNYLAWYQQKPGQPPKLLI |
| | | | | | YGASTRDSGVPDRFTGSGSGADFTLTISSVQAEDLA |
| | | | | | VYYCQNDHSFPPTFGAGTKLELKR |
| 123 | DVD722H | AB083VH | HG- | AB083VH | KIQLVQSGPELKKPGETVKISCKASGYTFTNYGMNW |
| | | | short | | VKQAPGKGLKWMGWININTGEPTYAEEFKGRFAFSL |
| | | | | | ETSATTAFLQINNLKNEDTATYLCARDSYSGGFDYW |
| | | | | | GQGTIVTVSSASTKGPKIQLVQSGPELKKPGETVKI |
| | | | | | SCKASGYTFTNYGMNWVKQAPGKGLKWMGWININTG |
| | | | | | EPTYAEEFKGRFAFSLETSATTAFLQINNLKNEDTA |
| | | | | | TYLCARDSYSGGFDYWGQGTIVTVSS |
| 124 | DVD722L | AB083VL | LK- | AB083VL | DIVMTQSPSSLSVSAGEKVTLSCKSSQSLLISGDQK |
| | | | short | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPDIVMTQSPSSLSVSAGEKVTLSCKS |
| | | | | | SQSLLISGDQKNYLAWYQQKPGQPPKLLIYGASTRD |
| | | | | | SGVPDRFTGSGSGADFTLTISSVQAEDLAVYYCQND |
| | | | | | HSFPPTFGAGTKLELKR |
| 125 | DVD723H | AB082VH | HG- | AB083VH | EVQLVESGGGLVQPGGSLKLSCAASGFTFNNYYMSW |
| | | | long | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPKIQLVQSGPELK |
| | | | | | KPGETVKISCKASGYTFTNYGMNWVKQAPGKGLKWM |
| | | | | | GWININTGEPTYAEEFKGRFAFSLETSATTAFLQIN |
| | | | | | NLKNEDTATYLCARDSYSGGFDYWGQGTIVTVSS |
| 126 | DVD723L | AB082VL | LK- | AB083VL | DIQMTQSPASLSASVGETVTITCRASENFYSYLAWY |
| | | | long | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPDIVMTQSPSSLSVSAGEKVTLSCK |
| | | | | | SSQSLLISGDQKNYLAWYQQKPGQPPKLLIYGASTR |
| | | | | | DSGVPDRFTGSGSGADFTLTISSVQAEDLAVYYCQN |
| | | | | | DHSFPPTFGAGTKLELKR |
| 127 | DVD724H | AB082VH | HG- | AB083VH | EVQLVESGGGLVQPGGSLKLSCAASGFTFNNYYMSW |
| | | | short | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPKIQLVQSGPELKKPGETVK |
| | | | | | ISCKASGYTFTNYGMNWVKQAPGKGLKWMGWININT |
| | | | | | GEPTYAEEFKGRFAFSLETSATTAFLQINNLKNEDT |
| | | | | | ATYLCARDSYSGGFDYWGQGTIVTVSS |
| 128 | DVD724L | AB082VL | LK- | AB083VL | DIQMTQSPASLSASVGETVTITCRASENFYSYLAWY |
| | | | short | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPDIVMTQSPSSLSVSAGEKVTLSCKSSQSLLI |
| | | | | | SGDQKNYLAWYQQKPGQPPKLLIYGASTRDSGVPDR |
| | | | | | FTGSGSGADFTLTISSVQAEDLAVYYCQNDHSFPPT |
| | | | | | FGAGTKLELKR |
| 129 | DVD725H | AB083VH | HG- | AB082VH | KIQLVQSGPELKKPGETVKISCKASGYTFTNYGMNW |
| | | | long | | VKQAPGKGLKWMGWININTGEPTYAEEFKGRFAFSL |
| | | | | | ETSATTAFLQINNLKNEDTATYLCARDSYSGGFDYW |
| | | | | | GQGTIVTVSSASTKGPSVFPLAPEVQLVESGGGLVQ |
| | | | | | PGGSLKLSCAASGFTFNNYYMSWVRQTPERRLEWVA |
| | | | | | YISSSGGSTYYSDSVRGRFTISRDTARNTLYLQMTS |
| | | | | | LKSEDTAMYYCARHFGDYSYFDYWGQGTTLTVSS |
| 130 | DVD725L | AB083VL | LK- | AB082VL | DIVMTQSPSSLSVSAGEKVTLSCKSSQSLLISGDQK |
| | | | long | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPSVFIFPPDIQMTQSPASLSASVGET |
| | | | | | VTITCRASENFYSYLAWYQQKQGKSPQLLVYNAKTL |
| | | | | | AEGVPSRFSGSGSGTQFSLKINSLQPEDFGTYYCQH |
| | | | | | HYDIPLTFGAGTKLELKR |
| 131 | DVD726H | AB083VH | HG- | AB082VH | KIQLVQSGPELKKPGETVKISCKASGYTFTNYGMNW |
| | | | short | | VKQAPGKGLKWMGWININTGEPTYAEEFKGRFAFSL |
| | | | | | ETSATTAFLQINNLKNEDTATYLCARDSYSGGFDYW |
| | | | | | GQGTIVTVSSASTKGPEVQLVESGGGLVQPGGSLKL |
| | | | | | SCAASGFTFNNYYMSWVRQTPERRLEWVAYISSSGG |
| | | | | | STYYSDSVRGRFTISRDTARNTLYLQMTSLKSEDTA |
| | | | | | MYYCARHFGDYSYFDYWGQGTTLTVSS |
| 132 | DVD726L | AB083VL | LK- | AB082VL | DIVMTQSPSSLSVSAGEKVTLSCKSSQSLLISGDQK |
| | | | short | | NYLAWYQQKPGQPPKLLIYGASTRDSGVPDRFTGSG |
| | | | | | SGADFTLTISSVQAEDLAVYYCQNDHSFPPTFGAGT |
| | | | | | KLELKRTVAAPDIQMTQSPASLSASVGETVTITCRA |
| | | | | | SENFYSYLAWYQQKQGKSPQLLVYNAKTLAEGVPSR |
| | | | | | FSGSGSGTQFSLKINSLQPEDFGTYYCQHHYDIPLT |
| | | | | | FGAGTKLELKR |

TABLE 4
Dual Variable Domain sequences for binding NGAL and IL-18

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 133 | DVD727H | AB082VH | HG- | AB088VH | EVQLVESGGGLVQPGGSLKLSCAASGFTFNNYYMSW |
| | | | short | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLIVSSASTKGPQVQLQQPGSELVRPGASVK |
| | | | | | LSCKASGYTFTSYWMHWVKQRPGQGLEWIGNIYPGT |
| | | | | | VNTNYDEKFKNKATLTVDTSSSTAYMLLSSLTSEDS |
| | | | | | AVYYCTRDYYGGGLNYWGQGTTLTVSS |
| 134 | DVD727L | AB082VL | LK- | AB088VL | DIQMTQSPASLSASVGETVTITCRASENFYSYLAWY |
| | | | short | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSIVMTQTPKFLLVSAGDRVTITCKASQSVSN |
| | | | | | DVAWFQQKPGQSPKLLIYYASNRYAGVPDRFTGSGF |
| | | | | | GTDFTFTISTVQAEDLAVYFCHQDYSSPRTFGGGTK |
| | | | | | LEIKR |
| 135 | DVD728H | AB082VH | HG- | AB088VH | EVQLVESGGGLVQPGGSLKLSCAASGFTFNNYYMSW |
| | | | long | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPQVQLQQPGSELV |
| | | | | | RPGASVKLSCKASGYTFTSYWMHWVKQRPGQGLEWI |
| | | | | | GNIYPGTVNTNYDEKFKNKATLTVDTSSSTAYMLLS |
| | | | | | SLTSEDSAVYYCTRDYYGGGLNYWGQGTTLTVSS |
| 136 | DVD728L | AB082VL | LK- | AB088VL | DIQMTQSPASLSASVGETVTITCRASENFYSYLAWY |
| | | | long | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPSIVMTQTPKFLLVSAGDRVTITCK |
| | | | | | ASQSVSNDVAWFQQKPGQSPKLLIYYASNRYAGVPD |
| | | | | | RFTGSGFGTDFTFTISTVQAEDLAVYFCHQDYSSPR |
| | | | | | TFGGGTKLEIKR |
| 137 | DVD729H | AB082VH | HG- | AB088VH | EVQLVESGGGLVQPGGSLKLSCAASGFTFNNYYMSW |
| | | | longX2 | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSSASTKGPSVFPLAPASTKGPSVFPLA |
| | | | | | PQVQLQQPGSELVRPGASVKLSCKASGYTFTSYWMH |
| | | | | | WVKQRPGQGLEWIGNIYPGTVNTNYDEKFKNKATLT |
| | | | | | VDTSSSTAYMLLSSLTSEDSAVYYCTRDYYGGGLNY |
| | | | | | WGQGTTLTVSS |
| 138 | DVD729L | AB082VL | LK- | AB088VL | DIQMTQSPASLSASVGETVTITCRASENFYSYLAWY |
| | | | longX2 | | QQKQGKSPQLLVYNAKTLAEGVPSRFSGSGSGTQFS |
| | | | | | LKINSLQPEDFGTYYCQHHYDIPLTFGAGTKLELKR |
| | | | | | TVAAPSVFIFPPTVAAPSVFIFPPSIVMTQTPKFLL |
| | | | | | VSAGDRVTITCKASQSVSNDVAWFQQKPGQSPKLLI |
| | | | | | YYASNRYAGVPDRFTGSGFGTDFTFTISTVQAEDLA |
| | | | | | VYFCHQDYSSPRTFGGGTKLEIKR |
| 139 | DVD730H | AB088VH | HG- | AB082VH | QVQLQQPGSELVRPGASVKLSCKASGYTFTSYWMHW |
| | | | short | | VKQRPGQGLEWIGNIYPGTVNTNYDEKFKNKATLTV |
| | | | | | DTSSSTAYMLLSSLTSEDSAVYYCTRDYYGGGLNYW |
| | | | | | GQGTTLTVSSASTKGPEVQLVESGGGLVQPGGSLKL |
| | | | | | SCAASGFTFNNYYMSWVRQTPERRLEWVAYISSSGG |
| | | | | | STYYSDSVRGRFTISRDTARNTLYLQMTSLKSEDTA |
| | | | | | MYYCARHFGDYSYFDYWGQGTTLTVSS |
| 140 | DVD730L | AB088VL | LK- | AB082VL | SIVMTQTPKFLLVSAGDRVTITCKASQSVSNDVAWF |
| | | | short | | QQKPGQSPKLLIYYASNRYAGVPDRFTGSGFGTDFT |
| | | | | | FTISTVQAEDLAVYFCHQDYSSPRTFGGGTKLEIKR |
| | | | | | TVAAPDIQMTQSPASLSASVGETVTITCRASENFYS |
| | | | | | YLAWYQQKQGKSPQLLVYNAKTLAEGVPSRFSGSGS |
| | | | | | GTQFSLKINSLQPEDFGTYYCQHHYDIPLTFGAGTK |
| | | | | | LELKR |
| 141 | DVD731H | AB088VH | HG- | AB082VH | QVQLQQPGSELVRPGASVKLSCKASGYTFTSYWMHW |
| | | | long | | VKQRPGQGLEWIGNIYPGTVNTNYDEKFKNKATLTV |
| | | | | | DTSSSTAYMLLSSLTSEDSAVYYCTRDYYGGGLNYW |
| | | | | | GQGTTLTVSSASTKGPSVFPLAPEVQLVESGGGLVQ |
| | | | | | PGGSLKLSCAASGFTFNNYYMSWVRQTPERRLEWVA |
| | | | | | YISSSGGSTYYSDSVRGRFTISRDTARNTLYLQMTS |
| | | | | | LKSEDTAMYYCARHFGDYSYFDYWGQGTTLTVSS |
| 142 | DVD731L | AB088VL | LK- | AB082VL | SIVMTQTPKFLLVSAGDRVTITCKASQSVSNDVAWF |
| | | | long | | QQKPGQSPKLLIYYASNRYAGVPDRFTGSGFGTDFT |
| | | | | | FTISTVQAEDLAVYFCHQDYSSPRTFGGGTKLEIKR |
| | | | | | TVAAPSVFIFPPDIQMTQSPASLSASVGETVTITCR |
| | | | | | ASENFYSYLAWYQQKQGKSPQLLVYNAKTLAEGVPS |
| | | | | | RFSGSGSGTQFSLKINSLQPEDFGTYYCQHHYDIPL |
| | | | | | TFGAGTKLELKR |
| 143 | DVD732H | AB088VH | HG- | AB082VH | QVQLQQPGSELVRPGASVKLSCKASGYTFTSYWMHW |
| | | | longX2 | | VKQRPGQGLEWIGNIYPGTVNTNYDEKFKNKATLTV |
| | | | | | DTSSSTAYMLLSSLTSEDSAVYYCTRDYYGGGLNYW |
| | | | | | GQGTTLTVSSASTKGPSVFPLAPASTKGPSVFPLAP |
| | | | | | EVQLVESGGGLVQPGGSLKLSCAASGFTFNNYYMSW |
| | | | | | VRQTPERRLEWVAYISSSGGSTYYSDSVRGRFTISR |
| | | | | | DTARNTLYLQMTSLKSEDTAMYYCARHFGDYSYFDY |
| | | | | | WGQGTTLTVSS |
| 144 | DVD732L | AB088VL | LK- | AB082VL | SIVMTQTPKFLLVSAGDRVTITCKASQSVSNDVAWF |
| | | | longX2 | | QQKPGQSPKLLIYYASNRYAGVPDRFTGSGFGTDFT |
| | | | | | FTISTVQAEDLAVYFCHQDYSSPRTFGGGTKLEIKR |
| | | | | | TVAAPSVFIFPPTVAAPSVFIFPPDIQMTQSPASLS |
| | | | | | ASVGETVTITCRASENFYSYLAWYQQKQGKSPQLLV |
| | | | | | YNAKTLAEGVPSRFSGSGSGTQFSLKINSLQPEDFG |
| | | | | | TYYCQHHYDIPLTFGAGTKLELKR |

TABLE 5
Dual Variable Domain sequences for binding BNP

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 145 | DVD733H | AB089VH | HG- | AB089VH | QIQLVQSGPELRKPGETVKISCKGSGYTFTHYGINW |
| | | | long | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPSVFPLAPQIQLVQSGPELRKP |
| | | | | | GETVKISCKGSGYTFTHYGINWVKQTPRKDLKWMGW |
| | | | | | INTHTGEAYYADDFKGRFAFSLETSANTAYLQINNL |
| | | | | | NNGDMGTYFCTRSHRFGLDYWGQGTSVTVSS |
| 146 | DVD733L | AB089VL | LK- | AB089VL | DNVLTQSPPSLAVSLGQRATISCKANWPVDYNGDSY |
| | | | long | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPSVFIFPPDNVLTQSPPSLAVSLGQRAT |
| | | | | | ISCKANWPVDYNGDSYLNWYQQKPGQPPKFLIYAAS |
| | | | | | NLESGIPARFSGSGSGTDFNLNIHPVEEEDAATYYC |
| | | | | | QQSNEDPFTFGSGTKLEIKR |
| 147 | DVD734H | AB089VH | HG- | AB089VH | QIQLVQSGPELRKPGETVKISCKGSGYTFTHYGINW |
| | | | short | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPQIQLVQSGPELRKPGETVKIS |
| | | | | | CKGSGYTFTHYGINWVKQTPRKDLKWMGWINTHTGE |
| | | | | | AYYADDFKGRFAFSLETSANTAYLQINNLNNGDMGT |
| | | | | | YFCTRSHRFGLDYWGQGTSVTVSS |
| 148 | DVD734L | AB089VL | LK- | AB089VL | DNVLTQSPPSLAVSLGQRATISCKANWPVDYNGDSY |
| | | | short | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPDNVLTQSPPSLAVSLGQRATISCKANW |
| | | | | | PVDYNGDSYLNWYQQKPGQPPKFLIYAASNLESGIP |
| | | | | | ARFSGSGSGTDFNLNIHPVEEEDAATYYCQQSNEDP |
| | | | | | FTFGSGTKLEIKR |
| 149 | DVD735H | AB090VH | HG- | AB090VH | QVQLQQPGAELVRPGASVKLSCKASGYTFTSYWMNW |
| | | | long | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPQVQLQQPGAELVRPGASVK |
| | | | | | LSCKASGYTFTSYWMNWVKQRPEQGLEWIGRIDPYD |
| | | | | | SETHYNQKFKDKAILTVDKSSSTAFVQLTSLTSEDS |
| | | | | | AVYYCVSDGYWGAGTTVTVSS |
| 150 | DVD735L | AB090VL | LK- | AB090VL | DVVMTQTPLTLSVTTGQPASISCKSSQSLLDSDGKT |
| | | | long | | YLNWLFQRPGESPKLLIYVVSKLESGVPDRFTGSGS |
| | | | | | GTDFTLKISRVEAEDLGVYYCLQATHFPWTFGGGTK |
| | | | | | LEIKRTVAAPSVFIFPPDVVMTQTPLTLSVTTGQPA |
| | | | | | SISCKSSQSLLDSDGKTYLNWLFQRPGESPKLLIYV |
| | | | | | VSKLESGVPDRFTGSGSGTDFTLKISRVEAEDLGVY |
| | | | | | YCLQATHFPWTFGGGTKLEIKR |
| 151 | DVD736H | AB090VH | HG- | AB089VH | QVQLQQPGAELVRPGASVKLSCKASGYTFTSYWMNW |
| | | | long | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPQIQLVQSGPELRKPGETVK |
| | | | | | ISCKGSGYTFTHYGINWVKQTPRKDLKWMGWINTHT |
| | | | | | GEAYYADDFKGRFAFSLETSANTAYLQINNLNNGDM |
| | | | | | GTYFCTRSHRFGLDYWGQGTSVTVSS |
| 152 | DVD736L | AB090VL | LK- | AB089VL | DVVMTQTPLTLSVTTGQPASISCKSSQSLLDSDGKT |
| | | | long | | YLNWLFQRPGESPKLLIYVVSKLESGVPDRFTGSGS |
| | | | | | GTDFTLKISRVEAEDLGVYYCLQATHFPWTFGGGTK |
| | | | | | LEIKRTVAAPSVFIFPPDNVLTQSPPSLAVSLGQRA |
| | | | | | TISCKANWPVDYNGDSYLNWYQQKPGQPPKFLIYAA |
| | | | | | SNLESGIPARFSGSGSGTDFNLNIHPVEEEDAATYY |
| | | | | | CQQSNEDPFTFGSGTKLEIKR |
| 153 | DVD737H | AB090VH | HG- | AB089VH | QVQLQQPGAELVRPGASVKLSCKASGYTFTSYWMNW |
| | | | longX2 | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPASTKGPSVFPLAPQIQLVQ |
| | | | | | SGPELRKPGETVKISCKGSGYTFTHYGINWVKQTPR |
| | | | | | KDLKWMGWINTHTGEAYYADDFKGRFAFSLETSANT |
| | | | | | AYLQINNLNNGDMGTYFCTRSHRFGLDYWGQGTSVT |
| | | | | | VSS |
| 154 | DVD737L | AB090VL | LK- | AB089VL | DVVMTQTPLTLSVTTGQPASISCKSSQSLLDSDGKT |
| | | | longX2 | | YLNWLFQRPGESPKLLIYVVSKLESGVPDRFTGSGS |
| | | | | | GTDFTLKISRVEAEDLGVYYCLQATHFPWTFGGGTK |
| | | | | | LEIKRTVAAPSVFIFPPTVAAPSVFIFPPDNVLTQS |
| | | | | | PPSLAVSLGQRATISCKANWPVDYNGDSYLNWYQQK |
| | | | | | PGQPPKFLIYAASNLESGIPARFSGSGSGTDFNLNI |
| | | | | | HPVEEEDAATYYCQQSNEDPFTFGSGTKLEIKR |
| 155 | DVD738H | AB089VH | HG- | AB090VH | QIQLVQSGPELRKPGETVKISCKGSGYTFTHYGINW |
| | | | long | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPSVFPLAPQVQLQQPGAELVRP |
| | | | | | GASVKLSCKASGYTFTSYWMNWVKQRPEQGLEWIGR |
| | | | | | IDPYDSETHYNQKFKDKAILTVDKSSSTAFVQLTSL |
| | | | | | TSEDSAVYYCVSDGYWGAGTTVTVSS |
| 156 | DVD738L | AB089VL | LK- | AB090VL | DNVLTQSPPSLAVSLGQRATISCKANWPVDYNGDSY |
| | | | long | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPSVFIFPPDVVMTQTPLTLSVTTGQPAS |
| | | | | | ISCKSSQSLLDSDGKTYLNWLFQRPGESPKLLIYVV |
| | | | | | SKLESGVPDRFTGSGSGTDFTLKISRVEAEDLGVYY |
| | | | | | CLQATHFPWTFGGGTKLEIKR |
| 157 | DVD739H | AB089VH | HG- | AB090VH | QIQLVQSGPELRKPGETVKISCKGSGYTFTHYGINW |
| | | | longX2 | | VKQTPRKDLKWMGWINTHTGEAYYADDFKGRFAFSL |
| | | | | | ETSANTAYLQINNLNNGDMGTYFCTRSHRFGLDYWG |
| | | | | | QGTSVTVSSASTKGPSVFPLAPASTKGPSVFPLAPQ |
| | | | | | VQLQQPGAELVRPGASVKLSCKASGYTFTSYWMNWV |
| | | | | | KQRPEQGLEWIGRIDPYDSETHYNQKFKDKAILTVD |
| | | | | | KSSSTAFVQLTSLTSEDSAVYYCVSDGYWGAGTTVT |
| | | | | | VSS |
| 158 | DVD739L | AB089VL | LK- | AB090VL | DNVLTQSPPSLAVSLGQRATISCKANWPVDYNGDSY |
| | | | longX2 | | LNWYQQKPGQPPKFLIYAASNLESGIPARFSGSGSG |
| | | | | | TDFNLNIHPVEEEDAATYYCQQSNEDPFTFGSGTKL |
| | | | | | EIKRTVAAPSVFIFPPTVAAPSVFIFPPDVVMTQTP |
| | | | | | LTLSVTTGQPASISCKSSQSLLDSDGKTYLNWLFQR |
| | | | | | PGESPKLLIYVVSKLESGVPDRFTGSGSGTDFTLKI |
| | | | | | SRVEAEDLGVYYCLQATHFPWTFGGGTKLEIKR |
| 159 | DVD742H | AB092VH | HG- | AB092VH | QVQLQQPGAELVRPGASVKLSCKASGYTFTSYWMNW |
| | | | long | | VKQRPEQGLEWIGRIDPYDSETHYNQKFKDKAILTV |
| | | | | | DKSSSTAFVQLTSLTSEDSAVYYCVSDGYWGAGTTV |
| | | | | | TVSSASTKGPSVFPLAPQVQLQQPGAELVRPGASVK |
| | | | | | LSCKASGYTFTSYWMNWVKQRPEQGLEWIGRIDPYD |
| | | | | | SETHYNQKFKDKAILTVDKSSSTAFVQLTSLTSEDS |
| | | | | | AVYYCVSDGYWGAGTTVTVSS |
| 160 | DVD742L | AB092VL | LK- | AB092VL | DVVMTQTPLTLSVTTGQPASISCKSSQSLLDSDGKT |
| | | | long | | YLNWLFQRPGESPKLLIYVTDILESGVPDRFTGSGS |
| | | | | | GTDFILKISRVEAEDLGVYYCLQATHFPWTFGGGTK |
| | | | | | LEIKRTVAAPSVFIFPPDVVMTQTPLTLSVTTGQPA |
| | | | | | SISCKSSQSLLDSDGKTYLNWLFQRPGESPKLLIYV |
| | | | | | TDILESGVPDRFTGSGSGTDFTLKISRVEAEDLGVY |
| | | | | | YCLQATHFPWTFGGGTKLEIKR |

TABLE 6
Dual Variable Domain sequences for binding TnI

| | DVD | Outer | | Inner | |
|---|---|---|---|---|---|
| SEQ | Variable | Variable | | Variable | |
| ID | Domain | Domain | | Domain | Sequence |
| NO | Name | Name | Linker | Name | 123456789012345678901234567890123456 |
| 161 | DVD743H | AB093 | HG- | AB093VH | EVQLQQSGPDLVKPGASVRISCKASGYTFTDY |
| | | VH | long | | NLHWVKQSHGKSLEWIGYIYPYNGITGYNQKF |
| | | | | | KSKATLTVDSSSNTAYMDLRSLTSEDSAVYFC |
| | | | | | ARDAYDYDYLTDWGQGTLVTVSAASTKGPSVF |
| | | | | | PLAPEVQLQQSGPDLVKPGASVRISCKASGYT |
| | | | | | FTDYNLHWVKQSHGKSLEWIGYIYPYNGITGY |
| | | | | | NQKFKSKATLTVDSSSNTAYMDLRSLTSEDSA |
| | | | | | VYFCARDAYDYDYLTDWGQGTLVTVSA |
| 162 | DVD743L | AB093 | LK- | AB093VL | DILLTQSPVILSVSPGERVSFSCRTSKNVGTN |
| | | VL | long | | IHWYQQRTNGSPRLLIKYASERLPGIPSRFSG |
| | | | | | SGSGTDFTLSINSVESEDIADYYCQQSNNWPY |
| | | | | | TFGGGTKLEIKRTVAAPSVFIFPPDILLTQSP |
| | | | | | VILSVSPGERVSFSCRTSKNVGTNIHWYQQRT |
| | | | | | NGSPRLLIKYASERLPGIPSRFSGSGSGTDFT |
| | | | | | LSINSVESEDIADYYCQQSNNWPYTFGGGTKL |
| | | | | | EIKR |

### A5. Domain Antibodies

Parent binding proteins for use in the TVD binding proteins of the instant invention may include heavy chain antigen binding domains and light chain antigen binding domains wherein the antigen binding domain is a domain antibody. Domain antibodies are known in the art and methods to screen for domain antibodies that bind to specific epitopes are provided, for example in U.S. Pat. No. 7,829,096 (incorporated herein by reference). Many domain antibody sequences are publicly available, for example, in U.S. Pat. Nos. 7,696,320 and 7,829,096; and US Patent Publications 20100266616, 20100234570, 20100028354, 20060002935, which are all incorporated by reference herein in their entirety.

### A7. Receptor Immunoglobulins

Parent binding proteins for use in the TVD binding proteins of the instant invention may include heavy chain antigen binding domains and light chain antigen binding domains wherein the antigen binding domain is a receptor sequence. Many receptor sequences are known in the art and can be identified using BLAST or any of a number of publicly available databases. Additional receptor sequences include those immunoglobulin molecules provided in US Patent Application 2002/0127231, which is incorporated herein by reference including sequence listings. Receptor sequences can be incorporated into the half-Ig binding proteins of the instant invention using the same molecular biology techniques used to generate half-bodies including other variable domain sequences. Exemplary receptor sequences suitable for use in the TVD binding molecules of the present invention include, for example, CTLA4 (AMHVAQPAVVLASSRGIASFVCEYASPGKATEVRVTVLRQADSQVTEVCAATYMMGN ELTFLDDSICTGTSSGNQVNLTIQGLRAMDTGLYICKVELMYPPPYYLGIGNGTQIYVIDP EPCPDSD; SEQ ID NO:56) and TNFRSF1B (AQVAPTPYAPEPGSTCRLREYYDQTAQMCCSKCSPGQHAKVFCTKTSDTVCDSCEDSTY TQLWNVVVPECLSCGSRCSSDQVETQACTREQNRICTCRPGWYCALSKQEGCRLCAPLRK CRPGFGVARPGTETSDVVCKPCAPGTFSNTTSSTDICRPHQIC; SEQ ID NO:57).

### A8. Scaffold Antigen Binding Proteins

Parent binding proteins for use in the TVD binding proteins of the instant invention may include heavy chain antigen binding domains and light chain antigen binding domains wherein the antigen binding domain is a scaffold antigen binding protein. Scaffold antigen binding proteins are known in the art, for example, fibronectin and designed ankyrin-repeat proteins (DARPins) have been used as alternative scaffolds for antigen-binding domains, see, e.g., Gebauer and Skerra. Engineered protein scaffolds as next-generation antibody therapeutics. Curr Opin Chem Biol 13:245-255 (2009) and Stumpp et al., Darpins: A new generation of protein therapeutics. Drug Discov Today 13: 695-701 (2008), both of which are incorporated herein by reference in their entirety.

### A9. Half-Ig Binding Proteins

Parent binding proteins for use in the TVD binding proteins of the instant invention may include heavy chain antigen binding domains and light chain antigen binding domains derived from Half Iummunoglobulin binding proteins or Half-Ig provided in U.S. Patent Application Nos. 61/426,207, filed on Dec. 22, 2010 and 61/539,130, filed Sep. 26, 2011, and U.S. Pat. No. ______ being filed on the same day as the instant application in the name of the same assignee. The entire contents of each of the foregoing applications are incorporated herein by reference, including the sequence listings.

### B. Criteria for Selecting Parent Binding Proteins

One embodiment of the present disclosure pertains to selecting a parent binding protein, e.g., antibody or antibodies; variable domain(s) and/or receptor(s) with one or more properties desired in the TVD binding proteins. In one embodiment, the desired property is selected from one or more binding protein, e.g., antibody parameters. In another embodiment, the binding protein parameters are selected from the group consisting of antigen specificity, affinity to antigen, potency, biological function, epitope recognition, stability, solubility, production efficiency, immunogenicity, pharmacokinetics, bioavailability, tissue cross reactivity, and orthologous antigen-binding.

### B1. Affinity to Antigen

The desired affinity of a therapeutic binding protein, e.g., monoclonal antibody, may depend upon the nature of the antigen and the desired therapeutic end-point. In one embodiment, monoclonal antibodies have higher affinities (K_{d}=0.01-0.50 μM) when blocking a cytokine-cytokine receptor interaction as such interactions are usually high affinity interactions (e.g., <pM-<nM ranges). In such instances, the monoclonal antibody affinity for its target should be equal to or better than the affinity of the cytokine (ligand) for its receptor. On the other hand, a monoclonal antibody with lesser affinity (>nM range) could be therapeutically effective, e.g., in clearing circulating potentially pathogenic proteins, e.g., monoclonal antibodies that bind to, sequester, and clear circulating species of Aβ amyloid. In other instances, reducing the affinity of an existing high affinity monoclonal antibody by site-directed mutagenesis or using a monoclonal antibody with lower affinity for its target could be used to avoid potential side-effects, e.g., a high affinity monoclonal antibody may sequester/neutralize all of its intended target, thereby completely depleting/eliminating the function(s) of the targeted protein. In this scenario, a low affinity monoclonal antibody may sequester/neutralize a fraction of the target that may be responsible for the disease symptoms (the pathological or over-produced levels), thus allowing a fraction of the target to continue to perform its normal physiological function(s). Therefore, it may be possible to reduce the K_{d} to adjust dose and/or reduce side-effects. The affinity of the parental monoclonal antibody might play a role in appropriately targeting cell surface molecules to achieve desired therapeutic out-come. For example, if a target is expressed on cancer cells with high density and on normal cells with low density, a lower affinity monoclonal antibody will bind a greater number of targets on tumor cells than normal cells, resulting in tumor cell elimination via ADCC or CDC, and therefore might have therapeutically desirable effects. Thus selecting a monoclonal antibody with desired affinity may be relevant for both soluble and surface targets.

Signaling through a receptor upon interaction with its ligand may depend upon the affinity of the receptor-ligand interaction. Similarly, it is conceivable that the affinity of a monoclonal antibody for a surface receptor could determine the nature of intracellular signaling and whether the monoclonal antibody may deliver an agonist or an antagonist signal. The affinity-based nature of monoclonal antibody-mediated signaling may have an impact of its side-effect profile. Therefore, the desired affinity and desired functions of therapeutic monoclonal antibodies need to be determined carefully by in vitro and in vivo experimentation.

The desired K_{d} of a binding protein (e.g., an antibody) may be determined experimentally depending on the desired therapeutic outcome. In one embodiment, parent binding proteins, e.g., antibodies, with affinity (K_{d}) for a particular target antigen equal to, or better than, the desired affinity of the TVD binding protein for the same antigen are selected. The parent binding proteins, e.g., antibodies, for a given TVD binding protein can be the same binding protein, e.g., antibody, or different binding proteins, e.g., antibodies. The antigen-binding affinity and kinetics are assessed by Biacore or another similar technique. In one embodiment, each parent binding protein, e.g., antibody, has a dissociation constant (K_{d}) to its target antigen selected from the group consisting of: at most about 10⁻⁷ M; at most about 10⁻⁸ M; at most about 10⁻⁹ M; at most about 10⁻¹⁰ M; at most about 10⁻¹¹ M; at most about 10⁻¹² M; and at most 10⁻¹³M. The parent binding proteins, e.g., antibody(s), from which the variable domains are obtained may have similar or different affinity (K_{D}) for their respective target antigen(s). Each parent binding protein, e.g., antibody, has an on rate constant (Kₒₙ) to the antigen selected from the group consisting of: at least about 10² M⁻¹s⁻¹; at least about 10³M⁻¹s⁻¹; at least about 10⁴ M⁻¹s⁻¹; at least about 10⁵ M⁻¹s⁻¹; and at least about 10⁶ M⁻¹s⁻¹, as measured by surface plasmon resonance. The parent binding protein(s), e.g., antibody(s), from which the variable domains are obtained may have similar or different on rate constant (Kₒₙ) for their respective target antigen. In one embodiment, each parent binding protein, e.g., antibody, has an off rate constant (K_{off}) to the antigen selected from the group consisting of: at most about 10⁻³s⁻¹; at most about 10⁻⁴s⁻¹; at most about 10⁻⁵s⁻¹; and at most about 10⁻⁶s⁻¹, as measured by surface plasmon resonance. The parent binding protein(s), e.g., antibody(s) from which the variable domains are obtained may have similar or different off rate constants (K_{off}) for the respective antigen.

### B2. Potency

The desired affinity/potency of parental binding proteins, e.g., monoclonal antibodies, will depend on the desired therapeutic outcome. For example, for receptor-ligand (R-L) interactions the affinity (k_{d}) is equal to or better than the R-L k_{d} (pM range). For simple clearance of a pathologic circulating protein, the k_{d} could be in low nM range, e.g., clearance of various species of circulating A-β peptide. In addition, the k_{d} will also depend on whether the target expresses multiple copies of the same epitope, e.g., a monoclonal antibody targeting conformational epitope in Aβ oligomers.

Where, for example, three of the variable domains of the binding proteins of the invention bind the same target antigen but distint epitopes, a TVD binding protein will contain at least three binding sites for the same antigen, thus increasing avidity and thereby the apparent kd of the TVD binding protein. In one embodiment parent binding proteins, e.g., antibodies, with equal or lower k_{d} than that desired in the TVD binding protein are chosen. The affinity considerations of a parental monoclonal binding protein(s), e.g., antibody(s), may also depend upon whether the TVD binding protein contains three or more identical antigen-binding sites (e.g., a TVD-Ig protein in which three of the variable domains (heavy and light) are obtained from a single monoclonal antibody). In this case, the apparent k_{d} would be greater than the monoclonal antibody due to avidity. Such TVD binding proteins can be employed for cross-linking surface receptor, increase neutralization potency, enhance clearance of pathological proteins, etc.

In one embodiment, parent binding proteins, e.g., antibodies, with neutralization potency for specific antigen equal to or better than the desired neutralization potential of the TVD binding protein for the same antigen are selected. The neutralization potency can be assessed by a target-dependent bioassay where cells of appropriate type produce a measurable signal (i.e. proliferation or cytokine production) in response to target stimulation, and target neutralization by the monoclonal antibody can reduce the signal in a dose-dependent manner.

### B3. Biological Functions

Binding proteins, e.g., monoclonal antibodies, can perform potentially several functions. Some of these functions are listed in Table 7. These functions can be assessed by both in vitro assays (e.g., cell-based and biochemical assays) and in vivo animal models.

TABLE 7
Some Potential Applications For Therapeutic Binding Proteins

| Target (Class) | Mechanism of Action (target) |
|---|---|
| Soluble | Neutralization of activity (e.g., a cytokine) |
| (cytokines, other) | Enhance clearance (e.g., Aβ oligomers) |
| | Increase half-life (e.g., GLP 1) |
| Cell Surface | Agonist (e.g., GLP1 R; CTLA4, TNFSR1; EPO R; |
| (Receptors, other) | etc.) |
| | Antagonist (e.g., integrins; etc.) |
| | Cytotoxic (CD 20; etc.) |
| Protein deposits | Enhance clearance/degradation (e.g., Aβ plaques, |
| | amyloid deposits) |

Binding proteins, e.g., monoclonal antibodies, with distinct functions described in the examples herein in, for example, Tables 1-6, 8, 11, 13, 14, and 15 can be selected to achieve desired therapeutic outcomes. One or more selected parent binding proteins, e.g., monoclonal antibodies, can then be used in TVD binding protein format to achieve one or more distinct functions in a single TVD binding protein. For example, a TVD binding protein can be generated by selecting one or more parent binding proteins, e.g., monoclonal antibodies, that neutralize function of a specific cytokine, and selecting one or more parent binding proteins, e.g., monoclonal antibodies, that enhance clearance of a pathological protein. Similarly, two parent binding proteins, e.g., monoclonal antibodies, that recognize two different cell surface receptors can be selected, e.g., one mAb with an agonist function on one receptor and the other mAb with an antagonist function on a different receptor. These two selected monoclonal antibodies, each with a distinct function, can be used to construct a single TVD binding protein that will possess the two distinct functions (agonist and antagonist) of the selected monoclonal antibodies in a single molecule. Similarly, two antagonistic binding proteins, e.g., monoclonal antibodies, to cell surface receptors, each blocking binding of respective receptor ligands (e.g., EGF and IGF), can be used in a TVD binding protein format. Conversely, an antagonistic anti-receptor mAb (e.g., anti-EGFR) and a neutralizing anti-soluble mediator (e.g., anti-IGF1/2) mAb can be selected to make a TVD binding protein.

TVD binding proteins may also be generated by selecting one parent binding protein, e.g., monoclonal antibody, that neutralizes function of a specific cytokine, selecting a parent binding protein, e.g., monoclonal antibody, that enhances clearance of a pathological protein, and a third parent binding protein, e.g., monoclonal antibody, that is selectively cytotoxic. Similarly, three parent binding proteins, e.g., monoclonal antibodies, that recognize three different cell surface receptors can be selected, e.g., one monoclonal antibody with an agonist function on one receptor, one monoclonal antibody with an antagonist function on a different receptor, and one monoclonal antibody that enhances clearance of a pathological protein. These three selected binding proteins, each with a distinct function, can be used to construct a single TVD binding protein that will possess the three distinct functions (agonist and antagonist) of the selected binding proteins in a single molecule. Similarly, three antagonistic binding proteins, e.g., monoclonal antibodies, to cell surface receptors, each blocking binding of respective receptor ligands (e.g., EGF, IGF, and PDGF), can be used in a TVD binding protein format. Additionally, an antagonistic anti-receptor binding protein, e.g., monoclonal antibody (e.g., anti-EGFR), a first neutralizing anti-soluble mediator (e.g., anti-IGF1) binding protein, e.g., monoclonal antibody, and a second neutralizing anti-soluble mediator (e.g., anti-IGF2) can be selected to make a TVD binding protein.

### B4. Epitope Recognition

Different regions of proteins may perform different functions. For example, specific regions of a cytokine interact with the cytokine receptor to bring about receptor activation, whereas other regions of the protein may be required for stabilizing the cytokine. In this instance one may select one or more binding proteins, e.g., monoclonal antibodies, that bind specifically to the receptor interacting region(s) on the cytokine and thereby block cytokine-receptor interaction. In some cases, for example, certain chemokine receptors that bind multiple ligands, a binding protein, e.g., monoclonal antibody, that binds to the epitope (region on chemokine receptor) that interacts with only one ligand can be selected. In other embodiments, a binding protein, e.g., a monoclonal antibody, that binds to an epitope on, for example, a chemokine receptor, that interacts with more than one ligand can be selected. In other instances, binding proteins, e.g., monoclonal antibodies, can bind to epitopes on a target that are not directly responsible for physiological functions of the protein, but binding of a monoclonal antibody to these regions could either interfere with physiological functions (steric hindrance) or alter the conformation of the protein such that the protein cannot function (monoclonal antibody to receptors with multiple ligand which alter the receptor conformation such that none of the ligand can bind). Anti-cytokine binding proteins, e.g., monoclonal antibodies, that do not block binding of the cytokine to its receptor, but block signal transduction, have also been identified (e.g., 125-2H, an anti-IL-18 monoclonal antibody).

Examples of epitopes and binding protein, e.g., monoclonal antibody, functions include, but are not limited to, blocking Receptor-Ligand (R-L) interaction (neutralizing monoclonal antibody that binds R-interacting site); e.g., steric hindrance resulting in diminished or no R-binding. An antibody can bind the target at a site other than a receptor binding site, but still interfere with receptor binding and functions of the target by inducing conformational change and eliminating function (e.g., Xolair), e.g., binding to R but blocking signaling (125-2H).

In one embodiment, the parental monoclonal binding protein, e.g., antibody, needs to target the appropriate epitope for maximum efficacy. Such epitope should be conserved in the TVD binding protein. The binding epitope of a binding protein, e.g., monoclonal antibody, can be determined by several approaches, including co-crystallography, limited proteolysis of monoclonal antibody-antigen complex plus mass spectrometric peptide mapping (Legros, V. et al. (2000) Protein Sci. 9: 1002-10), phage displayed peptide libraries (O'Connor, K. H. et al. (2005) J. Immunol. Methods 299: 21-35), as well as mutagenesis (Wu C. et al. (2003) J. Immunol. 170:5571-7).

### B5. Physicochemical and Pharmaceutical Properties

Therapeutic treatment with binding proteins, e.g., antibodies, often requires administration of high doses, often several mg/kg (due to a low potency on a mass basis as a consequence of a typically large molecular weight). In order to accommodate patient compliance and to address adequately chronic disease therapies and outpatient treatment, subcutaneous (s.c.) or intramuscular (i.m.) administration of therapeutic binding proteins, e.g., monoclonal antibodies, is desirable. For example, the maximum desirable volume for s.c. administration is ˜1.0 mL, and therefore, concentrations of >100 mg/mL are desirable to limit the number of injections per dose. In one embodiment, the therapeutic binding protein, e.g., antibody, is administered in one dose. The development of such formulations is constrained, however, by protein-protein interactions (e.g., aggregation, which potentially increases immunogenicity risks) and by limitations during processing and delivery (e.g., viscosity). Consequently, the large quantities required for clinical efficacy and the associated development constraints limit full exploitation of the potential of antibody formulation and s.c. administration in high-dose regimens. It is apparent that the physicochemical and pharmaceutical properties of a protein molecule and the protein solution are of utmost importance, e.g., stability, solubility and viscosity features.

### B5.1. Stability

A “stable” binding protein, e.g., antibody, formulation is one in which the binding protein, e.g., antibody, therein essentially retains its physical stability and/or chemical stability and/or biological activity upon storage. Stability can be measured at a selected temperature for a selected time period. In one embodiment the binding protein, e.g., antibody, in the formulation is stable at room temperature (about 30° C.) or at 40° C. for at least 1 month and/or stable at about 2-8° C. for at least 1 year, such as for at least 2 years. Furthermore, in one embodiment the formulation is stable following freezing (to, e.g., −70° C.) and thawing of the formulation, hereinafter referred to as a “freeze/thaw cycle.” In another example, a “stable” formulation may be one wherein less than about 10% and less than about 5% of the protein is present as an aggregate in the formulation.

A TVD binding protein that is stable in vitro at various temperatures for an extended time period is desirable. One can achieve this by rapid screening of parental binding proteins, e.g., monoclonal antibodies, that are stable in vitro at elevated temperature, e.g., at 40° C. for 2-4 weeks, and then assess stability. During storage at 2-8° C., the protein reveals stability for at least 12 months, e.g., at least 24 months. Stability (% of monomeric, intact molecule) can be assessed using various techniques, such as cation exchange chromatography, size exclusion chromatography, SDS-PAGE, as well as bioactivity testing. For a more comprehensive list of analytical techniques that may be employed to analyze covalent and conformational modifications please see Jones, A. J. S. (1993) Analytical methods for the assessment of protein formulations and delivery systems. In: Formulation and delivery of peptides and proteins, Cleland and Langer, eds. 1^{st} edition, ACS, Washington, pg. 22-45; and Pearlman, R. and Nguyen, T. H. (1990) Analysis of protein drugs. In: Peptide and protein drug delivery, Lee, ed. 1st edition, Marcel Dekker, Inc., New York, pg. 247-301.

Heterogeneity and aggregate formation: stability of the binding protein, e.g., antibody, may be such that the formulation may reveal less than about 10%, such as less than about 5%, such as less than about 2%, or within the range of 0.5% to 1.5% or less in the GMP binding protein, e.g., antibody, material that is present as aggregate. Size exclusion chromatography is a method that is sensitive, reproducible, and very robust in the detection of protein aggregates.

In addition to low aggregate levels, the binding protein, e.g., antibody, must, in one embodiment, be chemically stable. Chemical stability may be determined by ion exchange chromatography (e.g., cation or anion exchange chromatography), hydrophobic interaction chromatography, or other methods, such as isoelectric focusing or capillary electrophoresis. For instance, chemical stability of the binding protein, e.g., antibody, may be such that after storage of at least 12 months at 2-8° C. the peak representing unmodified antibody in a cation exchange chromatography may increase not more than 20%, such as not more than 10%, or not more than 5% as compared to the antibody solution prior to storage testing.

In one embodiment, the parent binding proteins, e.g., antibodies, display structural integrity; correct disulfide bond formation, and correct folding. Chemical instability due to changes in secondary or tertiary structure of a binding protein, e.g., an antibody, may impact antibody activity. For instance, stability, as indicated by activity of the antibody, may be such that, after storage of at least 12 months at 2-8° C., the activity of the antibody may decrease not more than 50%, such as not more than 30%, not more than 10%, or not more than 5% or 1% as compared to the antibody solution prior to storage testing. Suitable antigen-binding assays can be employed to determine antibody activity.

### B5.2. Solubility

The “solubility” of a binding protein, e.g., monoclonal antibody, correlates with the production of correctly folded, monomeric IgG. The solubility of the IgG may therefore be assessed by HPLC. For example, soluble (monomeric) IgG will give rise to a single peak on the HPLC chromatograph, whereas insoluble (e.g., multimeric and aggregated) will give rise to a plurality of peaks. A person skilled in the art will, therefore, be able to detect an increase or decrease in solubility of an IgG using routine HPLC techniques. For a more comprehensive list of analytical techniques that may be employed to analyze solubility, see Jones, A. G. Dep. Chem. Biochem. Eng., Univ. Coll. London, London, UK. Editor(s): Shamlou, P. Ayazi. Process. Solid-Liq. Suspensions (1993), 93-117. Publisher: Butterworth-Heinemann, Oxford, UK; and Pearlman and Nguyen (1990) Advances in Parenteral Sciences, 4 (Pept. Protein Drug Delivery), 247-301). Solubility of a therapeutic monoclonal antibody is critical for formulating to high concentration often required for adequate dosing. As outlined herein, solubilities of >100 mg/mL may be required to accommodate efficient antibody dosing. For instance, antibody solubility may be not less than about 5 mg/mL in early research phase, such as not less than about 25 mg/mL in advanced process science stages, such as not less than about 100 mg/mL, or not less than about 150 mg/mL. It is obvious to a person skilled in the art that the intrinsic properties of a protein molecule are important to the physico-chemical properties of the protein solution, e.g., stability, solubility, viscosity. However, a person skilled in the art will appreciate that a broad variety of excipients exist that may be used as additives to beneficially impact the characteristics of the final protein formulation. These excipients may include: (i) liquid solvents, cosolvents (e.g., alcohols, such as ethanol); (ii) buffering agents (e.g., phosphate, acetate, citrate, and amino acid buffers); (iii) sugars or sugar alcohols (e.g., sucrose, trehalose, fructose, raffinose, mannitol, sorbitol, and dextrans); (iv) surfactants (e.g., polysorbate 20, 40, 60, and 80, and poloxamers); (v) isotonicity modifiers (e.g., salts, such as NaCl, sugars, and sugar alcohols); and (vi) others (e.g., preservatives, chelating agents, antioxidants, chelating substances (e.g., EDTA), biodegradable polymers, and carrier molecules (e.g., HSA, and PEGs).

Viscosity is a parameter of high importance with regard to antibody manufacture and antibody processing (e.g., diafiltration/ultrafiltration), fill-finish processes (pumping aspects, filtration aspects) and delivery aspects (syringeability, sophisticated device delivery). Low viscosities enable the liquid solution of the binding protein, e.g., antibody, having a higher concentration. This enables the same dose may be administered in smaller volumes. Small injection volumes inhere the advantage of lower pain on injection sensations, and the solutions not necessarily have to be isotonic to reduce pain on injection in the patient. The viscosity of the binding protein, e.g., antibody, solution may be such that, at shear rates of 100 (1/s), antibody solution viscosity is below 200 mPa s, such as below 125 mPa s, such as below 70 mPa s, such as below 25 mPa s, or even below 10 mPa s.

### B 5.3. Production Efficiency

The generation of a TVD binding protein that is efficiently expressed in mammalian cells, such as Chinese hamster ovary cells (CHO) will, in one embodiment, require three parental binding proteins, e.g., monoclonal antibodies, which are, themselves, expressed efficiently in mammalian cells. The production yield from a stable mammalian line (i.e., CHO) should be above about 0.5 g/L, such as above about 1 g/L, such as in the range of from about 2-5 g/L or more (Kipriyanov, S. M and Little M. (1999) Mol. Biotechnol. 12: 173-201; Carroll, S. and Al-Rubeai, M. (2004) Expert. Opin. Biol. Ther. 4: 1821-9).

Production of binding protein, e.g., antibodies and Ig fusion proteins, in mammalian cells is influenced by several factors. Engineering of the expression vector via incorporation of strong promoters, enhancers and selection markers can maximize transcription of the gene of interest from an integrated vector copy. The identification of vector integration sites that are permissive for high levels of gene transcription can augment protein expression from a vector (Wurm et al. (2004) Nature Biotechnol. 22(11): 1393-1398). Furthermore, levels of production are affected by the ratio of antibody heavy and light chains and various steps in the process of protein assembly and secretion (Jiang et al. (2006) Biotechnol. Prog. 22(1): 313-8).

### B 6. Immunogenicity

Administration of a therapeutic binding protein, e.g., monoclonal antibody, may result in certain incidence of an immune response (i.e., the formation of endogenous antibodies directed against the therapeutic monoclonal antibody). Potential elements that might induce immunogenicity should be analyzed during selection of the parental binding proteins, e.g., monoclonal antibodies, and steps to reduce such risk can be taken to optimize the parental binding proteins, e.g., monoclonal antibodies, prior to TVD binding protein construction. Mouse-derived antibodies have been found to be highly immunogenic in patients. The generation of chimeric antibodies comprised of mouse variable and human constant regions presents a logical next step to reduce the immunogenicity of therapeutic antibodies. Alternatively, immunogenicity can be reduced by transferring murine CDR sequences into a human antibody framework (reshaping/CDR grafting/humanization), as described for a therapeutic antibody by Riechmann et al. (1988) Nature 332: 323-327. Another method is referred to as “resurfacing” or “veneering,” starting with the rodent variable light and heavy domains, only surface-accessible framework amino acids are altered to human ones, while the CDR and buried amino acids remain from the parental rodent antibody (Roguska et al. (1996) Prot. Engineer 9: 895-904). In another type of humanization, instead of grafting the entire CDRs, one technique grafts only the “specificity-determining regions” (SDRs), defined as the subset of CDR residues that are involved in binding of the antibody to its target (Kashmiri et al. (2005) Methods 36(1): 25-34). This necessitates identification of the SDRs either through analysis of available three-dimensional structures of antibody-target complexes or mutational analysis of the antibody CDR residues to determine which interact with the target. Alternatively, fully human antibodies may have reduced immunogenicity compared to murine, chimeric or humanized antibodies.

Another approach to reduce the immunogenicity of therapeutic binding protein, e.g., antibodies, is the elimination of certain specific sequences that are predicted to be immunogenic. In one approach, after a first generation biologic has been tested in humans and found to be unacceptably immunogenic, the B-cell epitopes can be mapped and then altered to avoid immune detection. Another approach uses methods to predict and remove potential T-cell epitopes. Computational methods have been developed to scan and to identify the peptide sequences of biologic therapeutics with the potential to bind to MHC proteins (Desmet et al. (2005) Proteins 58: 53-69). Alternatively a human dendritic cell-based method can be used to identify CD4⁺ T-cell epitopes in potential protein allergens (Stickler et al. (2000) J. Immunother. 23: 654-60; S. L. Morrison and J. Schlom (1990) Important Adv. Oncol. 3-18; Riechmann et al. (1988) Nature 332: 323-327; Roguska et al. (1996) Protein Engineer. 9: 895-904; Kashmiri et al. (2005) Methods 36(1): 25-34; Desmet et al. (2005) Proteins 58: 53-69; and Stickler et al. (2000) J. Immunotherapy 23: 654-60.)

### B 7. In Vivo Efficacy

To generate a TVD binding protein with desired in vivo efficacy, it is important to generate and select binding proteins, e.g., monoclonal antibodies, with similarly desired in vivo efficacy when given in combination. However, in some instances the TVD binding protein may exhibit in vivo efficacy that cannot be achieved with the combination of two or more separate binding proteins, e.g., monoclonal antibodies. For instance, a TVD binding protein may bring two or more targets in close proximity leading to an activity that cannot be achieved with the combination of two or more separate monoclonal antibodies. This is useful for treatment of, for example, an oncological disorder, when it is beneficial to specifically target tumor cells and bring immune effector cells into close proximity of the tumor to initiate and/or enhance an immune response to the tumor.

Accordingly, in one embodiment, the TVD binding proteins of the present invention bind CD3 and two different cell surface molecules present on heterogeneous cells of a tumor (e.g., a tumor having a mixture of cell types). In another embodiment, the TVD binding proteins of the present invention bind an immune cell receptor, such as NKG2D or an Fc gamma receptor and two different cell surface molecules present on heterogeneous cells of a tumor (e.g., a tumor having a mixture of cell types).

Additional desirable biological functions are described herein in section B 3. Parent binding proteins, e.g., antibodies, with characteristics desirable in the TVD binding protein may be selected based on factors such as pharmacokinetic t ½; tissue distribution; soluble versus cell surface targets; and target concentration-soluble/density-surface.

### B 8. In Vivo Tissue Distribution

To generate a TVD binding protein with desired in vivo tissue distribution, in one embodiment, parent binding proteins, e.g., monoclonal antibodies, with similar desired in vivo tissue distribution profile must be selected. In this regard, two or more of the parent binding proteins, e.g., monoclonal antibodies, can be the same antibody or different binding proteins, e.g., antibodies. Alternatively, based on the mechanism of the tri-specific targeting strategy, it may at other times not be required to select two or more parent binding proteins, e.g., monoclonal antibodies, with the similarly desired in vivo tissue distribution when given in combination (e.g., in the case of a TVD binding protein in which one binding component targets the TVD binding protein to a specific site thereby bringing a second (and/or third) binding component to the same target site). For example, one or more binding specificity of a TVD binding protein could target pancreas (islet cells) and another (one or more) specificity could bring GLP1 to the pancreas to induce insulin.

### B 9. Isotype

To generate a TVD binding protein with desired properties including, but not limited to, isotype, effector functions, and the circulating half-life, in one embodiment, one or more parent binding proteins, e.g., monoclonal antibodies, with appropriate Fc-effector functions depending on the therapeutic utility and the desired therapeutic end-point are selected. Two or more of the parent binding proteins, e.g., monoclonal antibodies, can be the same antibody or different antibodies. There are five main heavy-chain classes or isotypes, some of which have several sub-types, and these determine the effector functions of an antibody molecule. These effector functions reside in the hinge region, CH2 and CH3 domains of the antibody molecule. However, residues in other parts of an antibody molecule may have effects on effector functions as well. The hinge region Fc-effector functions include: (i) antibody-dependent cellular cytotoxicity, (ii) complement (C1q) binding, activation and complement-dependent cytotoxicity (CDC), (iii) phagocytosis/clearance of antigen-antibody complexes, and (iv) cytokine release in some instances. These Fc-effector functions of an antibody molecule are mediated through the interaction of the Fc-region with a set of class-specific cell surface receptors. Antibodies of the IgG1 isotype are most active, while IgG2 and IgG4 having minimal or no effector functions. The effector functions of the IgG antibodies are mediated through interactions with three structurally homologous cellular Fc receptor types (and sub-types) (FcgR1, FcgRII and FcgRIII). These effector functions of an IgG1 can be eliminated by mutating specific amino acid residues in the lower hinge region (e.g., L234A, L235A) that are required for FcgR and C1q binding Amino acid residues in the Fc region, in particular the CH2—CH3 domains, also determine the circulating half-life of the antibody molecule. This Fc function is mediated through the binding of the Fc-region to the neonatal Fc receptor (FcRn), which is responsible for recycling of antibody molecules from the acidic lysosomes back to the general circulation.

Whether a monoclonal antibody should have an active or an inactive isotype will depend on the desired therapeutic end-point for an antibody. Some examples of usage of isotypes and desired therapeutic outcome are listed below:
a) if the desired end-point is functional neutralization of a soluble cytokine, then an inactive isotype may be used;
b) if the desired out-come is clearance of a pathological protein, an active isotype may be used;
c) if the desired out-come is clearance of protein aggregates, an active isotype may be used;
d) if the desired outcome is to antagonize a surface receptor, an inactive isotype is used (Tysabri, IgG4; OKT3, mutated IgG1);
e) if the desired outcome is to eliminate target cells, an active isotype is used (Herceptin, IgG1 (and with enhanced effector functions); and
f) if the desired outcome is to clear proteins from circulation without entering the CNS, an IgM isotype may be used (e.g., clearing circulating Ab peptide species).
   The Fc effector functions of a parental binding protein, e.g., monoclonal antibody, can be determined by various in vitro methods well known in the art.

As discussed, the selection of isotype, and thereby the effector functions will depend upon the desired therapeutic end-point. In cases where simple neutralization of a circulating target is desired, for example, blocking receptor-ligand interactions, the effector functions may not be required. In such instances isotypes or mutations in the Fc-region of an antibody that eliminate effector functions are desirable. In other instances, where elimination of target cells is the therapeutic end-point, for example, elimination of tumor cells, isotypes or mutations or de-fucosylation in the Fc-region that enhance effector functions are desirable (Presta, G. L. (2006) Adv. Drug Deliv. Rev. 58:640-656 and Satoh, M. et al. (2006) Expert Opin. Biol. Ther. 6: 1161-1173) Similarly, depending up on the therapeutic utility, the circulating half-life of an antibody molecule can be reduced/prolonged by modulating antibody-FcRn interactions by introducing specific mutations in the Fc region (Dall'Acqua, W. F. et al. (2006) J. Biol. Chem. 281: 23514-23524; Petkova, S. B. (2006) et al., Internat. Immunol. 18:1759-1769; Vaccaro, C. et al. (2007) Proc. Natl. Acad. Sci. USA 103: 18709-18714).

The published information on the various residues that influence the different effector functions of a normal therapeutic monoclonal antibody may need to be confirmed for TVD binding protein. It may be possible that in a TVD binding protein format additional (different) Fc-region residues, other than those identified for the modulation of monoclonal antibody effector functions, may be important.

Overall, the decision as to which Fc-effector functions (isotype) will be critical in the final TVD binding protein format will depend upon the disease indication, therapeutic target, and desired therapeutic end-point and safety considerations. Listed below are exemplary appropriate heavy chain and light chain constant regions including, but not limited to:
IgG1—allotype: G1mz
IgG1 mutant—A234, A235
IgG2—allotype: G2m(n-)
Kappa—Km3
Lambda

Fc Receptor and C1q Studies: The possibility of unwanted antibody-dependent cell-mediated cytotoxicity (ADCC) and complement-dependent cytotoxicity (CDC) by antibody complexing to any overexpressed target on cell membranes can be abrogated by the (for example, L234A, L235A) hinge-region mutations. These substituted amino acids, present in the IgG1 hinge region of monoclonal antibody, are expected to result in diminished binding of monoclonal antibody to human Fc receptors (but not FcRn), as FcgR binding is thought to occur within overlapping sites on the IgG1 hinge region. This feature of monoclonal antibody may lead to an improved safety profile over antibodies containing a wild-type IgG. Binding of monoclonal antibody to human Fc receptors can be determined by flow cytometry experiments using cell lines (e.g., THP-1, K562) and an engineered CHO cell line that expresses FcgRIIb (or other FcgRs). Compared to IgG1 control monoclonal antibodies, monoclonal antibody show reduced binding to FcgRI and FcgRIIa, whereas binding to FcgRIIb is unaffected. The binding and activation of C1q by antigen/IgG immune complexes triggers the classical complement cascade with consequent inflammatory and/or immunoregulatory responses. The C1q binding site on IgGs has been localized to residues within the IgG hinge region. C1q binding to increasing concentrations of monoclonal antibody was assessed by C1q ELISA. The results demonstrate that monoclonal antibody is unable to bind to C1q, as expected when compared to the binding of a wildtype control IgG1. Overall, the L234A, L235A hinge region mutation abolishes binding of monoclonal antibody to FcgRI, FcgRIIa and C1q but does not impact the interaction of monoclonal antibody with FcgRIIb. These data suggest that in vivo monoclonal antibody with mutant Fc will interact normally with the inhibitory FcgRIIb but will likely fail to interact with the activating FcgRI and FcgRIIa receptors or C1q.

Human FcRn binding: The neonatal receptor (FcRn) is responsible for transport of IgG across the placenta and to control the catabolic half-life of the IgG molecules. It might be desirable to increase the terminal half-life of an antibody to improve efficacy, to reduce the dose or frequency of administration, or to improve localization to the target. Alternatively, it might be advantageous to do the converse, that is, to decrease the terminal half-life of an antibody to reduce whole body exposure or to improve the target-to-non-target binding ratios. Tailoring the interaction between IgG and its salvage receptor, FcRn, offers a way to increase or decrease the terminal half-life of IgG. Proteins in the circulation, including IgG, are taken up in the fluid phase through micropinocytosis by certain cells, such as those of the vascular endothelia. IgG can bind FcRn in endosomes under slightly acidic conditions (pH 6.0-6.5) and can recycle to the cell surface, where it is released under almost neutral conditions (pH 7.0-7.4). Mapping of the Fc-region-binding site on FcRn80, 16, 17 showed that two histidine residues that are conserved across species, His310 and His435, are responsible for the pH dependence of this interaction. Using phage-display technology, a mouse Fc-region mutation that increases binding to FcRn and extends the half-life of mouse IgG was identified (see Victor, G. et al. (1997) Nature Biotechnol. 15(7): 637-640). Fc-region mutations that increase the binding affinity of human IgG for FcRn at pH 6.0, but not at pH 7.4, have also been identified (see Dall'Acqua, William F., et al. (2002) J. Immunol. 169(9): 5171-80). Moreover, in one case, a similar pH-dependent increase in binding (up to 27-fold) was also observed for rhesus FcRn, and this resulted in a twofold increase in serum half-life in rhesus monkeys compared with the parent IgG (see Hinton, Paul R. et al. (2004) J. Biol. Chem. 279(8): 6213-6216). These findings indicate that it is feasible to extend the plasma half-life of antibody therapeutics by tailoring the interaction of the Fc region with FcRn. Conversely, Fc-region mutations that attenuate interaction with FcRn can reduce antibody half-life.

### B.10 Pharmacokinetics (PK)

To generate a TVD binding protein with desired pharmacokinetic profile, in one embodiment, two or more parent binding proteins, e.g., monoclonal antibodies, with the similarly desired pharmacokinetic profile are selected. One consideration is that immunogenic response to binding proteins, e.g., monoclonal antibodies (i.e., HAHA, human anti-human antibody response; HACA, human anti-chimeric antibody response), further complicates the pharmacokinetics of these therapeutic agents. In one embodiment, binding proteins, e.g., monoclonal antibodies, with minimal or no immunogenicity are used for constructing TVD binding proteins, such that the resulting TVD binding proteins will also have minimal or no immunogenicity. Some of the factors that determine the PK of a monoclonal antibody include, but are not limited to, intrinsic properties of the monoclonal antibody (VH amino acid sequence), immunogenicity, FcRn binding, and Fc functions.

The PK profile of selected parental binding proteins, e.g., monoclonal antibodies, can be easily determined in rodents as the PK profile in rodents correlates well with (or closely predicts) the PK profile of monoclonal antibodies in cynomolgus monkey and humans. The PK profile may be determined using methods routin to one of ordinary skill in the art.

After the parental binding proteins, e.g., monoclonal antibodies, with desired PK characteristics (and other desired functional properties as discussed herein) are selected, the TVD binding protein is constructed. As the TVD binding protein contains three or more antigen-binding domains from one or more parental binding proteins, e.g., monoclonal antibodies, the PK properties of the TVD-Ig proteins are assessed as well. Therefore, while determining the PK properties of the TVD binding protein, PK assays may be employed that determine the PK profile based on functionality of three antigen-binding domains derived from the one or more parent binding proteins, e.g., monoclonal antibodies. Additional factors that may impact the PK profile of TVD binding proteins include the antigen-binding domain (CDR) orientation, linker size, and Fc/FcRn interactions. PK characteristics of parent binding proteins, e.g., antibodies, can be evaluated by assessing the following parameters: absorption, distribution, metabolism, and excretion.

Absorption: To date, administration of therapeutic monoclonal antibodies is via parenteral routes (e.g., intravenous (IV), subcutaneous (SC), or intramuscular (IM)). Absorption of a binding protein, e.g., monoclonal antibody, into the systemic circulation following either SC or IM administration from the interstitial space is primarily through the lymphatic pathway. Saturable, presystemic, proteolytic degradation may result in variable absolute bioavailability following extravascular administration. Usually, increases in absolute bioavailability with increasing doses of monoclonal antibodies may be observed due to saturated proteolytic capacity at higher doses. The absorption process for a monoclonal antibody is usually quite slow as the lymph fluid drains slowly into the vascular system, and the duration of absorption may occur over hours to several days. The absolute bioavailability of monoclonal antibodies following SC administration generally ranges from 50% to 100%.

Distribution: Following IV administration, binding proteins, e.g., monoclonal antibodies, usually follow a biphasic serum (or plasma) concentration-time profile, beginning with a rapid distribution phase, followed by a slow elimination phase. In general, a biexponential pharmacokinetic model best describes this kind of pharmacokinetic profile. The volume of distribution in the central compartment (Vc) for a monoclonal antibody is usually equal to or slightly larger than the plasma volume (2-3 liters). A distinct biphasic pattern in serum (plasma) concentration versus time profile may not be apparent with other parenteral routes of administration, such as IM or SC, because the distribution phase of the serum (plasma) concentration-time curve is masked by the long absorption portion. Many factors, including physicochemical properties, site-specific and target-oriented receptor mediated uptake, binding capacity of tissue, and monoclonal antibody dose can influence biodistribution of a monoclonal antibody. Some of these factors can contribute to nonlinearity in biodistribution for a monoclonal antibody.

Metabolism and Excretion: Due to the molecular size, intact binding proteins, e.g., monoclonal antibodies, are not excreted into the urine via kidney. They are primarily inactivated by metabolism (e.g., catabolism). For IgG-based therapeutic monoclonal antibodies, half-lives typically ranges from hours or 1-2 days to over 20 days. The elimination of a monoclonal antibody can be affected by many factors, including, but not limited to, affinity for the FcRn receptor, immunogenicity of the monoclonal antibody, the degree of glycosylation of the monoclonal antibody, the susceptibility for the monoclonal antibody to proteolysis, and receptor-mediated elimination.

### B.11 Tissue Cross-Reactivity Pattern on Human and Tox Species:

Identical staining pattern suggests that potential human toxicity can be evaluated in tox species. Tox species are those animal in which unrelated toxicity is studied.

The individual binding proteins, e.g., antibodies, are selected to meet two criteria: (1) tissue staining appropriate for the known expression of the antibody target and (2) similar staining pattern between human and tox species tissues from the same organ.

Criterion 1: Immunizations and/or antibody selections typically employ recombinant or synthesized antigens (proteins, carbohydrates or other molecules). Binding to the natural counterpart and counterscreen against unrelated antigens are often part of the screening funnel for therapeutic antibodies. However, screening against a multitude of antigens is often unpractical. Therefore, tissue cross-reactivity studies with human tissues from all major organs serve to rule out unwanted binding of the antibody to any unrelated antigens.

Criterion 2: Comparative tissue cross reactivity studies with human and tox species tissues (cynomolgus monkey, dog, possibly rodents and others, the same 36 or 37 tissues are being tested as in the human study) help to validate the selection of a tox species. In the typical tissue cross-reactivity studies on frozen tissue sections therapeutic antibodies may demonstrate the expected binding to the known antigen and/or to a lesser degree binding to tissues based either on low level interactions (unspecific binding, low level binding to similar antigens, low level charge based interactions, etc.). In any case the most relevant toxicology animal species is the one with the highest degree of coincidence of binding to human and animal tissue.

Tissue cross-reactivity studies follow the appropriate regulatory guidelines including EC CPMP Guideline III/5271/94 “Production and quality control of monoclonal antibodies” and the 1997 U.S. FDA/CBER “Points to Consider in the Manufacture and Testing of Monoclonal Antibody Products for Human Use.” Cryosections (5 μm) of human tissues obtained at autopsy or biopsy were fixed and dried on object glass. The peroxidase staining of tissue sections was performed, using the avidin-biotin system (FDA's Guidance “Points to Consider in the Manufacture and Testing of Monoclonal Antibody Products for Human Use.

Tissue cross-reactivity studies are often done in two stages, with the first stage including cryosections of 32 tissues (typically: Adrenal Gland, Gastrointestinal Tract, Prostate, Bladder, Heart, Skeletal Muscle, Blood Cells, Kidney, Skin, Bone Marrow, Liver, Spinal Cord, Breast, Lung, Spleen, Cerebellum, Lymph Node, Testes, Cerebral Cortex, Ovary, Thymus, Colon, Pancreas, Thyroid, Endothelium, Parathyroid, Ureter, Eye, Pituitary, Uterus, Fallopian Tube and Placenta) from one human donor. In the second phase a full cross-reactivity study is performed with up to 38 tissues (including adrenal, blood, blood vessel, bone marrow, cerebellum, cerebrum, cervix, esophagus, eye, heart, kidney, large intestine, liver, lung, lymph node, breast mammary gland, ovary, oviduct, pancreas, parathyroid, peripheral nerve, pituitary, placenta, prostate, salivary gland, skin, small intestine, spinal cord, spleen, stomach, striated muscle, testis, thymus, thyroid, tonsil, ureter, urinary bladder, and uterus) from 3 unrelated adults. Studies are done typically at minimally two dose levels.

The therapeutic binding protein, e.g., antibody, (i.e., test article) and isotype matched control antibody may be biotinylated for avidin-biotin complex (ABC) detection; other detection methods may include tertiary antibody detection for a FITC (or otherwise) labeled test article, or precomplexing with a labeled anti-human IgG for an unlabeled test article.

Briefly, cryosections (about 5 μm) of human tissues obtained at autopsy or biopsy are fixed and dried on object glass. The peroxidase staining of tissue sections is performed, using the avidin-biotin system. First (in case of a precomplexing detection system), the test article is incubated with the secondary biotinylated anti-human IgG and developed into immune complex. The immune complex at the final concentrations of 2 and 10 μg/mL of test article is added onto tissue sections on object glass and then the tissue sections are reacted for 30 minutes with a avidin-biotin-peroxidase kit. Subsequently, DAB (3,3′-diaminobenzidine), a substrate for the peroxidase reaction, is applied for 4 minutes for tissue staining. Antigen-Sepharose beads are used as positive control tissue sections.

Any specific staining is judged to be either an expected (e.g., consistent with antigen expression) or unexpected reactivity based upon known expression of the target antigen in question. Any staining judged specific is scored for intensity and frequency. Antigen or serum competion or blocking studies can assist further in determining whether observed staining is specific or nonspecific.

If two selected binding proteins, e.g., antibodies, are found to meet the selection criteria—appropriate tissue staining and matching staining between human and toxicology animal specific tissue—they can be selected for TVD binding protein generation.

The tissue cross-reactivity study has to be repeated with the final TVD binding protein construct but, while these studies follow the same protocol as outline herein, they are more complex to evaluate because any binding can come from any of the parent binding proteins, e.g., antibodies, and any unexplained binding needs to be confirmed with complex antigen competition studies.

It is readily apparent that the complex undertaking of tissue crossreactivity studies with a multispecific molecule like a TVD binding protein is greatly simplified if one or more of the parental binding proteins, e.g., antibodies, are selected for (1) lack of unexpected tissue cross reactivity findings and (2) for appropriate similarity of tissue cross reactivity findings between the corresponding human and toxicology animal species tissues.

### B.12 Specificity and Selectivity

To generate a TVD binding protein with desired specificity and selectivity, one needs to generate and select parent binding proteins, e.g., monoclonal antibodies, with the similarly desired specificity and selectivity profile. In this regard, two or more of the parent binding proteins, e.g., monoclonal antibodies, can be the same antibody or different binding proteins, e.g., antibodies.

Binding studies for specificity and selectivity with a TVD binding protein can be complex due to the three or more binding sites. Briefly, binding studies using ELISA (enzyme linked immunosorbent assay), BIAcore, KinExA or other interaction studies with a TVD binding protein need to monitor the binding of one, two, three, four, five, or six antigens to the TVD binding protein. While BIAcore technology can resolve the sequential, independent binding of multiple antigens, more traditional methods, including ELISA, or more modern techniques, like KinExA, cannot. Therefore, careful characterization of each parent binding protein, e.g., antibody, is critical. After each individual binding protein, e.g., antibody, has been characterized for specificity, confirmation of specificity retention of the individual binding sites in the TVD binding protein is greatly simplified.

It is readily apparent that the complex undertaking of determining the specificity of a TVD binding protein is greatly simplified if the parental binding proteins, e.g., antibodies, are selected for specificity prior to being combined into a TVD binding protein.

Antigen-binding protein, e.g., antibody, interaction studies can take many forms, including many classical protein-protein interaction studies, ELISA, mass spectrometry, chemical cross-linking, SEC with light scattering, equilibrium dialysis, gel permeation, ultrafiltration, gel chromatography, large-zone analytical SEC, micropreparative ultracentrigugation (sedimentation equilibrium), spectroscopic methods, titration microcalorimetry, sedimentation equilibrium (in analytical ultracentrifuge), sedimentation velocity (in analytical centrifuge), and surface plasmon resonance (including BIAcore). Relevant references include “Current Protocols in Protein Science,” Coligan, J. E. et al. (eds.) Volume 3, chapters 19 and 20, published by John Wiley & Sons Inc., and “Current Protocols in Immunology,” Coligan, J. E. et al. (eds.) published by John Wiley & Sons Inc., and relevant references included therein.

Cytokine Release in Whole Blood: The interaction of binding protein, e.g., monoclonal antibody, with human blood cells can be investigated by a cytokine release assay (Wing, M. G. (1995) Therapeut. Immunol. 2(4): 183-190; “Current Protocols in Pharmacology,” Enna, S. J. et al. (eds.) published by John Wiley & Sons Inc; Madhusudan, S. (2004) Clin. Cancer Res. 10(19): 6528-6534; Cox, J. (2006) Methods 38(4): 274-282; Choi, I. (2001) Eur. J. Immunol. 31(1): 94-106). Briefly, various concentrations of binding protein, e.g., monoclonal antibody, are incubated with human whole blood for 24 hours. The concentration tested should cover a wide range including final concentrations mimicking typical blood levels in patients (including, but not limited to, 100 ng/ml-100 μg/ml). Following the incubation, supernatants and cell lysates were analyzed for the presence of various cytokines. Cytokine concentration profiles generated for monoclonal antibody were compared to profiles produced by a negative human IgG control and a positive LPS or PHA control. The cytokine profile displayed by monoclonal antibody from both cell supernatants and cell lysates was comparable to control human IgG. In one embodiment, the binding protein, e.g., monoclonal antibody, does not interact with human blood cells to release spontaneously inflammatory cytokines.

Cytokine release studies for a TVD binding protein are complex due to the three or more binding sites. Briefly, cytokine release studies as described herein measure the effect of the whole TVD binding protein on whole blood or other cell systems, but can not resolve which portion of the molecule causes cytokine release. Once cytokine release has been detected, the purity of the TVD binding protein preparation has to be ascertained, because some co-purifying cellular components can cause cytokine release on their own. If purity is not the issue, fragmentation of TVD binding protein (including, but not limited to, removal of Fc portion, separation of binding sites, etc.), binding site mutagenesis or other methods may need to be employed to deconvolute any observations. It is readily apparent that this complex undertaking is greatly simplified if the parental binding proteins, e.g., antibodies, are selected for lack of cytokine release prior to being combined into a TVD binding protein.

### B.13 Cross Reactivity to Other Species for Toxicological Studies

In one embodiment, the individual binding proteins, e.g., antibodies, are selected with sufficient cross-reactivity to appropriate tox species, for example, cynomolgus monkey. Parental binding proteins, e.g., antibodies, need to bind to orthologous species target (i.e., cynomolgus monkey) and elicit appropriate response (modulation, neutralization, activation). In one embodiment, the cross-reactivity (affinity/potency) to orthologous species target should be within 10-fold of the human target. In practice, the parental binding proteins, e.g., antibodies, are evaluated for multiple species, including mouse, rat, dog, monkey (and other non-human primates), as well as disease model species (i.e., sheep for asthma model). The acceptable cross-reactivity to tox species from the parental binding proteins, e.g., monoclonal antibodies, allows future toxicology studies of TVD binding proteins in the same species. For that reason, the parental binding proteins, e.g., monoclonal antibodies, should have acceptable cross-reactivity for a common tox species, thereby allowing toxicology studies of TVD binding protein in the same species.

Parent binding proteins, e.g., monoclonal antibodies, may be selected from various monoclonal antibodies that can bind specific targets and are well known in the art. The parent binding proteins, e.g., antibodies, can be the same or different. These include, but are not limited to anti-PGE2 antibody, anti-TNF antibody (U.S. Pat. No. 6,258,562), anti-IL-12 and/or anti-IL-12p40 antibody (U.S. Pat. No. 6,914,128); anti-IL-18 antibody (U.S. Patent Publication No. 2005/0147610), anti-05, anti-CBL, anti-CD147, anti-gp120, anti-VLA-4, anti-CD11a, anti-CD18, anti-VEGF, anti-CD40L, anti CD-40 (e.g., see PCT Publication No. WO 2007/124299) anti-Id, anti-ICAM-1, anti-CXCL13, anti-CD2, anti-EGFR, anti-TGF-beta 2, anti-HGF, anti-cMet, anti DLL-4, anti-NPR1, anti-PLGF, anti-ErbB3, anti-E-selectin, anti-Fact VII, anti-Her2/neu, anti-F gp, anti-CD11/18, anti-CD14, anti-ICAM-3, anti-RON, anti-SOST, anti CD-19, anti-CD80 (e.g., see PCT Publication No. WO 2003/039486, anti-CD4, anti-CD3, anti-CD23, anti-beta2-integrin, anti-alpha4beta7, anti-CD52, anti-HLA DR, anti-CD22 (e.g., see U.S. Pat. No. 5,789,554), anti-CD20, anti-MIF, anti-CD64 (FcR), anti-TCR alpha beta, anti-CD2, anti-Hep B, anti-CA 125, anti-EpCAM, anti-gp120, anti-CMV, anti-gpIIbIIIa, anti-IgE, anti-CD25, anti-CD33, anti-HLA, anti-IGF1,2, anti IGFR, anti-VNRintegrin, anti-IL-1alpha, anti-IL-1beta, anti-IL-1 receptor, anti-IL-2 receptor, anti-IL-4, anti-IL-4 receptor, anti-IL5, anti-IL-5 receptor, anti-IL-6, anti-IL-8, anti-IL-9, anti-IL-13, anti-IL-13 receptor, anti-IL-17, and anti-IL-23 (see Presta, L. G. (2005) J. Allergy Clin. Immunol. 116: 731-6 and www.path.cam.ac.uk/˜mrc7/humanisation/antibodies.html).

Parent binding proteins, e.g., monoclonal antibodies, may also be selected from various therapeutic antibodies approved for use, in clinical trials, or in development for clinical use. Such therapeutic antibodies include, but are not limited to, rituximonoclonal antibody (Rituxan®, IDEC/Genentech/Roche) (see, for example, U.S. Pat. No. 5,736,137), a chimeric anti-CD20 antibody approved to treat Non-Hodgkin's lymphoma; HuMax-CD20, an anti-CD20 currently being developed by Genmonoclonal antibody, an anti-CD20 antibody described in U.S. Pat. No. 5,500,362, AME-133 (Applied Molecular Evolution), hA20 (Immunomedics, Inc.), HumaLYM (Intracel), and PRO70769 (PCT Application No. PCT/US2003/040426), trastuzumonoclonal antibody (Herceptin®, Genentech) (see, for example, U.S. Pat. No. 5,677,171), a humanized anti-Her2/neu antibody approved to treat breast cancer; pertuzumonoclonal antibody (rhuMonoclonal antibody-2C4, Omnitarg®), currently being developed by Genentech; an anti-Her2 antibody (U.S. Pat. No. 4,753,894; cetuximonoclonal antibody (Erbitux®, Imclone) (U.S. Pat. No. 4,943,533; PCT Publication No. WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Pat. No. 7,247,301), currently being developed by Genmonoclonal antibody; 425, EMD55900, EMD62000, and EMD72000 (Merck KGaA) (U.S. Pat. No. 5,558,864; Murthy, et al. (1987) Arch. Biochem. Biophys. 252(2): 549-60; Rodeck, et al. (1987) J. Cell. Biochem. 35(4): 315-20; Kettleborough, et al. (1991) Protein Eng. 4(7): 773-83); ICR62 (Institute of Cancer Research) (PCT Publication No. WO 95/20045; Modjtahedi, et al. (1993) J. Cell. Biophys. 22(1-3): 129-46; Modjtahedi, et al. (1993) Br. J. Cancer 67(2): 247-53; Modjtahedi, et al. (1996) Br. J. Cancer 73(2): 228-35; Modjtahedi, et al. (2003) Int. J. Cancer 105(2): 273-80); TheraCIM hR3 (YM Biosciences, Canada and Centro de Immunologia Molecular, Cuba (U.S. Pat. No. 5,891,996; U.S. Pat. No. 6,506,883; Mateo, et al. (1997) Immunotechnol. 3(1): 71-81); monoclonal antibody-806 (Ludwig Institue for Cancer Research, Memorial Sloan-Kettering) (Jungbluth, et al. (2003) Proc. Natl. Acad. Sci. USA. 100(2): 639-44); KSB-102 (KS Biomedix); MR1-1 (IVAX, National Cancer Institute) (PCT Publication No. WO 01/62931A2); and SC100 (Scancell) (PCT Publication No. WO 01/88138); alemtuzumonoclonal antibody (Campath®, Millenium), a humanized monoclonal antibody currently approved for treatment of B-cell chronic lymphocytic leukemia; muromonab-CD3 (Orthoclone OKT3®), an anti-CD3 antibody developed by Ortho Biotech/Johnson & Johnson, ibritumomonoclonal antibody tiuxetan (Zevalin®), an anti-CD20 antibody developed by IDEC/Schering AG, gemtuzumonoclonal antibody ozogamicin (Mylotarg®), an anti-CD33 (p67 protein) antibody developed by Celltech/Wyeth, alefacept (Amevive®), an anti-LFA-3 Fc fusion developed by Biogen), abciximonoclonal antibody (ReoPro®), developed by Centocor/Lilly, basiliximonoclonal antibody (Simulect®), developed by Novartis, palivizumonoclonal antibody (Synagis®), developed by Medimmune, infliximonoclonal antibody (Remicade®), an anti-TNFalpha antibody developed by Centocor, adalimumonoclonal antibody (Humira®), an anti-TNFalpha antibody developed by Abbott, Humicade®, an anti-TNFalpha antibody developed by Celltech, golimumonoclonal antibody (CNTO-148), a fully human TNF antibody developed by Centocor, etanercept (Enbrel®), an p75 TNF receptor Fc fusion developed by Immunex/Amgen, lenercept, an p55TNF receptor Fc fusion previously developed by Roche, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomonoclonal antibody (R1549, 90Y-muHMFG1), an anti-MUC1 in development by Antisoma, Therex (R1550), an anti-MUC1 antibody being developed by Antisoma, AngioMonoclonal antibody (AS1405), being developed by Antisoma, HuBC-1, being developed by Antisoma, Thioplatin (AS1407) being developed by Antisoma, Antegren® (natalizumonoclonal antibody), an anti-alpha-4-beta-1 (VLA-4) and alpha-4-beta-7 antibody being developed by Biogen, VLA-1 monoclonal antibody, an anti-VLA-1 integrin antibody being developed by Biogen, LTBR monoclonal antibody, an anti-lymphotoxin beta receptor (LTBR) antibody being developed by Biogen, CAT-152, an anti-TGF-β2 antibody being developed by Cambridge Antibody Technology, ABT 874 (J695), an anti-IL-12 p40 antibody being developed by Abbott, CAT-192, an anti-TGFβ1 antibody being developed by Cambridge Antibody Technology and Genzyme, CAT-213, an anti-Eotaxin1 antibody being developed by Cambridge Antibody Technology, LymphoStat-B® an anti-Blys antibody being developed by Cambridge Antibody Technology and Human Genome Sciences Inc., TRAIL-Rlmonoclonal antibody, an anti-TRAIL-R1 antibody being developed by Cambridge Antibody Technology and Human Genome Sciences, Inc., Avastin® bevacizumonoclonal antibody, rhuMonoclonal antibody-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair® (Omalizumonoclonal antibody), an anti-IgE antibody being developed by Genentech, Raptiva® (Efalizumonoclonal antibody), an anti-CD11a antibody being developed by Genentech and Xoma, MLN-02 Antibody (formerly LDP-O₂), being developed by Genentech and Millenium Pharmaceuticals, HuMax CD4, an anti-CD4 antibody being developed by Genmonoclonal antibody, HuMax-IL15, an anti-IL15 antibody being developed by Genmonoclonal antibody and Amgen, HuMax-Inflam, being developed by Genmonoclonal antibody and Medarex, HuMax-Cancer, an anti-Heparanase I antibody being developed by Genmonoclonal antibody and Medarex and Oxford GcoSciences, HuMax-Lymphoma, being developed by Genmonoclonal antibody and Amgen, HuMax-TAC, being developed by Genmonoclonal antibody, IDEC-131, and anti-CD40L antibody being developed by IDEC Pharmaceuticals, IDEC-151 (Clenoliximonoclonal antibody), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumonoclonal antibody), an anti-carcinoembryonic antigen (CEA) antibody being developed by Immunomedics, LymphoCide® (Epratuzumonoclonal antibody), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-1), and anti-Her2 antibody being developed by Medarex and Immuno-Designed Molecules, HuMax®-CD4, an anti-CD4 antibody being developed by Medarex and Genmonoclonal antibody, HuMax-IL15, an anti-IL15 antibody being developed by Medarex and Genmonoclonal antibody, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumonoclonal antibody), an anti-CD3 antibody being developed by Protein Design Labs, HuZAF®, an anti-gamma interferon antibody being developed by Protein Design Labs, Anti-α 5β1 Integrin, being developed by Protein Design Labs, anti-IL-12, being developed by Protein Design Labs, ING-1, an anti-Ep-CAM antibody being developed by Xoma, Xolair® (Omalizumonoclonal antibody) a humanized anti-IgE antibody developed by Genentech and Novartis, and MLN01, an anti-Beta2 integrin antibody being developed by Xoma. In another embodiment, the therapeutics include KRN330 (Kirin); huA33 antibody (A33, Ludwig Institute for Cancer Research); CNTO 95 (alpha V integrins, Centocor); MEDI-522 (alpha Vβ3 integrin, Medimmune); volociximonoclonal antibody (alpha Vβ1 integrin, Biogen/PDL); Human monoclonal antibody 216 (B cell glycosolated epitope, NCI); BiTE MT103 (bispecific CD19×CD3, Medimmune); 4G7xH22 (Bispecific BcellxFcgammaR1, Medarex/Merck KGa); rM28 (Bispecific CD28×MAPG, EP Patent No. EP1444268); MDX447 (EMD 82633) (Bispecific CD64×EGFR, Medarex); Catumaxomonoclonal antibody (removab) (Bispecific EpCAM×anti-CD3, Trion/Fres); Ertumaxomonoclonal antibody (bispecific HER2/CD3, Fresenius Biotech); oregovomonoclonal antibody (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumonoclonal antibody (MEDI-507) (CD2, Medimmune); Ofatumumonoclonal antibody (Humax-CD20) (CD20, Genmonoclonal antibody); Rituximonoclonal antibody (Rituxan) (CD20, Genentech); veltuzumonoclonal antibody (hA20) (CD20, Immunomedics); Epratuzumonoclonal antibody (CD22, Amgen); lumiliximonoclonal antibody (IDEC 152) (CD23, Biogen); muromonab-CD3 (CD3, Ortho); HuM291 (CD3 fc receptor, PDL Biopharma); HeFi-1, CD30, NCI); MDX-060 (CD30, Medarex); MDX-1401 (CD30, Medarex); SGN-30 (CD30, Seattle Genentics); SGN-33 (Lintuzumonoclonal antibody) (CD33, Seattle Genentics); Zanolimumonoclonal antibody (HuMax-CD4) (CD4, Genmonoclonal antibody); HCD122 (CD40, Novartis); SGN-40 (CD40, Seattle Genentics); Campathlh (Alemtuzumonoclonal antibody) (CD52, Genzyme); MDX-1411 (CD70, Medarex); hLL1 (EPB-1) (CD74.38, Immunomedics); Galiximonoclonal antibody (IDEC-144) (CD80, Biogen); MT293 (TRC093/D93) (cleaved collagen, Tracon); HuLuc63 (CS1, PDL Pharma); ipilimumonoclonal antibody (MDX-010) (CTLA4, Brystol Myers Squibb); Tremelimumonoclonal antibody (Ticilimumonoclonal antibody, CP-675,2) (CTLA4, Pfizer); HGS-ETR1 (Mapatumumonoclonal antibody) (DR4TRAIL-R1 agonist, Human Genome Science/Glaxo Smith Kline); AMG-655 (DR5, Amgen); Apomonoclonal antibody (DR5, Genentech); CS-1008 (DR5, Daiichi Sankyo); HGS-ETR2 (lexatumumonoclonal antibody) (DR5TRAIL-R2 agonist, HGS); Cetuximonoclonal antibody (Erbitux) (EGFR, Imclone); IMC-11F8, (EGFR, Imclone); Nimotuzumonoclonal antibody (EGFR, YM Bio); Panitumumonoclonal antibody (Vectabix) (EGFR, Amgen); Zalutumumonoclonal antibody (HuMaxEGFr) (EGFR, Genmonoclonal antibody); CDX-110 (EGFRvIII, AVANT Immunotherapeutics); adecatumumonoclonal antibody (MT201) (Epcam, Merck); edrecolomonoclonal antibody (Panorex, 17-1A) (Epcam™, Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumonoclonal antibody (Herceptin) (HER2, Celldex); Pertuzumonoclonal antibody (rhuMonoclonal antibody 2C4) (HER2 (DI), Genentech); apolizumonoclonal antibody (HLA-DR beta chain, PDL Pharma); AMG-479 (IGF-1R, Amgen); anti-IGF-1R R1507 (IGF1-R, Roche); CP 751871 (IGF1-R, Pfizer); IMC-A12 (IGF1-R, Imclone); BIIB022 (IGF-1R, Biogen); Mik-beta-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTβR, Biogen); HuHMFG1 (MUC1, Antisoma/NCI); RAV12 (N-linked carbohydrate epitope, Raven); CAL (parathyroid hormone-related protein (PTH-rP), University of California); CT-011 (PD1, CureTech); MDX-1106 (ono-4538) (PD1, Medarex/Ono); Monoclonal antibody CT-011 (PD1, Curetech); IMC-3G3 (PDGFRa, Imclone); bavituximonoclonal antibody (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximonoclonal antibody (Remicade) (TNFa, Centocor); A27.15 (transferrin receptor, Salk Institute, INSERN WO 2005/111082); E2.3 (transferrin receptor, Salk Institute); Bevacizumonoclonal antibody (Avastin) (VEGF, Genentech); HuMV833 (VEGF, Tsukuba Research Lab, PCT Publication No. WO/2000/034337, University of Texas); IMC-18F1 (VEGFR1, Imclone); IMC-1121 (VEGFR2, Imclone).

### C. Construction of TVD Binding Proteins

The tri-variable domain binding protein is designed such that three different light chain variable domains (VD_{L}) from three parent binding proteins, e.g., monoclonal antibodies, which can be the same or different, are linked in tandem directly or via a short linker by recombinant DNA techniques, followed by the light chain constant domain, and optionally, an Fc region. Similarly, the heavy chain comprises three different heavy chain variable domains (VD_{H}) linked in tandem, followed by a constant domain and Fc region (FIG. 1).

In certain embodiments, together one or more of the heavy and light variable domains in the first and second polypeptides (or second and fourth polypeptide chains) are complementary variable domains and form a single functional antigen binding site. In certain embodiments, the variable domains form complete, independent antigen binding sites on each polypeptide chain. For example, when each of the three heavy chain antigen binding domains are independently selected from domain antibody, rececptor, and scFv, three complete, independent antigen binding sites are present on the polypeptide chain.

The variable domains can be obtained using recombinant DNA techniques from one or more parent binding proteins, e.g., antibodies, generated by any one of the methods described herein. In one embodiment, the variable domain is a murine heavy or light chain variable domain. In another embodiment, the variable domain is a CDR grafted or a humanized variable heavy or light chain domain. In one embodiment, the variable domain is a human heavy or light chain variable domain.

In one embodiment, the first and second variable domains are linked directly to each other using recombinant DNA techniques. In another embodiment, the second and third variable domains are linked directly to each other using recombinant DNA techniques. In another embodiment, the first, the second, and the third variable domains are linked directly to each other using recombinant DNA techniques. In one embodiment the first and second variable domains are linked via a linker sequence. In another embodiment the second and third variable domains are linked via a linker sequence. In another embodiment the first, second, and third variable domains are linked via a linker sequence. The variable domains may bind the same antigen or may bind different antigens. TVD binding proteins of the present disclosure may include an immunoglobulin variable domain and/or a non-immunoglobulin variable domain, such as a ligand binding domain of a receptor or an active domain of an enzyme. TVD binding proteins may also comprise three or more non-Ig domains.

The linker sequence may be a single amino acid or a polypeptide sequence. In one embodiment, the linker sequences are selected from the group consisting of AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9), SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); GHEAAAVMQVQYPAS (SEQ ID NO: 26); TVAAPSVFIFPPTVAAPSVFIFPP (SEQ ID NO: 27); and ASTKGPSVFPLAPASTKGPSVFPLAP (SEQ ID NO: 28).

The choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of CL/CH1 domain. TVD binding proteins of the present disclosure were generated using N-terminal 5-6 amino acid residues, or 11-12 amino acid residues, of CL or CH1 as linker in light chain and heavy chain of TVD binding protein, respectively. The N-terminal residues of the CL or CH1 domain, particularly the first 5-6 amino acid residues, adopt a loop conformation without strong secondary structure, and, therefore, can act as a flexible linker between the two variable domains. The N-terminal residues of the CL or CH1 domain are a natural extension of the variable domains, as they are part of the Ig sequences, and, therefore, minimize to a large extent any immunogenicity potentially arising from the linkers and junctions.

Other linker sequences may include any sequence of any length of the CL/CH1 domain but not all residues of the CL/CH1 domain (for example, the first 5-12 amino acid residues of the CL/CH1 domains); the light chain linkers can be from Cκ or Cλ; and the heavy chain linkers can be derived from CH1 of any isotypes, including Cγ1, Cγ2, Cγ3, Cγ4, Cα1, Cα2, Cδ, Cε, and Cμ. Linker sequences may also be derived from other proteins, such as Ig-like proteins (e.g., TCR, FcR, KIR); G/S based sequences (e.g., G4S repeats (SEQ ID NO:204)); hinge region-derived sequences; and other natural sequences from other proteins.

In one embodiment, a constant domain is linked to the three linked variable domains using recombinant DNA techniques. In one embodiment, sequence comprising linked heavy chain variable domains is linked to a heavy chain constant domain and sequence comprising linked light chain variable domains is linked to a light chain constant domain. In one embodiment, the constant domains are human heavy chain constant domain and human light chain constant domain, respectively. In one embodiment, the TVD molecule heavy chain is further linked to an Fc region. The Fc region may be a native sequence Fc region, or a variant Fc region. In another embodiment, the Fc region is a human Fc region. In another embodiment the Fc region includes Fc region from IgG1, IgG2, IgG3, IgG4, IgA, IgM, IgE, or IgD.

In another embodiment, two heavy chain TVD polypeptides and two light chain TVD polypeptides are combined to form a TVD-Ig protein. Tables 1-6, 8, 11, 13, 14, and 15 list amino acid sequences of VH and VL regions of exemplary binding proteins, e.g., antibodies, for targets useful for treating disease, e.g., for treating an inflammatory disease or disorder. In one embodiment, the present disclosure provides a TVD binding protein comprising three of the VH and/or VL regions listed in, for example, Tables 1-6, 8, 11, 13, 14, and 15, in any orientation. Detailed descriptions of specific TVD binding proteins that can bind specific targets, and methods of making the same, are provided in the Examples section below.

### D. Production of TVD Binding Proteins

TVD binding proteins of the present disclosure may be produced by any of a number of techniques known in the art. For example, expression from host cells, wherein expression vector(s) encoding the TVD binding protein heavy and TVD binding protein light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the TVD proteins of the present disclosure in either prokaryotic or eukaryotic host cells, TVD proteins are expressed in eukaryotic cells, for example, mammalian host cells, because such eukaryotic cells (and in particular mammalian cells) are more likely than prokaryotic cells to assemble and secrete a properly folded and immunologically active TVD protein.

Exemplary mammalian host cells for expressing the recombinant binding proteins, e.g., antibodies, of the present disclosure include Chinese Hamster Ovary (CHO cells) (including dhfr-CHO cells, described in Urlaub and Chasin (1980) Proc. Natl. Acad. Sci. USA 77: 4216-4220, used with a DHFR selectable marker, e.g., as described in Kaufman, R. J. and Sharp, P. A. (1982) Mol. Biol. 159: 601-621), NS0 myeloma cells, COS cells, SP2 and PER.C6 cells. When recombinant expression vectors encoding TVD binding proteins are introduced into mammalian host cells, the TVD proteins are produced by culturing the host cells for a period of time sufficient to allow for expression of the TVD binding proteins in the host cells or secretion of the TVD binding proteins into the culture medium in which the host cells are grown. TVD binding proteins can be recovered from the culture medium using standard protein purification methods.

In an exemplary system for recombinant expression of TVD binding proteins of the present disclosure, a recombinant expression vector encoding the TVD heavy chain and the TVD light chain is introduced into dhfr-CHO cells by calcium phosphate-mediated transfection. Within the recombinant expression vector, the TVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the TVD heavy and light chains and intact TVD binding protein is recovered from the culture medium. Standard molecular biology techniques are used to prepare the recombinant expression vector, transfect the host cells, select for transformants, culture the host cells and recover the TVD protein from the culture medium. Still further the present disclosure provides a method of synthesizing a TVD binding protein of the present disclosure by culturing a host cell of the present disclosure in a suitable culture medium until a TVD binding protein of the present disclosure is synthesized. The method can further comprise isolating the TVD binding protein from the culture medium.

An important feature of TVD binding protein is that it can be produced and purified in a similar way as a conventional antibody. The production of TVD binding protein results in a homogeneous, single major product with desired specific activity(s), without any sequence modification of the constant region or chemical modifications of any kind. Other previously described methods to generate “bi-specific,” “multi-specific,” and “multi-specific multivalent” full-length binding proteins do not lead to a single primary product but, instead, lead to the intracellular or secreted production of a mixture of assembled inactive, mono-specific, multi-specific, multivalent, fulllength binding proteins, and multivalent full-length binding proteins with combination of different binding sites. As an example, based on the design described by Miller and Presta (PCT Publication No. WO 2001/077342, there are 16 possible combinations of heavy and light chains. Consequently, only 6.25% of protein is likely to be in the desired active form, and not as a single major product or single primary product compared to the other 15 possible combinations. Separation of the desired, fully active forms of the protein from inactive and partially active forms of the protein using standard chromatography techniques, typically used in large scale manufacturing, is yet to be demonstrated.

Surprisingly, as described below, the design of the “multi- (e.g., “tri-”) specific multivalent full length binding proteins” of the present disclosure leads to a multi- (e.g., tri-) variable domain light chain and a tri-variable domain heavy chain, which assemble primarily to the desired “multi- (e.g., “tri-”) specific multivalent full-length binding proteins.”

At least 50%, at least 75% and at least 90% of the assembled, and expressed multi- (e.g., tri-) variable domain immunoglobulin molecules are the desired multi- (e.g., tri-) specific multivalent protein. This aspect particularly enhances the commercial utility of the present disclosure. Therefore, the present disclosure includes a method to express a multi- (e.g., tri-) variable domain light chain and a multi- (e.g., “tri-”) variable domain heavy chain in a single cell leading to a single primary product of a “multi- (e.g., “tri-”) specific sextavalent full length binding protein.”

The present disclosure provides a method of expressing a multi- (e.g., tri-) variable domain light chain and a multi- (e.g., tri-) variable domain heavy chain in a single cell leading to a “primary product” of a “multi- (e.g., “tri-”) specific sextavalent full length binding protein,” where the “primary product” is more than 50% of all assembled protein, comprising a multi- (e.g., tri-) variable domain light chain and a multi- (e.g., tri-) variable domain heavy chain.

The present disclosure provides a method of expressing a multi- (e.g., tri-) variable domain light chain and a multi- (e.g., tri-) variable domain heavy chain in a single cell leading to a single “primary product” of a “multi- (e.g., “tri-”) specific sextavalent full length binding protein,” where the “primary product” is more than 75% of all assembled protein, comprising a multi- (e.g., tri-) variable domain light chain and a multi- (e.g., tri-) variable domain heavy chain.

The present disclosure provides a method of expressing a multi- (e.g., tri-) variable domain light chain and a multi- (e.g., tri-) variable domain heavy chain in a single cell leading to a single “primary product” of a “multi- (e.g., “tri-”) specific sextavalent full length binding protein,” where the “primary product” is more than 90% of all assembled protein, comprising a multi- (e.g., tri-) variable domain light chain and a multi- (e.g., tri-) variable domain heavy chain.

### E. Derivatized TVD Binding Proteins

One embodiment provides a labeled binding protein wherein the binding protein of the present disclosure is derivatized or linked to another functional molecule (e.g., another peptide or protein). For example, a labeled binding protein of the present disclosure can be derived by functionally linking a binding protein of the present disclosure (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the binding protein with another molecule (such as a streptavidin core region or a polyhistidine tag).

Useful detectable agents with which a binding protein of the present disclosure may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin, and the like. A binding protein may also be derivatized with detectable enzymes, such as alkaline phosphatase, horseradish peroxidase, glucose oxidase and the like. When a binding protein is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding protein may also be derivatized with biotin, and detected through indirect measurement of avidin or streptavidin binding.

Another embodiment of the present disclosure provides a crystallized binding protein and formulations and compositions comprising such crystals. In one embodiment the crystallized binding protein has a greater half-life in vivo than the soluble counterpart of the binding protein. In another embodiment the binding protein retains biological activity after crystallization.

Crystallized binding protein of the present disclosure may be produced according to methods known in the art and as disclosed in PCT Publication No. WO 02/072636.

Another embodiment of the present disclosure provides a glycosylated binding protein wherein the binding protein, e.g., antibody, or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have an important effect on the effector function of the Fc domain, with minimal effect on antigen binding or half-life of the antibody (Jefferis, R. (2005) Biotechnol. Prog. 21:11-16). In contrast, glycosylation of the variable domain may have an effect on the antigen-binding activity of the antibody. Glycosylation in the variable domain may have a negative effect on antibody binding affinity, likely due to steric hindrance (Co, M. S. et al. (1993) Mol. Immunol. 30: 1361-1367), or result in increased affinity for the antigen (Wallick, S. C. et al. (1988) Exp. Med. 168: 1099-1109; Wright, A. et al. (1991) EMBO J. 10: 2717 2723).

One aspect of the present disclosure is directed to generating glycosylation site mutants in that the O- or N-linked glycosylation site of the binding protein has been mutated. One skilled in the art can generate such mutants using standard well-known technologies. Glycosylation site mutants that retain the biological activity, but have increased or decreased binding activity, are another object of the present disclosure.

In still another embodiment, the glycosylation of the binding protein, e.g., antibody, or antigen-binding portion of the present disclosure is modified. For example, an aglycoslated binding protein, e.g., antibody, can be made (i.e., the antibody lacks glycosylation). Glycosylation can be altered to, for example, increase the affinity of the e.g., antibody, for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in PCT Publication No. WO 2003/016466, and U.S. Pat. Nos. 5,714,350 and 6,350,861.

Additionally or alternatively, a modified binding protein of the present disclosure can be made that has an altered type of glycosylation, such as a hypofucosylated antibody having reduced amounts of fucosyl residues (see Kanda et al. (2007) J. Biotechnol. 130(3): 300-310.) or an antibody having increased bisecting GlcNAc structures. Such altered glycosylation patterns have been demonstrated to increase the ADCC ability of binding proteins, e.g., antibodies. Such carbohydrate modifications can be accomplished by, for example, expressing the binding protein, e.g., antibody, in a host cell with altered glycosylation machinery. Cells with altered glycosylation machinery have been described in the art and can be used as host cells in which to express recombinant binding proteins, e.g., antibodies, of the present disclosure to thereby produce a binding protein with altered glycosylation. See, for example, Shields, R. L. et al. (2002) J. Biol. Chem. 277: 26733-26740; Umana et al. (1999) Nat. Biotech. 17: 176-1, as well as, EU Patent No. EP 1,176,195; and PCT Publication Nos. WO 03/035835 and WO 99/54342 80.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the present disclosure may include, but are not limited to, glucose, galactose, mannose, fucose, n-acetylglucosamine and sialic acid. In one embodiment, the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may choose a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate binding proteins, e.g., antibodies, or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. Pat. Nos. 7,449,308 and 7,029,872; and PCT Publication No. WO 2005/100584).

In addition to the binding proteins, the present disclosure is also directed to anti-idiotypic (anti-Id) antibodies specific for such binding proteins of the present disclosure. An anti-Id antibody is an antibody, which recognizes unique determinants generally associated with the antigen-binding region of another antibody. The anti-Id can be prepared by immunizing an animal with the binding protein or a CDR containing region thereof. The immunized animal will recognize and respond to the idiotypic determinants of the immunizing antibody and produce an anti-Id antibody. It is readily apparent that it may be easier to generate anti-idiotypic antibodies to the multiple parent binding proteins, e.g., antibodies, incorporated into a TVD binding protein; and confirm binding studies by methods well recognized in the art (e.g., BIAcore, ELISA) to verify that anti-idiotypic antibodies specific for the idiotype of each parent antibody also recognize the idiotype (e.g., antigen-binding site) in the context of the TVD binding protein. The anti-idiotypic antibodies specific for each of the three or more antigen-binding sites of a TVD binding protein provide ideal reagents to measure TVD binding protein concentrations of a human TVD binding protein in patrient serum; TVD binding protein concentration assays can be established using a “sandwich assay ELISA format” with an antibody to a first antigen-binding region coated on the solid phase (e.g., BIAcore chip, ELISA plate etc.), rinsing with rinsing buffer, incubating with the serum sample, rinsing again, and ultimately incubating with another anti-idiotypic antibody to the another antigen-binding site, itself labeled with an enzyme for quantitation of the binding reaction. In one embodiment, for a TVD binding protein with more than three different binding sites, anti-idiotypic antibodies to the two outermost binding sites (most distal and proximal from the constant region) will not only help in determining the TVD binding protein concentration in human serum but also document the integrity of the molecule in vivo. Each anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. In one embodiment, the protein having a particularly selected novel glycosylation pattern exhibits improved or altered biological properties.

### II. Uses of TVD Binding Proteins

Given their ability to bind to multiple antigens, the binding proteins of the present disclosure can be used to detect the antigens (e.g., in a biological sample, such as serum or plasma), using a conventional assay, e.g., an immunoassay, such as an ELISA, a radioimmunoassay (RIA), or tissue immunohistochemistry. The TVD binding protein is directly or indirectly labeled with a detectable substance to facilitate detection of the bound or unbound antibody. Suitable detectable substances include various enzymes, prosthetic groups, fluorescent materials, luminescent materials, and radioactive materials. Examples of suitable enzymes include horseradish peroxidase, alkaline phosphatase, (3-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In one embodiment the binding proteins of the present disclosure can neutralize the activity of the antigens both in vitro and in vivo. Accordingly, such TVD binding proteins can be used to inhibit antigen activity, e.g., in a cell culture containing the antigens, in human subjects or in other mammalian subjects having the antigens with which a binding protein of the present disclosure cross-reacts. In another embodiment, the present disclosure provides a method for reducing antigen activity in a subject suffering from a disease or disorder in which the antigen activity is detrimental. A binding protein of the present disclosure can be administered to a human subject for therapeutic purposes.

As used herein, the term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins of the present disclosure include those disorders discussed below and in the section pertaining to pharmaceutical compositions of the binding proteins of the present disclosure.

The TVD binding proteins of the present disclosure may bind one target or multiple target antigens. Such target antigens include, but are not limited to, the targets listed in the following databases. These target databases include those listings:

Therapeutic targets (xin.cz3.nus.edu.sg/group/cjttd/ttd.asp);
Cytokines and cytokine receptors (www.cytokinewebfacts.com, www.copewithcytokines.de/cope.cgi, and cmbi.bjmu.edu.cn/cmbidata/cgf/CGF_Database/cytokine.medic.kumamoto-u.ac.jp/CFC/indexR.html);
Chemokines (cytokine.medic.kumamoto-u.ac.jp/CFC/CK/Chemokine.html);
Chemokine receptors and GPCRs (csp.medic.kumamoto-u.ac.jp/CSP/Receptor.html, and www.gper.org/7tm/);
Olfactory Receptors (senselab.med.yale.edu/senselab/ORDB/default.asp);
Receptors (www.iuphar-db.org/iuphar-rd/list/index.htm);
Cancer targets (cged.hgc.jp/cgi-bin/input.cgi);
Secreted proteins as potential targets (spd.cbi.pku.edu.cn/);
Protein kinases (spd.cbi.pku.edu.cn/), and
Human CD markers (content.labvelocity.com/tools/6/1226/CD table final locked.pdf) and (Zola H (2005) Blood 106: 3123-6).

TVD binding proteins are useful as therapeutic agents to block simultaneously two or more different targets to enhance efficacy/safety and/or increase patient coverage. Such targets may include soluble targets (e.g., TNF) and cell surface receptor targets (e.g., VEGFR and EGFR). It can also be used to induce redirected cytotoxicity between tumor cells and T cells (e.g., Her2 and CD3) for cancer therapy, or between autoreactive cell and effector cells for autoimmune disease or transplantation, or between any target cell and effector cell to eliminate disease-causing cells in any given disease.

In addition, TVD binding proteins can be used to trigger receptor clustering and activation when it is designed to target two or more different epitopes on the same receptor. For example, this may have benefit in making agonistic and antagonistic anti-GPCR therapeutics. In this case, TVD binding proteins can be used to target two or more different epitopes (including epitopes on both the loop regions and the extracellular domain) on one cell for clustering/signaling (two cell surface molecules) or signaling (on one molecule). Similarly, a TVD binding protein can be designed to trigger CTLA-4 ligation, and a negative signal by targeting two different epitopes (or 2 copies of the same epitope) of CTLA-4 extracellular domain, leading to down regulation of the immune response. CTLA-4 is a clinically validated target for therapeutic treatment of a number of immunological disorders. CTLA-4/B7 interactions negatively regulate T cell activation by attenuating cell cycle progression, IL-2 production, and proliferation of T cells following activation, and CTLA-4 (CD152) engagement can down-regulate T cell activation and promote the induction of immune tolerance. However, the strategy of attenuating T cell activation by agonistic antibody engagement of CTLA-4 has been unsuccessful since CTLA-4 activation requires ligation. The molecular interaction of CTLA-4/B7 is in “skewed zipper” arrays, as demonstrated by crystal structural analysis (Stamper (2001) Nature 410: 608). However, none of the currently available CTLA-4 binding reagents have ligation properties, including anti-CTLA-4 monoclonal antibodies. There have been several attempts to address this issue. In one case, a cell member-bound single chain antibody was generated, and significantly inhibited allogeneic rejection in mice (Hwang (2002) J. Immunol. 169: 633). In a separate case, artificial APC surface-linked single-chain antibody to CTLA-4 was generated and demonstrated to attenuate T cell responses (Griffin (2000) J. Immunol. 164: 4433).

In both cases, CTLA-4 ligation was achieved by closely localized member-bound antibodies in artificial systems. While these experiments provide proof-of-concept for immune down-regulation by triggering CTLA-4 negative signaling, the reagents used in these reports are not suitable for therapeutic use. To this end, CTLA-4 ligation may be achieved by using a TVD binding protein that targets two different epitopes (or 2 copies of the same epitope) of a CTLA-4 extracellular domain. The rationale is that the distance spanning two binding sites of an IgG, approximately 150-170 Å, is too large for active ligation of CTLA-4 (30-50 Å between 2 CTLA-4 homodimer). However, the distance between the three binding sites on a TVD binding protein (one arm) is much shorter, also in the range of 30-50 Å, allowing proper ligation of CTLA-4. Similarly, TVD binding proteins can target two different members of a cell surface receptor complex (e.g., IL-12R alpha and beta). Furthermore, TVD binding proteins can target CR1 and a soluble protein/pathogen to drive rapid clearance of the target soluble protein/pathogen.

Additionally, TVD binding proteins of the present disclosure can be employed for tissue-specific delivery (target a tissue marker and a disease mediator for enhanced local PK, thus higher efficacy and/or lower toxicity), including intracellular delivery (targeting an internalizing receptor and a intracellular molecule) and delivery to inside of the brain (targeting transferrin receptor and a CNS disease mediator for crossing the blood-brain barrier). TVD binding proteins can also serve as a carrier protein to deliver an antigen to a specific location via binding to a non-neutralizing epitope of that antigen and also to increase the half-life of the antigen. Furthermore, TVD binding proteins can be designed to either be physically linked to medical devices implanted into patients or target these medical devices (see Burke, S. E. et al. (2006) Adv. Drug Deliv. Rev. 58(3): 437-446; Hildebrand, H. F. et al. (2006) Surface and Coatings Technol. 200(22-23): 6318-6324; Wu, P. et al. (2006) Biomaterials 27(11): 2450-2467; Marques, A. P. et al. (2005) Biodegrad. Syst. Tissue Eng. Regen. Med. 377-397). Briefly, directing appropriate types of cell to the site of medical implant may promote healing and restoring normal tissue function. Alternatively, inhibition of mediators (including, but not limited to, cytokines), released upon device implantation by a TVD molecule coupled to or target to a device is also provided. For example, stents have been used for years in interventional cardiology to clear blocked arteries and to improve the flow of blood to the heart muscle. However, traditional bare metal stents have been known to cause restenosis (re-narrowing of the artery in a treated area) in some patients and can lead to blood clots. Recently, an anti-CD34 antibody coated stent has been described which reduced restenosis and prevents blood clots from occurring by capturing endothelial progenitor cells (EPC) circulating throughout the blood. Endothelial cells are cells that line blood vessels, allowing blood to flow smoothly. The EPCs adhere to the hard surface of the stent forming a smooth layer that not only promotes healing but prevents restenosis and blood clots, complications previously associated with the use of stents (Aoji, et al. (2005) J. Am. Coll. Cardiol. 45(10): 1574-9). In addition to improving outcomes for patients requiring stents, there are also implications for patients requiring cardiovascular bypass surgery. For example, a prosthetic vascular conduit (artificial artery) coated with anti-EPC antibodies would eliminate the need to use arteries from patients' legs or arms for bypass surgery grafts. This would reduce surgery and anesthesia times, which, in turn, will reduce coronary surgery deaths. A TVD binding protein is designed in such a way that it binds to a cell surface marker (such as CD34) as well as a protein (or an epitope of any kind including, but not limited to, proteins, lipids and polysaccharides) that has been coated on the implanted device to facilitate the cell recruitment. Such approaches can also be applied to other medical implants in general. Alternatively, TVD binding proteins can be coated on medical devices and, upon implantation and releasing all TVD binding proteins from the device (or any other need, which may require additional fresh TVD binding protein, including aging and denaturation of the already loaded TVD binding protein), the device could be reloaded by systemic administration of fresh TVD binding protein to the patient, where the TVD binding protein is designed to bind to two or more targets of interest (a cytokine, a cell surface marker (such as CD34), etc.) with one set of binding sites and to a target coated on the device (including a protein and an epitope of any kind including, but not limited to, lipids, polysaccharides and polymers) with another. This technology has the advantage of extending the usefulness of coated implants.

### A. Use of TVD Binding Proteins in Various Diseases

TVD binding proteins of the present disclosure are also useful as therapeutic molecules to treat various diseases. Such TVD binding proteins may bind one or more targets involved in a specific disease. Examples of such targets in various diseases are described below.

### 1. Human Autoimmune and Inflammatory Response

Many proteins have been implicated in general autoimmune and inflammatory responses, including C5, CCL1 (1-309), CCL11 (eotaxin), CCL13 (mcp-4), CCL15 (MIP-1d), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (1-TAC/IP-9), CXCL12 (SDF1), CXCL13, CXCL14, CXCL2, CXCL3, CXCL5 (ENA-78/LIX), CXCL6 (GCP-2), CXCL9, IL13, IL8, CCL13 (mcp-4), CCR1, CCR2, CCR3, CCR4, CCR5, CCR6, CCR7, CCR8, CCR9, CX3CR1, IL8RA, XCR1 (CCXCR1), IFNA2, IL10, IL13, IL17C, IL1A, IL1B, IL1F10, IL1F5, IL1F6, IL1F7, IL1F8, IL1F9, IL22, IL5, IL8, IL9, LTA, LTB, MIF, SCYE1 (endothelial Monocyte-activating cytokine), SPP1, TNF, TNFSF5, IFNA2, IL10RA, IL10RB, IL13, IL13RA1, IL5RA, IL9, IL9R, ABCF1, BCL6, C3, C4A, CEBPB, CRP, ICEBERG, IL1R1, IL1RN, IL8RB, LTB4R, TOLLIP, FADD, IRAK1, IRAK2, MYD88, NCK2, TNFAIP3, TRADD, TRAF1, TRAF2, TRAF3, TRAF4, TRAF5, TRAF6, ACVR1, ACVR1B, ACVR2, ACVR2B, ACVRL1, CD28, CD3E, CD3G, CD3Z, CD69, CD80, CD86, CNR1, CTLA4, CYSLTR1, FCER1A, FCER2, FCGR3A, GPR44, HAVCR2, OPRD1, P2RX7, TLR2, TLR3, TLR4, TLR5, TLR6, TLR7, TLR8, TLR9, TLR10, BLR1, CCL1, CCL2, CCL3, CCL4, CCL5, CCL7, CCL8, CCL11, CCL13, CCL15, CCL16, CCL17, CCL18, CCL19, CCL20, CCL21, CCL22, CCL23, CCL24, CCL25, CCR1, CCR2, CCR3, CCR4, CCR5, CCR6, CCR7, CCR8, CCR9, CX3CL1, CX3CR1, CXCL1, CXCL2, CXCL3, CXCL5, CXCL6, CXCL10, CXCL11, CXCL12, CXCL13, CXCR4, GPR2, SCYE1, SDF2, XCL1, XCL2, XCR1, AMH, AMHR2, BMPR1A, BMPR1B, BMPR2, C19orf10 (IL27w), CER1, CSF1, CSF2, CSF3, CTLA4, DKFZp451J0118, E-selectin, L-selectin, Fc gamma receptor, FGF2, GFI1, HMGB1, IFNA1, IFNB1, IFNG, IGF1, IL1A, IL1B, IL1R1, IL1R2, IL2, IL2RA, IL2RB, IL2RG, IL3, IL4, IL4R, IL5, IL5RA, IL6, IL6R, IL6ST, IL7, IL8, IL8RA, IL8RB, IL9, IL9R, IL10, IL10RA, IL10RB, IL11, IL11RA, IL-12, IL12A, IL12B, IL12RB1, IL12RB2, IL13, IL13RA1, IL13RA2, IL15, IL15RA, IL16, IL17, IL17R, IL18, IL18R1, IL19, IL20, KITLG, LEP, LTA, LTB, LTB4R, LTB4R2, LTBR, MIF, NGF, NKG2D, NPPB, PDGFB, PGE2, RAGE, TBX21, TDGF1, TGFA, TGFB1, TGFB1I1, TGFB2, TGFB3, TGFBI, TGFBR1, TGFBR2, TGFBR3, TH1L, TNF, TNFα, TNFRSF1A, TNFRSF1B, TNFRSF7, TNFRSF8, TNFRSF9, TNFRSF11A, TNFRSF21, TNFSF4, TNFSF5, TNFSF6, TNFSF11, VEGF, ZFPM2, RNF110 (ZNF144), NKG2D, Fc gamma receptor, glycoprotein (GP) IIb/IIIa, thrombomodulin, thrombin, TREM, PAI-I, αVβ3, uPA, Her2, IGF1R, EGFR, CD3, substance P, CGRP, Protein C, Factor VII, Factor IX, plasminogen activator, Factor V, Factor VIIa, Factor Factor X, Factor XII, Factor XIII, C1q, C1r C1s, C4a, C4b, C2a, C2b, C, C3a and C3b. In one aspect, TVD binding proteins that can bind one or more of the targets listed herein are provided.

### 2. Asthma

Allergic asthma is characterized by the presence of eosinophilia, goblet cell metaplasia, epithelial cell alterations, airway hyperreactivity (AHR), and Th2 and Th1 cytokine expression, as well as elevated serum IgE levels. It is now widely accepted that airway inflammation is the key factor underlying the pathogenesis of asthma, involving a complex interplay of inflammatory cells such as T cells, B cells, eosinophils, mast cells and macrophages, and of their secreted mediators including cytokines and chemokines. Corticosteroids are the most important anti-inflammatory treatment for asthma today; however, their mechanism of action is non-specific and safety concerns exist, especially in the juvenile patient population. The development of more specific and targeted therapies is therefore warranted. There is increasing evidence that IL-13 in mice mimics many of the features of asthma, including AHR, mucus hypersecretion and airway fibrosis, independently of eosinophilic inflammation (Finotto, et al. (2005) Internat. Immunol. 17(8): 993-1007; Padilla, et al. (2005) J. Immunol. 174(12): 8097-8105).

IL-13 has been implicated as having a pivotal role in causing pathological responses associated with asthma. The development of anti-IL-13 monoclonal antibody therapy to reduce the effects of IL-13 in the lung is an exciting new approach that offers considerable promise as a novel treatment for asthma. However, other mediators of differential immunological pathways are also involved in asthma pathogenesis, and blocking these mediators, in addition to IL-13, may offer additional therapeutic benefit. Such target sets include, but are not limited to, IL-13 and a pro-inflammatory cytokine, such as IL-18 and tumor necrosis factor-α (TNF-α). TNF-α may amplify the inflammatory response in asthma and may be linked to disease severity (McDonnell, et al. (2001) Progr. Respir. Res. 31: 247-250). IL-18 activates mast cells and basophils. This suggests that blocking IL-13, IL-18, and TNF-α may have beneficial effects, particularly in severe airway disease. In one embodiment, the TVD binding proteins of the present disclosure bind the targets IL-13, IL-18, and TNFα and is used for treating asthma.

Animal models such as OVA-induced asthma mouse model, where both inflammation and AHR can be assessed, are known in the art and may be used to determine the ability of various TVD binding proteins to treat asthma Animal models for studying asthma are disclosed in Coffman, et al. (2005) J. Exp. Med. 201(12): 1875-1879; Lloyd et al. (2001) Adv. Immunol. 77: 263-295; Boyce et al. (2005) J. Exp. Med. 201(12): 1869-1873; and Snibson et al. (2005) J. Brit. Soc. Allerg. Clin. Immunol. 35(2): 146-52. In addition to routine safety assessments of these target pairs, specific tests for the degree of immunosuppression may be warranted and helpful in selecting the best target pairs (see Luster et al. (1994) Toxicology 92(1-3): 229-43; Descotes, et al. (1992) Devel. Biol. Stand. 77: 99-102; Hart et al. (2001) J. Allerg. Clin. Immunol. 108(2): 250-257).

Based on the rationale disclosed herein, and using the same evaluation model for efficacy and safety, other sets of targets that TVD binding proteins can bind and that can be useful to treat asthma may be determined In one embodiment, such targets include, but are not limited to, IL-13 and IL-1beta, since IL-1beta is also implicated in inflammatory response in asthma; IL-13 and cytokines and chemokines that are involved in inflammation, such as IL-13 and IL-9; IL-13 and IL-4; IL-13 and IL-5; IL-13 and IL-25; IL-13 and TARC; IL-13 and MDC; IL-13 and MIF; IL-13 and TGF-β; IL-13 and LHR agonist; IL-13 and CL25; IL-13 and SPRR2a; IL-13 and SPRR2b; and IL-13 and ADAM8. The present disclosure also provides TVD binding proteins that can bind one or more targets involved in asthma selected from the group consisting of CSF1 (MCSF), CSF2 (GM-CSF), CSF3 (GCSF), FGF2, IFNA1, IFNB1, IFNG, histamine and histamine receptors, IL1A, IL1B, IL2, IL3, IL4, IL5, IL6, IL7, IL8, IL9, IL10, IL11, IL12A, IL12B, IL13, IL14, IL15, IL16, IL17, IL18, IL19, KITLG, PDGFB, IL2RA, IL4R, IL5RA, IL8RA, IL8RB, IL12RB1, IL12RB2, IL13RA1, IL13RA2, IL18R1, TSLP, CCL1, CCL2, CCL3, CCL4, CCL5, CCL7, CCL8, CCL13, CCL17, CCL18, CCL19, CCL20, CCL22, CCL24, CX3CL1, CXCL1, CXCL2, CXCL3, XCL1, CCR2, CCR3, CCR4, CCR5, CCR6, CCR7, CCR8, CX3CR1, GPR2, XCR1, FOS, GATA3, JAK1, JAK3, STAT6, TBX21, TGFB1, TNF, TNFSF6, YY1, CYSLTR1, FCER1A, FCER2, LTB4R, TB4R2, LTBR, and Chitinase.

### 3. Rheumatoid Arthritis

Rheumatoid arthritis (RA), a systemic disease, is characterized by a chronic inflammatory reaction in the synovium of joints and is associated with degeneration of cartilage and erosion of juxta-articular bone. Many pro-inflammatory cytokines including TNF, chemokines, and growth factors are expressed in diseased joints. Systemic administration of anti-TNF antibody or sTNFR fusion protein to mouse models of RA was shown to be anti-inflammatory and joint protective. Clinical investigations in which the activcity of TNF in RA patients was blocked with intravenously administered infliximonoclonal antibody (Harriman, G. et al. (1999) Ann. Rheum. Dis. 58 (Suppl 1): 161-4), a chimeric anti-TNF monoclonal antibody, has provided evidence that TNF regulates IL-6, IL-8, MCP-1, and VEGF production, recruitment of immune and inflammatory cells into joints, angiogenesis, and reduction of blood levels of matrix metalloproteinases-1 and -3. A better understanding of the inflammatory pathway in rheumatoid arthritis has led to identification of other therapeutic targets involved in rheumatoid arthritis. Promising treatments, such as interleukin-6 antagonists (IL-6 receptor antibody MRA, developed by Chugai, Roche (see Nishimoto, N. et al. (2004) Arthrit. Rheum. 50(6): 1761-1769), CTLA4Ig (abatacept, Genovese, M. et al. (2005) N. Engl. J. Med. 353: 1114-23.), and anti-B cell therapy (rituximonoclonal antibody; Okamoto, H. and Kamatani, N. (2004) N. Engl. J. Med. 351: 1909), have already been tested in randomized controlled trials over the past year. Other cytokines have been identified and have been shown to be of benefit in animal models, including interleukin-15 (therapeutic antibody HuMax-IL_{—}15, AMG 714 (see Baslund, B. et al. (2005) Arthrit. Rheum. 52(9): 2686-2692)), interleukin-17, and interleukin-18, and clinical trials of these agents are currently under way. Multi- (e.g., tri-) specific antibody therapy, combining anti-TNF and other mediators, has great potential in enhancing clinical efficacy and/or patient coverage. For example, blocking both TNF and VEGF can potentially eradicate inflammation and angiogenesis, both of which are involved in pathophysiology of RA. Blocking other sets of targets involved in RA including, but not limited to, NGF, TNF, and PGE2; IL1A, IL-1B, and PGE2; TNF and IL-18; TNF and IL-12; TNF and IL-23; TNF and IL-1beta; TNF and MIF; TNF and IL-17; TNF and IL-15, TNF and SOST with specific TVD binding proteins is also contemplated. In one embodiment, the binding proteins of the present invention bind the targets selected from the group consisting of: NGF, TNF, and PGE2; and IL-1α, IL-1β, and PGE2.

Additionally, high levels of expression of NGF and IL-1β are associated with pain in osteoarthritis. Accordingly, in one embodiment, the binding proteins of the present invention bind the targets selected from the group consisting of: IL-1α, IL-1β, and NGF; IL-1α, IL-1β, and PGE2.

In addition to routine safety assessments of these target sets, specific tests for the degree of immunosuppression may be warranted and helpful in selecting the best target sets (see Luster et al. (1994) Toxicol. 92(1-3): 229-43; Descotes et al. (1992) Devel. Biol. Stand. 77: 99-102; Hart et al. (2001) J. Allerg. Clin. Immunol. 108(2): 250-257). Whether a TVD binding protein will be useful for the treatment of rheumatoid arthritis can be assessed using pre-clinical animal RA models such as the collagen-induced arthritis mouse model. Other useful models are also well known in the art (see Brand, D. D. (2005) Comp. Med. 55(2): 114-22). Based on the cross-reactivity of the parental binding proteins, e.g., antibodies, for human and mouse othologues (e.g., reactivity for human and mouse TNF, human and mouse IL-15, etc.) validation studies in the mouse CIA model may be conducted with “matched surrogate antibody” derived TVD binding proteins; briefly, a TVD binding protein based on two or more mouse target specific binding proteins, e.g., antibodies, may be matched to the extent possible to the characteristics of the parental human or humanized binding proteins, e.g., antibodies, used for human TVD binding protein construction (similar affinity, similar neutralization potency, similar half-life etc.).

### 4. SLE

The immunopathogenic hallmark of SLE is the polyclonal B cell activation, which leads to hyperglobulinemia, autoantibody production and immune complex formation. The fundamental abnormality appears to be the failure of T cells to suppress the forbidden B cell clones due to generalized T cell dysregulation. In addition, B and T-cell interaction is facilitated by several cytokines, such as IL-10, as well as co-stimulatory molecules, such as CD40, CD40L, B7, CD28, and CTLA-4, which initiate the second signal. These interactions, together with impaired phagocytic clearance of immune complexes and apoptotic material, perpetuate the immune response with resultant tissue injury. The following targets may be involved in SLE and can potentially be used for a TVD binding protein approach for therapeutic intervention: B cell targeted therapies: CD-20, CD-22, CD-19, CD28, CD4, CD80, HLA-DRA, IL10, IL2, IL4, TNFRSF5, TNFRSF6, TNFSF5, TNFSF6, BLR1, HDAC4, HDAC5, HDAC7A, HDAC9, ICOSL, IGBP1, MS4A1, RGS1, SLA2, CD81, IFNB1, IL10, TNFRSF5, TNFRSF7, TNFSF5, AICDA, BLNK, GALNAC4S-6ST, HDAC4, HDAC5, HDAC7A, HDAC9, IL10, IL11, IL4, INHA, INHBA, KLF6, TNFRSF7, CD28, CD38, CD69, CD80, CD83, CD86, DPP4, FCER2, IL2RA, TNFRSF8, TNFSF7, CD24, CD37, CD40, CD72, CD74, CD79A, CD79B, CR2, IL1R2, ITGA2, ITGA3, MS4A1, ST6GAL1, CD1C, CHST10, HLA-A, HLA-DRA, and NT5E; co-stimulatory signals: CTLA4 or B7.1/B7.2; inhibition of B cell survival: BlyS or BAFF; Complement inactivation: C5; Cytokine modulation: the key principle is that the net biologic response in any tissue is the result of a balance between local levels of proinflammatory or anti-inflammatory cytokines (see Sfikakis, P. P. et al. (2005) Curr. Opin. Rheumatol. 17: 550-7). SLE is considered to be a Th-2 driven disease with documented elevations in serum IL-4, IL-6, and IL-10. TVD binding proteins that can bind two or more targets selected from the group consisting of IL-4, IL-6, IL-10, IFN-α, and TNF-α are also contemplated. Combination of targets discussed herein will enhance therapeutic efficacy for SLE, which can be tested in a number of lupus preclinical models (see Peng, S. L. (2004) Methods Mol. Med. 102: 227-72). Based on the cross-reactivity of the parental binding proteins, e.g., antibodies, for human and mouse othologues (e.g., reactivity for human and mouse CD20, human and mouse Interferon alpha, etc.) validation studies in a mouse lupus model may be conducted with “matched surrogate antibody” derived TVD binding proteins. Briefly, a TVD binding protein based two or more mouse target specific binding proteins, e.g., antibodies, may be matched to the extent possible to the characteristics of the parental human or humanized binding proteins, e.g., antibodies, used for human TVD binding protein construction (similar affinity, similar neutralization potency, similar half-life etc.).

### 5. Multiple Sclerosis

Multiple sclerosis (MS) is a complex human autoimmune-type disease with a predominantly unknown etiology. Immunologic destruction of myelin basic protein (MBP) throughout the nervous system is the major pathology of multiple sclerosis. MS is a disease of complex pathologies, which involves infiltration by CD4+ and CD8+ T cells and response within the central nervous system. Expression in the CNS of cytokines, reactive nitrogen species and costimulator molecules have all been described in MS. Of major consideration are immunological mechanisms that contribute to the development of autoimmunity. In particular, antigen expression, cytokine and leukocyte interactions, and regulatory T-cells, which help balance/modulate other T-cells, such as Th1 and Th2 cells, are important areas for therapeutic target identification.

IL-12 is a proinflammatory cytokine that is produced by APC and promotes differentiation of Th1 effector cells. IL-12 is produced in the developing lesions of patients with MS as well as in EAE-affected animals. Previously it was shown that interference in IL-12 pathways effectively prevents EAE in rodents, and that in vivo neutralization of IL-12p40 using a anti-IL-12 monoclonal antibody has beneficial effects in the myelin-induced EAE model in common marmosets.

TWEAK is a member of the TNF family, constitutively expressed in the central nervous system (CNS), with pro-inflammatory, proliferative or apoptotic effects depending upon cell types. Its receptor, Fn14, is expressed in CNS by endothelial cells, reactive astrocytes and neurons. TWEAK and Fn14 mRNA expression increased in spinal cord during experimental autoimmune encephalomyelitis (EAE). Anti-TWEAK antibody treatment in myelin oligodendrocyte glycoprotein (MOG) induced EAE in C57BL/6 mice resulted in a reduction of disease severity and leukocyte infiltration when mice were treated after the priming phase.

One aspect of the present disclosure pertains to TVD binding proteins that can bind two or more, for example three, targets selected from the group consisting of IL-12, TWEAK, IL-23, CXCL13, CD40, CD40L, IL-18, VEGF, VLA-4, TNF, CD45RB, CD200, IFNgamma, GM-CSF, FGF, C5, CD52, and CCR2.

Several animal models for assessing the usefulness of the TVD binding proteins to treat MS are known in the art (see Steinman. L. et al. (2005) Trends Immunol. 26(11): 565-71; Lublin, F. D. et al. (1985) Springer Semin Immunopathol. 8(3): 197-208; Genain, C. P. et al. (1997) J. Mol. Med. 75(3): 187-97; Tuohy, V. K. et al. (1999) J. Exp. Med. 189(7): 1033-42; Owens, T. et al. (1995) Neurol. Clin.13(1): 51-73; and Hart, B. A. et al. (2005) J. Immunol. 175(7): 4761-8. Based on the cross-reactivity of the parental binding proteins, e.g., antibodies, for human and animal species othologues (e.g., reactivity for human and mouse IL-12, human and mouse TWEAK etc.), validation studies in the mouse EAE model may be conducted with “matched surrogate antibody” derived TVD binding protein. Briefly, a TVD binding protein based on two or more mouse target specific binding proteins, e.g., antibodies, may be matched to the extent possible to the characteristics of the parental human or humanized binding proteins, e.g., antibodies, used for human TVD binding protein construction (similar affinity, similar neutralization potency, similar half-life etc.). The same concept applies to animal models in other non-rodent species, where a “matched surrogate antibody” derived TVD binding protein would be selected for the anticipated pharmacology and possibly safety studies. In addition to routine safety assessments of these target pairs specific tests for the degree of immunosuppression may be warranted and helpful in selecting the best target pairs (see Luster et al. (1994) Toxicol. 92(1-3): 229-43; Descotes et al. (1992) Devel. Biol. Stand. 77: 99-102; Jones, R. (2000) IDrugs 3(4): 442-6).

### 6. Sepsis

The pathophysiology of sepsis is initiated by the outer membrane components of both gram-negative organisms (lipopolysaccharide (LPS), lipid A, endotoxin) and gram-positive organisms (lipoteichoic acid, peptidoglycan). These outer membrane components are able to bind to the CD14 receptor on the surface of monocytes. By virtue of the recently described toll-like receptors, a signal is then transmitted to the cell, leading to the eventual production of the proinflammatory cytokines tumor necrosis factor-alpha (TNF-alpha) and interleukin-1 (IL-1). Overwhelming inflammatory and immune responses are essential features of septic shock and play a central part in the pathogenesis of tissue damage, multiple organ failure, and death induced by sepsis. Cytokines, especially tumor necrosis factor (TNF) and interleukin (IL-1), have been shown to be critical mediators of septic shock. These cytokines have a direct toxic effect on tissues; they also activate phospholipase A2. These and other effects lead to increased concentrations of platelet-activating factor, promotion of nitric oxide synthase activity, promotion of tissue infiltration by neutrophils, and promotion of neutrophil activity.

The treatment of sepsis and septic shock remains a clinical conundrum, and recent prospective trials with biological response modifiers (i.e., anti-TNF and anti-MIF) aimed at the inflammatory response have shown only modest clinical benefit. Recently, interest has shifted toward therapies aimed at reversing the accompanying periods of immune suppression. Studies in experimental animals and critically ill patients have demonstrated that increased apoptosis of lymphoid organs and some parenchymal tissues contribute to this immune suppression, anergy, and organ system dysfunction. During sepsis syndromes, lymphocyte apoptosis can be triggered by the absence of IL-2 or by the release of glucocorticoids, granzymes, or the so-called ‘death’ cytokines: tumor necrosis factor alpha or Fas ligand. Apoptosis proceeds via auto-activation of cytosolic and/or mitochondrial caspases, which can be influenced by the pro- and anti-apoptotic members of the Bcl-2 family. In experimental animals, not only can treatment with inhibitors of apoptosis prevent lymphoid cell apoptosis; it may also improve outcome. Although clinical trials with anti-apoptotic agents remain distant due in large part to technical difficulties associated with their administration and tissue targeting, inhibition of lymphocyte apoptosis represents an attractive therapeutic target for the septic patient. Likewise, a multispecific agent targeting both inflammatory mediator and an apoptotic mediator, may have added benefit. One aspect of the present disclosure pertains to TVD binding protein that can bind two or more targets involved in sepsis. In one embodiment, two or more targets are selected from the group consisting of TNF, IL-1, MIF, IL-6, IL-8, IL-18, IL-12, IL-23, FasL, LPS, Toll-like receptors, TLR-4, tissue factor, MIP-2, ADORA2A, CASP1, CASP4, IL-10, IL-1B, NFKB1, PROC, TNFRSF1A, CSF3, CCR3, IL1RN, MIF, NFKB1, PTAFR, TLR2, TLR4, GPR44, HMOX1, midkine, IRAK1, NFKB2, SERPINA1, SERPINE1, TREM1, TNF (e.g., TNFα), PGE2, IL-12, IL-13, IL-18, HMGB1, VEGF, RAGE, NGF, IL-1α, IL-1β, E-selectin, L-selectin, glycoprotein (GP) thrombomodulin, thrombin, TREM1, PAI-I, αVβ3, uPA, a component of the coagulation cascade, e.g., Protein C, Factor VII, Factor IX, plasminogen activator, Factor V, Factor VIIa, Factor Factor X, Factor XII, and Factor XIII, and a complement component, e.g., C1q, C1r C1s, C4a, C4b, C2a, C2b, C, C3a and C3b. In one embodiment, the TVD binding proteins of the present invention bind three targets selected from the group consisting of: HMGB1, VEGF, and TNF (e.g., TNFα); RAGE, VEGF, and TNF (e.g., TNFα); NGF, TNF (e.g., TNFα), and PGE2; IL-1α, IL-1β, and PGE2; and IL-1α, IL-1β, and NGF. The efficacy of such TVD binding proteins for sepsis can be assessed in preclinical animal models known in the art (see Buras, J. A. et al. (2005) Nat. Rev. Drug Discov. 4(10): 854-65; and Calandra, T. et al. (2000) Nat. Med. 6(2): 164-70).

### 7. Neurological Disorders

### 7.1. Neurodegenerative Diseases

Chronic neurodegenerative diseases are usually age-dependent diseases characterized by progressive loss of neuronal functions (neuronal cell death, demyelination), loss of mobility and loss of memory. Emerging knowledge of the mechanisms underlying chronic neurodegenerative diseases (e.g., Alzheimer's disease disease) show a complex etiology, and a variety of factors have been recognized to contribute to their development and progression e.g., age, glycemic status, amyloid production and multimerization, accumulation of advanced glycation-end products (AGE), which bind to their receptor RAGE (receptor for AGE), increased brain oxidative stress, decreased cerebral blood flow, neuroinflammation including release of inflammatory cytokines and chemokines, neuronal dysfunction and microglial activation. Thus, these chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments fail to stop disease progression. Recent studies suggest that more targeted therapies, such as antibodies to soluble Aβ peptide (including the Aβ oligomeric forms) can not only help stop disease progression but may help maintain memory as well. These preliminary observations suggest that specific therapies targeting more than one disease mediator (e.g., Aβ and one or more of a pro-inflammatory cytokine, such as TNF) may provide even better therapeutic efficacy for chronic neurodegenerative diseases than observed with targeting a single disease mechanism (e.g., soluble Aβ alone) (see Nelson, R. B. (2005) Curr. Pharm. Des. 11: 3335; Klein. W. (2002) Neurochem. Int. 41: 345; Janelsins, M. C. et al. (2005) J. Neuroinflamm. 2: 23; Soloman, B. (2004) Curr. Alzheimer Res. 1: 149; Klyubin, I. et al. (2005) Nat. Med. 11: 556-61; Bornemann, K. D et al. (2001) Am. J. Pathol. 158: 63; Deane, R. et al. (2003) Nat. Med. 9: 907-13; and Masliah, E. et al. (2005) Neuron. 46: 857).

The TVD binding proteins of the present disclosure can bind two or more targets involved in chronic neurodegenerative diseases, such as Alzheimers. Such targets include, but are not limited to, any mediator, soluble or cell surface, implicated in AD pathogenesis, e.g., AGE (S100 A, amphoterin), pro-inflammatory cytokines (e.g., IL-1), chemokines (e.g., MCP 1), molecules that inhibit nerve regeneration (e.g., Nogo, RGM A), and molecules that enhance neurite growth (neurotrophins). The efficacy of TVD binding proteins can be validated in pre-clinical animal models, such as the transgenic mice that over-express amyloid precursor protein or RAGE and develop Alzheimer's disease-like symptoms. In addition, TVD binding proteins can be constructed and tested for efficacy in the animal models, and the best therapeutic TVD binding proteins can be selected for testing in human patients. TVD binding proteins can also be employed for treatment of other neurodegenerative diseases, such as Parkinson's disease. Alpha-Synuclein is involved in Parkinson's pathology. A TVD binding protein that can target alpha-synuclein and inflammatory mediators, such as TNF, IL-1, MCP-1, can prove effective therapy for Parkinson's disease and are contemplated in the present disclosure.

### 7.2 Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries, and the primary injury is usually followed by secondary injury mechanisms (inflammatory mediators, e.g., cytokines and chemokines) that worsen the initial injury and result in significant enlargement of the lesion area, sometimes more than 10-fold. These primary and secondary mechanisms in SCI are very similar to those in brain injury caused by other means, e.g., stroke. No satisfying treatment exists and high dose bolus injection of methylprednisolone (MP) is the only used therapy within a narrow time window of 8 h post injury. This treatment, however, is only intended to prevent secondary injury without causing any significant functional recovery. It is heavily critisized for the lack of unequivocal efficacy and severe adverse effects, like immunosuppression with subsequent infections and severe histopathological muscle alterations. No other drugs, biologics or small molecules, stimulating the endogenous regenerative potential are approved, but promising treatment principles and drug candidates have shown efficacy in animal models of SCI in recent years. To a large extent the lack of functional recovery in human SCI is caused by factors inhibiting neurite growth, at lesion sites, in scar tissue, in myelin as well as on injury-associated cells. Such factors are the myelin-associated proteins NogoA, OMgp and MAG, RGM A, the scar-associated CSPG (Chondroitin Sulfate Proteoglycans) and inhibitory factors on reactive astrocytes (some semaphorins and ephrins). However, at the lesion site not only growth inhibitory molecules are found but also neurite growth stimulating factors like neurotrophins, laminin, L1 and others. This ensemble of neurite growth inhibitory and growth promoting molecules may explain that blocking single factors, like NogoA or RGM A, resulted in significant functional recovery in rodent SCI models, because a reduction of the inhibitory influences could shift the balance from growth inhibition to growth promotion. However, recoveries observed with blocking a single neurite outgrowth inhibitory molecule were not complete. To achieve faster and more pronounced recoveries either blocking two neurite outgrowth inhibitory molecules e.g., Nogo and RGM A, or blocking a neurite outgrowth inhibitory molecule and enhancing functions of a neurite outgrowth enhancing molecule, e.g., Nogo, and neurotrophin(s), or blocking a neurite outgrowth inhibitory moleclule, e.g., Nogo, and a pro-inflammatory molecule(s), e.g., TNF, may be desirable (see McGee, A. W. et al. (2003) Trends Neurosci. 26: 193; Domeniconi, M. et al. (2005) J. Neurol. Sci. 233: 43; Makwanal, M. et al. (2005) FEBS J. 272: 2628; Dickson, B. J. (2002) Science 298: 1959; Yu, F. and Teng, H. et al. (2005) J. Neurosci. Res. 79: 273; Karnezis, T. et al. (2004) Nature Neurosci. 7: 736; Xu, G. et al. (2004) J. Neurochem. 91: 1018).

In one aspect, TVD binding proteins that can bind target sets, such as NgR and RGM A; NogoA and RGM A; MAG and RGM A; OMGp and RGM A; RGM A and RGM B; CSPGs and RGM A; aggrecan, midkine, neurocan, versican, phosphacan, Te38 and TNF-α; and Aβ globulomer-specific antibodies combined with antibodies promoting dendrite and axon sprouting, are provided. Dendrite pathology is a very early sign of AD, and it is known that NOGO A restricts dendrite growth. One can combine one such type of Aβ with any one or more of the SCI-candidate (myelin-proteins) Abs. Other TVD binding protein targets may include any combination of NgR-p75, NgR-Troy, NgR-Nogo66 (Nogo), NgR-Lingo, Lingo-Troy, Lingo-p75, MAG and Omgp. Additionally, targets may also include any mediator, soluble or cell surface, implicated in inhibition of neurite, e.g., Nogo, Ompg, MAG, RGM A, semaphorins, ephrins, soluble A-b, pro-inflammatory cytokines (e.g., IL-1), chemokines (e.g., MIP 1a), and molecules that inhibit nerve regeneration. The efficacy of such TVD binding proteins can be validated in pre-clinical animal models of spinal cord injury. In addition, these TVD binding proteins can be constructed and tested for efficacy in the animal models, and the best therapeutic TVD binding protein can be selected for testing in human patients. In addition, TVD binding proteins can be constructed that target two distinct ligand binding sites on a single receptor, e.g., Nogo receptor, which binds the three ligands Nogo, Ompg, and MAG, and RAGE that binds Aβ and S100 A. Furthermore, neurite outgrowth inhibitors, e.g., Nogo and Nogo receptor, also play a role in preventing nerve regeneration in immunological diseases like multiple sclerosis Inhibition of Nogo-Nogo receptor interaction has been shown to enhance recovery in animal models of multiple sclerosis. Therefore, TVD binding proteins that can block the function of two immune mediator, e.g., a cytokine, like IL-12 and TNFα, and a neurite outgrowth inhibitor molecule, e.g., Nogo or RGM, may offer faster and greater efficacy than blocking either an immune or a neurite outgrowth inhibitor molecule alone.

### 8. Oncological Disorders

Monoclonal antibody therapy has emerged as an important therapeutic modality for cancer (von Mehren M, et al. (2003) Annu. Rev. Med. 54: 343-69). Antibodies may exert antitumor effects by inducing apoptosis, re-directing cytotoxicity, interfering with ligand-receptor interactions, or preventing the expression of proteins that are critical to the neoplastic phenotype. In addition, antibodies can target components of the tumor microenvironment, perturbing vital structures, such as the formation of tumor-associated vasculature. Antibodies can also target receptors whose ligands are growth factors, such as the epidermal growth factor receptor. The antibody thus inhibits natural ligands that stimulate cell growth from binding to targeted tumor cells. Alternatively, antibodies may induce an anti-idiotype network, complement-mediated cytotoxicity, or antibody-dependent cellular cytotoxicity (ADCC). The use of multispecific binding protein, e.g., antibody, that targets two or more separate tumor mediators will likely give additional benefit compared to a mono-specific therapy. TVD binding proteins that can bind the following sets of targets to treat oncological disease are also contemplated: NGF, Her2, and VEGF; NGF, EGFR, and IGF1R; NGF, EGFR, and VEGF; EGFR, Her2, and VEGF; IGF1 and IGF2; IGF1/2 and HER-2; VEGFR and EGFR; CD20 and CD3; CD138 and CD20; CD38 and CD20; CD38 and CD138; CD40 and CD20; CD138 and CD40; CD38 and CD40; CD-20 and CD-19; CD-20 and EGFR; CD-20 and CD-80; CD-20 and CD-22; CD-3 and HER-2; CD-3 and CD-19; EGFR and HER-2; EGFR and CD-3; EGFR and IGF1,2; EGFR and IGF1R; EGFR and RON; EGFR and HGF; EGFR and c-MET; HER-2 and IGF1,2; HER-2 and IGF1R; RON and HGF; VEGF and EGFR; VEGF and HER-2; VEGF and CD-20; VEGF and IGF1,2; VEGF and DLL4; VEGF and HGF; VEGF and RON; VEGF and NRP1; CD20 and CD3; VEGF and PLGF; DLL4 and PLGF; ErbB3 and EGFR; HGF and ErbB3, HER-2 and ErbB3; c-Met and ErbB3; HER-2 and PLGF; HER-2 and HER-2; TNF and SOST.

Other target combinations include two or more members of the EGF/erb-2/erb-3 family. Other targets (one or more) involved in oncological diseases that TVD binding proteins may bind include, but are not limited to, those selected from the group consisting of: CD52, CD20, CD19, CD3, CD4, CD8, BMP6, IL12A, IL1A, IL1B, IL2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, GRP, IGF1, IGF2, IL12A, IL1A, IL1B, IL2, INHA, TGFA, TGFB1, TGFB2, TGFB3, VEGF, CDK2, FGF10, FGF18, FGF2, FGF4, FGF7, IGF1R, IL2, BCL2, CD164, CDKN1A, CDKN1B, CDKN1C, CDKN2A, CDKN2B, CDKN2C, CDKN3, GNRH1, IGFBP6, IL1A, IL1B, ODZ1, PAWR, PLG, TGFB1I1, AR, BRCA1, CDK3, CDK4, CDK5, CDK6, CDK7, CDK9, E2F1, EGFR, ENO1, ERBB2, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR113, NR2F6, NR4A3, ESR1, ESR2, NR0B1, NR0B2, NR1D2, NR1H2, NR1H4, NR1I2, NR2C1, NR2C2, NR2E1, NR2E3, NR2F1, NR2F2, NR3C1, NR3C2, NR4A1, NR4A2, NR5A1, NR5A2, NR6A1, PGR, RARB, FGF1, FGF2, FGF6, KLK3, KRT1, APOC1, BRCA1, CHGA, CHGB, CLU, COL1A1, COL6A1, EGF, ERBB2, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, GNRH1, IGF1, IGF2, IGFBP3, IGFBP6, IL12A, IL1A, IL1B, IL2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ILK, ITGA1, APC, CD164, COL6A1, MTSS1, PAP, TGFB1I1, AGR2, AIG1, AKAP1, AKAP2, CANT1, CAV1, CDH12, CLDN3, CLN3, CYB5, CYC1, DAB2IP, DES, DNCL1, ELAC2, ENO2, ENO3, FASN, FLJ12584, FLJ25530, GAGEB1, GAGEC1, GGT1, GSTP1, HIP1, HUMCYT2A, IL29, K6HF, KAI1, KRT2A, MIB1, PART1, PATE, PCA3, PIAS2, PIK3CG, PPID, PR1, PSCA, SLC2A2, SLC33A1, SLC43A1, STEAP, STEAP2, TPM1, TPM2, TRPC6, ANGPT1, ANGPT2, ANPEP, ECGF1, EREG, FGF1, FGF2, FIGF, FLT1, JAG1, KDR, LAMA5, NRP1, NRP2, PGF, PLXDC1, STAB1, VEGF, VEGFC, ANGPTL3, BAIL COL4A3, IL8, LAMA5, NRP1, NRP2, STAB1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, IL6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL18A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kip1), CDKN2A (p16INK4a), COL6A1, CTNNB1 (b-catenin), CTSB (cathepsin B), ERBB2 (Her-2), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MKI67 (Ki-67), NGFB (NGF), NGFR, NME1 (NM23A), PGR, PLAU (uPA), PTEN, SERPINB5 (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), ERBB2 (Her-2), FGF1, FLRT1 (fibronectin), GABRP (GABAa), GNAS1, ID2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-III), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, and TNFAIP2 (B94), RON, c-Met, CD64, DLL4, PLGF, CTLA4, phophatidylserine, ROBO4, CD80, CD22, CD40, CD23, CD28, CD80, CD55, CD38, CD70, CD74, CD30, CD138, CD56, CD33, CD2, CD137, DR4, DR5, RANKL, VEGFR2, PDGFR, VEGFR1, MTSP1, MSP, EPHB2, EPHA1, EPHA2, EpCAM, PGE2, NKG2D, LPA, SIP, APRIL, BCMA, MAPG, FLT3, PDGFR alpha, PDGFR beta, ROR1, PSMA, PSCA, SCD1, and CD59.

In another embodiment, the TVD binding proteins of the present invention specifically target tumor cells and bring immune effector cells into close proximity of the tumor to initiate and/or enhance an immune response to the tumor. In one embodiment, the TVD binding proteins of the present invention bind CD3 and two different cell surface molecules present on heterogeneous cells of a tumor (e.g., a tumor having a mixture of cell types). In another embodiment, the TVD binding proteins of the present invention bind an immune cell receptor, such as NKG2D or an Fc gamma receptor and two different cell surface molecules present on heterogeneous cells of a tumor (e.g., a tumor having a mixture of cell types).

### 9. Other Diseases, Disorders and Conditions

Nerve growth factor (NGF) is known to influence inflammatory and neuropathic pain, and anti-NGF therapy has been shown to alleviate both of these. Accordingly, NGF can be employed in the treatment of sepsis, and rheumatoid arthritis (as discussed above) and also in the treatment of pain and osteoarthritis. Other factors shown to be involved in pain include, for example, TNF, IL-1α, IL-1β, IL-6, CGRP, substance P, and prostaglandin E2 (PGE2). Accordingly, in one embodiment, the binding proteins of the present invention bind the targets selected from the group consisting of: IL-1α, IL-1β, and NGF; IL-1α, IL-1β, and PGE2; IL-1α, NGF, and substance P; and IL-1α, NGF, and CGRP.

Additionally, high levels of expression of NGF and IL-1β are associated with pain in osteoarthritis. Accordingly, in one embodiment, the binding proteins of the present invention bind the targets selected from the group consisting of: IL-1α, IL-1β, and NGF; IL-1α, IL-1β, and PGE2.

BNP has been implicated in heart function. Among other diseases, BNP TVD binding proteins potentially can be employed in the treatment of cardiovascular disease, including various clinical diseases, disorders or conditions involving the heart, blood vessels or circulation. The diseases, disorders or conditions may be due to atherosclerotic impairment of coronary, cerebral or peripheral arteries. Such potentially treatable cardiovascular disease includes, but are not limited to, coronary artery disease, peripheral vascular disease, hypertension, myocardial infarction, heart failure, and the like. Likewise, HIV TVD binding proteins potentially can be employed in the treatment of AIDS, or symptoms of AIDS.

IL-18 has been determined to be a marker for various conditions or disease states, including, but not limited to, inflammatory disorders, e.g., allergy and autoimmune disease (Kawashima et al. (1997) J. Educ. Inform. Rheumatol. 26(2): 77), acute kidney injury (Parikh et al. (2005) J. Am. Soc. Nephrol. 16: 3046-3052; and Parikh et al. (2006) Kidney Int'l. 70: 199-203), chronic kidney disease (such as when used as part of a panel assay), minimal-change nephritic syndrome (MCNS) (Matsumoto et al. (2001) Nephron 88: 334-339), adult-onset Still's disease (Kawaguchi et al. (2001) Arthrit. Rheum. 44(7): 1716-1717), juvenile atopic dermatitis (Hon et al. (2004) Ped. Derm. 21(6): 619-622), haemophagocytic lymphohistiocytosis (HLH) (Takeda et al. (1999) Brit. J. Haematol. 106(1): 182-189), juvenile idiopathic arthritis (Lotito et al. (2007) J. Rheumatol. 34(4): 823-830), ovarian cancer (Le Page et al. (20060 Int'l J. Cancer 118: 1750-1758), systemic lupus erythematosus (Amerio et al. (2002) Clin. Exp. Rheum. 20(4): 535-538), and future cardiovascular events (Blankenberg et al. (2003) Circul. 108(20): 2453-2459).

NGAL is an early marker for acute renal injury or disease. In addition to being secreted by specific granules of activated human neutrophils, NGAL is also produced by nephrons in response to tubular epithelial damage and is a marker of tubulointerstitial (TI) injury. NGAL levels rise in acute tubular necrosis (ATN) from ischemia or nephrotoxicity, even after mild “subclinical” renal ischemia. Moreover, NGAL is known to be expressed by the kidney in cases of chronic kidney disease (CKD) and acute kidney injury ((AKI); see, e.g., Devarajan et al. (2008) Amer. J. Kidn. Dis. 52(3): 395-399 and Bolignano et al. (2008) Amer. J. Kidn. Dis. 52(3): 595-605). Elevated urinary NGAL levels have been suggested as predictive of progressive kidney failure. It has been previously demonstrated that NGAL is markedly expressed by kidney tubules very early after ischemic or nephrotoxic injury in both animal and human models. NGAL is rapidly secreted into the urine, where it can be easily detected and measured, and precedes the appearance of any other known urinary or serum markers of ischemic injury. The protein is resistant to proteases, suggesting that it can be recovered in the urine as a faithful marker of NGAL expression in kidney tubules. Further, NGAL derived from outside of the kidney, for example, filtered from the blood, does not appear in the urine, but rather is quantitatively taken up by the proximal tubule. NGAL is also a marker in the diagnosis and/or prognosis of a number of other diseases (see, e.g., Xu et al. (2000) Biochim et Biophys. Acta 1482: 298-307), disorders, and conditions, including inflammation, such as that associated with infection. It is a marker for irritable bowel syndrome (see, e.g., U.S. Patent Publication Nos. 2008/0166719 and 2008/0085524); renal disorders, diseases and injuries (see, e.g., U.S. Patent Publication Nos. 2008/0090304, 2008/0014644, 2008/0014604, 2007/0254370, and 2007/0037232); systemic inflammatory response syndrome (SIRS), sepsis, severe sepsis, septic shock and multiple organ dysfunction syndrome (MODS) (see, e.g., U.S. Patent Publication Nos. 2008/0050832 and 2007/0092911; see, also, U.S. Pat. No. 6,136,526); periodontal disease (see, e.g., U.S. Pat. No. 5,866,432); and venous thromboembolic disease (see, e.g., U.S. Patent Publication No. 2007/0269836), among others. In its free, uncomplexed form it is a marker for ovarian cancer, invasive and noninvasive breast cancer, and atypical ductal hyperplasia, which is a major risk factor for breast cancer (see, e.g., U.S. Patent Publication No. 2007/0196876; see, also, U.S. Pat. Nos. 5,627,034 and 5,846,739 with regard to assessing the proliferative status of a carcinoma). It also is a marker for colon (Nielsen et al. (1996) Gut 38: 414-420), pancreatic (Furutani et al. (1998) Canc. Lett. 122: 209-214), and esophageal cancer. When complexed with MMP-9, it also is a marker for conditions associated with tissue remodeling (see, e.g., U.S. Pat. Nos. 7,432,066 and 7,153,660). A high level of NGAL (e.g., approximately 350 μg/L (Xu et al. (1995) Scand. J. Clin. Lab. Invest. 55: 125-131) also can be indicative of a bacterial infection as opposed to a viral infection (see, e.g., U.S. Pat. No. 7,056,702).

Among other diseases, IL-18 and NGAL TVD binding proteins potentially can be employed in the treatment of renal disease, including any disease, disorder, or damage to or injury of the kidney, including, for example, acute renal failure, acute nephritic syndrome, analgesic nephropathy, atheroembolic renal disease, chronic renal failure, chronic nephritis, congenital nephritic syndrome, end-stage renal disease, Goodpasture syndrome, interstitial nephritis, renal cancer, renal damage, renal infection, renal injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephropathy−IgA, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis, and the like.

### III. Pharmaceutical Compositions

The present disclosure also provides pharmaceutical compositions comprising a binding protein of the present disclosure and a pharmaceutically acceptable carrier. The pharmaceutical compositions comprising binding proteins of the present disclosure are for use in, but not limited to, diagnosing, detecting, or monitoring a disorder, in preventing (e.g., inhibiting or delaying the onset of a disease, disorder or other condition), treating, managing, or ameliorating a disorder or one or more symptoms thereof, and/or in research. In a specific embodiment, a composition comprises one or more binding proteins of the present disclosure. In another embodiment the pharmaceutical composition comprises one or more binding proteins of the present disclosure and one or more prophylactic or therapeutic agents other than binding proteins of the present disclosure for treating a disorder. In one embodiment the prophylactic or therapeutic agents are those that are known to be useful for or have been or currently are being used in the prevention (e.g., the inhibition or delay of onset of a disease, disorder or other condition), treatment, management, or amelioration of a disorder or one or more symptoms thereof. In accordance with these embodiments, the composition may further comprise a carrier, diluent or excipient.

The binding proteins of the present disclosure can be incorporated into pharmaceutical compositions suitable for administration to a subject. Typically, the pharmaceutical composition comprises a binding protein of the present disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In some embodiments, isotonic agents, for example, sugars, polyalcohols such as mannitol, sorbitol, or sodium chloride, are included in the composition. Pharmaceutically acceptable carriers may further comprise minor amounts of auxiliary substances, such as wetting or emulsifying agents, preservatives or buffers, which enhance the shelf life or effectiveness of the antibody or antigen-binding fragments thereof.

Various delivery systems are known and can be used to administer one or more binding proteins, e.g., antibodies, of the present disclosure or the combination of one or more binding proteins, e.g., antibodies, of the present disclosure and a prophylactic agent or therapeutic agent useful for preventing (e.g., inhibiting or delaying the onset of a disease, disorder or other condition), managing, treating, or ameliorating a disorder or one or more symptoms thereof, e.g., encapsulation in liposomes, microparticles, microcapsules, recombinant cells that can express the antibody or antibody fragment, receptor-mediated endocytosis (see, e.g., Wu and Wu (1987) J. Biol. Chem. 262:4429-4432), and construction of a nucleic acid as part of a retroviral or other vector, etc. Methods of administering a prophylactic or therapeutic agent of the present disclosure include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer and a formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In one embodiment a binding protein of the present disclosure, combination therapy, or a composition of the present disclosure is administered using Alkermes AIR® pulmonary drug delivery technology (Alkermes, Inc., Cambridge, Mass.). In a specific embodiment, prophylactic or therapeutic agents of the present disclosure are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the present disclosure locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more binding proteins of the present disclosure antagonists is administered locally to the affected area to a subject to prevent, treat, manage, and/or ameliorate a disorder or a symptom thereof. In another embodiment, an effective amount of one or more binding proteins of the present disclosure is administered locally to the affected area in combination with an effective amount of one or more therapies (e.g., one or more prophylactic or therapeutic agents) other than a binding protein of the present disclosure of a subject to prevent, treat, manage, and/or ameliorate a disorder or one or more symptoms thereof.

In another embodiment, the prophylactic or therapeutic agent can be delivered in a controlled release or sustained release system. In one embodiment a pump may be used to achieve controlled or sustained release (see Langer, supra; Sefton (1987) CRC Crit. Ref. Biomed. Eng. 14: 20; Buchwald et al. (1980) Surgery 88: 507; Saudek et al. (1989) N. Engl. J. Med. 321: 574). In another embodiment, polymeric materials can be used to achieve controlled or sustained release of the therapies of the present disclosure (see e.g., Medical Applications of Controlled Release, Langer and Wise (eds.), CRC Pres., Boca Raton, Ha. (1974); Controlled Drug Bioavailability, Drug Product Design and Performance, Smolen and Ball (eds.), Wiley, New York (1984); Ranger and Peppas (1983) J., Macromol. Sci. Rev. Macromol. Chem. 23:61; see also Levy et al. (1985) Science 228: 190; During et al. (1989) Ann. Neurol. 25: 351; Howard et al. (1989) J. Neurosurg. 71: 105); U.S. Pat. Nos. 5,679,377; 5,916,597; 5,912,015; 5,989,463; and 5,128,326; and PCT Publication Nos. WO 99/15154; WO 99/20253. Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990) Science 249: 1527-1533). Any technique known to one of skill in the art can be used to produce sustained release formulations comprising one or more therapeutic agents of the present disclosure. See, e.g., U.S. Pat. No. 4,526,938; PCT Publication Nos. WO 91/05548; WO 96/20698, Ning et al. (1996) Radiotherap. Oncol. 39: 179-189; Song et al. (1995) PDA J. Pharma. Sci. Tech. 50:372-397; Cleek et al. (1997) Pro. Intl Symp. Control. Rel. Bioact. Matter. 24: 853-854, and Lam et al. (1997) Proc. Int'l. Symp. Control Rel. Bioact. Matter. 24:759-760.

In a specific embodiment, where the composition of the present disclosure is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide, which is known to enter the nucleus (see, e.g., Joliot et al. (1991) Proc. Natl. Acad. Sci. USA 88: 1864-1868). Alternatively, a nucleic acid can be introduced intracellularly and incorporated within host cell DNA for expression by homologous recombination.

A pharmaceutical composition of the present disclosure is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic, such as lignocamne, to ease pain at the site of the injection.

If the compositions of the present disclosure are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). In one embodiment for non-sprayable topical dosage forms, viscous to semi-solid or solid forms comprising a carrier or one or more excipients compatible with topical application and having a dynamic viscosity greater than water are employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, In one embodiment, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the present disclosure comprises intranasal administration of a composition, the composition can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base, such as lactose or starch.

If the method of the present disclosure comprises oral administration, compositions can be formulated orally in the form of tablets, capsules, cachets, gelcaps, solutions, suspensions, and the like. Tablets or capsules can be prepared by conventional means with pharmaceutically acceptable excipients, such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives, such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the present disclosure may comprise pulmonary administration, e.g., by use of an inhaler or nebulizer, of a composition formulated with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In a specific embodiment, a binding protein of the present disclosure, combination therapy, and/or composition of the present disclosure is administered using Alkermes AIR® pulmonary drug delivery technology (Alkermes, Inc., Cambridge, Mass.).

The method of the present disclosure may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents, such as suspending, stabilizing and/or dispersing agents. Alternatively, the active ingredient may be in powder form for constitution with a suitable vehicle (e.g., sterile pyrogen-free water) before use.

The methods of the present disclosure may additionally comprise administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the present disclosure encompass administration of compositions formulated as neutral or salt forms. Pharmaceutically acceptable salts include those formed with anions, such as those derived from hydrochloric, phosphoric, acetic, oxalic, and tartaric acids, etc., and those formed with cations, such as those derived from sodium, potassium, ammonium, calcium, ferric hydroxides, isopropylamine, triethylamine, 2-ethylamino ethanol, histidine, and procaine, etc.

Generally, the ingredients of compositions are supplied either separately or mixed together in unit dosage form, for example, as a dry lyophilized powder or water-free concentrate in a hermetically sealed container, such as an ampoule or sachette indicating the quantity of active agent. Where the mode of administration is infusion, the composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the present disclosure also provides that one or more of the prophylactic or therapeutic agents, or a pharmaceutical composition of the present disclosure, is packaged in a hermetically sealed container, such as an ampoule or sachette indicating the quantity of the agent. In one embodiment, one or more of the prophylactic or therapeutic agents, or a pharmaceutical composition of the present disclosure, is supplied as a dry sterilized lyophilized powder or water-free concentrate in a hermetically sealed container and can be reconstituted (e.g., with water or saline) to the appropriate concentration for administration to a subject. In one embodiment, one or more of the prophylactic or therapeutic agents or pharmaceutical compositions of the present disclosure is supplied as a dry sterile lyophilized powder in a hermetically sealed container at a unit dosage of at least 5 mg, at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the present disclosure, should be stored at between 2° C. and 8° C. in their original containers and the prophylactic or therapeutic agents, or pharmaceutical compositions of the present disclosure, should be administered within 1 week, e.g., within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the present disclosure is supplied in liquid form in a hermetically sealed container indicating the quantity and concentration of the agent. In one embodiment, the liquid form of the administered composition is supplied in a hermetically sealed container at a concentration of at least 0.25 mg/ml, at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The binding proteins of the present disclosure can be incorporated into a pharmaceutical composition suitable for parenteral administration. In one embodiment, the binding protein, or antigen-binding fragment thereof, will be prepared as an injectable solution containing 0.1-250 mg/ml binding protein. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include, but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine and arginine, either of which can be included at a concentration of 0-0.05%, and polysorbate-80 (optimally included at a concentration of 0.005-0.01%). Additional surfactants include, but are not limited to, polysorbate 20 and BRIJ surfactants. The pharmaceutical composition comprising the binding proteins of the present disclosure prepared as an injectable solution for parenteral administration can further comprise an agent useful as an adjuvant, such as those used to increase the absorption, or dispersion of a therapeutic protein (e.g., antibody). A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e., greater than 1 ml) with less pain and discomfort, and minimum incidence of injection site reactions (see PCT Publication No. WO 2004/078140, and U.S. Patent Publication No. 2006/104968).

The compositions of this present disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form chosen depends on the intended mode of administration and therapeutic application. Typical compositions are in the form of injectable or infusible solutions, such as compositions similar to those used for passive immunization of humans with other antibodies. The chosen mode of administration is parenteral (e.g., intravenous, subcutaneous, intraperitoneal, intramuscular). In one embodiment, the binding protein is administered by intravenous infusion or injection. In another embodiment, the binding protein is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (binding protein, or antigen-binding fragments thereof) in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated herein. In the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The binding proteins of the present disclosure can be administered by a variety of methods known in the art, although for many therapeutic applications, in one embodiment, the route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a binding protein of the present disclosure may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the present disclosure by other than parenteral administration, it may be necessary to coat the compound with, or co-administer the compound with, a material to prevent its inactivation.

Supplementary active compounds can also be incorporated into the compositions. In certain embodiments, a binding protein of the present disclosure is coformulated with and/or coadministered with one or more additional therapeutic agents that are useful for treating disorders with a binding protein of the present disclosure. For example, a binding protein of the present disclosure may be coformulated and/or coadministered with one or more additional binding proteins, e.g., antibodies, that bind other targets (e.g., antibodies that bind other cytokines or that bind cell surface molecules). Furthermore, one or more binding proteins of the present disclosure may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, a binding protein is linked to a half-life extending vehicle known in the art. Such vehicles include, but are not limited to, the Fc domain, polyethylene glycol, and dextran. Such vehicles are described, e.g., in U.S. Pat. No. 6,660,843 and published PCT Publication No. WO 99/25044.

In a specific embodiment, nucleic acid sequences encoding a binding protein of the present disclosure or another prophylactic or therapeutic agent of the present disclosure are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the present disclosure the nucleic acids produce their encoded binding protein or prophylactic or therapeutic agent of the present disclosure that mediates a prophylactic or therapeutic effect.

Any of the methods for gene therapy available in the art can be used according to the present disclosure. For general reviews of the methods of gene therapy, see Goldspiel et al. (1993) Clin. Pharm. 12: 488-505; Wu and Wu (1991) Biotherapy 3: 87-95; Tolstoshev (1993) Ann. Rev. Pharmacol. Toxicol. 32: 573-596; Mulligan (1993) Science 260: 926-932; and Morgan and Anderson (1993) Ann. Rev. Biochem. 62: 191-217; May (1993) TIBTECH 11(5):155-215. Methods commonly known in the art of recombinant DNA technology which can be used are described in Ausubel et al. (eds.), Current Protocols in Molecular Biology, John Wiley &Sons, NY (1993); and Kriegler, Gene Transfer and Expression, A Laboratory Manual, Stockton Press, NY (1990). Detailed descriptions of various methods of gene therapy are disclosed in U.S. Patent Publication No. 20090297514.

The binding proteins of the present disclosure are useful in treating various diseases wherein the targets that are recognized by the binding proteins are detrimental. Such diseases include, but are not limited to, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, and xenograft rejection of any organ or tissue (see PCT Publication Nos. WO 2002/097048; WO 95/24918, and WO 00/56772).

The binding proteins of the present disclosure can be used to treat humans suffering from autoimmune diseases, in particular those associated with inflammation, including, rheumatoid arthritis, spondylitis, allergy, autoimmune diabetes, autoimmune uveitis. In one embodiment the binding proteins of the present disclosure, or antigen-binding portions thereof, are used to treat rheumatoid arthritis, Crohn's disease, multiple sclerosis, insulin dependent diabetes mellitus, and psoriasis.

In one embodiment, diseases that can be treated or diagnosed with the compositions and methods of the present disclosure include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

In one embodiment, the binding proteins of the present disclosure, or antigen-binding portions thereof, are used to treat cancer or inhibit metastases from the tumors described herein, either when used alone or in combination with radiotherapy and/or other chemotherapeutic agents.

The binding proteins of the present disclosure, or antigen-binding portions thereof, may be combined with agents that include, but are not limited to, antineoplastic agents, radiotherapy, chemotherapy, such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

A binding protein of the present disclosure also can be administered with one or more additional therapeutic agents useful in the treatment of various diseases.

A binding protein of the present disclosure can be used alone or in combination to treat such diseases. It should be understood that the binding proteins may be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the binding protein of the present disclosure. The additional agent also can be an agent that imparts a beneficial attribute to the therapeutic composition, e.g., an agent which affects the viscosity of the composition.

It should further be understood that the combinations, which are to be included within this present disclosure, are those combinations useful for their intended purpose. The agents set forth below are illustrative and are not intended to be limited. The combinations, which are part of this present disclosure, can be the binding proteins of the present disclosure and at least one additional agent selected from the lists below. The combination can also include more than one additional agent, e.g., two or three additional agents, if the combination is such that the formed composition can perform its intended function.

Combinations to treat autoimmune and inflammatory diseases are non-steroidal anti-inflammatory drug(s), also referred to as NSAIDS, which include drugs like ibuprofen. Other combinations are corticosteroids including prednisolone; the well known side-effects of steroid use can be reduced or even eliminated by tapering the steroid dose required when treating patients in combination with the TVD binding proteins of this present disclosure. Non-limiting examples of therapeutic agents for rheumatoid arthritis with which an binding protein, or antigen-binding fragments thereof, of the present disclosure can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Binding proteins of the present disclosure, or antigen-binding portions thereof, can be combined with antibodies to cell surface molecules, such as CD2, CD3, CD4, CD8, CD25, CD28, CD30, CD40, CD45, CD69, CD80 (B7.1), CD86 (B7.2), CD90, and CTLA, or their ligands including CD154 (gp39 or CD40L).

Combinations of therapeutic agents may interfere at different points in the autoimmune and subsequent inflammatory cascade; examples include TNF antagonists like chimeric, humanized or human TNF antibodies, ADALIMUMONOCLONAL ANTIBODY, (PCT Publication No. WO 97/29131), CA2 (Remicade™), CDP 571, and soluble p55 or p75 TNF receptors, derivatives, thereof, (p75TNFR1gG (Enbrel™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other combinations include Interleukin 11. Yet another combination includes key players of the autoimmune response, which may act parallel to, dependent on, or in concert with, IL-12 function, especially IL-18 antagonists including IL-18 antibodies, soluble IL-18 receptors, and IL-18 binding proteins. It has been shown that IL-12 and IL-18 have overlapping but distinct functions and a combination of antagonists to both may be most effective. Yet another combination is non-depleting anti-CD4 inhibitors. Yet other combinations include antagonists of the co-stimulatory pathway CD80 (B7.1) or CD86 (B7.2) including antibodies, soluble receptors, and antagonistic ligands.

The binding proteins of the present disclosure may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines, such as TNF-α or IL-1 (e.g., IRAK, NIK, IKK, p38 or MAP kinase inhibitors), IL-1β converting enzyme inhibitors, TNFaconverting enzyme (TACE) inhibitors, T-cell signalling inhibitors, such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™ and p55TNFRIgG (Lenercept)), sIL-1R1, sIL-1R11, and sIL-6R), antiinflammatory cytokines (e.g., IL-4, IL-10, IL-11, IL-13 and TGFβ), celecoxib, folic acid, hydroxychloroquine sulfate, rofecoxib, etanercept, infliximonoclonal antibody, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximonoclonal antibody, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram. Combinations include methotrexate or leflunomide and, in moderate or severe rheumatoid arthritis cases, cyclosporine.

Nonlimiting additional agents, which can also be used in combination with a binding protein to treat rheumatoid arthritis include, but are not limited to, the following: non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2/infliximonoclonal antibody (chimeric anti-TNFα antibody; Centocor); 75 kdTNFR-IgG/etanercept (75 kD TNF receptor-IgG fusion protein; Immunex; see e.g., (1994) Arthr. Rheum. 37: 5295; (1996) J. Invest. Med. 44: 235A); 55 kdTNF-IgG (55 kD TNF receptor-IgG fusion protein; Hoffmann-LaRoche); IDEC-CE9.1/SB 210396 (non-depleting primatized anti-CD4 antibody; IDEC/SmithKline; see e.g., (1995) Arthr. Rheum. 38: S185); DAB 486-IL-2 and/or DAB 389-IL-2 (IL-2 fusion proteins; Seragen; see e.g., (1993) Arthrit. Rheum. 36: 1223); Anti-Tac (humanized anti-IL-2Rα; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-1RA (IL-1 receptor antagonist; Synergen/Amgen); anakinra (Kineret®/Amgen); TNF-bp/s-TNF (soluble TNF binding protein; see e.g., (1996) Arthr. Rheum. 39(9 (supplement)): S284; (1995) Amer. J. Physiol.—Heart and Circ. Physiol. 268: 37-42); R973401 (phosphodiesterase Type IV inhibitor; see e.g., (1996) Arthr. Rheum. 39(9 (supplement): S282); MK-966 (COX-2 Inhibitor; see e.g., (1996) Arthr. Rheum. 39(9 (supplement): S81); Iloprost (see e.g., (1996) Arthr. Rheum. 39(9 (supplement): S82); methotrexate; thalidomide (see e.g., (1996) Arthr. Rheum. 39(9 (supplement): S282) and thalidomide-related drugs (e.g., Celgen); leflunomide (anti-inflammatory and cytokine inhibitor; see e.g., (1996) Arthr. Rheum. 39(9 (supplement): S131; (1996) Inflamm. Res. 45: 103-107); tranexamic acid (inhibitor of plasminogen activation; see e.g., (1996) Arthr. Rheum. 39(9 (supplement): S284); T-614 (cytokine inhibitor; see e.g., (1996) Arthr. Rheum. 39(9 (supplement): S282); prostaglandin E1 (see e.g., (1996) Arthr. Rheum. 39(9 (supplement): S282); Tenidap (non-steroidal anti-inflammatory drug; see e.g., (1996) Arthr. Rheum. 39(9 (supplement): S280); Naproxen (non-steroidal anti-inflammatory drug; see e.g., (1996) Neuro. Report 7: 1209-1213); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine (see e.g., (1996) Arthr. Rheum. 39(9 (supplement): S281); Azathioprine (see e.g., (1996) Arthr. Rheum. 39(9 (supplement): S281); ICE inhibitor (inhibitor of the enzyme interleukin-1β converting enzyme); zap-70 and/or lck inhibitor (inhibitor of the tyrosine kinase zap-70 or lck); VEGF inhibitor and/or VEGF-R inhibitor (inhibitors of vascular endothelial cell growth factor or vascular endothelial cell growth factor receptor; inhibitors of angiogenesis); corticosteroid anti-inflammatory drugs (e.g., SB203580); TNF-convertase inhibitors; anti-IL-12 antibodies; anti-IL-18 antibodies; interleukin-11 (see e.g., (1996) Arthr. Rheum. 39(9 (supplement): S296); interleukin-13 (see e.g., (1996) Arthr. Rheum. 39(9 (supplement): S308); interleukin-17 inhibitors (see e.g., (1996) Arthr. Rheum. 39(9 (supplement): S120); gold; penicillamine; chloroquine; chlorambucil; hydroxychloroquine; cyclosporine; cyclophosphamide; total lymphoid irradiation; anti-thymocyte globulin; anti-CD4 antibodies; CD5-toxins; orally-administered peptides and collagen; lobenzarit disodium; Cytokine Regulating Agents (CRAs) HP228 and HP466 (Houghten Pharmaceuticals, Inc.); ICAM-1 antisense phosphorothioate oligo-deoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids; see e.g., DeLuca et al. (1995) Rheum. Dis. Clin. North Am. 21: 759-777); auranofin; phenylbutazone; meclofenamic acid; flufenamic acid; intravenous immune globulin; zileuton; azaribine; mycophenolic acid (RS-61443); tacrolimus (FK-506); sirolimus (rapamycin); amiprilose (therafectin); cladribine (2-chlorodeoxyadenosine); methotrexate; bcl-2 inhibitors (see Bruncko, M. et al. (2007) J. Med. Chem. 50(4): 641-662); and antivirals and immune-modulating agents.

In one embodiment, the binding protein, or antigen-binding portion thereof, is administered in combination with one of the following agents for the treatment of rheumatoid arthritis: small molecule inhibitor of KDR, small molecule inhibitor of Tie-2; methotrexate; prednisone; celecoxib; folic acid; hydroxychloroquine sulfate; rofecoxib; etanercept; infliximonoclonal antibody; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximonoclonal antibody; IL-1 TRAP; MRA; CTLA4-IG; IL-18 BP; IL-12/23; anti-IL 18; anti-IL 15; BIRB-796; SCIO-469; VX-702; AMG-548; VX-740; Roflumilast; IC-485; CDC-801; and mesopram.

Non-limiting examples of therapeutic agents for inflammatory bowel disease with which a binding protein of the present disclosure can be combined include the following: budenoside; epidermal growth factor; corticosteroids; cyclosporin; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; and antibodies to, or antagonists of, other human cytokines or growth factors, for example, TNF, LT, IL-1, IL-2, IL-6, IL-7, IL-8, IL-15, IL-16, IL-17, IL-18, EMAP-II, GM-CSF, FGF, and PDGF. Binding proteins of the present disclosure, or antigen-binding portions thereof, can be combined with antibodies to cell surface molecules, such as CD2, CD3, CD4, CD8, CD25, CD28, CD30, CD40, CD45, CD69, and CD90 or any of their ligands. The binding proteins of the present disclosure, or antigen-binding portions thereof, may also be combined with agents, such as methotrexate, cyclosporin, FK506, rapamycin, mycophenolate mofetil, leflunomide, NSAIDs such as ibuprofen, corticosteroids, such as prednisolone, phosphodiesterase inhibitors, adenosine agonists, antithrombotic agents, complement inhibitors, adrenergic agents, agents, which interfere with signalling by proinflammatory cytokines, such as TNFα or IL-1 (e.g., IRAK, NIK, IKK, p38 or MAP kinase inhibitors), IL-1β converting enzyme inhibitors, TNFα converting enzyme inhibitors, T-cell signalling inhibitors, such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1R1, sIL-1R11, and sIL-6R), antiinflammatory cytokines (e.g., IL-4, IL-10, IL-11, IL-13 and TGFβ), and bcl-2 inhibitors.

Examples of therapeutic agents for Crohn's disease in which a binding protein can be combined include the following: TNF antagonists, for example, anti-TNF antibodies, ADALIMUMONOCLONAL ANTIBODY (PCT Publication No. WO 97/29131; HUMIRA), CA2 (REMICADE), CDP 571, TNFR-Ig constructs, (p75TNFRIgG (ENBREL) and p55TNFRIgG (LENERCEPT)) inhibitors and PDE4 inhibitors. Binding proteins of the present disclosure, or antigen-binding portions thereof, can be combined with corticosteroids, for example, budenoside and dexamethasone. Binding proteins of the present disclosure, or antigen-binding portions thereof, may also be combined with agents, such as sulfasalazine, 5-aminosalicylic acid and olsalazine, and agents, which interfere with synthesis or action of proinflammatory cytokines, such as IL-1, for example, IL-1β converting enzyme inhibitors and IL-1ra. Binding proteins of the present disclosure, or antigen-binding portion thereof, may also be used with T cell signaling inhibitors, for example, tyrosine kinase inhibitors 6-mercaptopurines. Binding proteins of the present disclosure, or antigen-binding portions thereof, can be combined with IL-11. Binding proteins of the present disclosure, or antigen-binding portions thereof, can be combined with mesalamine, prednisone, azathioprine, mercaptopurine, infliximonoclonal antibody, methylprednisolone sodium succinate, diphenoxylate/atrop sulfate, loperamide hydrochloride, methotrexate, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, celecoxib, polycarbophil, propoxyphene napsylate, hydrocortisone, multivitamins, balsalazide disodium, codeine phosphate/apap, colesevelam hcl, cyanocobalamin, folic acid, levofloxacin, methylprednisolone, natalizumonoclonal antibody, and interferon-gamma.

Non-limiting examples of therapeutic agents for multiple sclerosis with which binding proteins of the present disclosure can be combined include the following: corticosteroids; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-1F (Serono/Inhale Therapeutics), Peginterferon a 2b (Enzon/Schering-Plough), Copolymer 1 (Cop-1; COPAXONE; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-23, IL-15, IL-16, IL-18, EMAP-II, GM-CSF, FGF, and PDGF. Binding proteins of the present disclosure can be combined with antibodies to cell surface molecules, such as CD2, CD3, CD4, CD8, CD19, CD20, CD25, CD28, CD30, CD40, CD45, CD69, CD80, CD86, CD90 or their ligands. Binding proteins of the present disclosure may also be combined with agents, such as methotrexate, cyclosporine, FK506, rapamycin, mycophenolate mofetil, leflunomide, NSAIDs, for example, ibuprofen, corticosteroids, such as prednisolone, phosphodiesterase inhibitors, adensosine agonists, antithrombotic agents, complement inhibitors, adrenergic agents, agents which interfere with signalling by proinflammatory cytokines, such as TNFα or IL-1 (e.g., IRAK, NIK, IKK, p38 or MAP kinase inhibitors), IL-1β converting enzyme inhibitors, TACE inhibitors, T-cell signaling inhibitors, such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1R1, sIL-1R11, and sIL-6R), antiinflammatory cytokines (e.g., IL-4, IL-10, IL-13 and TGFβ) and bcl-2 inhibitors.

Examples of therapeutic agents for multiple sclerosis in which binding proteins of the present disclosure can be combined include interferon-β, for example, IFNβ1a and IFNβ1b; copaxone, corticosteroids, caspase inhibitors, for example, inhibitors of caspase-1, IL-1 inhibitors, TNF inhibitors, and antibodies to CD40 ligand and CD80.

The binding proteins of the present disclosure may also be combined with agents, such as alemtuzumonoclonal antibody, dronabinol, Unimed, daclizumonoclonal antibody, mitoxantrone, xaliproden hydrochloride, fampridine, glatiramer acetate, natalizumonoclonal antibody, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists, and IL-4 agonists.

Non-limiting examples of therapeutic agents for Angina with which binding proteins of the present disclosure can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil hcl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril, and bisoprolol fumarate.

Non-limiting examples of therapeutic agents for Ankylosing Spondylitis with which binding proteins of the present disclosure can be combined include the following: ibuprofen, diclofenac and misoprostol, naproxen, meloxicam, indomethacin, diclofenac, celecoxib, rofecoxib, Sulfasalazine, Methotrexate, azathioprine, minocyclin, prednisone, etanercept, and infliximonoclonal antibody.

Non-limiting examples of therapeutic agents for Asthma with which binding proteins of the present disclosure can be combined include the following: albuterol, salmeterol/fluticasone, montelukast sodium, fluticasone propionate, budesonide, prednisone, salmeterol xinafoate, levalbuterol hcl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin hcl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, and metaproterenol sulfate.

Non-limiting examples of therapeutic agents for COPD with which binding proteins of the present disclosure can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol hcl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast, and Roflumilast.

Non-limiting examples of therapeutic agents for HCV with which binding proteins of the present disclosure can be combined include the following: Interferon-alpha-2a, Interferon-alpha-2b, Interferon-alpha con1, Interferon-alpha-n1, Pegylated interferon-alpha-2a, Pegylated interferon-alpha-2b, ribavirin, Peginterferon alfa-2b+ribavirin, Ursodeoxycholic Acid, Glycyrrhizic Acid, Thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which binding proteins of the present disclosure can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone hcl, potassium chloride, triamcinolone acetonide, tacrolimus anhydrous, calcium, interferon-alpha, methotrexate, mycophenolate mofetil, and Interferon-gamma-1β.

Non-limiting examples of therapeutic agents for Myocardial Infarction with which binding proteins of the present disclosure can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hcl/mag carb, bumetanide, alteplase, enalaprilat, amiodarone hydrochloride, tirofiban hcl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine hcl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for Psoriasis with which binding proteins of the present disclosure can be combined include the following: small molecule inhibitor of KDR, small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximonoclonal antibody, cyclosporine, alefacept, efalizumonoclonal antibody, tacrolimus, pimecrolimus, PUVA, UVB, and sulfasalazine.

Non-limiting examples of therapeutic agents for Psoriatic Arthritis with which binding proteins of the present disclosure can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximonoclonal antibody, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumonoclonal antibody, and bcl-2 inhibitors.

Non-limiting examples of therapeutic agents for Restenosis with which binding proteins of the present disclosure can be combined include the following: sirolimus, paclitaxel, everolimus, tacrolimus, Zotarolimus, and acetaminophen.

Non-limiting examples of therapeutic agents for Sciatica with which binding proteins of the present disclosure can be combined include the following: hydrocodone bitartrate/apap, rofecoxib, cyclobenzaprine hcl, methylprednisolone, naproxen, ibuprofen, oxycodone hcl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone hcl, tizanidine hcl, diclofenac sodium/misoprostol, propoxyphene napsylate/apap, asa/oxycod/oxycodone ter, ibuprofen/hydrocodone bit, tramadol hcl, etodolac, propoxyphene hcl, amitriptyline hcl, carisoprodol/codeine phos/asa, morphine sulfate, multivitamins, naproxen sodium, orphenadrine citrate, and temazepam.

Examples of therapeutic agents for SLE (Lupus) in which binding proteins of the present disclosure can be combined include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; and inhibitors of PDE4 or a purine synthesis inhibitor, for example, Cellcept. Binding proteins of the present disclosure may also be combined with agents, such as sulfasalazine, 5-aminosalicylic acid, olsalazine, Imuran and agents, which interfere with synthesis, production or action of proinflammatory cytokines, such as IL-1, for example, caspase inhibitors like IL-1β converting enzyme inhibitors and IL-1ra. Binding proteins of the present disclosure may also be used with T cell signaling inhibitors, for example, tyrosine kinase inhibitors, or molecules that target T cell activation molecules, for example, CTLA-4-IgG or anti-B7 family antibodies and anti-PD-1 family antibodies. Binding proteins of the present disclosure can be combined with IL-11 or anti-cytokine antibodies, for example, fonotolizumonoclonal antibody (anti-IFNg antibody), or anti-receptor receptor antibodies, for example, anti-IL-6 receptor antibody and antibodies to B-cell surface molecules. Antibodies of the present disclosure, or antigen-binding portion thereof, may also be used with LJP 394 (abetimus), agents that deplete or inactivate B-cells, for example, Rituximonoclonal antibody (anti-CD20 antibody), lymphostat-B (anti-BlyS antibody), TNF antagonists, for example, anti-TNF antibodies, Adalimumonoclonal antibody (PCT Publication No. WO 97/29131; HUMIRA), CA2 (REMICADE), CDP 571, TNFR-Ig constructs, (p75TNFRIgG (ENBREL) and p55TNFRIgG (LENERCEPT)) and bcl-2 inhibitors, because bcl-2 overexpression in transgenic mice has been demonstrated to cause a lupus like phenotype (see Marquina, R. et al. (2004) J. Immunol. 172(11): 7177-7185), therefore inhibition is expected to have therapeutic effects.

The pharmaceutical compositions of the present disclosure may include a “therapeutically effective amount” or a “prophylactically effective amount” of a binding protein of the present disclosure. A “therapeutically effective amount” refers to an amount effective, at dosages and for periods of time necessary, to achieve the desired therapeutic result. A therapeutically effective amount of the binding protein may be determined by a person skilled in the art and may vary according to factors such as the disease state, age, sex, and weight of the individual, and the ability of the binding protein to elicit a desired response in the individual. A therapeutically effective amount is also one in which any toxic or detrimental effects of the binding protein, or antigen-binding fragments thereof, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein of the present disclosure is 0.1-20 mg/kg, for example, 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### IV. Diagnostics

The disclosure herein also provides diagnostic applications. This is further elucidated below.

### A. Diagnostic Methods

The present disclosure also provides a method for determining the presence, amount or concentration of an analyte (or a fragment thereof) in a test sample using at least one TVD binding protein as described herein. Any suitable assay as is known in the art can be used in the method. Examples include, but are not limited to, immunoassay, such as sandwich immunoassay (e.g., monoclonal, polyclonal and/or TVD binding protein sandwich immunoassays or any variation thereof (e.g., monoclonal/TVD binding protein, TVD binding protein/polyclonal molecule, etc.), including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay a capture reagent that specifically binds an analyte (or a fragment thereof) of interest is attached to the surface of a mass spectrometry probe, such as a pre-activated protein chip array. The analyte (or a fragment thereof) is then specifically captured on the biochip, and the captured analyte (or a fragment thereof) is detected by mass spectrometry. Alternatively, the analyte (or a fragment thereof) can be eluted from the capture reagent and detected by traditional MALDI (matrix-assisted laser desorption/ionization) or by SELDI. A chemiluminescent microparticle immunoassay, in particular one employing the ARCHITECT® automated analyzer (Abbott Laboratories, Abbott Park, Ill.), is an example of a preferred immunoassay.

Methods well-known in the art for collecting, handling and processing urine, blood, serum and plasma, and other body fluids, are used in the practice of the present disclosure, for instance, when a TVD binding protein as described herein is employed as an immunodiagnostic reagent and/or in an analyte immunoassay kit. The test sample can comprise further moieties in addition to the analyte of interest, such as antibodies, antigens, haptens, hormones, drugs, enzymes, receptors, proteins, peptides, polypeptides, oligonucleotides and/or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples), pretreatment optionally can be done (e.g., as part of a regimen on a commercial platform).

The pretreatment reagent can be any reagent appropriate for use with the immunoassay and kits of the present disclosure. The pretreatment optionally comprises: (a) one or more solvents (e.g., methanol and ethylene glycol) and optionally, salt, (b) one or more solvents and salt, and optionally, detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., (1990) Clin. Chem. 36: 1969-1973 and Wallemacq et al. (1999) Clin. Chem. 45: 432-435), and/or as commercially available. Additionally, pretreatment can be done as described in U.S. Pat. No. 5,135,875, EU Patent Pubublication No. EU0471293, U.S. Pat. No. 6,660,843, and U.S. Patent Application No. 2008/0020401. The pretreatment reagent can be a heterogeneous agent or a homogeneous agent.

With use of a heterogeneous pretreatment reagent, the pretreatment reagent precipitates analyte binding protein (e.g., protein that can bind to an analyte or a fragment thereof) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with a labeled specific binding partner for analyte (or a fragment thereof), such as a labeled anti-analyte antibody (or an antigenically reactive fragment thereof). The pretreatment reagent employed for such an assay typically is diluted in the pretreated test sample mixture, either before or during capture by the first specific binding partner. Despite such dilution, a certain amount of the pretreatment reagent is still present (or remains) in the test sample mixture during capture. According to the present disclosure, the labeled specific binding partner can be a TVD binding protein (or a fragment, a variant, or a fragment of a variant thereof).

In a heterogeneous format, after the test sample is obtained from a subject, a first mixture is prepared. The mixture contains the test sample being assessed for an analyte (or a fragment thereof) and a first specific binding partner, wherein the first specific binding partner and any analyte contained in the test sample form a first specific binding partner-analyte complex. Preferably, the first specific binding partner is an anti-analyte antibody or a fragment thereof. The first specific binding partner can be a TVD binding protein (or a fragment, a variant, or a fragment of a variant thereof) as described herein. The order in which the test sample and the first specific binding partner are added to form the mixture is not critical. Preferably, the first specific binding partner is immobilized on a solid phase. The solid phase used in the immunoassay (for the first specific binding partner and, optionally, the second and/or third specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-analyte complex is formed, any unbound analyte is removed from the complex using any technique known in the art. For example, the unbound analyte can be removed by washing. Desirably, however, the first specific binding partner is present in excess of any analyte present in the test sample, such that all analyte that is present in the test sample is bound by the first specific binding partner.

After any unbound analyte is removed, a second specific binding partner is added to the mixture to form a first specific binding partner-analyte-second specific binding partner complex. The second specific binding partner is preferably an anti-analyte antibody that binds to an epitope on analyte that differs from the epitope on analyte bound by the first specific binding partner. Moreover, also preferably, the second specific binding partner is labeled with or contains a detectable label as described above. The second specific binding partner can be a TVD binding protein (or a fragment, a variant, or a fragment of a variant thereof) as described herein. After the mixture containing the second specific binding partner-analyte complex is formed, any unbound analyte may be removed from the complex using any technique known in the art.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896; 5,573,904; 5,496,925; 5,359,093; and 5,352,803. An acridinium compound can be used as a detectable label in a homogeneous or heterogeneous chemiluminescent assay (see, e.g., Adamczyk et al. (2006) Bioorg. Med. Chem. Lett. 16: 1324-1328; Adamczyk et al. (2004) Bioorg. Med. Chem. Lett. 4: 2313-2317; Adamczyk et al. (2004) Biorg. Med. Chem. Lett. 14: 3917-3921; and Adamczyk et al. (2003) Org. Lett. 5: 3779-3782).

A preferred acridinium compound is an acridinium-9-carboxamide. Methods for preparing acridinium 9-carboxamides are described in Mattingly (1991) J. Biolumin Chemilumin 6: 107-114; Adamczyk et al. (1998) J. Org. Chem. 63: 5636-5639; Adamczyk et al. (1999) Tetrahedron 55: 10899-10914; Adamczyk et al. (1999) Org. Lett. 1: 779-781; Adamczyk et al. (2000) Biocon. Chem. 11: 714-724; Mattingly et al., In Luminescence Biotechnology: Instruments and Applications; Dyke, K. V. Ed.; CRC Press: Boca Raton, pp. 77-105 (2002); Adamczyk et al. (2003) Org. Lett. 5: 3779-3782; and U.S. Pat. Nos. 5,468,646; 5,543,524; and 5,783,699. Another preferred acridinium compound is an acridinium-9-carboxylate aryl ester. An example of an acridinium-9-carboxylate aryl ester is 10-methyl-9-(phenoxycarbonyliacridinium fluorosulfonate (available from Cayman Chemical, Ann Arbor, Mich.). Methods for preparing acridinium 9-carboxylate aryl esters are described in McCapra et al. (1965) Photochem. Photobiol. 4: 1111-21; Razavi et al. (2000) Luminescence 15: 245-249; Razavi et al. (2000) Luminescence 15: 239-244; and U.S. Pat. No. 5,241,070. Further details regarding acridinium-9-carboxylate aryl ester and its use are set forth in US Patent Publication No. 20080248493.

Chemiluminescent assays (e.g., using acridinium as described above or other chemiluminescent agents) can be performed in accordance with the methods described in Adamczyk et al. (2006) Anal. Chim Acta 579(1): 61-67. While any suitable assay format can be used, a microplate chemiluminometer (Mithras LB-940, Berthold Technologies U.S.A., LLC, Oak Ridge, Tenn.) enables the assay of multiple samples of small volumes rapidly.

The order in which the test sample and the specific binding partner(s) are added to form the mixture for chemiluminescent assay is not critical. If the first specific binding partner is detectably labeled with a chemiluminescent agent such as an acridinium compound, detectably labeled first specific binding partner-analyte complexes form. Alternatively, if a second specific binding partner is used and the second specific binding partner is detectably labeled with a chemiluminescent agent such as an acridinium compound, detectably labeled first specific binding partner-analyte-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture (e.g., the source of the hydrogen peroxide being one or more buffers or other solutions that are known to contain hydrogen peroxide) before, simultaneously with, or after the addition of an above-described acridinium compound. Hydrogen peroxide can be generated in situ in a number of ways such as would be apparent to one skilled in the art.

Upon the simultaneous or subsequent addition of at least one basic solution to the sample, a detectable signal, namely, a chemiluminescent signal, indicative of the presence of analyte is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of analyte in the sample can be quantified. Specifically, the amount of analyte in the sample is proportional to the intensity of the signal generated. The amount of analyte present can be quantified by comparing the amount of light generated to a standard curve for analyte or by comparison to a reference standard. The standard curve can be generated using serial dilutions or solutions of known concentrations of analyte by mass spectroscopy, gravimetric methods, and other techniques known in the art. While the above is described with emphasis on use of an acridinium compound as the chemiluminescent agent, one of ordinary skill in the art can readily adapt this description for use of other chemiluminescent agents.

Analyte immunoassays generally can be conducted using any format known in the art, such as, but not limited to, a sandwich format. Specifically, in one immunoassay format, at least two binding proteins, e.g., antibodies, are employed to separate and quantify analyte, such as human analyte, or a fragment thereof in a sample. More specifically, the at least two binding proteins, e.g., antibodies, bind to different epitopes on an analyte (or a fragment thereof) forming an immune complex, which is referred to as a “sandwich.” Generally, in the immunoassays one or more antibodies can be used to capture the analyte (or a fragment thereof) in the test sample (these antibodies are frequently referred to as a “capture” antibody or “capture” antibodies) and one or more binding proteins, e.g., antibodies, can be used to bind a detectable (namely, quantifiable) label to the sandwich (these antibodies are frequently referred to as the “detection antibody,” the “detection antibodies,” the “conjugate,” or the “conjugates”). Thus, in the context of a sandwich immunoassay format, a binding protein or a TVD binding protein (or a fragment, a variant, or a fragment of a variant thereof) as described herein can be used as a capture antibody, a detection antibody, or both. For example, one binding protein or TVD binding protein having a domain that can bind a first epitope on an analyte (or a fragment thereof) can be used as a capture agent and/or another binding protein or TVD binding protein having a domain that can bind a second epitope on an analyte (or a fragment thereof) can be used as a detection agent. In this regard, a binding protein or a TVD binding protein having a first domain that can bind a first epitope on an analyte (or a fragment thereof) and a second domain that can bind a second epitope on an analyte (or a fragment thereof) can be used as a capture agent and/or a detection agent. Alternatively, one binding protein or TVD binding protein having a first domain that can bind an epitope on a first analyte (or a fragment thereof) and a second domain that can bind an epitope on a second analyte (or a fragment thereof) can be used as a capture agent and/or a detection agent to detect, and optionally quantify, two or more analytes. In the event that an analyte can be present in a sample in more than one form, such as a monomeric form and a dimeric/multimeric form, which can be homomeric or heteromeric, one binding protein or TVD binding protein having a domain that can bind an epitope that is only exposed on the monomeric form and another binding protein or TVD binding protein having a domain that can bind an epitope on a different part of a dimeric/multimeric form can be used as capture agents and/or detection agents, thereby enabling the detection, and optional quantification, of different forms of a given analyte. Furthermore, employing binding proteins or TVD binding proteins with differential affinities within a single binding protein or TVD binding proteins and/or between binding proteins or TVD binding proteins can provide an avidity advantage. In the context of immunoassays as described herein, it generally may be helpful or desired to incorporate one or more linkers within the structure of a binding protein or a TVD binding protein. When present, optimally the linker should be of sufficient length and structural flexibility to enable binding of an epitope by the inner domains as well as binding of another epitope by the outer domains. In this regard, when a binding protein or a TVD binding protein can bind two different analytes and one analyte is larger than the other, desirably the larger analyte is bound by the outer domains.

Generally speaking, a sample being tested for (for example, suspected of containing) analyte (or a fragment thereof) can be contacted with at least one capture agent (or agents) and at least one detection agent (which can be a second detection agent or a third detection agent or even a successively numbered agent, e.g., as where the capture and/or detection agent comprises multiple agents) either simultaneously or sequentially and in any order. For example, the test sample can be first contacted with at least one capture agent and then (sequentially) with at least one detection agent. Alternatively, the test sample can be first contacted with at least one detection agent and then (sequentially) with at least one capture agent. In yet another alternative, the test sample can be contacted simultaneously with a capture agent and a detection agent.

In the sandwich assay format, a sample suspected of containing analyte (or a fragment thereof) is first brought into contact with at least one first capture agent under conditions that allow the formation of a first agent/analyte complex. If more than one capture agent is used, a first capture agent/analyte complex comprising two or more capture agents is formed. In a sandwich assay, the agents, i.e., preferably, the at least one capture agent, are used in molar excess amounts of the maximum amount of analyte (or a fragment thereof) expected in the test sample. For example, from about 5 μg to about 1 mg of agent per mL of buffer (e.g., microparticle coating buffer) can be used.

Competitive inhibition immunoassays, which are often used to measure small analytes because binding by only one antibody (i.e., a binding protein and/or a TVD binding protein in the context of the present disclosure) is required, comprise sequential and classic formats. In a sequential competitive inhibition immunoassay a capture agent to an analyte of interest is coated onto a well of a microtiter plate or other solid support. When the sample containing the analyte of interest is added to the well, the analyte of interest binds to the capture agent. After washing, a known amount of labeled (e.g., biotin or horseradish peroxidase (HRP)) analyte capable of binding the capture antibody is added to the well. A substrate for an enzymatic label is necessary to generate a signal. An example of a suitable substrate for HRP is 3,3′,5,5′-tetramethylbenzidine (TMB). After washing, the signal generated by the labeled analyte is measured and is inversely proportional to the amount of analyte in the sample. In a classic competitive inhibition immunoassay typically an antibody (i.e., a binding protein and/or a TVD binding protein in the context of the present disclosure) to an analyte of interest is coated onto a solid support (e.g., a well of a microtiter plate). However, unlike the sequential competitive inhibition immunoassay, the sample and the labeled analyte are added to the well at the same time. Any analyte in the sample competes with labeled analyte for binding to the capture agent. After washing, the signal generated by the labeled analyte is measured and is inversely proportional to the amount of analyte in the sample. Of course, there are many variations of these formats—e.g., such as when binding to the solid substrate takes place, whether the format is one-step, two-step, delayed two-step, and the like—and these would be recognized by one of ordinary skill in the art.

Optionally, prior to contacting the test sample with the at least one capture agent (for example, the first capture agent), the at least one capture agent can be bound to a solid support, which facilitates the separation of the first agent/analyte (or a fragment thereof) complex from the test sample. The substrate to which the capture agent is bound can be any suitable solid support or solid phase that facilitates separation of the capture agent-analyte complex from the sample.

Examples include a well of a plate, such as a microtiter plate, a test tube, a porous gel (e.g., silica gel, agarose, dextran, or gelatin), a polymeric film (e.g., polyacrylamide), beads (e.g., polystyrene beads or magnetic beads), a strip of a filter/membrane (e.g., nitrocellulose or nylon), microparticles (e.g., latex particles, magnetizable microparticles (e.g., microparticles having ferric oxide or chromium oxide cores and homo- or hetero-polymeric coats and radii of about 1-10 microns). The substrate can comprise a suitable porous material with a suitable surface affinity to bind antigens and sufficient porosity to allow access by detection antibodies. A microporous material is generally preferred, although a gelatinous material in a hydrated state can be used. Such porous substrates are preferably in the form of sheets having a thickness of about 0.01 to about 0.5 mm, preferably about 0.1 mm. While the pore size may vary quite a bit, preferably the pore size is from about 0.025 to about 15 microns, more preferably from about 0.15 to about 15 microns. The surface of such substrates can be passively coated or activated by chemical processes that cause covalent linkage of an antibody to the substrate. Irreversible binding, generally by adsorption through hydrophobic forces, of the antigen or the antibody to the substrate results; alternatively, a chemical coupling agent or other means can be used to bind covalently the antibody to the substrate, provided that such binding does not interfere with the ability of the antibody to bind to analyte. Alternatively, the antibody (i.e., binding protein and/or TVD binding protein in the context of the present disclosure) can be bound with microparticles, which have been previously coated with streptavidin (e.g., DYNAL® Magnetic Beads, Invitrogen, Carlsbad, Calif.) or biotin (e.g., using Power-Bind™-SA-MP streptavidin-coated microparticles (Seradyn, Indianapolis, Ind.)) or anti-species-specific monoclonal antibodies (i.e., binding proteins and/or TVD binding proteins in the context of the present disclosure). If necessary or desired, the substrate (e.g., for the label) can be derivatized to allow reactivity with various functional groups on the antibody (i.e., binding protein or TVD binding protein in the context of the present disclosure). Such derivatization requires the use of certain coupling agents, examples of which include, but are not limited to, maleic anhydride, N-hydroxysuccinimide, and 1-ethyl-3-(3-dimethylaminopropyl) carbodiimide. If desired, one or more capture agents, such as antibodies (or fragments thereof) (i.e., binding proteins and/or TVD binding proteins in the context of the present disclosure), each of which is specific for analyte(s) can be attached to solid phases in different physical or addressable locations (e.g., such as in a biochip configuration (see, e.g., U.S. Pat. No. 6,225,047; PCT Publication No. WO 99/51773; U.S. Pat. No. 6,329,209; PCT Publication No. WO 00/56934, and U.S. Pat. No. 5,242,828). If the capture agent is attached to a mass spectrometry probe as the solid support, the amount of analyte bound to the probe can be detected by laser desorption ionization mass spectrometry. Alternatively, a single column can be packed with different beads, which are derivatized with the one or more capture agents, thereby capturing the analyte in a single place (see, antibody-derivatized, bead-based technologies, e.g., the xMAP technology of Luminex (Austin, Tex.)).

After the test sample being assayed for analyte (or a fragment thereof) is brought into contact with the at least one capture agent (for example, the first capture agent), the mixture is incubated in order to allow for the formation of a first capture agent (or multiple capture agent)-analyte (or a fragment thereof) complex. The incubation can be carried out at a pH of from about 4.5 to about 10.0, at a temperature of from about 2° C. to about 45° C., and for a period from at least about one (1) minute to about eighteen (18) hours, preferably from about 1 to about 24 minutes, most preferably for about 4 to about 18 minutes. The immunoassay described herein can be conducted in one step (meaning the test sample, at least one capture agent and at least one detection agent are all added sequentially or simultaneously to a reaction vessel) or in more than one step, such as two steps, three steps, etc.

After formation of the (first or multiple) capture agent/analyte (or a fragment thereof) complex, the complex is then contacted with at least one detection agent under conditions which allow for the formation of a (first or multiple) capture agent/analyte (or a fragment thereof)/second detection agent complex). While captioned for clarity as the “second” agent (e.g., second detection agent), in fact, where multiple agents are used for capture and/or detection, the at least one detection agent can be the second, third, fourth, etc. agents used in the immunoassay. If the capture agent/analyte (or a fragment thereof) complex is contacted with more than one detection agent, then a (first or multiple) capture agent/analyte (or a fragment thereof)/(multiple) detection agent complex is formed. As with the capture agent (e.g., the first capture agent), when the at least one (e.g., second and any subsequent) detection agent is brought into contact with the capture agent/analyte (or a fragment thereof) complex, a period of incubation under conditions similar to those described above is required for the formation of the (first or multiple) capture agent/analyte (or a fragment thereof)/(second or multiple) detection agent complex. Preferably, at least one detection agent contains a detectable label. The detectable label can be bound to the at least one detection agent (e.g., the second detection agent) prior to, simultaneously with, or after the formation of the (first or multiple) capture agent/analyte (or a fragment thereof)/(second or multiple) detection agent complex. Any detectable label known in the art can be used (see discussion above, including of the Polak and Van Noorden (1997) and Haugland (1996) references).

The detectable label can be bound to the agents either directly or through a coupling agent. An example of a coupling agent that can be used is EDAC (1-ethyl-3-(3-dimethylaminopropyl) carbodiimide, hydrochloride), which is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the agent, such as CPSP-Acridinium Ester (i.e., 9-[N-tosyl-N-(3-carboxypropyl)]-10-(3-sulfopropyl)acridinium carboxamide) or SPSP-Acridinium Ester (i.e., N10-(3-sulfopropyl)-N-(3-sulfopropyl)-acridinium-9-carboxamide).

The (first or multiple) capture agent/analyte/(second or multiple) detection agent complex can be, but does not have to be, separated from the remainder of the test sample prior to quantification of the label. For example, if the at least one capture agent (e.g., the first capture agent, such as a binding protein and/or a TVD binding protein in accordance with the present disclosure) is bound to a solid support, such as a well or a bead, separation can be accomplished by removing the fluid (of the test sample) from contact with the solid support. Alternatively, if the at least first capture agent is bound to a solid support, it can be simultaneously contacted with the analyte-containing sample and the at least one second detection agent to form a first (multiple) agent/analyte/second (multiple) agent complex, followed by removal of the fluid (test sample) from contact with the solid support. If the at least one first capture agent is not bound to a solid support, then the (first or multiple) capture agent/analyte/(second or multiple) detection agent complex does not have to be removed from the test sample for quantification of the amount of the label.

After formation of the labeled capture agent/analyte/detection agent complex (e.g., the first capture agent/analyte/second detection agent complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using appropriate means, such as a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified by detecting the light emitted either visually or by using luminometers, x-ray film, high speed photographic film, a CCD camera, etc. Once the amount of the label in the complex has been quantified, the concentration of analyte or a fragment thereof in the test sample is determined by appropriate means, such as by use of a standard curve that has been generated using serial dilutions of analyte or a fragment thereof of known concentration. Other than using serial dilutions of analyte or a fragment thereof, the standard curve can be generated gravimetrically, by mass spectroscopy and by other techniques known in the art.

In a chemiluminescent microparticle assay employing the ARCHITECT® analyzer, the conjugate diluent pH should be about 6.0+/−0.2, the microparticle coating buffer should be maintained at about room temperature (i.e., at from about 17 to about 27° C.), the microparticle coating buffer pH should be about 6.5+/−0.2, and the microparticle diluent pH should be about 7.8+/−0.2. Solids preferably are less than about 0.2%, such as less than about 0.15%, less than about 0.14%, less than about 0.13%, less than about 0.12%, or less than about 0.11%, such as about 0.10%.

FPIAs are based on competitive binding immunoassay principles. A fluorescently labeled compound, when excited by a linearly polarized light, will emit fluorescence having a degree of polarization inversely proportional to its rate of rotation. When a fluorescently labeled tracer-antibody complex is excited by a linearly polarized light, the emitted light remains highly polarized because the fluorophore is constrained from rotating between the time light is absorbed and the time light is emitted. When a “free” tracer compound (i.e., a compound that is not bound to an antibody) is excited by linearly polarized light, its rotation is much faster than the corresponding tracer-antibody conjugate (or tracer-binding protein and/or tracer-TVD binding protein in accordance with the present disclosure) produced in a competitive binding immunoassay. FPIAs are advantageous over RIAs inasmuch as there are no radioactive substances requiring special handling and disposal. In addition, FPIAs are homogeneous assays that can be easily and rapidly performed.

In view of the above, a method of determining the presence, amount, or concentration of analyte (or a fragment thereof) in a test sample is provided. The methods include assaying the test sample for an analyte (or a fragment thereof) by an assay (i) employing (i′) at least one of an binding protein, e.g., antibody, a fragment of an antibody that can bind to an analyte, a variant of an binding protein, e.g., antibody, that can bind to an analyte, a fragment of a variant of an antibody that can bind to an analyte, a binding protein as disclosed herein, and a TVD binding protein (or a fragment, a variant, or a fragment of a variant thereof) that can bind to an analyte, and (ii′) at least one detectable label and (ii) comprising comparing a signal generated by the detectable label as a direct or indirect indication of the presence, amount or concentration of analyte (or a fragment thereof) in the test sample to a signal generated as a direct or indirect indication of the presence, amount or concentration of analyte (or a fragment thereof) in a control or calibrator. The calibrator is optionally part of a series of calibrators, in which each of the calibrators differs from the other calibrators by the concentration of analyte.

The methods may include (i) contacting the test sample with at least one capture agent, which binds to an epitope on the antigen, or fragment thereof, so as to form a capture agent/antigen, or fragment thereof, complex, (ii) contacting the capture agent/antigen, or fragment thereof, complex with at least one detection agent, which comprises a detectable label and binds to an epitope on the antigen, or fragment thereof, that is not bound by the capture agent, to form a capture agent/antigen, or fragment thereof/detection agent complex, and (iii) determining the presence, amount or concentration of the antigen, or fragment thereof, in the test sample based on the signal generated by the detectable label in the capture agent/antigen, or a fragment thereof/detection agent complex formed in (ii), whereupon the presence, amount or concentration of the antigen, or a fragment thereof, in the test sample is determined, wherein at least one capture agent and/or at least one detection agent is the at least one binding protein.

A method in which at least one first specific binding partner for analyte (or a fragment thereof), and at least one second specific binding partner for analyte (or a fragment thereof), is a binding protein as disclosed herein or a TVD binding protein (or a fragment, a variant, or a fragment of a variant thereof) as described herein can be preferred.

Alternatively, the methods may include (i) contacting the test sample with at least one capture agent, which binds to an epitope on the antigen, or fragment thereof, so as to form a capture agent/antigen, or fragment thereof, complex, and simultaneously or sequentially, in either order, contacting the test sample with detectably labeled antigen, or fragment thereof, which can compete with any antigen, or fragment thereof, in the test sample for binding to the at least one capture agent, wherein any antigen (or fragment thereof) present in the test sample and the detectably labeled antigen compete with each other to form a capture agent/antigen, or fragment thereof, complex and a capture agent/detectably labeled antigen, or fragment thereof, complex, respectively, and (ii) determining the presence, amount or concentration of the antigen, or fragment thereof, in the test sample based on the signal generated by the detectable label in the capture agent/detectably labeled antigen, or fragment thereof, complex formed in (ii), wherein at least one capture agent is the at least one binding protein, wherein the signal generated by the detectable label in the capture agent/detectably labeled antigen, or fragment thereof, complex is inversely proportional to the amount or concentration of antigen, or fragment thereof, in the test sample, whereupon the presence, amount or concentration of antigen, or fragment thereof, in the test sample is determined.

The above methods can further comprise diagnosing, prognosticating, or assessing the efficacy of a therapeutic/prophylactic treatment of a patient from whom the test sample was obtained. If the method further comprises assessing the efficacy of a therapeutic/prophylactic treatment of the patient from whom the test sample was obtained, the method optionally further comprises modifying the therapeutic/prophylactic treatment of the patient as needed to improve efficacy. The method can be adapted for use in an automated system or a semi-automated system.

More specifically, a method of determining the presence, amount or concentration of an antigen (or a fragment thereof) in a test sample is provided. The antigen (or fragment thereof) is selected from the group consisting of IL-12, IL-13, IL-18, TNFα, and PGE2. The method comprises assaying the test sample for the antigen (or a fragment thereof) by an immunoassay. The immunoassay (i) employs at least one binding protein and at least one detectable label and (ii) comprises comparing a signal generated by the detectable label as a direct or indirect indication of the presence, amount or concentration of the antigen (or a fragment thereof) in the test sample to a signal generated as a direct or indirect indication of the presence, amount or concentration of the antigen (or a fragment thereof) in a control or a calibrator. The calibrator is optionally part of a series of calibrators in which each of the calibrators differs from the other calibrators in the series by the concentration of the antigen (or a fragment thereof).

In one embodiment, one of the at least one binding proteins (i′) comprises one or more polypeptide chains comprising VD1-(X1)n-VD2-(X2)n-VD3-C-(X3)n, wherein, VD1 is a first heavy chain variable domain obtained from a first parent binding protein, e.g., antibody, or antigen-binding portion thereof, VD2 is a second heavy chain variable domain obtained from a second parent binding protein, e.g., antibody, or antigen-binding portion thereof, VD3 is a third heavy chain variable domain obtained from a third parent binding protein, e.g., antibody, or antigen-binding portion thereof, C is a heavy chain constant domain, X1 is a first linker, X1 is a second linker, X3 is an Fc region; and n is 0 or 1.

In another embodiment, one of the at least one binding proteins (i′) comprises one or more polypeptide chains comprising VD1-(X1)n-VD2-(X2)n-VD3-C-(X3)n, wherein, VD1 is a first light chain variable domain obtained from a first parent binding protein, e.g., antibody, or antigen-binding portion thereof, VD2 is a second light chain variable domain obtained from a second parent binding protein, e.g., antibody, or antigen-binding portion thereof, VD3 is a third light chain variable domain obtained from a third parent binding protein, e.g., antibody, or antigen-binding portion thereof, C is a light chain constant domain, X1 is a first linker, X1 is a second linker, X3 is an Fc region; and n is 0 or 1.

In another embodiment, one of the at least one binding proteins comprises four polypeptide chains, wherein each of the first and third polypeptide chains independently comprise VD1-(X1)n-VD2-(X2)n-VD3-C-(X3)n, wherein VD1 is a first heavy chain variable domain, VD2 is a second heavy chain variable domain, VD3 is a third heavy chain variable domain, C is a heavy chain constant domain, X1 is a first linker, X2 is a second linker, X3 is an Fc region and wherein each of the second and fourth polypeptide chains independently comprise VD1-(X1)n-VD2-(X2)n-VD3-C-(X3)n, wherein VD1 is a first light chain variable domain, VD2 is a second light chain variable domain, VD3 is a third light chain variable domain, C is a light chain constant domain, X1 is a linker, X2 is a second linker X3 does not comprise an Fc region and n is 0 or 1.

The test sample can be from a patient, in which case the method can further comprise diagnosing, prognosticating, or assessing the efficacy of therapeutic/prophylactic treatment of the patient. If the method further comprises assessing the efficacy of therapeutic/prophylactic treatment of the patient, the method optionally further comprises modifying the therapeutic/prophylactic treatment of the patient as needed to improve efficacy. The method can be adapted for use in an automated system or a semi-automated system.

With regard to the methods of assay (and kit therefor), it may be possible to employ commercially available anti-analyte antibodies or methods for production of anti-analyte as described in the literature. Commercial supplies of various antibodies include, but are not limited to, Santa Cruz Biotechnology Inc. (Santa Cruz, Calif.), GenWay Biotech, Inc. (San Diego, Calif.), and R&D Systems (RDS; Minneapolis, Minn.).

Generally, a predetermined level can be employed as a benchmark against which to assess results obtained upon assaying a test sample for analyte or a fragment thereof, e.g., for detecting disease or risk of disease. Generally, in making such a comparison, the predetermined level is obtained by running a particular assay a sufficient number of times and under appropriate conditions such that a linkage or association of analyte presence, amount or concentration with a particular stage or endpoint of a disease, disorder or condition or with particular clinical indicia can be made. Typically, the predetermined level is obtained with assays of reference subjects (or populations of subjects). The analyte measured can include fragments thereof, degradation products thereof, and/or enzymatic cleavage products thereof.

In particular, with respect to a predetermined level as employed for monitoring disease progression and/or treatment, the amount or concentration of analyte or a fragment thereof may be “unchanged,” “favorable” (or “favorably altered”), or “unfavorable” (or “unfavorably altered”). “Elevated” or “increased” refers to an amount or a concentration in a test sample that is higher than a typical or normal level or range (e.g., predetermined level), or is higher than another reference level or range (e.g., earlier or baseline sample). The term “lowered” or “reduced” refers to an amount or a concentration in a test sample that is lower than a typical or normal level or range (e.g., predetermined level), or is lower than another reference level or range (e.g., earlier or baseline sample). The term “altered” refers to an amount or a concentration in a sample that is altered (increased or decreased) over a typical or normal level or range (e.g., predetermined level), or over another reference level or range (e.g., earlier or baseline sample).

The typical or normal level or range for analyte is defined in accordance with standard practice. Because the levels of analyte in some instances will be very low, a so-called altered level or alteration can be considered to have occurred when there is any net change as compared to the typical or normal level or range, or reference level or range, that cannot be explained by experimental error or sample variation. Thus, the level measured in a particular sample will be compared with the level or range of levels determined in similar samples from a so-called normal subject. In this context, a “normal subject” is an individual with no detectable disease, for example, and a “normal” (sometimes termed “control”) patient or population is/are one(s) that exhibit(s) no detectable disease, respectively, for example. Furthermore, given that analyte is not routinely found at a high level in the majority of the human population, a “normal subject” can be considered an individual with no substantial detectable increased or elevated amount or concentration of analyte, and a “normal” (sometimes termed “control”) patient or population is/are one(s) that exhibit(s) no substantial detectable increased or elevated amount or concentration of analyte. An “apparently normal subject” is one in which analyte has not yet been or currently is being assessed. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having, or at risk of having, a particular disease, disorder, or condition. The method of assay can also involve the assay of other markers and the like.

Accordingly, the methods described herein also can be used to determine whether or not a subject has or is at risk of developing a given disease, disorder or condition. Specifically, such a method can comprise the steps of (a) determining the concentration or amount in a test sample from a subject of analyte (or a fragment thereof) (e.g., using the methods described herein, or methods known in the art); and (b) comparing the concentration or amount of analyte (or a fragment thereof) determined in step (a) with a predetermined level, wherein, if the concentration or amount of analyte determined in step (a) is favorable with respect to a predetermined level, then the subject is determined not to have or be at risk for a given disease, disorder or condition. However, if the concentration or amount of analyte determined in step (a) is unfavorable with respect to the predetermined level, then the subject is determined to have or be at risk for a given disease, disorder or condition.

Additionally, provided herein is method of monitoring the progression of disease in a subject. The methods include the steps of: (a) determining the concentration or amount in a test sample from a subject of analyte, (b) determining the concentration or amount in a later test sample from the subject of analyte and (c) comparing the concentration or amount of analyte as determined in step (b) with the concentration or amount of analyte determined in step (a), wherein if the concentration or amount determined in step (b) is unchanged or is unfavorable when compared to the concentration or amount of analyte determined in step (a), then the disease in the subject is determined to have continued, progressed or worsened. By comparison, if the concentration or amount of analyte as determined in step (b) is favorable when compared to the concentration or amount of analyte as determined in step (a), then the disease in the subject is determined to have discontinued, regressed or improved.

Optionally, the method further comprises comparing the concentration or amount of analyte as determined in step (b), for example, with a predetermined level. Further, optionally the method comprises treating the subject with one or more pharmaceutical compositions for a period of time if the comparison shows that the concentration or amount of analyte as determined in step (b), for example, is unfavorably altered with respect to the predetermined level.

Still further, the methods can be used to monitor treatment in a subject receiving treatment with one or more pharmaceutical compositions. Specifically, such methods involve providing a first test sample from a subject before the subject has been administered one or more pharmaceutical compositions. Next, the concentration or amount in a first test sample from a subject of analyte is determined (e.g., using the methods described herein or as known in the art). After the concentration or amount of analyte is determined, optionally the concentration or amount of analyte is then compared with a predetermined level. If the concentration or amount of analyte as determined in the first test sample is lower than the predetermined level, then the subject is not treated with one or more pharmaceutical compositions. However, if the concentration or amount of analyte as determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about seven (7) days to about two years, preferably from about fourteen (14) days to about one (1) year).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of analyte is determined in the second or subsequent test sample is determined (e.g., using the methods described herein or as known in the art). The concentration or amount of analyte as determined in each of the second and subsequent test samples is then compared with the concentration or amount of analyte as determined in the first test sample (e.g., the test sample that was originally optionally compared to the predetermined level). If the concentration or amount of analyte as determined in step (c) is favorable when compared to the concentration or amount of analyte as determined in step (a), then the disease in the subject is determined to have discontinued, regressed or improved, and the subject should continue to be administered the one or pharmaceutical compositions of step (b). However, if the concentration or amount determined in step (c) is unchanged or is unfavorable when compared to the concentration or amount of analyte as determined in step (a), then the disease in the subject is determined to have continued, progressed or worsened, and the subject should be treated with a higher concentration of the one or more pharmaceutical compositions administered to the subject in step (b) or the subject should be treated with one or more pharmaceutical compositions that are different from the one or more pharmaceutical compositions administered to the subject in step (b). Specifically, the subject can be treated with one or more pharmaceutical compositions that are different from the one or more pharmaceutical compositions that the subject had previously received to decrease or lower said subject's analyte level.

Generally, for assays in which repeat testing may be done (e.g., monitoring disease progression and/or response to treatment), a second or subsequent test sample is obtained at a period in time after the first test sample has been obtained from the subject. Specifically, a second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years after the first test sample from the subject is obtained.

When used to monitor disease progression, the above assay can be used to monitor the progression of disease in subjects suffering from acute conditions. Acute conditions, also known as critical care conditions, refer to acute, life-threatening diseases or other critical medical conditions involving, for example, the cardiovascular system or excretory system. Typically, critical care conditions refer to those conditions requiring acute medical intervention in a hospital-based setting (including, but not limited to, the emergency room, intensive care unit, trauma center, or other emergent care setting) or administration by a paramedic or other field-based medical personnel. For critical care conditions, repeat monitoring is generally done within a shorter time frame, namely, minutes, hours or days (e.g., about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days), and the initial assay likewise is generally done within a shorter timeframe, e.g., about minutes, hours or days of the onset of the disease or condition.

The assays also can be used to monitor the progression of disease in subjects suffering from chronic or non-acute conditions. Non-critical care or, non-acute conditions, refers to conditions other than acute, life-threatening disease or other critical medical conditions involving, for example, the cardiovascular system and/or excretory system. Typically, non-acute conditions include those of longer-term or chronic duration. For non-acute conditions, repeat monitoring generally is done with a longer timeframe, e.g., hours, days, weeks, months or years (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years), and the initial assay likewise generally is done within a longer time frame, e.g., about hours, days, months or years of the onset of the disease or condition.

Furthermore, the above assays can be performed using a first test sample obtained from a subject where the first test sample is obtained from one source, such as urine, serum or plasma. Optionally, the above assays can then be repeated using a second test sample obtained from the subject where the second test sample is obtained from another source. For example, if the first test sample was obtained from urine, the second test sample can be obtained from serum or plasma. The results obtained from the assays using the first test sample and the second test sample can be compared. The comparison can be used to assess the status of a disease or condition in the subject.

Moreover, the present disclosure also relates to methods of determining whether a subject predisposed to or suffering from a given disease, disorder or condition will benefit from treatment. In particular, the disclosure relates to analyte companion diagnostic methods and products. Thus, the method of “monitoring the treatment of disease in a subject” as described herein further optimally also can encompass selecting or identifying candidates for therapy.

Thus, in particular embodiments, the disclosure also provides a method of determining whether a subject having, or at risk for, a given disease, disorder or condition is a candidate for therapy. Generally, the subject is one who has experienced some symptom of a given disease, disorder or condition or who has actually been diagnosed as having, or being at risk for, a given disease, disorder or condition, and/or who demonstrates an unfavorable concentration or amount of analyte or a fragment thereof, as described herein.

The method optionally comprises an assay as described herein, where analyte is assessed before and following treatment of a subject with one or more pharmaceutical compositions (e.g., particularly with a pharmaceutical related to a mechanism of action involving analyte), with immunosuppressive therapy, or by immunoabsorption therapy, or where analyte is assessed following such treatment and the concentration or the amount of analyte is compared against a predetermined level. An unfavorable concentration of amount of analyte observed following treatment confirms that the subject will not benefit from receiving further or continued treatment, whereas a favorable concentration or amount of analyte observed following treatment confirms that the subject will benefit from receiving further or continued treatment. This confirmation assists with management of clinical studies, and provision of improved patient care.

It goes without saying that, while certain embodiments herein are advantageous when employed to assess a given disease, disorder or condition as discussed herein, the assays and kits can be employed to assess analyte in other diseases, disorders and conditions. The method of assay can also involve the assay of other markers and the like.

The method of assay also can be used to identify a compound that ameliorates a given disease, disorder or condition. For example, a cell that expresses analyte can be contacted with a candidate compound. The level of expression of analyte in the cell contacted with the compound can be compared to that in a control cell using the method of assay described herein.

### B. Kits

A kit for assaying a test sample for the presence, amount or concentration of an analyte (or a fragment thereof) in a test sample is also provided. The kit comprises at least one component for assaying the test sample for the analyte (or a fragment thereof) and instructions for assaying the test sample for the analyte (or a fragment thereof). The at least one component for assaying the test sample for the analyte (or a fragment thereof) can include a composition comprising a binding protein as disclosed herein and/or an anti-analyte TVD binding protein (or a fragment, a variant, or a fragment of a variant thereof), which is optionally immobilized on a solid phase.

The kit can comprise at least one component for assaying the test sample for an analyte by immunoassay, e.g., chemiluminescent microparticle immunoassay, and instructions for assaying the test sample for an analyte by immunoassay, e.g., chemiluminescent microparticle immunoassay. For example, the kit can comprise at least one specific binding partner for an analyte, such as an anti-analyte, monoclonal/polyclonal antibody (or a fragment thereof that can bind to the analyte, a variant thereof that can bind to the analyte, or a fragment of a variant that can bind to the analyte), a binding protein as disclosed herein, or an anti-analyte TVD binding protein (or a fragment, a variant, or a fragment of a variant thereof), either of which can be detectably labeled. Alternatively or additionally, the kit can comprise detectably labeled analyte (or a fragment thereof that can bind to an anti-analyte, monoclonal/polyclonal antibody, a binding protein as disclosed herein, or an anti-analyte TVD binding protein (or a fragment, a variant, or a fragment of a variant thereof)), which can compete with any analyte in a test sample for binding to an anti-analyte, monoclonal/polyclonal antibody (or a fragment thereof that can bind to the analyte, a variant thereof that can bind to the analyte, or a fragment of a variant that can bind to the analyte), a binding protein as disclosed herein, or an anti-analyte TVD binding protein (or a fragment, a variant, or a fragment of a variant thereof), either of which can be immobilized on a solid support. The kit can comprise a calibrator or control, e.g., isolated or purified analyte. The kit can comprise at least one container (e.g., tube, microtiter plates or strips, which can be already coated with a first specific binding partner, for example) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions can be in paper form or computer-readable form, such as a disk, CD, DVD, or the like.

More specifically, provided is a kit for assaying a test sample for an antigen (or a fragment thereof). The kit comprises at least one component for assaying the test sample for an antigen (or a fragment thereof) and instructions for assaying the test sample for an antigen (or a fragment thereof), wherein the at least one component includes at least one composition comprising a binding protein, which (i′) comprises one or more polypeptide chains comprising VD1-(X1)n-VD2-(X2)n-VD3-C-(X3)n, wherein, VD1 is a first heavy chain variable domain obtained from a first parent binding protein, e.g., antibody, or antigen-binding portion thereof, VD2 is a second heavy chain variable domain obtained from a second parent binding protein, e.g., antibody, or antigen-binding portion thereof, VD3 is a third heavy chain variable domain obtained from a third parent binding protein, e.g., antibody, or antigen-binding portion thereof, C is a heavy chain constant domain, X1 is a first linker, X1 is a second linker, X3 is an Fc region; and n is 0 or 1, and (ii′) can bind a triplet of antigens selected from the group consisting of prostaglandin E2 (PGE2), interleukin 13 (IL-13), and interleukin 18 (IL-18); and Tumor Necrosis factor alpha (TNFα), interleukin 13 (IL-13), and interleukin 18 (IL-18).

In another embodiment, the binding protein (i′) comprises one or more polypeptide chains comprising VD1-(X1)n-VD2-(X2)n-VD3-C-(X3)n, wherein, VD1 is a first light chain variable domain obtained from a first parent binding protein, e.g., antibody, or antigen-binding portion thereof, VD2 is a second light chain variable domain obtained from a second parent binding protein, e.g., antibody, or antigen-binding portion thereof, VD3 is a third light chain variable domain obtained from a third parent binding protein, e.g., antibody, or antigen-binding portion thereof, C is a heavy chain constant domain, X1 is a first linker, X1 is a second linker, X3 is an Fc region; and n is 0 or 1.

In another embodiment, the binding protein comprises four polypeptide chains, wherein each of the first and third polypeptide chains independently comprise VD1-(X1)n-VD2-(X2)n-VD3-C-(X3)n, wherein VD1 is a first heavy chain variable domain, VD2 is a second heavy chain variable domain, VD3 is a third heavy chain variable domain, C is a heavy chain constant domain, X1 is a first linker, X2 is a second linker, X3 is an Fc region and wherein each of the second and fourth polypeptide chains independently comprise VD1-(X1)n-VD2-(X2)n-VD3-C-(X3)n, wherein VD1 is a first light chain variable domain, VD2 is a second light chain variable domain, VD3 is a third light chain variable domain, C_{L} is a light chain constant domain, X1 is a linker, X2 is a second linker X3 does not comprise an Fc region and n is 0 or 1.

The binding protein(s) used in the kits of the inventin may be optionally detectably labeled.

Any binding proteins, e.g., antibodies, such as an anti-analyte antibody, any binding proteins as disclosed herein, any anti-analyte TVD binding proteins, or tracers can incorporate a detectable label as described herein, such as a fluorophore, a radioactive moiety, an enzyme, a biotin/avidin label, a chromophore, a chemiluminescent label, or the like, or the kit can include reagents for carrying out detectable labeling. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, enzyme substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the test sample. The kit can also include one or more instruments for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, a solution, and/or at least one basic solution. If desired, the kit can contain a solid phase, such as a magnetic particle, bead, test tube, microtiter plate, cuvette, membrane, scaffolding molecule, film, filter paper, disc or chip.

### C. Adaptation of Kit and Methods

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., an anti-analyte, monoclonal/polyclonal antibody (or a fragment thereof, a variant thereof, or a fragment of a variant thereof), a binding protein as disclosed herein, or an anti-analyte TVD binding protein (or a fragment thereof, a variant thereof, or a fragment of a variant thereof) is attached; either way, sandwich formation and analyte reactivity can be impacted), and the length and timing of the capture, detection and/or any optional wash steps. Whereas a non-automated format, such as an ELISA, may require a relatively longer incubation time with sample and capture reagent (e.g., about 2 hours), an automated or semi-automated format (e.g., ARCHITECT®, Abbott Laboratories) may have a relatively shorter incubation time (e.g., approximately 18 minutes for ARCHITECT®). Similarly, whereas a non-automated format, such as an ELISA, may incubate a detection antibody, such as the conjugate reagent, for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081; 7,419,821; and 7,682,833; and U.S. Patent Publication Nos. 20040018577 and 2006/0160164.

In particular, with regard to the adaptation of an analyte assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized anti-analyte, monoclonal/polyclonal antibody (or a fragment thereof, a variant thereof, or a fragment of a variant thereof), a binding protein as disclosed herein, or anti-analyte TVD binding protein (or a fragment thereof, a variant thereof, or a fragment of a variant thereof), are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising a specific binding partner for an analyte, such as an anti-analyte, monoclonal/polyclonal antibody (or a fragment thereof, a variant thereof, or a fragment of a variant thereof that can bind the analyte), a binding protein as disclosed herein or an anti-analyte TVD binding protein (or a fragment thereof, a variant thereof, or a fragment of a variant thereof that can bind the analyte), either of which can be detectably labeled. Within the fluid pouch of the cartridge is an aqueous reagent that includes p-aminophenol phosphate.

In operation, a sample suspected of containing an analyte is added to the holding chamber of the test cartridge, and the cartridge is inserted into the I-STAT® reader. After the specific binding partner for an analyte has dissolved into the sample, a pump element within the cartridge forces the sample into a conduit containing the chip. Here it is oscillated to promote formation of the sandwich. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of analyte in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, microparticle diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an I-Stat cartridge format, using a nucleic acid sequence linked to the signal antibody as a signal amplifier.

It will be readily apparent to those skilled in the art that other suitable modifications and adaptations of the methods described herein are obvious and may be made using suitable equivalents without departing from the scope of the claimed invention or the embodiments disclosed herein. Having now described the present invention in detail, the same will be more clearly understood by reference to the following examples, which are included for purposes of illustration only and are not intended to be limiting of the claimed invention.

### EXAMPLES

### Example 1

### Generation of Multi- (i.e., Tri-) Variable Domain Immunoglobulin (TVD-Ig) Molecules that Recognize PGE2, IL-12, and IL-18

The triple variable domain immunoglobulin (TVD-Ig) molecule is designed such that three different light chain variable domains (VL) from one or more parent monoclonal binding proteins, e.g., antibodies, are linked in tandem directly or via a short linker by recombinant DNA techniques, followed by the light chain constant domain. Similarly, the heavy chain comprises three different heavy chain variable domains (VH) from one or more parent monoclonal binding proteins, e.g., antibodies, linked in tandem, followed by the constant domain CH1 and Fc region (FIG. 1).

### Example 1.1

### Construction, Expression, and Purification of Multi- (i.e., Tri-) Variable Domain immunoglobulin (TVD-Ig) molecules that recognize PGE2, IL-12, and IL-18

The TVD-Ig protein was designed as an IgG-like molecule except that each light chain and heavy chain of a TVD-Ig protein has three variable domains in tandem instead of one variable domain in an IgG. These three variable domains are separated by short linkers. The linker sequences, which are derived either from the N-terminal sequence of human CH1/Cκ, are the following:

| (SEQ ID NO: 13) | |
|---|---|
| Light chain (κ), Short linker. | TVAAP |
| (SEQ ID NO: 21) | |
| Heavy chain (γl), Short linker. | ASTKGP |

These linker sequences, selected from the N-termini of human Cκ and CH1 are natural extension of the variable domains and exhibit a flexible conformation without significant secondary structures based on the analysis of several Fab crystal structures.

Parent binding proteins, i.e., monoclonal antibodies, including three high affinity monoclonal antibodies, anti-PGE2 (2B5), anti-hIL-12 (1D4.1), and anti-hIL-18 (mAb 2.5), which were previously disclosed. The VL/VH domains of these three monoclonal antibodies were fused together via short linkers (the linker sequence is TVAAP (SEQ ID NO:13) for VL and ASTKGP (SEQ ID NO:21) for VH) by overlapping PCR, in a domain order of 5′VD (1D4.1)-SL-VD (mAb 2.5)-SL-VD (2B5) 3′, followed by constant regions, in both HC and LC, named TVD-Ig 001. A different TVD-Ig (TVD-Ig 002) was also generated with a domain order of 5′ VD (2B5)-SL-VD (1D4.1)-SL-VD (mAb 2.5) 3′, followed by constant regions, in both HC and LC. The detailed procedures of the PCR cloning is described below.

To generate heavy chain constructs TVD-Ig 001, VH domain of the 1D4.1-SL-mAb 2.5 TVD-Ig protein was PCR amplified using specific primers 1 and 4 (primers contained the SL sequence); meanwhile VH domain of the Anti-PGE2 antibody was amplified using specific primers 2 and 3 (primers contained the SL sequence). Both PCR reactions are performed according to standard PCR techniques and procedures. The two PCR products are gel-purified, and used together as overlapping template for the subsequent overlapping PCR reaction using standard PCR conditions and primers 3 and 4. The overlapping PCR products are subcloned into FspA1 and Sal I double digested pHybE-hCg mut (234, 235), z non-a mammalian expression vector (Abbott) by using standard homologous recombination approach.

To generate light chain constructs TVD001, VL domain of the 1D4.1-SL-mAb 2.5 DVD-Ig was PCR amplified using specific primers 6 and 8 (primers contained the SL sequence); meanwhile VL domain of the anti-PGE2 antibody 2B5 was amplified using specific primers 5 and 7 (primers contained the SL sequence). Both PCR reactions are performed according to standard PCR techniques and procedures. The two PCR products are gel-purified, and used together as overlapping template for the subsequent overlapping PCR reaction using standard PCR conditions and primers 7 and 8. The overlapping PCR products are subcloned into BsiWI and NruI double digested pHybE-hCk mammalian expression vector by using standard homologous recombination approach.

To generate heavy chain constructs TVD002, VH domain of the 1D4.1-SL-mAb 2.5 TVD-Ig was PCR amplified using specific primers 9 and 12 (primers contained the SL sequence); meanwhile VH domain of the anti-PGE2 antibody 2B5 was amplified using specific primers 10 and 11 (primers contained the SL sequence); Both PCR reactions are performed according to standard PCR techniques and procedures. The two PCR products are gel-purified, and used together as overlapping template for the subsequent overlapping PCR reaction using standard PCR conditions and primers 11 and 12. The overlapping PCR products are subcloned into FspA1 and Sal I double digested pHybE-hCg mut (234, 235), z non-a mammalian expression vector by using standard homologous recombination approach.

To generate light chain constructs TVD002, VL domain of the 1D4.1-SL-mAb 2.5 DVD-Ig was PCR amplified using specific primers 13 and 15 (primers contained the SL sequence); meanwhile VL domain of the Anti-PGE2 antibody was amplified using specific primers 14 and 16 (primers contained the SL sequence). Both PCR reactions are performed according to standard PCR techniques and procedures. The two PCR products are gel-purified, and used together as overlapping template for the subsequent overlapping PCR reaction using standard PCR conditions and primers 15 and 16. The overlapping PCR products are subcloned into BsiW I and Nru I double digested pHybE-hCk mammalian expression vector (Abbott) by using standard homologous recombination approach.

Primer 1: 3′inside VH for 1D4.1-SL-mAb
2.5-SL-2B5-8.1 VH pHybE
(SEQ ID NO: 29)
GCACCTCTGGGCCCTTGGTCGACGCTGAAGAGACGGTGACCATTGTCCCT
TGGCCCCAGATA
Primer 2: 5′ inside VH for 1D4.1-SL-mAb
2.5-SL-2B5-8.1 VH pHybE
(SEQ ID NO: 30)
ACCGTCTCTTCAGCGTCGACCAAGGGCCCAGAGGTGCAGCTGGTGCAGAG
CGGCGCCGAGGTGAA
Primer 3: 3′ outside VH for 1D4.1-SL-mAb
2.5-SL-2B5-8.1 VH pHybE
(SEQ ID NO: 31)
AGGGTGCCAGGGGGAAGACCGATGGGCCCTTGGTCGACGCGCTGCTCACG
GTCACCAGGGTGCCC
Primer 4: 5′ outside VH for 1D4.1-SL-mAb
2.5-SL-2B5-8.1 VH pHybE
(SEQ ID NO: 32)
TGGCTTTTTCTTGTCGCGATTTTAAAAGGTGTCCAGTGCGAGGTCACCTT
GAGGGAGTCTGGTC
Primer 5: 5′ inside VL for 1D4.1-SL-mAb
2.5-SL-2B5-8.2 VL pHybE
(SEQ ID NO: 33)
GGAGATTAAACGAACGGTGGCTGCACCAGACGTGCTGATGACCCAGACCC
CCCTGAGCCTGCCCG
Primer 6: 3′ inside VL for 1D4.1-SL-mAb
2.5-SL-2B5-8.2 VL pHybE
(SEQ ID NO: 34)
CATCAGCACGTCTGGTGCAGCCACCGTTCGTTTAATCTCCAGTCGTGTCC
CTTGGCCGAAGGTGAT
Primer 7: 3′ outside VL for 1D4.1-SL-mAb
2.5-SL-2B5-8.2 VL pHybE
(SEQ ID NO: 35)
CATCAGATGGCGGGAAGATGAAGACAGATGGTGCAGCCACCGTCCGCTTG
ATCTCCACCTTGGTGC
Primer 8: 5′ outside VL for 1D4.1-SL-mAb
2.5-SL-2B5-8.2 VL pHybE
(SEQ ID NO: 36)
GGGCCTGCTGCTGCTGTGGTTCCCCGGCTCGCGATGCGACATCGTGATGA
CCCAGTCTCCAGACT
Primer 9: 5′ inside 2B5-8.1-SL-1D4.1-SL-325
VH pHybE
(SEQ ID NO: 37)
GAGCAGCGCGTCGACCAAGGGCCCAGAGGTCACCTTGAGGGAGTCTGGTC
CTGCGCTGGTGAAA
Primer 10: 3′ inside 2B5-8.1-SL-1D4.1-SL-325
VH pHybE
(SEQ ID NO: 38)
AGGTGACCTCTGGGCCCTTGGTCGACGCGCTGCTCACGGTCACCAGGGTG
CCCTGGCCCCAGTAG
Primer 11: 5′ outside 2B5-8.1-SL-1D4.1-SL-325
VH pHybE
(SEQ ID NO: 39)
TGGCTTTTTCTTGTCGCGATTTTAAAAGGTGTCCAGTGCGAGGTGCAGCT
GGTGCAGAGCGGCGC
Primer 12: 3′ outside 2B5-8.1-SL-1D4.1-SL-325
VH pHybE
(SEQ ID NO: 40)
GTGCCAGGGGGAAGACCGATGGGCCCTTGGTCGACGCTGAAGAGACGGTG
ACCATTGTCCCTTGG
Primer 13: 5′ inside 2B5-8.2-SL-1D4.1-B3-SL-325
VL: pHybE
(SEQ ID NO: 41)
AGATCAAGCGGACGGTGGCTGCACCAGACATCGTGATGACCCAGTCTCCA
GACTCCCTGGCTGTG
Primer 14: 3′ inside 2B5-8.2-SL-1D4.1-B3-SL-325
VL: pHybE
(SEQ ID NO: 42)
CACGATGTCTGGTGCAGCCACCGTCCGCTTGATCTCCACCTTGGTGCCGC
CGCCGAAGGTGTAGG
Primer 15: 5′ outside 2B5-8.2-SL-1D4.1-B3-SL-325
VL: pHybE
(SEQ ID NO: 43)
TGCTGGGCCTGCTGCTGCTGTGGTTCCCCGGCTCGCGATGCGACGTGCTG
ATGACCCAGACCCCC
Primer 16: 3′ outside 2B5-8.2-SL-1D4.1-B3-SL-325
VL: pHybE
(SEQ ID NO: 44)
TGGCGGGAAGATGAAGACAGATGGTGCAGCCACCGTACGTCGTTTAATCT
CCAGTCGTGTCCCTTG

The TVD-Ig vector constructs were tranfected into 293 cells for production of TVD-Ig proteins. The 293 transient transfection procedure used is a modification of the methods published in Durocher et al. (2002) Nucleic Acids Res. 30(2):E9 and Pham et al. (2005) Biotech. Bioengineering 90(3):332-44. Reagents that were used in the transfection included:
HEK 293-6E cells (human embryonic kidney cell line stably expressing EBNA1; obtained from National Research Council Canada) cultured in disposable Erlenmeyer flasks in a humidified incubator set at 130 rpm, 37° C. and 5% CO2.
Culture medium: FreeStyle 293 Expression Medium (Invitrogen 12338-018) plus 25 μg/mL Geneticin (G418) (Invitrogen 10131-027) and 0.1% Pluronic F-68 (Invitrogen 24040-032).
Transfection medium: FreeStyle 293 Expression Medium plus 10 mM HEPES (Invitrogen 15630-080).
Polyethylenimine (PEI) stock: 1 mg/mL sterile stock solution, pH 7.0, prepared with linear 25 kDa PEI (Polysciences) and stored at less than −15° C.
Tryptone Feed Medium: 5% w/v sterile stock of Tryptone N1 (Organotechnie, 19554) in FreeStyle 293 Expression Medium.

Cell preparation for transfection: Approximately 2-4 hours prior to transfection, HEK 293-6E cells are harvested by centrifugation and resuspended in culture medium at a cell density of approximately 1 million viable cells per mL. For each transfection, 400 mL of the cell suspension is transferred into a disposable 2-L Erlenmeyer flask and incubated for 2-4 hours.

Transfection: The transfection medium and PEI stock are prewarmed to room temperature (RT). For each transfection, 250 μg of plasmid DNA and 500 μg of polyethylenimine (PEI) are combined in 50 mL of transfection medium and incubated for 15-20 minutes at RT to allow the DNA:PEI complexes to form. Each 50-mL DNA:PEI complex mixture is added to a 400-mL culture prepared previously and returned to the humidified incubator set at 130 rpm, 37° C. and 5% CO2. After 20-28 hours, 50 mL of Tryptone Feed Medium is added to each transfection and the cultures are continued for six days.

Purification: Supernatants from 293 transfections were purified using an AKTA purifier (Amersham biosciences). IgG binding buffer (Pierce #21007) was added to the supernatants at 10% and this load was filtered with 0.2 mM CA filter. A 5 mL protein A column was equilibrated with IgG binding buffer S. Samples were loaded onto the column at a flow rate of 15 mL/min. The column was then washed with 8 column volumes of 1× TBS. The sample was eluted at a flow rate of 10 mL/min in IgG Elution buffer (Pierce) and 2.5 mL fractions collected. The column was then regenerated using 6M guanidine and the lines were cleaned with 0.5M NaOH. Fractions were collected and OD 280 nm was taken. Those fractions containing an OD >0.1 were neutralized with 10% 2M Tris pH 7.5 and analyzed on SEC. The fractions containing TVD-Ig with low aggregation were then pooled, concentrated, and dialyzed into buffer (10 mM Na2HP04, 10 mM NaCitrate pH 6.0) overnight at 4° C. on stir plate. OD 280 nm of the samples were determined and proteins characterized.

TABLE 8
Amino acid sequence of TVD-Igs capable of
binding PGE2, IL-12 and IL-18 Protein

| Protein | Sequence | Sequence |
|---|---|---|
| region | Identifier | 12345678901234567890 |
| TVD-Ig 001 | SEQ ID NO: 45 | EVTLRESGPALVKPTQTLTL |
| HEAVY | | TCTFSGFSLSKSVMGVSWIR |
| VARIABLE | | QPPGKALEWLAHIYWDDDKY |
| | | YNPSLKSRLTISKDTSKNQV |
| | | VLTMTNMDPVDTATYYCARR |
| | | GIRSAMDYWGQGTTVTVSS |
| | | ASTKGPEVQLVQSGTEVKKP |
| | | GESLKISCKGSGYTVTSYWI |
| | | GWVRQMPGKGLEWMGFIYPG |
| | | DSETRYSPTFQGQVTISADK |
| | | SFNTAFLQWSSLKASDTAMY |
| | | YCARVGSGWYPYTFDIWGQG |
| | | TMVTVSSASTKGPEVQLVQS |
| | | GAEVKKPGASVKVSCKASGY |
| | | TFTKYWLGWVRQAPGQGLEW |
| | | MGDIYPGYDYTHYNEKFKDR |
| | | VTLTTDTSTSTAYMELRSLR |
| | | SDDTAVYYCARSDGSSTYWG |
| | | QGTLVTVSS |
| 1D4.1 VH | SEQ ID NO: 46 | EVTLRESGPALVKPTQTLTL |
| | | TCTFSGFSLSKSVMGVSWIR |
| | | QPPGKALEWLAHIYWDDDKY |
| | | YNPSLKSRLTISKDTSKNQV |
| | | VLTMTNMDPVDTATYYCARR |
| | | GIRSAMDYWGQGTTVTVSS |
| LINKER | SEQ ID NO: 21 | ASTKGP |
| MAB 2.5 VH | SEQ ID NO: 47 | EVQLVQSGTEVKKPGESLKI |
| | | SCKGSGYTVTSYWIGWVRQM |
| | | PGKGLEWMGFIYPGDSETRY |
| | | SPTFQGQVTISADKSFNTAF |
| | | LQWSSLKASDTAMYYCARVG |
| | | SGWYPYTFDIWGQGTMVTVS |
| | | S |
| LINKER | SEQ ID NO: 21 | ASTKGP |
| 2B5 VH | SEQ ID NO: 48 | EVQLVQSGAEVKKPGASVKV |
| | | SCKASGYTFTKYWLGWVRQA |
| | | PGQGLEWMGDIYPGYDYTHY |
| | | NEKFKDRVTLTTDTSTSTAY |
| | | MELRSLRSDDTAVYYCARSD |
| | | GSSTYWGQGTLVTVSS |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 001 | SEQ ID NO: 50 | DIVMTQSPDSLAVSLGERAT |
| LIGHT | | INCKASQSVSNDVAWYQQKP |
| VARIABLE | | GQPPKLLIYYASNRYTGVPD |
| | | RFSGSGSGTDFTLTISSLQA |
| | | EDVAVYYCQQDYNSPWTFGG |
| | | GTKVEIKRTVAAPEIVMTQS |
| | | PATLSVSPGERATLSCRASE |
| | | SISSNLAWYQQKPGQAPRLF |
| | | IYTASTRATDIPARFSGSGS |
| | | GTEFTLTISSLQSEDFAVYY |
| | | CQQYNNWPSITFGQGTRLEI |
| | | KRTVAAPDVLMTQTPLSLPV |
| | | TPGEPASISCTSSQNIVHSN |
| | | GNTYLEWYLQKPGQSPQLLI |
| | | YKVSNRFSGVPDRFSGSGSG |
| | | TDFTLKISRVEAEDVGVYYC |
| | | FQVSHVPYTFGGGTKVEIKR |
| 1D4.1 VL | SEQ ID NO: 51 | DIVMTQSPDSLAVSLGERAT |
| | | INCKASQSVSNDVAWYQQKP |
| | | GQPPKLLIYYASNRYTGVPD |
| | | RFSGSGSGTDFTLTISSLQA |
| | | EDVAVYYCQQDYNSPWTFGG |
| | | GTKVEIKR |
| LINKER | SEQ ID NO: 13 | TVAAP |
| MAB 2.5 VL | SEQ ID NO: 52 | EIVMTQSPATLSVSPGERAT |
| | | LSCRASESISSNLAWYQQKP |
| | | GQAPRLFIYTASTRATDIPA |
| | | RFSGSGSGTEFTLTISSLQS |
| | | EDFAVYYCQQYNNWPSITFG |
| | | QGTRLEIKR |
| LINKER | SEQ ID NO: 13 | TVAAP |
| 2B5 | SEQ ID NO: 53 | DVLMTQTPLSLPVTPGEPAS |
| | | ISCTSSQNIVHSNGNTYLEW |
| | | YLQKPGQSPQLLIYKVSNRF |
| | | SGVPDRFSGSGSGTDFTLKI |
| | | SRVEAEDVGVYYCFQVSHVP |
| | | YTFGGGTKVEIKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| TVD-Ig 002 | SEQ ID NO: 55 | EVQLVQSGAEVKKPGASVKV |
| HEAVY | | SCKASGYTFTKYWLGWVRQA |
| VARIABLE | | PGQGLEWMGDIYPGYDYTHY |
| | | NEKFKDRVTLTTDTSTSTAY |
| | | MELRSLRSDDTAVYYCARSD |
| | | GSSTYWGQGTLVTVSSASTK |
| | | GPEVQLVQSGTEVKKPGESL |
| | | KISCKGSGYTVTSYWIGWVR |
| | | QMPGKGLEWMGFIYPGDSET |
| | | RYSPTFQGQVTISADKSFNT |
| | | AFLQWSSLKASDTAMYYCAR |
| | | VGSGWYPYTFDIWGQGTMVT |
| | | VSSASTKGPEVTLRESGPAL |
| | | VKPTQTLTLTCTFSGFSLSK |
| | | SVMGVSWIRQPPGKALEWLA |
| | | HIYWDDDKYYNPSLKSRLTI |
| | | SKDTSKNQVVLTMTNMDPVD |
| | | TATYYCARRGIRSAMDYWGQ |
| | | GTTVTVSS |
| 2B5 VH | SEQ ID NO: 48 | EVQLVQSGAEVKKPGASVKV |
| | | SCKASGYTFTKYWLGWVRQA |
| | | PGQGLEWMGDIYPGYDYTHY |
| | | NEKFKDRVTLTTDTSTSTAY |
| | | MELRSLRSDDTAVYYCARSD |
| | | GSSTYWGQGTLVTVSS |
| LINKER | SEQ ID NO: 21 | ASTKGP |
| 1D4.1 VH | SEQ ID NO: 46 | EVTLRESGPALVKPTQTLTL |
| | | TCTFSGFSLSKSVMGVSWIR |
| | | QPPGKALEWLAHIYWDDDKY |
| | | YNPSLKSRLTISKDTSKNQV |
| | | VLTMTNMDPVDTATYYCARR |
| | | GIRSAMDYWGQGTTVTVSS |
| LINKER | SEQ ID NO: 21 | ASTKGP |
| MAB 2.5 VH | SEQ ID NO: 47 | EVQLVQSGTEVKKPGESLKI |
| | | SCKGSGYTVTSYWIGWVRQM |
| | | PGKGLEWMGFIYPGDSETRY |
| | | SPTFQGQVTISADKSFNTAF |
| | | LQWSSLKASDTAMYYCARVG |
| | | SGWYPYTFDIWGQGTMVTVS |
| | | S |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 002 | SEQ ID NO: 58 | DVLMTQTPLSLPVTPGEPAS |
| LIGHT | | ISCTSSQNIVHSNGNTYLEW |
| VARIABLE | | YLQKPGQSPQLLIYKVSNRF |
| | | SGVPDRFSGSGSGTDFTLKI |
| | | SRVEAEDVGVYYCFQVSHVP |
| | | YTFGGGTKVEIKRTVAAPEI |
| | | VMTQSPATLSVSPGERATLS |
| | | CRASESISSNLAWYQQKPGQ |
| | | APRLFIYTASTRATDIPARF |
| | | SGSGSGTEFTLTISSLQSED |
| | | FAVYYCQQYNNWPSITFGQG |
| | | TRLEIKRTVAAPDIVMTQSP |
| | | DSLAVSLGERATINCKASQS |
| | | VSNDVAWYQQKPGQPPKLLI |
| | | YYASNRYTGVPDRFSGSGSG |
| | | TDFTLTISSLQAEDVAVYYC |
| | | QQDYNSPWTFGGGTKVEIKR |
| 2B5 | SEQ ID NO: 53 | DVLMTQTPLSLPVTPGEPAS |
| | | ISCTSSQNIVHSNGNTYLEW |
| | | YLQKPGQSPQLLIYKVSNRF |
| | | SGVPDRFSGSGSGTDFTLKI |
| | | SRVEAEDVGVYYCFQVSHVP |
| | | YTFGGGTKVEIKR |
| LINKER | SEQ ID NO: 13 | TVAAP |
| 1D4.1 VL | SEQ ID NO: 59 | DIVMTQSPDSLAVSLGERAT |
| | | INCKASQSVSNDVAWYQQKP |
| | | GQPPKLLIYYASNRYTGVPD |
| | | RFSGSGSGTDFTLTISSLQA |
| | | EDVAVYYCQQDYNSPWTFGG |
| | | GTKVEIKR |
| LINKER | SEQ ID NO: 13 | TVAAP |
| MAB 2.5 VL | SEQ ID NO: 60 | EIVMTQSPATLSVSPGERAT |
| | | LSCRASESISSNLAWYQQKP |
| | | GQAPRLFIYTASTRATDIPA |
| | | RFSGSGSGTEFTLTISSLQS |
| | | EDFAVYYCQQYNNWPSITFG |
| | | QGTRLEIKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

### Example 1.2

### Characterization of Triple Variable Domain Immunoglobulin (TVD-Ig)

### Example 1.2.1

### Size Exclusion Chromatography and Mass Spectrometric analysis of TVD-Igs

Purified TVD-Ig 001 and TVD-Ig 002 were diluted in 10 mM Na2HP04, 10 mM NaCitrate pH 6.0 to a concentration of 0.2 mg/ml and 50 ml. were applied on a Supedex 200, 10/300 GL, column (Amersham Bioscience, Piscataway, N.J.). An HPLC instrument, Model 10A (Shimadzu, Columbia, Md.) was used for SEC. The mobile phase buffer was 211 mM Na2S04, 92 mM Na2HP04, 5 mM NaZ3, pH 7.04. All proteins were determined using UV detection at 280 nm and 214 nm. The elution was isocratic at a flow rate of 0.75 mL/min. The SEC profile indicated that both TVD-Ig 001 and 002 exhibited a majority monomeric peak of 77% and 86%, respectively, with minor aggregation. No smaller fragments were detected.

To determine the heavy chain and light chain molecular weight of the TVD-Ig proteins, 10 μl of TVD-Ig 001 or TVD-Ig 002 (0.1 mg/ml) was reduced by 1.0M DTT (5 uL). A Videx C4, 300 A, 1 mm, 4072041 column (The Nest Group, Southboro, Mass.) was used to separate heavy and light chain columns. An Agilent1200 was used with the mass spectrometer Agilent 6210 Time of Flight LC/MS (Agilent Technologies Inc., Palo Alto, Calif.). Buffer A was 0.01% TFA, 0.1% FA in HPLC grade H2O. Buffer B was 0.01% TFA, 0.1% FA in ACN. The flow rate was 50 mL/min, and the sample injection volume was 2.0 mL. All MS raw data were analyzed using Agilent MassHunter software. Based on the mass spec results, the HC M.W. of TVD-Ig 001 was 78119 dalton, consistent with the theoretical M.W. of 78115 dalton. Likewise, the LC M.W. of TVD-Ig 001 was 48625 dalton, consistent with the theoretical M.W. of 48624 dalton. The HC M.W. of TVD-Ig 002 was 78119 dalton, consistent with the theoretical M.W. of 78115 dalton. Likewise, the LC M.W. of TVD-Ig was 48781 dalton, consistent with the theoretical M.W. of 48780 dalton.

### Example 1.2.2

### Determination of IL-12 or IL-18 Binding Affinity of IL-12/IL-18/PGE2 TVD Igs by Biacore Analysis

Real-time binding interactions between captured TVD-Ig molecules (human TVD-1218PGE2 or TVD-PGE21218 captured on a biosensor matrix via goat anti-human IgG) and recombinant IL-12 or IL-18 was measured by surface plasmon resonance (SPR) using the BIAcore system (Biacore AB, Uppsala, Sweden) according to manufacturer's instructions and standard procedures. Briefly, recombinant IL-12 or IL-18 was diluted in HBS running buffer (Biacore AB) and 50 μl aliquots were injected through the immobilized protein matrices at a flow rate of 5 ml/min. The concentrations of recombinant IL-12 or IL-18 employed were 62.5, 125, 187.5, 250, 375, 500, 750, 1000, 1500 and 2000 nM. To determine the dissociation constant (off-rate), association constant (on-rate), BIAcore kinetic evaluation software (version 3.1) was used.

### Example 1.2.3

### Determination of PGE2 Binding Affinity of IL-12/IL-18/PGE2 TVD Igs by Radioimmunoassay (RIA)

The binding affinity of anti-IL-12/IL-18/PGE2 TVD-Igs to PGE2 was determined by radioimmunoassay using 3H-PGE2. Plates were coated with 5 μg/ml of goat anti-human IgG (Fc). TVD-1218PGE2 or TVD-PGE21218 was diluted to 0.04 μg/ml in PBST+10% Superblock and 50 ul of each was added to each well (2 ng/well) of the pre-blocked ELISA plate and was incubated for 1 hour at room temperature. Wells were washed 3 times with PBS+0.1% Tween-20. Prostaglandin E2 [5,6,8,11,12,14,15-3H(N)] (NET-428, PerkinElmer) was diluted in PBST+10% Superblock to 6 nM (2× stock) to different concentrations (20 nM, 10 nM, 5 nM, 2.5 nM, 1.25 nM, 0.625 nM, 0.3125 nM, 0.156 nM, 0.078 nM, 0.039 nM, 0.019 nM, 0.0098 nM) and was added to the plate to incubate for 1 hour at room temperature. Wells were washed 6 times with PBST+10% Superblock and 50 μl of scintillation fluid added to each well. Plates were read using the TopCount reader.

### Example 1.2.4

### Functional Analysis Using Cell-Based Bioassays

IL-12 binding ELISA: The binding of TVD-Ig to IL-12 was first determined by ELISA. Reacti-bind streptavidin coated 96 well plates (Pierce Cat #15124) were pre-blocked with Superblock. Plates were washed five times with 1× PBST. Biotinylated IL-12 was diluted to 100 ng/ml in 10% superblock/PBST and 100 ml was added to each well. Plates were incubated for 2 hours at room temperature and then washed five times with 1× PBST. Control antibodies (human IgG and 1D4.1) and TVD-Igs were diluted to 1000 ng/ml and serially diluted 1:3 in 10% SB/PBST. Samples were added to the plate at 100 ml per well and incubated for 1 hour at RT. Plates were then washed five times. Goat anti human IgG (H & L) Pierce #31410 diluted 1:10,000 was then added at 100 ml per well. Plates were incubated for 1 hour at Room Temperature and then washed five times. Plates were then developed using 1-step TMB (Pierce #34028) with an incubation of 15 minutes at room temperature prior to stopping with 2N sulfuric acid. Plates were read on a spectrophotometer at OD 450 nm. The EC50 values were determined using sigmoidal curve fit analysis from the binding curve.
PGE2 bioassay: To determine the potency of the TVD-Ig against PGE2, a FLIPR assay using EP4 HEKG a16#2 cells was run. EP4 HEKG a16#2 cells were plated at 3E4 cells per well in a black/clear Poly-D-lysine plate (Corning #3667). Cells were incubated for 15 minutes at room temperature prior to allow for even settling. Plates were incubated 0/N at 37° C., 5% CO2. The FLIPR was turned on 30 minutes prior to use. FLIPR buffer consisting of 1× HBSS (Invitrogen #14065-056), 20 mM HEPES (Invitrogen #15630-080) 0.1% BSA, and 2.5 mM Probenecid (Sigma #P-8761) was made. A 10× stock of No wash dye was prepared by adding 10 mL water to Mol Dev no wash dye powder and vortexed. The stock of no wash dye was diluted 1:10 in FLIPR buffer. Media was removed from plates and 80 ml of 1× dye was added per well. Samples were incubated on a slow rocker for 1.5 hours at room temperature. PGE2 in 200 proof Ethanol was diluted from a stock concentration of 5 mM. using FLIPR buffer. Antibodies (or TVD-Ig) were diluted in FLIPR buffer to a 1000 ng/ml. Antibodies (or TVD-Ig) were serially diluted 1:3. PGE2 and antibodies (or TVD-Ig) were combined and diluted in FLIPR buffer. To each well 20 ml of PGE2/antibody was added and samples read on the FLIPR.
KG-1 biossay: The potency of TVD-Ig against rhIL-18 was measured by KG-1 assay. KG-1 cell line is a human acute myelogeneous leukemia cell line (ATCC Cat#CCL-246). Serial dilutions of, mAb 2.5 or TVD-Ig was prepared in complete RPMI 1640 (10% FBS, 2 mM L-glutamine, 50 units/ml penicillin, 50 mg/ml streptomycin and 0.075% sodium bicarbonate). The antibody dilutions were pre-incubated with recombinant human IL-18 (2 μg/mL) for 1 hour at 37° C. in 100 μl in a 96 well tissue culture plate (Costar #3599). KG-1 cells (1000 at a density of 1.0−3.0×10⁵ cells/well were added in the presence of 20 ng/mL TNF-a and incubated for 16-20 hours at 37° C., 5% CO2. After incubation, cell free supernatant was harvested and the levels of human IFN-γ measured by standard ELISA (R&D Systems). Percent inhibition was plotted against antibody concentration relative to the 2 ng/ml rIL-18 control. The IC₅₀ values were determined using sigmoidal curve fit analysis from the inhibitory curve.
Human IL-12 bioassay: Human IFN-γ is released from PHA blast cells in response to human IL-12 stimulation in a concentration dependent mannor. To determine the neutralization potency, TVD-Igs were tested at a final concentration range of 1e-7 M to 1e-14 M in the assay, in the presence of 200 pg/mL rhIL-12. Fifty μL of TVD-Ig were preincubated for 1 hour at 37° C. with 50 μL of human IL-12 in RPMI complete medium in a 96-well, flat bottom microtiter plate. Frozen PHA blast cells were thawed and washed two times in culture media, and then trypan blue counted. The cells were adjusted to a density of 2.5e6 cells/mL in culture media. Subsequently, 100 μL of PHA blasts were added to the TVD-Ig+IL-12 mixture. The final concentration of human IL-12 in the assay was 200 pg/mL. The mixture was incubated for 18 hours at 37° C., 5% CO2, after which the IFN-γ levels in the supernatants were measured by human IFN-γ ELISA. The IC₅₀ values were generated from ploting IFN-γ concentrations versus Ig (TVD-Ig 003 or monoclonal antibody) concentrations (sigmoidal curve dose responses), using GraphPad Prism software. Each measurement was performed in quadruplicate, and each experiment was performed a minimum of two times.

TABLE 9
Characterization of TVD-Ig 001 and 002 by anti-IL12 ELISA

| Ig | ED50 (nM) |
|---|---|
| Control monoclonal antibody | 0.38 |
| TVD-Ig 001 | 0.66 |
| TVD-Ig 002 | NA |

TABLE 10
Characterization of TVD-Ig 001 and 002 neutralization potencies
(IC₅₀, nM) by cell-based bioassays

| Ig | IL-12 | IL-18 | PGE2 |
|---|---|---|---|
| Control monoclonal | 0.624 | 0.58 | 0.03 |
| antibodies | | | |
| TVD-Ig 001 | 0.559 | 0.29 | 0.074 |
| TVD-Ig 002 | NA | 0.21 | 0.05 |

N/A: no detecable activity observed within the TVD-Ig concentration range tested.

An initial IL-12 binding ELISA result indicated that TVD-Ig 001 was able to binding IL-12 with an ED50 similar to that of the parental monoclonal antibody 1D4.1 (Table 9), whereas binding of TVD-Ig 002 to IL-12 was not detected by this method. In subsequent cell-based bioassay, it was demonstrated that TVD-Ig 001 was able to neutralize IL-12, IL-18, and PGE2, with potencies similar to that of the parental monoclonal antibodies (Table 10) Similar to the ELISA data, inhibition of IL-12 by TVD-Ig 002 was not observed in the IL-12 bioassay within the concentration range tested. Nevertheless, TVD-Ig 002 was active in neutralizing both IL-18 and PGE2, with potencies similar to that of both parental monoclonal antibodies (Table 10).

EP4 assay: The ability of anti-PGE2 antibodies and anti-PGE2 containing anti-IL-12/IL-18/PGE2 TVD-Ig molecules to inhibit the cellular response of PGE2 was determined in a Ca++ flux assay using stably transfected human EP4 in HEK293 G□16 cells. Cells were plated in black/clear Poly-D-Lysine plates, (Corning #3667) and incubated with Ca++-sensitive dye (Molecular Devices) for 90 minutes. Stock PGE2 (in 200 proof ethanol) was diluted with FLIPR buffer (containing 1× HBSS, 20 mM HEPES, 0.1% BSA and 2.5 mM Probenecid). Anti-PGE2 antibodies, TVD-1218PGE2, TVD-PGE21218 or isotype matched control antibodies were also pre-diluted in FLIPR buffer. 25 μl of PGE2 or pre-incubated PGE2/antibody mixture or pre-incubated PGE2/TVD-Ig protein mixture was added to the wells pre-plated with cells. A dose response of PGE2 was done by a serial titration of PGE2 and was determined using FLIPR1 or Tetra (Molecular Devices). EC50 was determined using GraphPad Prism 5. For testing antibodies and TVD-Ig proteins, PGE2 at EC50 concentration was incubated with varying concentrations of test articles or isotype matched antibody (negative control) for 20 minutes, added to dye-loaded human EP4 in HEK293 Gα16 cells. Ca++ flux was monitored using FLIPR1 and data was analyzed using GraphPad Prism 5.
HuPBMC-SCID mouse model: Both IL-12 and IL-18 are required to produce optimal IFNγ in response to various stimuli. The biological activity of anti-IL-12/IL-18 DVD-Ig in vivo was determined using the huPBMC-SCID mouse model. In this model, anti-IL-12 antibody (1D4.1) anti-IL-18 antibody (mAb 2.5), DVD 1D4.1-mAb 2.5, the TVD-1218PGE2, or TVD-PGE21218 were injected i.p. or i.v. (250 mg/mouse each) followed by transfer of freshly purified human PBMCs (huPBMC) i.p. into SCID mice. Fifteen minutes later, mice were challenged with dried staphylococcus aureus cells (SAC) to induce human IFNγ production. Animals (n=7-8/group) were sacrificed 18-20 hrs later and serum huIFNγ levels were determined by ELISA. 1D4.1 and mAb 2.5 usually inhibited SAC-induced IFNγ by approximately 70% which represents maximum IFNγ inhibition with each compound in this model. However, treatment of mice with 1D4.1+mAb 2.5 and TVD-1218PGE2, or TVD-PGE21218 is expected to inhibit IFNγ production by almost 100%.
Carrageenan-induced paw edema model: The in vivo efficacy of mouse anti-PGE2 antibody 2B5-8.0, TVD-1218PGE2, or TVD-PGE21218 is assessed by determining carragenan-induced paw edema. The induction of paw inflammation with carrageenan is performed similarly as previously described (Joseph P. Portanova, et al. J. Exp. Med. 184: 883-891 (1996)). Female C57BL/6N mice are dosed i.p. with anti-PGE2 antibodies or TVD-1218PGE2, or TVD-PGE21218 molecules (30-1 mg/kg) 18 hours prior to challenge and then injected i.d. at T=0 in the footpad with 30 mL of either 0.9% saline in the left pad or 0.5% 1-carrageenan in 0.9% saline (FMC Corp., Rockland, Me.) in the right pad. Edema is measured by spring calipers at 2 and 4 hours and calculated as a change in thickness of the challenged foot minus the change in thickness of the control foot. Mice are euthanized at the end of treatment and sera collected to measure protein levels.

### Example 1.2.5

### Pharmacokinetic and Pharmacodynamic Studies of IL-12/IL-18/PGE2 TVD Ig

Pharmacokinetic properties of TVD-1218PGE2, or TVD-PGE21218 and the parental monoclonal antibodies 1D4.1, mAb 2.5 and Hu2B5.7 were assessed in male Sprague-Dawley rats. TVD-Ig and the mAbs were administered to male SD rats at a single intravenous dose of 4 mg/kg via a jugular cannula or subcutaneously under the dorsal skin. Serum samples were collected at different time points over a period of 37 days and analyzed by human IL-12 capture and/or human IL-18 capture ELISAs and Biotin-PGE2 ELISA. Briefly, ELISA plates were coated with goat anti-biotin antibody (5 μg/ml, 4° C., overnight), blocked with Superblock (Pierce), and incubated with biotinylated human IL-12 (IL-12 capture ELISA) or IL-18 (IL-18 capture ELISA) or Biotin-PGE2 (PGE2 ELISA) at 50 ng/ml in 10% Superblock TTBS at room temperature for 2 h. Serum samples were serially diluted (0.5% serum, 10% Superblock in TTBS) and incubated on the plate for 30 min at room temperature. Detection was carried out with HRP-labeled goat anti human antibody and concentrations were determined with the help of standard curves using the four parameter logistic fit.

### Example 2

### Generating Triple Variable Domain Immunoglobulin (TVD-Ig 003) that Recognizes TNF, IL-13, and IL-18

Parent binding protein, i.e., monoclonal antibody anti-hIL-18 (mAb 2.5), and an anti-TNF/IL-13 DVD-Ig molecule D2E7-SL-13C5.5L3F were previously disclosed. The VL/VH domains of these three monoclonal antibodies were fused together via short linkers (the linker sequence is TVAAP (SEQ ID NO:13) for VL and ASTKGP (SEQ ID NO:21) for VH) by overlapping PCR, in a domain order of 5′VD (D2E7)-SL-VD (13C5.5L3F)-SL-VD (mAb 2.5), followed by constant regions, in both HC and LC, termed TVD-Ig 003. The procedures of the PCR cloning, expression, and purification process were similar to that described for TVD-Ig 001 and 002, except the primer sequences were different due to VL/VH sequence differences.

### Example 2.1

### Construction, Expression, and Purification of Triple Variable Domain Immunoglobulin (TVD-Ig003)

To generate light chain constructs TVD-Ig 003, VL domain of the D2E7-SL-13C5.5L3F DVD-Ig was PCR amplified using specific primers 17 and 18; meanwhile VL domain of the Anti-IL-18 antibody mAb 2.5 was amplified using specific primers 19 and 20; Both PCR reactions are performed according to standard PCR techniques and procedures. The two PCR products are gel-purified, and used together as overlapping template for the subsequent overlapping PCR reaction using standard PCR conditions and primers 17 and 19. The overlapping PCR products are subcloned into FspA1 and Sal I double digested pHybE-hCg mut (234, 235), z non-a mammalian expression vector (Abbott) by using standard homologous recombination approach.

To generate heavy chain constructs TVD-Ig 003, VH domain of the D2E7-SL-13C5.5L3F DVD-Ig was PCR amplified using specific primers 21 and 22; meanwhile VH domain of the Anti-IL-18 antibody mAb 2.5 was amplified using specific primers 23 and 24; Both PCR reactions are performed according to standard PCR techniques and procedures. The two PCR products are gel-purified, and used together as overlapping template for the subsequent overlapping PCR reaction using standard PCR conditions and primers 21 and 23. The overlapping PCR products are subcloned into BsiW I and Nru I double digested pHybE-hCk mammalian expression vector (Abbott) by using standard homologous recombination approach.

Primer 17:
(SEQ ID NO: 61)
5′-AGCTGCTGGGCCTGCTGCTGCTGTGGTTCC-3
Primer 18:
(SEQ ID NO: 62)
5′-CACTATTTCAGGAGCTGCAACTGTCCGCTTGATCTCCACCTTGG-3′
Primer 19:
(SEQ ID NO: 63)
5′-TCCAGATTTCAACTGCTCATCAGA-3′
Primer 20:
(SEQ ID NO: 64)
5′-ACAGTTGCAGCTCCTGAAATAGTGATGACGCAGTC-3′
Primer 21:
(SEQ ID NO: 65)
5′-AGTTTGGGCTGAGCTGGCTTTTTCTTGTCG-3
Primer 22:
(SEQ ID NO: 66)
5′-CACCTCAGGTCCTTTAGTGCTTGCGCTGCTCACGGTCACCAGGG-3′
Primer 23:
(SEQ ID NO: 67)
5′-TGTGCCCCCAGAGGTGCTCTTGGAGGA-3′
Primer 24:
(SEQ ID NO: 68)
5′-GCAAGCACTAAAGGACCTGAGGTGCAGCTGGTGCAGTC-3′

TABLE 11
Amino acid sequence of TVD-Ig 003 capable of
binding TNF, IL-13 and IL-18 Protein

| Protein | Sequence | Sequence |
|---|---|---|
| region | Identifier | 12345678901234567890 |
| TVD-Ig 003 | SEQ ID NO: 69 | EVQLVESGGGLVQPGRSLRL |
| HEAVY | | SCAASGFTFDDYAMHWVRQA |
| VARIABLE | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | SASTKGPEVTLRESGPGLVK |
| | | PTQTLTLTCTLYGFSLSTSD |
| | | MGVDWIRQPPGKGLEWLAHI |
| | | WWDDVKRYNPALKSRLTISK |
| | | DTSKNQVVLKLTSVDPVDTA |
| | | TYYCARTVSSGYIYYAMDYW |
| | | GQGTLVTVSSASTKGPEVQL |
| | | VQSGTEVKKPGESLKISCKG |
| | | SGYTVTSYWIGWVRQMPGKG |
| | | LEWMGFIYPGDSETRYSPTF |
| | | QGQVTISADKSFNTAFLQWS |
| | | SLKASDTAMYYCARVGSGWY |
| | | PYTFDIWGQGTMVTVSS |
| D2E7 VH | SEQ ID NO: 70 | EVQLVESGGGLVQPGRSLRL |
| | | SCAASGFTFDDYAMHWVRQA |
| | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | S |
| LINKER | SEQ ID NO: 21 | ASTKGP |
| 13C5.5L3F VH | SEQ ID NO: 71 | EVTLRESGPGLVKPTQTLTL |
| | | TCTLYGFSLSTSDMGVDWIR |
| | | QPPGKGLEWLAHIWWDDVKR |
| | | YNPALKSRLTISKDTSKNQV |
| | | VLKLTSVDPVDTATYYCART |
| | | VSSGYIYYAMDYWGQGTLVT |
| | | VSS |
| LINKER | SEQ ID NO: 21 | ASTKGP |
| MAB 2.5 VH | SEQ ID NO: 47 | EVQLVQSGTEVKKPGESLKI |
| | | SCKGSGYTVTSYWIGWVRQM |
| | | PGKGLEWMGFIYPGDSETRY |
| | | SPTFQGQVTISADKSFNTAF |
| | | LQWSSLKASDTAMYYCARVG |
| | | SGWYPYTFDIWGQGTMVTVS |
| | | S |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 003 | SEQ ID NO: 72 | DIQMTQSPSSLSASVGDRVT |
| LIGHT | | ITCRASQGIRNYLAWYQQKP |
| VARIABLE | | GKAPKLLIYAASTLQSGVPS |
| | | RFSGSGSGTDFTLTISSLQP |
| | | EDVATYYCQRYNRAPYTFGQ |
| | | GTKVEIKRTVAAPDIQMTQS |
| | | PSSLSASVGDRVTISCRASQ |
| | | DIRNYLNWYQQKPGKAPKLL |
| | | IFYTSKLHSGVPSRFSGSGS |
| | | GTDYTLTISSLQPEDIATYY |
| | | CQQGLTPPLTFGGGTKVEIK |
| | | RTVAAPEIVMTQSPATLSVS |
| | | PGERATLSCRASESISSNLA |
| | | WYQQKPGQAPRLFIYTASTR |
| | | ATDIPARFSGSGSGTEFTLT |
| | | ISSLQSEDFAVYYCQQYNNW |
| | | PSITFGQGTRLEIKR |
| D2E7 VL | SEQ ID NO: 73 | DIQMTQSPSSLSASVGDRVT |
| | | ITCRASQGIRNYLAWYQQKP |
| | | GKAPKLLIYAASTLQSGVPS |
| | | RFSGSGSGTDFTLTISSLQP |
| | | EDVATYYCQRYNRAPYTFGQ |
| | | GTKVEIKR |
| LINKER | SEQ ID NO: 13 | TVAAP |
| 13C5.5L3F VL | SEQ ID NO: 74 | DIQMTQSPSSLSASVGDRVT |
| | | ISCRASQDIRNYLNWYQQKP |
| | | GKAPKLLIFYTSKLHSGVPS |
| | | RFSGSGSGTDYTLTISSLQP |
| | | EDIATYYCQQGLTPPLTFGG |
| | | GTKVEIKR |
| LINKER | SEQ ID NO: 13 | TVAAP |
| MAB 2.5 VL | SEQ ID NO: 52 | EIVMTQSPATLSVSPGERAT |
| | | LSCRASESISSNLAWYQQKP |
| | | GQAPRLFIYTASTRATDIPA |
| | | RFSGSGSGTEFTLTISSLQS |
| | | EDFAVYYCQQYNNWPSITFG |
| | | QGTRLEIKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

### Example 2.2

### Characterization of Multi- (i.e., Tri-) Variable Domain Immunoglobulin Molecule (TVD-Ig 003)

### Example 2.2.1

### Size Exclusion Chromatography and Mass Spectrometric analysis of TVD-Ig 003

Purified TVD-Ig 003 was analyzed by SEC and MS as described for TVD-Ig 001 and 002. TVD-Ig 003 exhibited a monomeric profile on SEC, apparently existed as a homogeneous protein (96.9% monomer). Based on the mass spec results, the HC M.W. of TVD-Ig 003 was 78854 dalton, consistent with the theoretical M.W. of 78873 dalton. Likewise, the LC M.W. was 48047 dalton, consistent with the theoretical M.W. of 48060 dalton.

### Example 2.2.2

### Determination of Biological Activity of TVD-Ig

To determine if TVD-Ig was able to inhibit TNF, IL-13, or IL-8-mediated signaling, 3 cell-based bioassays were used:

L929 bioassy: L929 cells were propagated in Eagle's minimal essential medium, supplemented with 2 mM 1-glutamine, and Earle's balanced salt solution adjusted to contain 1.5 g/liter sodium bicarbonate, 0.1 mM nonessential amino acids, 1.0 mM sodium pyruvate, 10% FBS, and 50 μg/ml gentamycin. Cells were maintained throughout the study at 37° C. and 5% CO2. A total of 35,000 L929 cells in complete growth media were added to each well of a 96-well plate and grown overnight. To assess neutralization potencies of TVD-Ig 003 and D2E7, samples (at various concentrations from 1e-7 M to 1e-14 M) were preincubated with TNF-α (350 pg/ml) at room temperature for 30 min. Just before TNF-α treatment, growth media were removed and replaced with 0.5 volume Eagle's minimal essential medium containing 10% FBS and 1 μg/ml actinomycin D. The TNF-α-TVD-Ig 003 complexes were next added to the plate immediately after changing the media so that the cells were exposed to actinomycin D for no longer than 15 min. Next, the L929 cells were incubated for 20-24 h at 37° C. The next day, 20 μl of WST-1 was added to each well and incubated for an additional 4 h at 37° C., and OD₄₅₀ were then read. IC₅₀s were determined from these readings by using prism 3.02 software (GraphPad, San Diego). Each measurement was performed in quadruplicate, and each experiment was performed a minimum of two times.
KG-1 bioassay: as described in section 1.2.4.
A-549 bioassay:
A-549 cells (ATCC cat#CCL-185, cultured in F12 base media with 10% fetal bovine serum & supplemented with 1% L-glutamine, 1% sodium bicarbonate, 50 units/mL penicillin, and 50 mg/mL Streptomycin) are human lung carcinomic epithelial cells that produce TARC in response to IL-13, in the presence of TNF. They were plated at 2×10⁵ cells per well (96-well plate) in a 100 μL volume and incubated overnight at 37° C., 5% CO₂. Following a 16-20 hr overnight incubation, the original 100 μl media seeding volume was removed and replaced with 200 μL fresh medium containing rhTNF-α (200 ng/mL), hIL-13 (5 ng/mL), and various concentrations of TVD-Ig 003 or control monoclonal antibody 13C5.5L3F (1e-7 M to 1e-14 M). After a 16-20 hr incubation, the well contents were collected for determining hTARC levels using standard ELISA (R&D Systems). Neutralization potencies of TVD-Ig 003 and 13C5.5L3F were determined by calculating IC₅₀ values generated from ploting TARC concentrations versus Ig (TVD-Ig 003 or monoclonal antibody) concentrations (sigmoidal curve dose responses), using GraphPad Prism software. Each measurement was performed in quadruplicate, and each experiment was performed a minimum of two times.

Based on functional characterizations using 3 different bioassays for TNF, IL-13, and IL-18, it was demonstrated that TVD-Ig 003 was capable of inhibiting TNF, IL-13, and IL-18, with potencies similar to that of the parental binding proteins, e.g., monoclonal antibodies (Table 12).

TABLE 12
Characterization of TVD-Ig 003 neutralization potencies (IC₅₀, nM)
against TNF, IL-13, and IL-18 by specific cell-based bioassays

| Ig | TNF | IL-13 | IL-18 |
|---|---|---|---|
| TVD-Ig 003 | 0.05 | 0.12 | 0.16 |
| Control monoclonal | 0.02 | 0.05 | 0.10 |
| antibodies | | | |

### Example 3

### Construction, Expression, and Purification of Tri-Variable Domain Immunoglobulin (TVD-Ig) Molecules that Recognize CD3, EGFR, and IGF1R

Parent monoclonal antibody anti-CD3, anti-EGFR, and an anti-IGF1R were used to construct TVD-Igs 003a and 004. The VL/VH domains of these three monoclonal antibodies were fused together via short linkers (the linker sequence is TVAAP (SEQ ID NO:13) for VL and ASTKGP (SEQ ID NO:21) for VH) by overlapping PCR, in a domain order of 5′VD (CD3)-SL-VD (EGFR)—SL-VD (IGF1R), followed by constant regions, in both HC and LC, termed TVD-Ig 003a. A different TVD-Ig (TVD-Ig 004) was also generated with a domain order of 5′ VD (CD3)-SL-VD (IGF1R)—SL-VD (EGFR), followed by constant regions, in both HC and LC. The procedures of the PCR cloning, expression, and purification process were similar to that described for TVD-Ig 001 and 002, except the primer sequences were different due to VL/VH sequence differences.

### Example 3.1

### Construction, Expression, and Purification of Triple Variable Domain Immunoglobulin (TVD-Ig003a and TVD004)

To generate light chain constructs TVD-Ig 003a, VL domain of the CD3 antibody was PCR amplified using specific primers 28 and 25; meanwhile VL domain of the EGFR SL-IGF1R DVD-Ig was amplified using specific primers 26 and 27; Both PCR reactions are performed according to standard PCR techniques and procedures. The two PCR products are gel-purified, and used together as overlapping template for the subsequent overlapping PCR reaction using standard PCR conditions and primers 27 and 28. The overlapping PCR products are subcloned into Nru I and Not I double digested pHybE-hCK mammalian expression vector (Abbott) by using standard homologous recombination approach.

To generate heavy chain constructs TVD-Ig 003a, VH domain of the EGFR-SL-IGFR DVD-Ig was PCR amplified using specific primers 32 and 29; meanwhile VH domain of the Anti-CD3 antibody was amplified using specific primers 31 and 30; Both PCR reactions are performed according to standard PCR techniques and procedures. The two PCR products are gel-purified, and used together as overlapping template for the subsequent overlapping PCR reaction using standard PCR conditions and primers 29 and 30. The overlapping PCR products are subcloned into Nru I and Not I double digested pHybE-hCg1, z-non-a mammalian expression vector (Abbott) by using standard homologous recombination approach.

To generate light chain constructs TVD-Ig 004, VL domain of the CD3 antibody was PCR amplified using specific primers 28 and 34; meanwhile VL domain of the IGF1R SL-EGFR DVD-Ig was amplified using specific primers 33 and 27; Both PCR reactions are performed according to standard PCR techniques and procedures. The two PCR products are gel-purified, and used together as overlapping template for the subsequent overlapping PCR reaction using standard PCR conditions and primers 27 and 28. The overlapping PCR products are subcloned into Nru I and Not I double digested pHybE-hCK mammalian expression vector (Abbott) by using standard homologous recombination approach.

To generate heavy chain constructs TVD-Ig 004, VH domain of the IGF1R-SL-EGFR DVD-Ig was PCR amplified using specific primers 36 and 29; meanwhile VH domain of the Anti-CD3 antibody was amplified using specific primers 35 and 30; Both PCR reactions are performed according to standard PCR techniques and procedures. The two PCR products are gel-purified, and used together as overlapping template for the subsequent overlapping PCR reaction using standard PCR conditions and primers 29 and 30. The overlapping PCR products are subcloned into Nru I and Not I double digested pHybE-hCg1, z-non-a mammalian expression vector (Abbott) by using standard homologous recombination approach.

Primer 25: TVD003a VL 3′ inside
(SEQ ID NO: 173)
TACAGGACTTTGTGTCAGAAGGATATCAGGAGCTGCGACGGTGCGATTGA
TCTCAAGCTTAGTGCCACTA
Primer 26: TVD003a VL 5′ inside
(SEQ ID NO: 174)
TAGTGGCACTAAGCTTGAGATCAATCGCACCGTCGCAGCTCCTGATATCC
TTCTGACACAAAGTCCTGTA
Primer 27: TVD-VL-CD3 3′ outside
(SEQ ID NO: 175)
GTCGAGGTCGGGGGATCCGGCCTTGCCGGCCTCGA
Primer 28: TVD-VL-CD3 5′ outside
(SEQ ID NO: 176)
CGCGTGCCCGCCCAGCTGCTGGGCCTGCTGCTGCTGTGGT
Primer 29: TVD VH-CD3 3′ outside
(SEQ ID NO: 177)
GTCGAGGTCGGGGGATCCGGCCTTGCCGGCCTCGA
Primer 30: TVD-VH CD3 5′ outside
(SEQ ID NO: 178)
ACCATGGAGTTTGGGCTGAGCTGGCTTTTTCTTGTCGCGATTTTA
Primer 31: TVD003 VH 3′ inside
(SEQ ID NO: 179)
GGCCCGCTCTGTTTGAGCTGAACCTGAGGCCCTTTCGTGGAGGCGGAGGA
CACGGTCAGAGTGGTACCCT
Primer 32: TVD003 VH 5′ inside
(SEQ ID NO: 180)
AGGGTACCACTCTGACCGTGTCCTCCGCCTCCACGAAAGGGCCTCAGGTT
CAGCTCAAACAGAGCGGGCC
Primer 33: TVD004 VL 5′ inside
(SEQ ID NO: 181)
TAGTGGCACTAAGCTTGAGATCAATCGCACCGTCGCAGCTCCTGATATCC
AGATGACTCAGTTTCCCAGC
Primer 34: TVD004VL 3′ inside
(SEQ ID NO: 182)
GCTGGGAAACTGAGTCATCTGGATATCAGGAGCTGCGACGGTGCGATTGA
TCTCAAGCTTAGTGCCACTA
Primer 35: TVD004 VH 3′ inside
(SEQ ID NO: 183)
GCCGCCCCCGGACTCAAGGAGCTGCACCTCAGGCCCTTTCGTGGAGGCGG
AGGACACGGTCAGAGTGGTACC
Primer 36: TVD004 VH 5′ inside
(SEQ ID NO: 184)
GGTACCACTCTGACCGTGTCCTCCGCCTCCACGAAAGGGCCTGAGGTGCA
GCTCCTTGAGTCCGGGGGCGGC

TABLE 13
Amino acid sequence of TVD-Igs capable of
binding CD3, EGFR and IGF1R Protein

| Protein | Sequence | Sequence |
|---|---|---|
| region | Identifier | 12345678901234567890 |
| TVD-Ig 003a | SEQ ID NO: 185 | QVQLQQSGAELARPGASVKM |
| HEAVY | | SCKASGYTFTRYTMHWVKQR |
| VARIABLE | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPQVQLKQSGPGLVQPS |
| | | QSLSITCTVSGFSLTNYGVH |
| | | WVRQSPGKGLEWLGVIWSGG |
| | | NTDYNTPFTSRLSINKDNSK |
| | | SQVFFKMNSLQSNDTAIYYC |
| | | ARALTYYDYEFAYWGQGTLV |
| | | TVSAASTKGPEVQLLESGGG |
| | | LVQPGGSLRLSCTASGFTFS |
| | | SYAMNWVRQAPGKGLEWVSA |
| | | ISGSGGTTFYADSVKGRFTI |
| | | SRDNSRTTLYLQMNSLRAED |
| | | TAVYYCAKDLGWSDSYYYYY |
| | | GMDVWGQGTTVTVSS |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| LINKER | SEQ ID NO: 21 | ASTKGP |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| LINKER | SEQ ID NO: 21 | ASTKGP |
| IGF1R VH | SEQ ID NO: 167 | EVQLLESGGGLVQPGGSLRL |
| | | SCTASGFTFSSYAMNWVRQA |
| | | PGKGLEWVSAISGSGGTTFY |
| | | ADSVKGRFTISRDNSRTTLY |
| | | LQMNSLRAEDTAVYYCAKDL |
| | | GWSDSYYYYYGMDVWGQGTT |
| | | VTVSS |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 003a | SEQ ID NO: 186 | QIVLTQSPAIMSASPGEKVT |
| LIGHT | | MTCRASSSVSYMNWYQQKSG |
| VARIABLE | | TSPKRWIYDTSKVASGVPYR |
| | | FSGSGSGTSYSLTISSMEAE |
| | | DAATYYCQQWSSNPLTFGSG |
| | | TKLEINRTVAAPDILLTQSP |
| | | VILSVSPGERVSFSCRASQS |
| | | IGTNIHWYQQRTNGSPRLLI |
| | | KYASESISGIPSRFSGSGSG |
| | | TDFTLSINSVESEDIADYYC |
| | | QQNNNWPTTFGAGTKLELKR |
| | | TVAAPDIQMTQFPSSLSASV |
| | | GDRVTITCRASQGIRNDLGW |
| | | YQQKPGKAPKRLIYAASRLH |
| | | RGVPSRFSGSGSGTEFTLTI |
| | | SSLQPEDFATYYCLQHNSYP |
| | | CSFGQGTKLEIKR |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| LINKER | SEQ ID NO: 13 | TVAAP |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| LINKER | SEQ ID NO: 13 | TVAAP |
| IGF1R VL | SEQ ID NO: 168 | DIQMTQFPSSLSASVGDRVT |
| | | ITCRASQGIRNDLGWYQQKP |
| | | GKAPKRLIYAASRLHRGVPS |
| | | RFSGSGSGTEFTLTISSLQP |
| | | EDFATYYCLQHNSYPCSFGQ |
| | | GTKLEIKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| TVD-Ig 004 | SEQ ID NO: 187 | QVQLQQSGAELARPGASVKM |
| HEAVY | | SCKASGYTFTRYTMHWVKQR |
| VARIABLE | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPEVQLLESGGGLVQPG |
| | | GSLRLSCTASGFTFSSYAMN |
| | | WVRQAPGKGLEWVSAISGSG |
| | | GTTFYADSVKGRFTISRDNS |
| | | RTTLYLQMNSLRAEDTAVYY |
| | | CAKDLGWSDSYYYYYGMDVW |
| | | GQGTTVTVSSASTKGPQVQL |
| | | KQSGPGLVQPSQSLSITCTV |
| | | SGFSLTNYGVHWVRQSPGKG |
| | | LEWLGVIWSGGNTDYNTPFT |
| | | SRLSINKDNSKSQVFFKMNS |
| | | LQSNDTAIYYCARALTYYDY |
| | | EFAYWGQGTLVTVSA |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| LINKER | SEQ ID NO: 21 | ASTKGP |
| IGF1R VH | SEQ ID NO: 167 | EVQLLESGGGLVQPGGSLRL |
| | | SCTASGFTFSSYAMNWVRQA |
| | | PGKGLEWVSAISGSGGTTFY |
| | | ADSVKGRFTISRDNSRTTLY |
| | | LQMNSLRAEDTAVYYCAKDL |
| | | GWSDSYYYYYGMDVWGQGTT |
| | | VTVSS |
| LINKER | SEQ ID NO: 21 | ASTKGP |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 004 | SEQ ID NO: 188 | QIVLTQSPAIMSASPGEKVT |
| LIGHT | | MTCRASSSVSYMNWYQQKSG |
| VARIABLE | | TSPKRWIYDTSKVASGVPYR |
| | | FSGSGSGTSYSLTISSMEAE |
| | | DAATYYCQQWSSNPLTFGSG |
| | | TKLEINRTVAAPDILLTQSP |
| | | VILSVSPGERVSFSCRASQS |
| | | IGTNIHWYQQRTNGSPRLLI |
| | | KYASESISGIPSRFSGSGSG |
| | | TDFTLSINSVESEDIADYYC |
| | | QQNNNWPTTFGAGTKLELKR |
| | | TVAAPDILLTQSPVILSVSP |
| | | GERVSFSCRASQSIGTNIHW |
| | | YQQRTNGSPRLLIKYASESI |
| | | SGIPSRFSGSGSGTDFTLSI |
| | | NSVESEDIADYYCQQNNNWP |
| | | TTFGAGTKLELKR |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| LINKER | SEQ ID NO: 13 | TVAAP |
| IGF1R VL | SEQ ID NO: 168 | DIQMTQFPSSLSASVGDRVT |
| | | ITCRASQGIRNDLGWYQQKP |
| | | GKAPKRLIYAASRLHRGVPS |
| | | RFSGSGSGTEFTLTISSLQP |
| | | EDFATYYCLQHNSYPCSFGQ |
| | | GTKLEIKR |
| LINKER | SEQ ID NO: 13 | TVAAP |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

### Example 3.2

### Characterization of Tri-Variable Domain Immunoglobulin Molecule (TVD-Ig 003a)

### Example 3.2.1

### Size Exclusion Chromatography Analysis of TVD-Ig 003a

Purified TVD-Ig 003a was analyzed by SEC as described for TVD-Ig 001 and 002. TVD-Ig 003a exhibited a monomeric profile on SEC, apparently existed as a homogeneous protein (72.4% monomer).

### Example 3.2.2

### Functional Analysis of TVD-Ig Using Redirected Cytotoxicity Assay (rCTL Assay)

To determine if TVD-Ig was able to inhibit redirected cytotoxity 2 cell-based bioassays were used:

### Example 3.2.2a

### FACS Based Redirected Cytotoxicity Assay (rCTL Assay)

Human CD3+ T cells are isolated from previously frozen isolated PBMC by a negative selection enrichment column (R&D Cat.#HTCC-525). T cells are stimulated for 4 days in flasks coated with 10 μg/mL anti-CD3 (OKT-3, BD) and 2 μg/mL anti-CD28 (CD28.2, Abcam) in complete RPMI media (L-glutamine, 55 mM β-ME, Pen/Strep, 10% FCS). T cells are rested overnight in 30 U/mL IL-2 (Peprotech) before using in assay. DoHH2 or Raji target cells are labeled with PKH26 (Sigma) according to manufacturer's instructions. RPMI 1640 media (no phenol, Invitrogen) containing L-glutamine and 10% FBS (Hyclone) is used throughout the rCTL assay.

Effector T cells (E) and targets (T) are plated at 10⁵ and 10⁴ cells/well in 96-well plates (Costar #3799), respectively to give an E:T ratio of 10:1. DVD-Ig molecules are appropriately diluted to obtain concentration-dependent titration curves. After an overnight incubation cells are pelleted and washed with PBS once before resuspending in PBS containing 0.1% BSA (Invitrogen) and 0.5 μg/mL propidium iodide (BD). FACS data is collected on a FACSCanto machine (BD) and analyzed in Flowjo (Treestar).

The percent live targets in the DVD-Ig treated samples divided by the percent total targets (control, no treatment) is calculated to determine percent specific lysis. The data is graphed and IC50s are calculated in Prism (Graphpad).

### Example 3.2.2b

### Impedence Based Redirected Cytotoxicity Assay (rCTL Assay)

T cells are prepared as above. EGFR-expressing target cells are allowed to adhere to ACEA RT-CES 96-well plates (ACEA Bio, San Diego) overnight. Effector T cells (E) and targets (T) are then plated at 2×10⁵ and 2×10⁴ cells/well to give an E:T ratio of 10:1. DVD-Ig molecules are appropriately diluted to obtain concentration-dependent titration curves. The cell indexes of targets in the DVD-Ig treated samples are divided by the cell indexes of control targets (no treatment) to calculate percent specific lysis. The data is graphed and IC50s are calculated in Prism (Graphpad).

Redirected cytotoxicity data for TVD003a can be found in Table 16.

### Example 4

### Construction, Expression, and Purification of Tri-Variable Domain Immunoglobulin (TVD-Ig) Molecules that Recognize CD3, EGFR, and HER2

Parent monoclonal antibody anti-CD3, anti-EGFR, and an anti-HER2 were used to construct TVD-Igs 005 and 006. The VL/VH domains of these three monoclonal antibodies were fused together via short linkers (the linker sequence is TVAAP (SEQ ID NO:13) for VL and ASTKGP (SEQ ID NO:21) for VH) by overlapping PCR, in a domain order of 5′VD (CD3)-SL-VD (EGFR)-SL-VD (Her2), followed by constant regions, in both HC and LC, termed TVD-Ig 005. A different TVD-Ig (TVD-Ig 006) was also generated with a domain order of 5′VD (CD3)-SL-VD (Her2)-SL-VD (EGFR), followed by constant regions, in both HC and LC. The procedures of the PCR cloning, expression, and purification process were similar to that described for TVD-Ig 001 and 002, except the primer sequences were different due to VL/VH sequence differences.

### Example 4.1

### Construction, Expression, and Purification of Triple Variable Domain Immunoglobulin (TVD-Ig005 and TVD006)

To generate light chain constructs TVD-Ig 005, VL domain of the CD3 antibody was PCR amplified using specific primers 28 and 25; meanwhile VL domain of the EGFR SL-Her2 DVD-Ig was amplified using specific primers 26 and 27; Both PCR reactions are performed according to standard PCR techniques and procedures. The two PCR products are gel-purified, and used together as overlapping template for the subsequent overlapping PCR reaction using standard PCR conditions and primers 27 and 28. The overlapping PCR products are subcloned into Nru I and Not I double digested pHybE-hCK mammalian expression vector (Abbott) by using standard homologous recombination approach.

To generate heavy chain constructs TVD-Ig 005, VH domain of the EGFR-SL-Her2 DVD-Ig was PCR amplified using specific primers 32 and 29; meanwhile VH domain of the Anti-CD3 antibody was amplified using specific primers 31 and 30; Both PCR reactions are performed according to standard PCR techniques and procedures. The two PCR products are gel-purified, and used together as overlapping template for the subsequent overlapping PCR reaction using standard PCR conditions and primers 29 and 30. The overlapping PCR products are subcloned into Nru I and Not I double digested pHybE-hCg1, z-non-a mammalian expression vector (Abbott) by using standard homologous recombination approach.

To generate light chain constructs TVD-Ig 006, VL domain of the CD3 antibody was PCR amplified using specific primers 28 and 39; meanwhile VL domain of the Her2 SL-EGFR DVD-Ig was amplified using specific primers 40 and 27; Both PCR reactions are performed according to standard PCR techniques and procedures. The two PCR products are gel-purified, and used together as overlapping template for the subsequent overlapping PCR reaction using standard PCR conditions and primers 27 and 28. The overlapping PCR products are subcloned into Nru I and Not I double digested pHybE-hCK mammalian expression vector (Abbott) by using standard homologous recombination approach.

To generate heavy chain constructs TVD-Ig 006, VH domain of the Her2-SL-EGFR DVD-Ig was PCR amplified using specific primers 38 and 29; meanwhile VH domain of the Anti-CD3 antibody was amplified using specific primers 37 and 30; Both PCR reactions are performed according to standard PCR techniques and procedures. The two PCR products are gel-purified, and used together as overlapping template for the subsequent overlapping PCR reaction using standard PCR conditions and primers 29 and 30. The overlapping PCR products are subcloned into Nru I and Not I double digested pHybE-hCg1, z-non-a mammalian expression vector (Abbott) by using standard homologous recombination approach.

Primer 37 TVD006 VH 3′ inside
(SEQ ID NO: 189)
CTCCACCAGCTGGACCTCAGGCCCTTTCGTGGAGGCGGAGGACACGGTCA
GAGTGGTACCCTGCCCCCAG
Primer 38: TVD006 VH 5′ inside
(SEQ ID NO: 190)
CTGGGGGCAGGGTACCACTCTGACCGTGTCCTCCGCCTCCACGAAAGGGC
CTGAGGTCCAGCTGGTGGAG
Primer 39: TVD006 VL 3′ inside
(SEQ ID NO: 191)
TGGATGGACTTTGCGTCATCTGTATATCAGGAGCTGCGACGGTGCGATTG
ATCTCAAGCTTAGTGCCACT
Primer 40: TVD006 VL 5′ inside
(SEQ ID NO: 192)
AGTGGCACTAAGCTTGAGATCAATCGCACCGTCGCAGCTCCTGATATACA
GATGACGCAAAGTCCATCCA

TABLE 14
Amino acid sequence of TVD-Igs capable of
binding CD3, EGFR and Her2 Protein

| Protein | Sequence | Sequence |
|---|---|---|
| region | Identifier | 12345678901234567890 |
| TVD-Ig 005 | SEQ ID NO: 193 | QVQLQQSGAELARPGASVKM |
| HEAVY | | SCKASGYTFTRYTMHWVKQR |
| VARIABLE | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPQVQLKQSGPGLVQPS |
| | | QSLSITCTVSGFSLTNYGVH |
| | | WVRQSPGKGLEWLGVIWSGG |
| | | NTDYNTPFTSRLSINKDNSK |
| | | SQVFFKMNSLQSNDTAIYYC |
| | | ARALTYYDYEFAYWGQGTLV |
| | | TVSAASTKGPEVQLVESGGG |
| | | LVQPGGSLRLSCAASGFNIK |
| | | DTYIHWVRQAPGKGLEWVAR |
| | | IYPTNGYTRYADSVKGRFTI |
| | | SADTSKNTAYLQMNSLRAED |
| | | TAVYYCSRWGGDGFYAMDYW |
| | | GQGTLVTVSS |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| LINKER | SEQ ID NO: 21 | ASTKGP |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| LINKER | SEQ ID NO: 21 | ASTKGP |
| HER2 VH | SEQ ID NO: 171 | EVQLVESGGGLVQPGGSLRL |
| | | SCAASGFNIKDTYIHWVRQA |
| | | PGKGLEWVARIYPTNGYTRY |
| | | ADSVKGRFTISADTSKNTAY |
| | | LQMNSLRAEDTAVYYCSRWG |
| | | GDGFYAMDYWGQGTLVTVSS |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 005 | SEQ ID NO: 194 | QIVLTQSPAIMSASPGEKVT |
| LIGHT | | MTCRASSSVSYMNWYQQKSG |
| VARIABLE | | TSPKRWIYDTSKVASGVPYR |
| | | FSGSGSGTSYSLTISSMEAE |
| | | DAATYYCQQWSSNPLTFGSG |
| | | TKLEINRTVAAPDILLTQSP |
| | | VILSVSPGERVSFSCRASQS |
| | | IGTNIHWYQQRTNGSPRLLI |
| | | KYASESISGIPSRFSGSGSG |
| | | TDFTLSINSVESEDIADYYC |
| | | QQNNNWPTTFGAGTKLELKR |
| | | TVAAPDIQMTQSPSSLSASV |
| | | GDRVTITCRASQDVNTAVAW |
| | | YQQKPGKAPKLLIYSASFLY |
| | | SGVPSRFSGSRSGTDFTLTI |
| | | SSLQPEDFATYYCQQHYTTP |
| | | PTFGQGTKVEIKR |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| LINKER | SEQ ID NO: 13 | TVAAP |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| LINKER | SEQ ID NO: 13 | TVAAP |
| HER2 VL | SEQ ID NO: 172 | DIQMTQSPSSLSASVGDRVT |
| | | ITCRASQDVNTAVAWYQQKP |
| | | GKAPKLLIYSASFLYSGVPS |
| | | RFSGSRSGTDFTLTISSLQP |
| | | EDFATYYCQQHYTTPPTFGQ |
| | | GTKVEIKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| TVD-Ig 006 | SEQ ID NO: 195 | QVQLQQSGAELARPGASVKM |
| HEAVY | | SCKASGYTFTRYTMHWVKQR |
| VARIABLE | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPEVQLVESGGGLVQPG |
| | | GSLRLSCAASGFNIKDTYIH |
| | | WVRQAPGKGLEWVARIYPTN |
| | | GYTRYADSVKGRFTISADTS |
| | | KNTAYLQMNSLRAEDTAVYY |
| | | CSRWGGDGFYAMDYWGQGTL |
| | | VTVSSASTKGPQVQLKQSGP |
| | | GLVQPSQSLSITCTVSGFSL |
| | | TNYGVHWVRQSPGKGLEWLG |
| | | VIWSGGNTDYNTPFTSRLSI |
| | | NKDNSKSQVFFKMNSLQSND |
| | | TAIYYCARALTYYDYEFAYW |
| | | GQGTLVTVSA |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| LINKER | SEQ ID NO: 21 | ASTKGP |
| HER2 VH | SEQ ID NO: 171 | EVQLVESGGGLVQPGGSLRL |
| | | SCAASGFNIKDTYIHWVRQA |
| | | PGKGLEWVARIYPTNGYTRY |
| | | ADSVKGRFTISADTSKNTAY |
| | | LQMNSLRAEDTAVYYCSRWG |
| | | GDGFYAMDYWGQGTLVTVSS |
| LINKER | SEQ ID NO: 21 | ASTKGP |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 006 | SEQ ID NO: 196 | QIVLTQSPAIMSASPGEKVT |
| LIGHT | | MTCRASSSVSYMNWYQQKSG |
| VARIABLE | | TSPKRWIYDTSKVASGVPYR |
| | | FSGSGSGTSYSLTISSMEAE |
| | | DAATYYCQQWSSNPLTFGSG |
| | | TKLEINRTVAAPDIQMTQSP |
| | | SSLSASVGDRVTITCRASQD |
| | | VNTAVAWYQQKPGKAPKLLI |
| | | YSASFLYSGVPSRFSGSRSG |
| | | TDFTLTISSLQPEDFATYYC |
| | | QQHYTTPPTFGQGTKVEIKR |
| | | TVAAPDILLTQSPVILSVSP |
| | | GERVSFSCRASQSIGTNIHW |
| | | YQQRTNGSPRLLIKYASESI |
| | | SGIPSRFSGSGSGTDFTLSI |
| | | NSVESEDIADYYCQQNNNWP |
| | | TTFGAGTKLELKR |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| LINKER | SEQ ID NO: 13 | TVAAP |
| HER2 VL | SEQ ID NO: 172 | DIQMTQSPSSLSASVGDRVT |
| | | ITCRASQDVNTAVAWYQQKP |
| | | GKAPKLLIYSASFLYSGVPS |
| | | RFSGSRSGTDFTLTISSLQP |
| | | EDFATYYCQQHYTTPPTFGQ |
| | | GTKVEIKR |
| LINKER | SEQ ID NO: 13 | TVAAP |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

### Example 5

### Construction, Expression, and Purification of Tri-Variable Domain Immunoglobulin (TVD-Ig) Molecules that Recognize CD3, CD20, and EGFR

Parent monoclonal antibody anti-CD3, anti-EGFR, and an anti-HER2 were used to construct TVD-Igs 007 and 008. The VL/VH domains of these three monoclonal antibodies were fused together via short linkers (the linker sequence is TVAAP (SEQ ID NO:13) for VL and ASTKGP (SEQ ID NO:21) for VH) by overlapping PCR, in a domain order of 5′VD (CD3)-SL-VD (CD20)-SL-VD (EGFR), followed by constant regions, in both HC and LC, termed TVD-Ig 007. A different TVD-Ig (TVD-Ig 008) was also generated with a domain order of 5′VD (CD3)-SL-VD (EGFR)-SL-VD (CD20), followed by constant regions, in both HC and LC. The procedures of the PCR cloning, expression, and purification process were similar to that described for TVD-Ig 001 and 002, except the primer sequences were different due to VL/VH sequence differences.

### Example 5.1

### Construction, Expression, and Purification of Triple Variable Domain Immunoglobulin (TVD-Ig007 and TVD008)

To generate light chain constructs TVD-Ig 007, VL domain of the CD3 antibody was PCR amplified using specific primers 28 and 41; meanwhile VL domain of the CD20 SL-EGFR DVD-Ig was amplified using specific primers 42 and 27; Both PCR reactions are performed according to standard PCR techniques and procedures. The two PCR products are gel-purified, and used together as overlapping template for the subsequent overlapping PCR reaction using standard PCR conditions and primers 27 and 28. The overlapping PCR products are subcloned into Nru I and Not I double digested pHybE-hCK mammalian expression vector (Abbott) by using standard homologous recombination approach.

To generate heavy chain constructs TVD-Ig 007, VH domain of the CD20-SL-EGFR DVD-Ig was PCR amplified using specific primers 44 and 29; meanwhile VH domain of the Anti-CD3 antibody was amplified using specific primers 43 and 30; Both PCR reactions are performed according to standard PCR techniques and procedures. The two PCR products are gel-purified, and used together as overlapping template for the subsequent overlapping PCR reaction using standard PCR conditions and primers 29 and 30. The overlapping PCR products are subcloned into Nru I and Not I double digested pHybE-hCg1, z-non-a mammalian expression vector (Abbott) by using standard homologous recombination approach.

To generate light chain constructs TVD-Ig 008, VL domain of the CD3 antibody was PCR amplified using specific primers 28 and 25; meanwhile VL domain of the EGFR SL-CD20 DVD-Ig was amplified using specific primers 26 and 27; Both PCR reactions are performed according to standard PCR techniques and procedures. The two PCR products are gel-purified, and used together as overlapping template for the subsequent overlapping PCR reaction using standard PCR conditions and primers 27 and 28. The overlapping PCR products are subcloned into Nru I and Not I double digested pHybE-hCK mammalian expression vector (Abbott) by using standard homologous recombination approach.

To generate heavy chain constructs TVD-Ig 008, VH domain of the EGFR-SL-CD/20 DVD-Ig was PCR amplified using specific primers 32 and 29; meanwhile VH domain of the Anti-CD3 antibody was amplified using specific primers 31 and 30; Both PCR reactions are performed according to standard PCR techniques and procedures. The two PCR products are gel-purified, and used together as overlapping template for the subsequent overlapping PCR reaction using standard PCR conditions and primers 29 and 30. The overlapping PCR products are subcloned into Nru I and Not I double digested pHybE-hCg1, z-non-a mammalian expression vector (Abbott) by using standard homologous recombination approach.

Primer 41: TVD007 VL 3′ inside
(SEQ ID NO: 197)
AGCGGGAGACTGGGAGAGCACGATTTGAGGAGCTGCGACGGTGCGATTGA
TCTCAAGCTTAGTGCCACTA′
Primer 42: TVD007 VL 5′ inside
(SEQ ID NO: 198)
TAGTGGCACTAAGCTTGAGATCAATCGCACCGTCGCAGCTCCTCAAATCG
TGCTCTCCCAGTCTCCCGCT
Primer 43: TVD007 VH 3′ inside
(SEQ ID NO: 199)
GCCCCAGGTTGCTGCAGCTGAACCTGAGGCCCTTTCGTGGAGGCGGAGGA
CACGGTCAGAGTGGTACCCT
Primer 44: TVD007 VH 5′ inside
(SEQ ID NO: 200)
AGGGTACCACTCTGACCGTGTCCTCCGCCTCCACGAAAGGGCCTCAGGTT
CAGCTGCAGCAACCTGGGGC

TABLE 15
Amino acid sequence of TVD-Igs capable of
binding CD3, EGFR and CD20 Protein

| Protein | Sequence | Sequence |
|---|---|---|
| region | Identifier | 12345678901234567890 |
| TVD-Ig 007 | SEQ ID NO: 201 | QVQLQQSGAELARPGASVKM |
| HEAVY | | SCKASGYTFTRYTMHWVKQR |
| VARIABLE | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPQVQLQQPGAELVKPG |
| | | ASVKMSCKASGYTFTSYNMH |
| | | WVKQTPGRGLEWIGAIYPGN |
| | | GDTSYNQKFKGKATLTADKS |
| | | SSTAYMQLSSLTSEDSAVYY |
| | | CARSTYYGGDWYFNVWGAGT |
| | | TVTVSAASTKGPQVQLKQSG |
| | | PGLVQPSQSLSITCTVSGFS |
| | | LTNYGVHWVRQSPGKGLEWL |
| | | GVIWSGGNTDYNTPFTSRLS |
| | | INKDNSKSQVFFKMNSLQSN |
| | | DTAIYYCARALTYYDYEFAY |
| | | WGQGTLVTVSA |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| LINKER | SEQ ID NO: 21 | ASTKGP |
| CD20 VH | SEQ ID NO: 169 | QVQLQQPGAELVKPGASVKM |
| | | SCKASGYTFTSYNMHWVKQT |
| | | PGRGLEWIGAIYPGNGDTSY |
| | | NQKFKGKATLTADKSSSTAY |
| | | MQLSSLTSEDSAVYYCARST |
| | | YYGGDWYFNVWGAGTTVTVS |
| | | A |
| LINKER | SEQ ID NO: 21 | ASTKGP |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 007 | SEQ ID NO: 202 | QIVLTQSPAIMSASPGEKVT |
| LIGHT | | MTCRASSSVSYMNWYQQKSG |
| VARIABLE | | TSPKRWIYDTSKVASGVPYR |
| | | FSGSGSGTSYSLTISSMEAE |
| | | DAATYYCQQWSSNPLTFGSG |
| | | TKLEINRTVAAPQIVLSQSP |
| | | AILSPSPGEKVTMTCRASSS |
| | | VSYIHWFQQKPGSSPKPWIY |
| | | ATSNLASGVPVRFSGSGSGT |
| | | SYSLTISRVEAEDAATYYCQ |
| | | QWTSNPPTFGGGTKLEIKRT |
| | | VAAPDILLTQSPVILSVSPG |
| | | ERVSFSCRASQSIGTNIHWY |
| | | QQRTNGSPRLLIKYASESIS |
| | | GIPSRFSGSGSGTDFTLSIN |
| | | SVESEDIADYYCQQNNNWPT |
| | | TFGAGTKLELKR |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| LINKER | SEQ ID NO: 13 | TVAAP |
| CD20 VL | SEQ ID NO: 170 | QIVLSQSPAILSPSPGEKVT |
| | | MTCRASSSVSYIHWFQQKPG |
| | | SSPKPWIYATSNLASGVPVR |
| | | FSGSGSGTSYSLTISRVEAE |
| | | DAATYYCQQWTSNPPTFGGG |
| | | TKLEIKR |
| LINKER | SEQ ID NO: 13 | TVAAP |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| TVD-Ig 008 | SEQ ID NO: 203 | QVQLQQSGAELARPGASVKM |
| HEAVY | | SCKASGYTFTRYTMHWVKQR |
| VARIABLE | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSSA |
| | | STKGPQVQLKQSGPGLVQPS |
| | | QSLSITCTVSGFSLTNYGVH |
| | | WVRQSPGKGLEWLGVIWSGG |
| | | NTDYNTPFTSRLSINKDNSK |
| | | SQVFFKMNSLQSNDTAIYYC |
| | | ARALTYYDYEFAYWGQGTLV |
| | | TVSAASTKGPQVQLQQPGAE |
| | | LVKPGASVKMSCKASGYTFT |
| | | SYNMHWVKQTPGRGLEWIGA |
| | | IYPGNGDTSYNQKFKGKATL |
| | | TADKSSSTAYMQLSSLTSED |
| | | SAVYYCARSTYYGGDWYFNV |
| | | WGAGTTVTVSA |
| CD3 VH | SEQ ID NO: 163 | QVQLQQSGAELARPGASVKM |
| | | SCKASGYTFTRYTMHWVKQR |
| | | PGQGLEWIGYINPSRGYTNY |
| | | NQKFKDKATLTTDKSSSTAY |
| | | MQLSSLTSEDSAVYYCARYY |
| | | DDHYCLDYWGQGTTLTVSS |
| LINKER | SEQ ID NO: 21 | ASTKGP |
| EGFR VH | SEQ ID NO: 165 | QVQLKQSGPGLVQPSQSLSI |
| | | TCTVSGFSLTNYGVHWVRQS |
| | | PGKGLEWLGVIWSGGNTDYN |
| | | TPFTSRLSINKDNSKSQVFF |
| | | KMNSLQSNDTAIYYCARALT |
| | | YYDYEFAYWGQGTLVTVSA |
| LINKER | SEQ ID NO: 21 | ASTKGP |
| CD20 VH | SEQ ID NO: 169 | QVQLQQPGAELVKPGASVKM |
| | | SCKASGYTFTSYNMHWVKQT |
| | | PGRGLEWIGAIYPGNGDTSY |
| | | NQKFKGKATLTADKSSSTAY |
| | | MQLSSLTSEDSAVYYCARST |
| | | YYGGDWYFNVWGAGTTVTVS |
| | | A |
| CH | SEQ ID NO: 49 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| TVD-Ig 008 | SEQ ID NO: 204 | QIVLTQSPAIMSASPGEKVT |
| LIGHT | | MTCRASSSVSYMNWYQQKSG |
| VARIABLE | | TSPKRWIYDTSKVASGVPYR |
| | | FSGSGSGTSYSLTISSMEAE |
| | | DAATYYCQQWSSNPLTFGSG |
| | | TKLEINRTVAAPDILLTQSP |
| | | VILSVSPGERVSFSCRASQS |
| | | IGTNIHWYQQRTNGSPRLLI |
| | | KYASESISGIPSRFSGSGSG |
| | | TDFTLSINSVESEDIADYYC |
| | | QQNNNWPTTFGAGTKLELKR |
| | | TVAAPQIVLSQSPAILSPSP |
| | | GEKVTMTCRASSSVSYIHWF |
| | | QQKPGSSPKPWIYATSNLAS |
| | | GVPVRFSGSGSGTSYSLTIS |
| | | RVEAEDAATYYCQQWTSNPP |
| | | TFGGGTKLEIKR |
| CD3 VL | SEQ ID NO: 164 | QIVLTQSPAIMSASPGEKVT |
| | | MTCRASSSVSYMNWYQQKSG |
| | | TSPKRWIYDTSKVASGVPYR |
| | | FSGSGSGTSYSLTISSMEAE |
| | | DAATYYCQQWSSNPLTFGSG |
| | | TKLEINR |
| LINKER | SEQ ID NO: 13 | TVAAP |
| EGFR VL | SEQ ID NO: 166 | DILLTQSPVILSVSPGERVS |
| | | FSCRASQSIGTNIHWYQQRT |
| | | NGSPRLLIKYASESISGIPS |
| | | RFSGSGSGTDFTLSINSVES |
| | | EDIADYYCQQNNNWPTTFGA |
| | | GTKLELKR |
| LINKER | SEQ ID NO: 13 | TVAAP |
| CD20 VL | SEQ ID NO: 170 | QIVLSQSPAILSPSPGEKVT |
| | | MTCRASSSVSYIHWFQQKPG |
| | | SSPKPWIYATSNLASGVPVR |
| | | FSGSGSGTSYSLTISRVEAE |
| | | DAATYYCQQWTSNPPTFGGG |
| | | TKLEIKR |
| CL | SEQ ID NO: 54 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

### Example 5.2

### Characterization of Tri-Variable Domain Immunoglobulin Molecule (TVD-Ig 008)

### Example 3.2.1

### Size Exclusion Chromatography Analysis of TVD-Ig 008

Purified TVD-Ig 008 was analyzed by SEC as described for TVD-Ig 001 and 002. TVD-Ig 008 exhibited a monomeric profile on SEC, apparently existed as a homogeneous protein (76.4% monomer).

### Example 5.2.2

### Functional Analysis of TVD-Ig Using Redirected Cytotoxicity Assay (rCTL Assay)

To determine if TVD-Ig was able to inhibit redirected cytotoxity 2 cell-based bioassays were used:

### Example 5.2.2a

### FACS Based Redirected Cytotoxicity Assay (rCTL Assay)

Human CD3+ T cells are isolated from previously frozen isolated PBMC by a negative selection enrichment column (R&D Cat.#HTCC-525). T cells are stimulated for 4 days in flasks coated with 10 μg/mL anti-CD3 (OKT-3, BD) and 2 μg/mL anti-CD28 (CD28.2, Abcam) in complete RPMI media (L-glutamine, 55 mM β-ME, Pen/Strep, 10% FCS). T cells are rested overnight in 30 U/mL IL-2 (Peprotech) before using in assay. DoHH2 or Raji target cells are labeled with PKH26 (Sigma) according to manufacturer's instructions. RPMI 1640 media (no phenol, Invitrogen) containing L-glutamine and 10% FBS (Hyclone) is used throughout the rCTL assay.

Effector T cells (E) and targets (T) are plated at 10⁵ and 10⁴ cells/well in 96-well plates (Costar #3799), respectively to give an E:T ratio of 10:1. DVD-Ig molecules are appropriately diluted to obtain concentration-dependent titration curves. After an overnight incubation cells are pelleted and washed with PBS once before resuspending in PBS containing 0.1% BSA (Invitrogen) and 0.5 μg/mL propidium iodide (BD). FACS data is collected on a FACSCanto machine (BD) and analyzed in Flowjo (Treestar).

The percent live targets in the DVD-Ig treated samples divided by the percent total targets (control, no treatment) is calculated to determine percent specific lysis. The data is graphed and IC50s are calculated in Prism (Graphpad).

### Example 5.2.2b

### Impedence Based Redirected Cytotoxicity Assay (rCTL Assay)

T cells are prepared as above. EGFR-expressing target cells are allowed to adhere to ACEA RT-CES 96-well plates (ACEA Bio, San Diego) overnight. Effector T cells (E) and targets (T) are then plated at 2×10⁵ and 2×10⁴ cells/well to give an E:T ratio of 10:1. TVD-Ig molecules are appropriately diluted to obtain concentration-dependent titration curves. The cell indexes of targets in the TVD-Ig treated samples are divided by the cell indexes of control targets (no treatment) to calculate percent specific lysis. The data is graphed and IC50s are calculated in Prism (Graphpad). Results for the impedence based rCTL assay can be found in Table 16 for both TVD003a and TVD008.

TABLE 16
rCTL assay of TVD-Ig Constructs using Impedence Based assay

| Parent Antibody | |
|---|---|
| or TVD-Ig ID | IC50 (nM) |
| TVD003a | 2.636 |
| TVD008 | 1.037 |

### Example 5.2.2.c

### Binding of TVD-Igs to the Surface of Human Tumor Cell Lines as Assessed by Flow Cytometry using FACSCanto

Stable cell lines overexpressing a cell-surface antigen of interest or human tumor cell lines were harvested from tissue culture flasks and resuspended in phosphate buffered saline (PBS) containing 5% fetal bovine serum (PBS/FBS). Prior to staining, human tumor cells were incubated on ice with (100 μl) human IgG at 5 μg/ml in PBS/FCS. 1−5×10⁵ cells were incubated with antibody or TVD-Ig (50 nM) in PBS/FBS for 30-60 minutes on ice. Cells were washed twice and 100 μl of F(ab′)2 goat anti human IgG, Fcγ-Dylight488 (1:200 dilution in PBS) (Jackson ImmunoResearch, West Grove, Pa., Cat.#109-486-098) was added. After 30 minutes incubation on ice, cells were washed twice and resuspended in PBS/FBS. Fluorescence was measured using a Becton Dickinson FACSCanto (Becton Dickinson, San Jose, Calif.).

The following table contains the FACS geometric mean of parent antibodies and TVD-Ig constructs at 50 nM. Table 17 represents the FACS binding data on three cell lines, Jurkat, A431, and Raji cells for TVD008.

TABLE 17
Fluorescent Activated Cell Sorting of TVD-Ig
Constructs using FACS Canto

| | | | |
|---|---|---|---|
| | | FACS | FACS |
| | | Geometric | Geometric |
| Parent Antibody | | Mean | Mean |
| or TVD-Ig ID | Cell line used | N-terminal | C-terminal |

| | | |
|---|---|---|
| CD3 mAb | Jurkat | 572 |
| TVD008 | Jurkat | 439 |
| EGFR mAb | A431 | 53006 |
| TVD008 | A431 | 1096 |
| CD20 mAb | Raji | 3484 |
| TVD008 | Raji | 176 |

TVD008 showed binding to cell surface targets. The N-terminal domain bound the target on the cell surface as well as or better than the parent antibody. Binding can be restored or improved by adjusting linker length.

### INCORPORATION BY REFERENCE

The present disclosure incorporates by reference in their entirety techniques well known in the field of molecular biology and drug delivery. These techniques include, but are not limited to, techniques described in the following publications:
Ausubel et al. (eds.), Current Protocols in Molecular Biology, John Wiley & Sons, NY (1993);
Ausubel et al. (eds.), Short Protocols In Molecular Biology, John Wiley & Sons, NY (4^{th} edition, 1999) (ISBN 0-471-32938-X);
Giege, R. and Ducruix, A. Barrett, Crystallization of Nucleic Acids and Proteins, a Practical Approach, 2nd ea., pp. 20 1-16, Oxford University Press, New York, N.Y., (1999);
Goodson, in Medical Applications of Controlled Release, vol. 2, pp. 115-138 (1984);
Hammerling et al., in: Monoclonal Antibodies and T-Cell Hybridomas 563-681 (Elsevier, N.Y., 1981;
Harlow et al., Antibodies: A Laboratory Manual, (Cold Spring Harbor Laboratory Press, 2nd ed. 1988);
Kabat et al., Sequences of Proteins of Immunological Interest (National Institutes of Health, Bethesda, Md. (1987) and (1991);
Kabat, E. A., et al. (1991) Sequences of Proteins of Immunological Interest, Fifth Edition, U.S. Department of Health and Human Services, NIH Publication No. 91-3242;
Kontermann and Dubel eds., Antibody Engineering (2001) Springer-Verlag. New York. 790 pp. (ISBN 3-540-41354-5);
Kriegler, Gene Transfer and Expression, A Laboratory Manual, Stockton Press, NY (1990);
Langer and Wise (eds.), Medical Applications of Controlled Release, CRC Press, Boca Raton, Fla. (1974);
Lu and Weiner eds., Cloning and Expression Vectors for Gene Function Analysis (2001) BioTechniques Press. Westborough, Mass. 298 pp. (ISBN 1-881299-21-X);
Old, R. W. & S. B. Primrose, Principles of Gene Manipulation: An Introduction To Genetic Engineering (3d Ed. 1985) Blackwell Scientific Publications, Boston. Studies in Microbiology; V.2:409 pp. (ISBN 0-632-01318-4); Robinson, J. R. (ed.), Sustained and Controlled Release Drug Delivery Systems, Marcel Dekker, Inc., NY (1978);
Ruan, Q., Skinner, J. P. and Tetin, S. Y. Using non-fluorescent FRET acceptors in protein binding studies. Analyt. Biochemistry (2009), 393, 196-204;
Sambrook, J. et al. eds., Molecular Cloning: A Laboratory Manual (2d Ed. 1989) Cold Spring Harbor Laboratory Press, NY. Vols. 1-3. (ISBN 0-87969-309-6);
Smolen and Ball (eds.), Controlled Drug Bioavailability, Drug Product Design and Performance, John Wiley & Sons, NY (1984);
Winnacker, E. L. From Genes To Clones: Introduction To Gene Technology (1987) VCH Publishers, NY (translated by Horst Ibelgaufts). 634 pp. (ISBN 0-89573-614-4).

The contents of all cited references (including literature references, patents, patent applications, databases, and websites) that are cited throughout this application are hereby expressly incorporated by reference herein in their entirety for any purpose, as are the references cited therein. The practice of the present disclosure will employ, unless otherwise indicated, conventional techniques of immunology, molecular biology and cell biology, which are well known in the art.

### EQUIVALENTS

The present disclosure may be embodied in other specific forms without departing from the spirit or essential characteristics thereof. The foregoing embodiments are therefore to be considered in all respects illustrative, rather than limiting, of the invention described herein. Scope of the invention is thus indicated by the appended claims, rather than by the foregoing description, and all changes that come within the meaning and range of equivalency of the claims are therefore intended to be embraced herein.

1. A binding protein comprising a polypeptide chain, wherein said polypeptide chain comprises VD1-(X1)n-VD2-(X2)n-VD3-C-(X3)n, wherein;
VD1 is a first heavy chain variable domain;
VD2 is a second heavy chain variable domain;
VD3 is a third heavy chain variable domain;
C is a heavy chain constant domain;
X1 is a first linker;
X2 is a second linker;
X3 is an Fc region; and
n is 0 or 1;
wherein the binding protein is capable of binding one to three target antigens.

2. A binding protein comprising a polypeptide chain, wherein said polypeptide chain comprises VD1-(X1)n-VD2-(X2)n-VD3-C-(X3)n, wherein;
VD1 is a first light chain variable domain;
VD2 is a second light chain variable domain;
VD3 is a third light chain variable domain;
C is a light chain constant domain;
X1 is a first linker;
X1 is a second linker;
X3 does not comprise an Fc region; and
n is 0 or 1;
wherein the binding protein is capable of binding one to three target antigens.

3. A binding protein comprising a first and a second polypeptide chain, wherein said first polypeptide chain comprises a first VD1-(X1)n-VD2-(X2)n-VD3-C-(X3)n, wherein
VD1 is a first heavy chain variable domain;
VD2 is a second heavy chain variable domain;
VD3 is a third heavy chain variable domain;
C is a heavy chain constant domain;
X1 is a first linker;
X2 is a second linker;
and
X3 is an Fc region; and
wherein said second polypeptide chain comprises a second VD1-(X1)n-VD2-(X2)n-VD3-C-(X3)n, wherein
VD1 is a first light chain variable domain;
VD2 is a second light chain variable domain;
VD3 is a third light chain variable domain;
C is a light chain constant domain;
X1 is a first linker;
X2 is a second linker; and
X3 does not comprise an Fc region; and
n is 0 or 1,
wherein the binding protein is capable of binding one to three target antigens.

4. A binding protein comprising four polypeptide chains, wherein each of the first and third polypeptide chains independently comprise VD1-(X1)n-VD2-(X2)n-VD3-C-(X3)n, wherein
VD1 is a first heavy chain variable domain;
VD2 is a second heavy chain variable domain;
VD3 is a third heavy chain variable domain;
C is a heavy chain constant domain;
X1 is a first linker;
X2 is a second linker;
X3 is an Fc region; and
wherein each of the second and fourth polypeptide chains independently comprise VD1-(X1)n-VD2-(X2)n-VD3-C-(X3)n, wherein
VD1 is a first light chain variable domain;
VD2 is a second light chain variable domain;
VD3 is a third light chain variable domain;
C is a light chain constant domain;
X1 is a linker;
X2 is a second linker
X3 does not comprise an Fc region; and
n is 0 or 1; wherein the binding protein is capable of binding one to six target antigens.

5. (canceled)

6. (canceled)

7. The binding protein of any one of claims 1-4, wherein two or more of VD1, VD2, and VD3 are independently obtained from a same or different parent binding protein, or antigen-binding portion thereof.

8. The binding protein of any one of claims 1-4, wherein each of VD1, VD2, and VD3 are independently obtained from a same or different parent binding protein, or antigen-binding portion thereof.

9.-16. (canceled)

17. The binding protein of claim 7 or 8, wherein said same parent binding protein, or antigen-binding portion thereof, is selected from the group consisting of a human antibody, a CDR grafted antibody, and a humanized antibody.

18. The binding protein of claim 7 or 8, wherein said same parent binding protein, or antigen-binding portion thereof, is selected from the group consisting of a Fab fragment; a F(ab′)₂ fragment; a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment; an isolated complementarity determining region (CDR); a single chain antibody; and a diabody.

19.-21. (canceled)

22. The binding protein of any one of claims 1-4, wherein said one or more of the target antigens is selected from the group consisting of ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; C1q; C1r; C1; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); Factor VII; Factor IX; Factor V; Factor VIIa; Factor Factor X; Factor XII; Factor XIII; FADD; FasL; FASN; FCER1A; FCER2; Fc gamma receptor; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; glycoprotein (GP) IIb/IIIa; HAVCR2; HDAC4; HDAC5; HDAC7A; HDAC9; Her2; HGF; HIF1A; HIP1; histamine and histamine receptors; HLA-A; HLA-DRA; HM74; HMGB1; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL-1α; IL-1β; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGS1; RGS13; RGS3; RNF110 (ZNF144); ROBO2; SIO0A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STAT6; STEAP; STEAP2; substamce P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase ha); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPRS/CCXCR1); YY1; and ZFPM2.

23.-25. (canceled)

26. The binding protein of any one of claims 1-4, wherein said binding protein is capable of binding three target antigens selected from the group consisting of prostaglandin E2 (PGE2), interleukin 13 (IL-13), interleukin 18 (IL-18), Tumor Necrosis factor alpha (TNFα), interleukin 23 (IL-23), IL-12, HMGB1, VEGF, RAGE, NGF, IL-1α, IL-1β, E-selectin, L-selectin, glycoprotein (GP) IIb/IIIa, thrombomodulin, thrombin, CGRP, TREM, PAI-I, αVβ3, uPA, Her2, IGF1R, EGFR, CD3, NKG2D, substance P, Protein C, Factor VII, Factor IX, plasminogen activator, Factor V, Factor VIIa, Factor Factor X, Factor XII, Factor XIII, C1q, C1r C1s, C4a, C4b, C2a, C2b, C, C3a and C3b.

27.-37. (canceled)

38. The binding protein of any one of claims 1-4, wherein the first and second linker comprise an amino acid sequence independently selected from the group consisting of AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); GHEAAAVMQVQYPAS (SEQ ID NO: 26); TVAAPSVFIFPPTVAAPSVFIFPP (SEQ ID NO: 27); and ASTKGPSVFPLAPASTKGPSVFPLAP (SEQ ID NO: 28).

39. A binding protein conjugate comprising a binding protein of any one of claims 1-4, and an agent selected from the group consisting of an immunoadhension molecule, an imaging agent, a therapeutic agent, and a cytotoxic agent.

40.-46. (canceled)

47. The binding protein of any one of claims 1-4, wherein said binding protein is produced according to a method comprising, culturing a host cell in culture medium under conditions sufficient to produce said binding protein, wherein said host cell comprises a vector, said vector comprising a nucleic acid encoding said binding protein.

48. A pharmaceutical composition comprising a binding protein of any one of claims 1-4, and a pharmaceutically acceptable carrier.

49.-51. (canceled)

52. A pharmaceutical composition comprising a binding protein conjugate according to claim 39, and a pharmaceutically acceptable carrier.

53.-61. (canceled)

62. An isolated nucleic acid molecule comprising a nucleotide sequence encoding a binding protein of any one of claims 1-4.

63. A vector comprising the isolated nucleic acid of claim 62.

64. (canceled)

65. A host cell comprising a vector of claim 63.

66.-75. (canceled)

76. A method of producing a binding protein, comprising culturing the host cell of claim 65 in culture medium under conditions sufficient to produce the binding protein.

77.-79. (canceled)

80. A protein produced according to the method of claim 76.

81. A method for treating a subject for a disease or a disorder, comprising administering to the subject a therapeutically effective amount of the binding protein of any one of claims 1-4, thereby treating the disease or disorder.

82. (canceled)

83. (canceled)

84. A method for generating a Tri-Variable Domain binding protein capable of binding three antigens, comprising
a) obtaining a first parent binding protein, or antigen-binding portion thereof, capable of binding a first target antigen;
b) obtaining a second parent binding protein, or antigen-binding portion thereof, capable of binding a second target antigen;
c) obtaining a third parent binding protein, or antigen-binding portion thereof, capable of binding a third target antigen;
c) constructing first and third polypeptide chains comprising VD1-(X1)n-VD2-(X2)n-VD3-C-(X3)n, wherein
VD1 is a first heavy chain variable domain obtained from said first parent binding protein, or antigen-binding portion thereof;
VD2 is a second heavy chain variable domain obtained from said second parent binding protein, or antigen-binding portion thereof;
VD3 is a third heavy chain variable domain obtained from said third parent binding protein, or antigen-binding portion thereof;
C is a heavy chain constant domain;
X2 is a first linker;
X1 is a second linker;
X3 is an Fc region; and n is 0 or 1; and
d) constructing second and fourth polypeptide chains comprising VD1-(X1)n-VD2-(X2)n-VD3-C-(X3)n, wherein
VD1 is a first light chain variable domain obtained from said first parent binding protein, or antigen-binding portion thereof;
VD2 is a second light chain variable domain obtained from said second parent binding protein, or antigen-binding portion thereof;
VD3 is a second light chain variable domain obtained from said third parent binding protein, or antigen-binding portion thereof;
C is a light chain constant domain;
X1 is a first linker;
X2 is a second linker;
X3 does not comprise an Fc region; and n is 0 or 1; and
e) expressing said first, second, third and fourth polypeptide chains;
such that a Tri-Variable Domain Immunoglobulin capable of binding said first, second, and third target antigens is generated.

85. The method of claim 84, wherein the VD1, VD2, and VD3 heavy chain variable domains comprise an amino acid sequence selected from the group consisting of SEQ ID NOs: 46, 47, 48, 70, 71, 163, 165, 167, 169, and 171, wherein the VD1, VD2, and VD3 light chain variable domains comprise an amino acid sequence selected from the group consisting of SEQ ID NOs: 51, 52, 53, 73, and 74, 164, 166, 168, 170, and 172.

86. (canceled)

87. (canceled)

88. The method of claim 84, wherein said first parent binding protein, or antigen-binding portion thereof possesses at least one desired property exhibited by the Tri-Variable Domain Immunoglobulin.

89. The method of claim 84, wherein said second parent binding protein, or antigen-binding portion thereof possesses at least one desired property exhibited by the Tri-Variable Domain Immunoglobulin.

90. The method of claim 84, wherein said third parent binding protein, or antigen-binding portion thereof possesses at least one desired property exhibited by the Tri-Variable Domain Immunoglobulin.

91.-96. (canceled)

97. A method of determining the presence, amount or concentration of an antigen, or fragment thereof, in a test sample,
wherein the antigen, or fragment thereof, is selected from the group consisting of prostaglandin E2 (PGE2), interleukin 13 (IL-13), Tumor Necrosis factor alpha (TNFα), interleukin 13 (IL-13), and interleukin 18 (IL-18),
which method comprises assaying the test sample for the antigen, or fragment thereof, by an immunoassay,
wherein the immunoassay (i) employs at least one binding protein and at least one detectable label and (ii) comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or a fragment thereof, in a control or a calibrator,
wherein the calibrator is optionally part of a series of calibrators in which each of the calibrators differs from the other calibrators in the series by the concentration of the antigen, or fragment thereof, and
wherein one of the at least one binding protein
(i′) comprises one or more polypeptide chains comprising VD1-(X1)n-VD2-(X2)n-VD3-C-(X3)n, wherein;
VD1 is a first heavy chain variable domain obtained from a first parent binding protein, or antigen-binding portion thereof;
VD2 is a second heavy chain variable domain obtained from a second parent binding protein, or antigen-binding portion thereof;
VD3 is a third heavy chain variable domain obtained from a third parent binding protein, or antigen-binding portion thereof;
C is a heavy chain constant domain;
X1 is a first linker;
X2 is a second linker;
X3 is an Fc region; and n is 0 or 1; and
(ii′) can bind a triplet of antigens selected from the group consisting of prostaglandin E2 (PGE2), interleukin 13 (IL-13), and interleukin 18 (IL-18); and
Tumor Necrosis factor alpha (TNFα), interleukin 13 (IL-13), and interleukin 18 (IL-18),
whereupon the presence, amount or concentration of an antigen, or a fragment thereof, in the test sample is determined.

98. A method of determining the presence, amount or concentration of an antigen, or fragment thereof, in a test sample,
wherein the antigen, or fragment thereof, is selected from the group consisting of prostaglandin E2 (PGE2), interleukin 13 (IL-13), Tumor Necrosis factor alpha (TNFα), interleukin 13 (IL-13), and interleukin 18 (IL-18),
which method comprises assaying the test sample for the antigen, or fragment thereof, by an immunoassay,
wherein the immunoassay (i) employs at least one binding protein and at least one detectable label and (ii) comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or a fragment thereof, in a control or a calibrator,
wherein the calibrator is optionally part of a series of calibrators in which each of the calibrators differs from the other calibrators in the series by the concentration of the antigen, or fragment thereof, and
wherein one of the at least one binding protein
(i′) comprises one or more polypeptide chains comprising VD1-(X1)n-VD2-(X2)n-VD3-C-(X3)n, wherein;
VD1 is a first heavy chain variable domain obtained from a first parent binding protein, or antigen-binding portion thereof;
VD2 is a second heavy chain variable domain obtained from a second parent binding protein, or antigen-binding portion thereof;
VD3 is a third heavy chain variable domain obtained from a third parent binding protein, or antigen-binding portion thereof;
C is a heavy chain constant domain;
X1 is a first linker;
X2 is a second linker;
X3 is an Fc region; and n is 0 or 1; and
(ii′) can bind a triplet of antigens selected from the group consisting of prostaglandin E2 (PGE2), interleukin 13 (IL-13), and interleukin 18 (IL-18); and
Tumor Necrosis factor alpha (TNFα), interleukin 13 (IL-13), and interleukin 18 (IL-18),
whereupon the presence, amount or concentration of an antigen, or a fragment thereof, in the test sample is determined.

99.-102. (canceled)

103. A kit for assaying a test sample for an antigen, or fragment thereof, which kit comprises at least one component for assaying the test sample for an antigen, or fragment thereof, and instructions for assaying the test sample for an antigen, or fragment thereof, wherein the at least one component includes at least one composition comprising a binding protein, which
(i′) comprises one or more polypeptide chains comprising VD1-(X1)n-VD2-(X2)n-VD3-C-(X3)n, wherein;
VD1 is a first heavy chain variable domain obtained from a first parent binding protein, or antigen-binding portion thereof;
VD2 is a second heavy chain variable domain obtained from a second parent binding protein, or antigen-binding portion thereof;
VD3 is a third heavy chain variable domain obtained from a third parent binding protein, or antigen-binding portion thereof;
C is a heavy chain constant domain;
X1 is a first linker;
X1 is a second linker;
X3 is an Fc region; and n is 0 or 1; and
(ii′) can bind a triplet of antigens selected from the group consisting of prostaglandin E2 (PGE2), interleukin 13 (IL-13), and interleukin 18 (IL-18); and
Tumor Necrosis factor alpha (TNFα), interleukin 13 (IL-13), and interleukin 18 (IL-18),
wherein the binding protein is optionally detectably labeled.

104. A kit for assaying a test sample for an antigen, or fragment thereof, which kit comprises at least one component for assaying the test sample for an antigen, or fragment thereof, and instructions for assaying the test sample for an antigen, or fragment thereof, wherein the at least one component includes at least one composition comprising a binding protein, which
(i′) comprises one or more polypeptide chains comprising VD1-(X1)n-VD2-(X2)n-VD3-C-(X3)n, wherein;
VD1 is a first heavy chain variable domain obtained from a first parent binding protein, or antigen-binding portion thereof;
VD2 is a second heavy chain variable domain obtained from a second parent binding protein, or antigen-binding portion thereof;
VD3 is a third heavy chain variable domain obtained from a third parent binding protein, or antigen-binding portion thereof;
C is a heavy chain constant domain;
X1 is a first linker;
X2 is a second linker;
X3 is an Fc region; and n is 0 or 1; and
(ii′) can bind a triplet of antigens selected from the group consisting of prostaglandin E2 (PGE2), interleukin 13 (IL-13), and interleukin 18 (IL-18); and
Tumor Necrosis factor alpha (TNFα), interleukin 13 (IL-13), and interleukin 18 (IL-18),
wherein the binding protein is optionally detectably labeled.


SEQUENCE LISTING (205 sequences)

SEQ ID NO: 1 - protein, 16 aa, Artificial Sequence
  Description of Artificial Sequence Synthetic peptide
AKTTPKLEEGEFSEAR

SEQ ID NO: 2 - protein, 17 aa, Artificial Sequence
  Description of Artificial Sequence Synthetic peptide
AKTTPKLEEGEFSEARV

SEQ ID NO: 3 - protein, 9 aa, Artificial Sequence
  Description of Artificial Sequence Synthetic peptide
AKTTPKLGG

SEQ ID NO: 4 - protein, 10 aa, Artificial Sequence
  Description of Artificial Sequence Synthetic peptide
SAKTTPKLGG

SEQ ID NO: 5 - protein, 6 aa, Artificial Sequence
  Description of Artificial Sequence Synthetic peptide
SAKTTP

SEQ ID NO: 6 - protein, 6 aa, Artificial Sequence
  Description of Artificial Sequence Synthetic peptide
RADAAP

SEQ ID NO: 7 - protein, 9 aa, Artificial Sequence
  Description of Artificial Sequence Synthetic peptide
RADAAPTVS

SEQ ID NO: 8 - protein, 12 aa, Artificial Sequence
  Description of Artificial Sequence Synthetic peptide
RADAAAAGGPGS

SEQ ID NO: 9 - protein, 27 aa, Artificial Sequence
  Description of Artificial Sequence Synthetic peptide
RADAAAAGGGGSGGGGSGGGGSGGGGS

SEQ ID NO: 10 - protein, 18 aa, Artificial Sequence
  Description of Artificial Sequence Synthetic peptide
SAKTTPKLEEGEFSEARV

SEQ ID NO: 11 - protein, 5 aa, Artificial Sequence
  Description of Artificial Sequence Synthetic peptide
ADAAP

SEQ ID NO: 12 - protein, 12 aa, Artificial Sequence
  Description of Artificial Sequence Synthetic peptide
ADAAPTVSIFPP

SEQ ID NO: 13 - protein, 5 aa, Artificial Sequence
  Description of Artificial Sequence Synthetic peptide
TVAAP

SEQ ID NO: 14 - protein, 12 aa, Artificial Sequence
  Description of Artificial Sequence Synthetic peptide
TVAAPSVFIFPP

SEQ ID NO: 15 - protein, 6 aa, Artificial Sequence
  Description of Artificial Sequence Synthetic peptide
QPKAAP

SEQ ID NO: 16 - protein, 13 aa, Artificial Sequence
  Description of Artificial Sequence Synthetic peptide
QPKAAPSVTLFPP

SEQ ID NO: 17 - protein, 6 aa, Artificial Sequence
  Description of Artificial Sequence Synthetic peptide
AKTTPP

SEQ ID NO: 18 - protein, 13 aa, Artificial Sequence
  Description of Artificial Sequence Synthetic peptide
AKTTPPSVTPLAP

SEQ ID NO: 19 - protein, 6 aa, Artificial Sequence
  Description of Artificial Sequence Synthetic peptide
AKTTAP

SEQ ID NO: 20 - protein, 13 aa, Artificial Sequence
  Description of Artificial Sequence Synthetic peptide
AKTTAPSVYPLAP

SEQ ID NO: 21 - protein, 6 aa, Artificial Sequence
  Description of Artificial Sequence Synthetic peptide
ASTKGP

SEQ ID NO: 22 - protein, 13 aa, Artificial Sequence
  Description of Artificial Sequence Synthetic peptide
ASTKGPSVFPLAP

SEQ ID NO: 23 - protein, 15 aa, Artificial Sequence
  Description of Artificial Sequence Synthetic peptide
GGGGSGGGGSGGGGS

SEQ ID NO: 24 - protein, 15 aa, Artificial Sequence
  Description of Artificial Sequence Synthetic peptide
GENKVEYAPALMALS

SEQ ID NO: 25 - protein, 15 aa, Artificial Sequence
  Description of Artificial Sequence Synthetic peptide
GPAKELTPLKEAKVS

SEQ ID NO: 26 - protein, 15 aa, Artificial Sequence
  Description of Artificial Sequence Synthetic peptide
GHEAAAVMQVQYPAS

SEQ ID NO: 27 - protein, 24 aa, Artificial Sequence
  Description of Artificial Sequence Synthetic peptide
TVAAPSVFIFPPTVAAPSVFIFPP

SEQ ID NO: 28 - protein, 26 aa, Artificial Sequence
  Description of Artificial Sequence Synthetic peptide
ASTKGPSVFPLAPASTKGPSVFPLAP

SEQ ID NO: 29 - DNA, 62 bp, Artificial Sequence
  Description of Artificial Sequence Synthetic primer
gcacctctgggcccttggtcgacgctgaagagacggtgaccattgtcccttggccccagata

SEQ ID NO: 30 - DNA, 65 bp, Artificial Sequence
  Description of Artificial Sequence Synthetic primer
accgtctcttcagcgtcgaccaagggcccagaggtgcagctggtgcagagcggcgccgaggtgaa

SEQ ID NO: 31 - DNA, 65 bp, Artificial Sequence
  Description of Artificial Sequence Synthetic primer
agggtgccagggggaagaccgatgggcccttggtcgacgcgctgctcacggtcaccagggtgccc

SEQ ID NO: 32 - DNA, 64 bp, Artificial Sequence
  Description of Artificial Sequence Synthetic primer
tggctttttcttgtcgcgattttaaaaggtgtccagtgcgaggtcaccttgagggagtctggtc

SEQ ID NO: 33 - DNA, 65 bp, Artificial Sequence
  Description of Artificial Sequence Synthetic primer
ggagattaaacgaacggtggctgcaccagacgtgctgatgacccagacccccctgagcctgcccg

SEQ ID NO: 34 - DNA, 66 bp, Artificial Sequence
  Description of Artificial Sequence Synthetic primer
catcagcacgtctggtgcagccaccgttcgtttaatctccagtcgtgtcccttggccgaaggtgat

SEQ ID NO: 35 - DNA, 66 bp, Artificial Sequence
  Description of Artificial Sequence Synthetic primer
catcagatggcgggaagatgaagacagatggtgcagccaccgtccgcttgatctccaccttggtgc

SEQ ID NO: 36 - DNA, 65 bp, Artificial Sequence
  Description of Artificial Sequence Synthetic primer
gggcctgctgctgctgtggttccccggctcgcgatgcgacatcgtgatgacccagtctccagact

SEQ ID NO: 37 - DNA, 64 bp, Artificial Sequence
  Description of Artificial Sequence Synthetic primer
gagcagcgcgtcgaccaagggcccagaggtcaccttgagggagtctggtcctgcgctggtgaaa

SEQ ID NO: 38 - DNA, 65 bp, Artificial Sequence
  Description of Artificial Sequence Synthetic primer
aggtgacctctgggcccttggtcgacgcgctgctcacggtcaccagggtgccctggccccagtag

SEQ ID NO: 39 - DNA, 65 bp, Artificial Sequence
  Description of Artificial Sequence Synthetic primer
tggctttttcttgtcgcgattttaaaaggtgtccagtgcgaggtgcagctggtgcagagcggcgc

SEQ ID NO: 40 - DNA, 65 bp, Artificial Sequence
  Description of Artificial Sequence Synthetic primer
gtgccagggggaagaccgatgggcccttggtcgacgctgaagagacggtgaccattgtcccttgg

SEQ ID NO: 41 - DNA, 65 bp, Artificial Sequence
  Description of Artificial Sequence Synthetic primer
agatcaagcggacggtggctgcaccagacatcgtgatgacccagtctccagactccctggctgtg

SEQ ID NO: 42 - DNA, 65 bp, Artificial Sequence
  Description of Artificial Sequence Synthetic primer
cacgatgtctggtgcagccaccgtccgcttgatctccaccttggtgccgccgccgaaggtgtagg

SEQ ID NO: 43 - DNA, 65 bp, Artificial Sequence
  Description of Artificial Sequence Synthetic primer
tgctgggcctgctgctgctgtggttccccggctcgcgatgcgacgtgctgatgacccagaccccc

SEQ ID NO: 44 - DNA, 66 bp, Artificial Sequence
  Description of Artificial Sequence Synthetic primer
tggcgggaagatgaagacagatggtgcagccaccgtacgtcgtttaatctccagtcgtgtcccttg

SEQ ID NO: 45 - protein, 368 aa, Artificial Sequence
  Description of Artificial Sequence Synthetic polypeptide
EVTLRESGPALVKPTQTLTLTCTFSGFSLSKSVMGVSWIRQPPGKALEWLAHIYWDDDKYYNPSLKSRLTISKDTSKNQVVLTMTNMDPVDTATYYCARRGIRSAMDYWGQGTTVTVSSASTKGPEVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPEVQLVQSGAEVKKPGASVKVSCKASGYTFTKYWLGWVRQAPGQGLEWMGDIYPGYDYTHYNEKFKDRVTLTTDTSTSTAYMELRSLRSDDTAVYYCARSDGSSTYWGQGTLVTVSS

SEQ ID NO: 46 - protein, 119 aa, Artificial Sequence
  Description of Artificial Sequence Synthetic polypeptide
EVTLRESGPALVKPTQTLTLTCTFSGFSLSKSVMGVSWIRQPPGKALEWLAHIYWDDDKYYNPSLKSRLTISKDTSKNQVVLTMTNMDPVDTATYYCARRGIRSAMDYWGQGTTVTVSS

SEQ ID NO: 47 - protein, 121 aa, Artificial Sequence
  Description of Artificial Sequence Synthetic polypeptide
EVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSS

SEQ ID NO: 48 - protein, 116 aa, Artificial Sequence
  Description of Artificial Sequence Synthetic polypeptide
EVQLVQSGAEVKKPGASVKVSCKASGYTFTKYWLGWVRQAPGQGLEWMGDIYPGYDYTHYNEKFKDRVTLTTDTSTSTAYMELRSLRSDDTAVYYCARSDGSSTYWGQGTLVTVSS

SEQ ID NO: 49 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 340 aa, Artificial Sequence
  Description of Artificial Sequence Synthetic polypeptide
DIVMTQSPDSLAVSLGERATINCKASQSVSNDVAWYQQKPGQPPKLLIYYASNRYTGVPDRFSGSGSGTDFTLTISSLQAEDVAVYYCQQDYNSPWTFGGGTKVEIKRTVAAPEIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPDVLMTQTPLSLPVTPGEPASISCTSSQNIVHSNGNTYLEWYLQKPGQSPQLLIYKVSNRFSGVPDRFSGSGSGTDFTLKISRVEAEDVGVYYCFQVSHVPYTFGGGTKVEIKR

SEQ ID NO: 51 - protein, 108 aa, Artificial Sequence
  Description of Artificial Sequence Synthetic polypeptide
DIVMTQSPDSLAVSLGERATINCKASQSVSNDVAWYQQKPGQPPKLLIYYASNRYTGVPDRFSGSGSGTDFTLTISSLQAEDVAVYYCQQDYNSPWTFGGGTKVEIKR

SEQ ID NO: 52 - protein, 109 aa, Artificial Sequence
  Description of Artificial Sequence Synthetic polypeptide
EIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKR

SEQ ID NO: 53 - protein, 113 aa, Artificial Sequence
  Description of Artificial Sequence Synthetic polypeptide
DVLMTQTPLSLPVTPGEPASISCTSSQNIVHSNGNTYLEWYLQKPGQSPQLLIYKVSNRFSGVPDRFSGSGSGTDFTLKISRVEAEDVGVYYCFQVSHVPYTFGGGTKVEIKR

SEQ ID NO: 54 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 368 aa, Artificial Sequence
  Description of Artificial Sequence Synthetic polypeptide
EVQLVQSGAEVKKPGASVKVSCKASGYTFTKYWLGWVRQAPGQGLEWMGDIYPGYDYTHYNEKFKDRVTLTTDTSTSTAYMELRSLRSDDTAVYYCARSDGSSTYWGQGTLVTVSSASTKGPEVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSSASTKGPEVTLRESGPALVKPTQTLTLTCTFSGFSLSKSVMGVSWIRQPPGKALEWLAHIYWDDDKYYNPSLKSRLTISKDTSKNQVVLTMTNMDPVDTATYYCARRGIRSAMDYWGQGTTVTVSS

SEQ ID NO: 56 - protein, 125 aa, Artificial Sequence
  Description of Artificial Sequence Synthetic polypeptide
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 57 - protein, 161 aa, Artificial Sequence
  Description of Artificial Sequence Synthetic polypeptide
AQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQIC

SEQ ID NO: 58 - protein, 340 aa, Artificial Sequence
  Description of Artificial Sequence Synthetic polypeptide
DVLMTQTPLSLPVTPGEPASISCTSSQNIVHSNGNTYLEWYLQKPGQSPQLLIYKVSNRFSGVPDRFSGSGSGTDFTLKISRVEAEDVGVYYCFQVSHVPYTFGGGTKVEIKRTVAAPEIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKRTVAAPDIVMTQSPDSLAVSLGERATINCKASQSVSNDVAWYQQKPGQPPKLLIYYASNRYTGVPDRFSGSGSGTDFTLTISSLQAEDVAVYYCQQDYNSPWTFGGGTKVEIKR

SEQ ID NO: 59 - protein, 108 aa, Artificial Sequence; identical to SEQ ID NO: 51
  Description of Artificial Sequence Synthetic polypeptide

SEQ ID NO: 60 - protein, 109 aa, Artificial Sequence; identical to SEQ ID NO: 52
  Description of Artificial Sequence Synthetic polypeptide

SEQ ID NO: 61 - DNA, 30 bp, Artificial Sequence
  Description of Artificial Sequence Synthetic primer
agctgctgggcctgctgctgctgtggttcc

SEQ ID NO: 62 - DNA, 44 bp, Artificial Sequence
  Description of Artificial Sequence Synthetic primer
cactatttcaggagctgcaactgtccgcttgatctccaccttgg

SEQ ID NO: 63 - DNA, 24 bp, Artificial Sequence
  Description of Artificial Sequence Synthetic primer
tccagatttcaactgctcatcaga

SEQ ID NO: 64 - DNA, 35 bp, Artificial Sequence
  Description of Artificial Sequence Synthetic primer
acagttgcagctcctgaaatagtgatgacgcagtc

SEQ ID NO: 65 - DNA, 30 bp, Artificial Sequence
  Description of Artificial Sequence Synthetic primer
agtttgggctgagctggctttttcttgtcg

SEQ ID NO: 66 - DNA, 44 bp, Artificial Sequence
  Description of Artificial Sequence Synthetic primer
cacctcaggtcctttagtgcttgcgctgctcacggtcaccaggg

SEQ ID NO: 67 - DNA, 27 bp, Artificial Sequence
  Description of Artificial Sequence Synthetic primer
tgtgcccccagaggtgctcttggagga

SEQ ID NO: 68 - DNA, 38 bp, Artificial Sequence
  Description of Artificial Sequence Synthetic primer
gcaagcactaaaggacctgaggtgcagctggtgcagtc

SEQ ID NO: 69 - protein, 377 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPEVTLRESGPGLVKPTQTLTLTCTLYGFSLSTSDMGVDWIRQPPGKGLEWLAHIWWDDVKRYNPALKSRLTISKDTSKNQVVLKLTSVDPVDTATYYCARTVSSGYIYYAMDYWGQGTLVTVSSASTKGPEVQLVQSGTEVKKPGESLKISCKGSGYTVTSYWIGWVRQMPGKGLEWMGFIYPGDSETRYSPTFQGQVTISADKSFNTAFLQWSSLKASDTAMYYCARVGSGWYPYTFDIWGQGTMVTVSS

SEQ ID NO: 70 - protein, 121 aa, Artificial Sequence [sequence omitted]

SEQ ID NO: 71 - protein, 123 aa, Artificial Sequence
  Description of Artificial Sequence Synthetic polypeptide
EVTLRESGPGLVKPTQTLTLTCTLYGFSLSTSDMGVDWIRQPPGKGLEWLAHIWWDDVKRYNPALKSRLTISKDTSKNQVVLKLTSVDPVDTATYYCARTVSSGYIYYAMDYWGQGTLVTVSS

SEQ ID NO: 72 - protein, 335 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPDIQMTQSPSSLSASVGDRVTISCRASQDIRNYLNWYQQKPGKAPKLLIFYTSKLHSGVPSRFSGSGSGTDYTLTISSLQPEDIATYYCQQGLTPPLTFGGGTKVEIKRTVAAPEIVMTQSPATLSVSPGERATLSCRASESISSNLAWYQQKPGQAPRLFIYTASTRATDIPARFSGSGSGTEFTLTISSLQSEDFAVYYCQQYNNWPSITFGQGTRLEIKR

SEQ ID NO: 73 - protein, 108 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 74 - protein, 108 aa, Artificial Sequence
  Description of Artificial Sequence Synthetic polypeptide
DIQMTQSPSSLSASVGDRVTISCRASQDIRNYLNWYQQKPGKAPKLLIFYTSKLHSGVPSRFSGSGSGTDYTLTISSLQPEDIATYYCQQGLTPPLTFGGGTKVEIKR

SEQ ID NO: 75 - protein, 119 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 76 - protein, 108 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 77 - protein, 119 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 78 - protein, 108 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 79 - protein, 118 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 80 - protein, 114 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 81 - protein, 122 aa, Artificial Sequence
  Description of Artificial Sequence Synthetic polypeptide
QIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSS

SEQ ID NO: 82 - protein, 108 aa, Artificial Sequence
  Description of Artificial Sequence Synthetic polypeptide
DTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELER

SEQ ID NO: 83 - protein, 123 aa, Artificial Sequence
  Description of Artificial Sequence Synthetic polypeptide
EVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSS

SEQ ID NO: 84 - protein, 108 aa, Artificial Sequence
  Description of Artificial Sequence Synthetic polypeptide
DIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKR

SEQ ID NO: 85 - protein, 122 aa, Artificial Sequence
  Description of Artificial Sequence Synthetic polypeptide
EVQLQQSGPELVQPGASMKISCKASGYSFTDYTMNWVKQSHGKNLEWIGLINPYNGGSRYNQKFMAKATLTVDKSSNTAYMELLSVTSEDSAVYYCARDAGYFGSGFYFDYWGQGTTLTVSS

SEQ ID NO: 86 - protein, 107 aa, Artificial Sequence
  Description of Artificial Sequence Synthetic polypeptide
DIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPTFGAGTKLELKR

SEQ ID NO: 87 - protein, 118 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 88 - protein, 108 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 89 - protein, 117 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 90 - protein, 112 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 91 - protein, 112 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 92 - protein, 113 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 93 - protein, 112 aa, Artificial Sequence; identical to SEQ ID NO: 91
  Description of Artificial Sequence Synthetic polypeptide

SEQ ID NO: 94 - protein, 113 aa, Artificial Sequence
  Description of Artificial Sequence Synthetic polypeptide
DVVMTQTPLTLSVTTGQPASISCKSSQSLLDSDGKTYLNWLFQRPGESPKLLIYVTDILESGVPDRFTGSGSGTDFTLKISRVEAEDLGVYYCLQATHFPWTFGGGTKLEIKR

SEQ ID NO: 95 - protein, 119 aa, Artificial Sequence
  Description of Artificial Sequence Synthetic polypeptide
EVQLQQSGPDLVKPGASVRISCKASGYTFTDYNLHWVKQSHGKSLEWIGYIYPYNGITGYNQKFKSKATLTVDSSSNTAYMDLRSLTSEDSAVYFCARDAYDYDYLTDWGQGTLVTVSA

SEQ ID NO: 96 - protein, 108 aa, Artificial Sequence
  Description of Artificial Sequence Synthetic polypeptide
DILLTQSPVILSVSPGERVSFSCRTSKNVGTNIHWYQQRTNGSPRLLIKYASERLPGIPSRFSGSGSGTDFTLSINSVESEDIADYYCQQSNNWPYTFGGGTKLEIKR

SEQ ID NO: 97 - protein, 244 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 98 - protein, 221 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 99 - protein, 251 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 100 - protein, 228 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 101 - protein, 264 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 102 - protein, 240 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 103 - protein, 255 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPEVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSS

SEQ ID NO: 104 - protein, 228 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPDIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKR

SEQ ID NO: 105 - protein, 268 aa, Artificial Sequence
  Description of Artificial Sequence Synthetic polypeptide
QVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSAASTKGPSVFPLAPASTKGPSVFPLAPEVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSS

SEQ ID NO: 106 - protein, 240 aa, Artificial Sequence
  Description of Artificial Sequence Synthetic polypeptide
DIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKRTVAAPSVFIFPPTVAAPSVFIFPPDIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKR

SEQ ID NO: 107 - protein, 255 aa, Artificial Sequence
  Description of Artificial Sequence Synthetic polypeptide
EVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSSASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 108 - protein, 228 aa, Artificial Sequence
  Description of Artificial Sequence Synthetic polypeptide
DIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKRTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 109 - protein, 268 aa, Artificial Sequence
  Description of Artificial Sequence Synthetic polypeptide
EVQLQQSGPELVKPGASMKISCKASDYSFTAYTIHWMKQSHGKNLEWIGLINPYNGGTSYNQKFQGRATLTVDKSSSIAYMELLSLTSEDSAVYYCARRGYDREGHYYAMDYWGQGTSVTVSSASTKGPSVFPLAPASTKGPSVFPLAPQVQLQQSGAELMKPGASVKISCKASGYTFTSYWIEWIKQRPGHGLEWIGEILPGTGSLNNNEKFRDKATFTADTSSNTAYMQLSSLTSEDSAVYYCARGYRYDGWFAYWGQGTLVTVSA

SEQ ID NO: 110 - protein, 240 aa, Artificial Sequence
  Description of Artificial Sequence Synthetic polypeptide
DIQMTQSPASLAASVGETVTITCRASENIYTFLAWYQQKQGKSPQLLVYTTKTLAEGVPSRFSGSGSGTQFSLKIKSLQPEDFGSYYCQHHYGLPLTFGAGTKLELKRTVAAPSVFIFPPTVAAPSVFIFPPDIQMTQSPASLSASVGETVTITCRTSENIYSYLAWYQQKPGKSPHLLVYNTKTLAEGVPSRFSGSGSGTQFSLKINSLQPEDFGSYYCQHHYDSPLTFGSGTKLELKR

SEQ ID NO: 111 - protein, 257 aa, Artificial Sequence
  Description of Artificial Sequence Synthetic polypeptide
QIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSSASTKGPSVFPLAPQIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSS

SEQ ID NO: 112 - protein, 228 aa, Artificial Sequence
  Description of Artificial Sequence Synthetic polypeptide
DTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELERTVAAPSVFIFPPDTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELER

SEQ ID NO: 113 - protein, 250 aa, Artificial Sequence
  Description of Artificial Sequence Synthetic polypeptide
QIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSSASTKGPQIQLVQSGPELKKPGETVKISCKASGYTFTDYSMHWVKQAPGKGLKWMGWIHTETGEPRYVDDFKGRFAFSLETSASTAYLQINNLKNEDTATYFCARDSYYFGSSYYFDYWGQGTTLTVSS

SEQ ID NO: 114 - protein, 221 aa, Artificial Sequence
  Description of Artificial Sequence Synthetic polypeptide
DTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELERTVAAPDTVMTQSHKFMSTSVGDRVSITCKASQDVSSAVAWYQQKPGQSPKLLIYSASYRYTGVPDRFTGSGSGMDFTFTISSVQAEDLAVYYCQQHYSTPLTFGAGTKLELER

SEQ ID NO: 115 - protein, 257 aa, Artificial Sequence
  Description of Artificial Sequence Synthetic polypeptide
EVQLQQSGPELVQPGASMKISCKASGYSFTDYTMNWVKQSHGKNLEWIGLINPYNGGSRYNQKFMAKATLTVDKSSNTAYMELLSVTSEDSAVYYCARDAGYFGSGFYFDYWGQGTTLTVSSASTKGPSVFPLAPEVQLQQSGPELVQPGASMKISCKASGYSFTDYTMNWVKQSHGKNLEWIGLINPYNGGSRYNQKFMAKATLTVDKSSNTAYMELLSVTSEDSAVYYCARDAGYFGSGFYFDYWGQGTTLTVSS

SEQ ID NO: 116 - protein, 226 aa, Artificial Sequence
  Description of Artificial Sequence Synthetic polypeptide
DIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPTFGAGTKLELKRTVAAPSVFIFPPDIVMTQSHKFMSTSVGDRVSITCKASQDVSTAVAWYQQKPGQSPKLLIYSASYRSTGVPDRFTGSGSGTDFTFTISSVQAEDLAVYYCQQHYSTPTFGAGTKLELKR

SEQ ID NO: 117 - protein, 251 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 118 - protein, 228 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 119 - protein, 244 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 120 - protein, 221 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 121 - protein, 249 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPSVFPLAPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 122 - protein, 240 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPSVFIFPPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 123 - protein, 242 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 124 - protein, 233 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 125 - protein, 250 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 126 - protein, 234 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 127 - protein, 243 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPKIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSS

SEQ ID NO: 128 - protein, 227 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPDIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKR

SEQ ID NO: 129 - protein, 250 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 130 - protein, 234 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 131 - protein, 243 aa, Artificial Sequence
  Description of Artificial Sequence Synthetic polypeptide
KIQLVQSGPELKKPGETVKISCKASGYTFTNYGMNWVKQAPGKGLKWMGWININTGEPTYAEEFKGRFAFSLETSATTAFLQINNLKNEDTATYLCARDSYSGGFDYWGQGTIVTVSSASTKGPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 132 - protein, 227 aa, Artificial Sequence
  Description of Artificial Sequence Synthetic polypeptide
DIVMTQSPSSLSVSAGEKVTLSCKSSQSLLISGDQKNYLAWYQQKPGQPPKLLIYGASTRDSGVPDRFTGSGSGADFTLTISSVQAEDLAVYYCQNDHSFPPTFGAGTKLELKRTVAAPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 133 - protein, 243 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPQVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 134 - protein, 221 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 135 - protein, 250 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPQVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 136 - protein, 228 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPSIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 137 - protein, 263 aa, Artificial Sequence
  Description of Artificial Sequence Synthetic polypeptide
EVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSSASTKGPSVFPLAPASTKGPSVFPLAPQVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSS

SEQ ID NO: 138 - protein, 240 aa, Artificial Sequence
  Description of Artificial Sequence Synthetic polypeptide
DIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKRTVAAPSVFIFPPTVAAPSVFIFPPSIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKR

SEQ ID NO: 139 - protein, 243 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSSASTKGPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 140 - protein, 221 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKRTVAAPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 141 - protein, 250 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSSASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 142 - protein, 228 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKRTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 143 - protein, 263 aa, Artificial Sequence
  Description of Artificial Sequence Synthetic polypeptide
QVQLQQPGSELVRPGASVKLSCKASGYTFTSYWMHWVKQRPGQGLEWIGNIYPGTVNTNYDEKFKNKATLTVDTSSSTAYMLLSSLTSEDSAVYYCTRDYYGGGLNYWGQGTTLTVSSASTKGPSVFPLAPASTKGPSVFPLAPEVQLVESGGGLVQPGGSLKLSCAASGFTFNNYYMSWVRQTPERRLEWVAYISSSGGSTYYSDSVRGRFTISRDTARNTLYLQMTSLKSEDTAMYYCARHFGDYSYFDYWGQGTTLTVSS

SEQ ID NO: 144 - protein, 240 aa, Artificial Sequence
  Description of Artificial Sequence Synthetic polypeptide
SIVMTQTPKFLLVSAGDRVTITCKASQSVSNDVAWFQQKPGQSPKLLIYYASNRYAGVPDRFTGSGFGTDFTFTISTVQAEDLAVYFCHQDYSSPRTFGGGTKLEIKRTVAAPSVFIFPPTVAAPSVFIFPPDIQMTQSPASLSASVGETVTITCRASENFYSYLAWYQQKQGKSPQLLVYNAKTLAEGVPSRFSGSGSGTQFSLKINSLQPEDFGTYYCQHHYDIPLTFGAGTKLELKR

SEQ ID NO: 145 - protein, 247 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPSVFPLAPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 146 - protein, 236 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPSVFIFPPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 147 - protein, 240 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 148 - protein, 229 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 149 - protein, 237 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSASTKGPSVFPLAPQVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 150 - protein, 238 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRTVAAPSVFIFPPDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 151 - protein, 242 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSASTKGPSVFPLAPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 152 - protein, 237 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRTVAAPSVFIFPPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 153 - protein, 255 aa, Artificial Sequence
  Description of Artificial Sequence Synthetic polypeptide
QVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSSASTKGPSVFPLAPASTKGPSVFPLAPQIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSS

SEQ ID NO: 154 - protein, 249 aa, Artificial Sequence
  Description of Artificial Sequence Synthetic polypeptide
DVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKRTVAAPSVFIFPPTVAAPSVFIFPPDNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKR

SEQ ID NO: 155 - protein, 242 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPSVFPLAPQVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 156 - protein, 237 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPSVFIFPPDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 157 - protein, 255 aa, Artificial Sequence
  Description of Artificial Sequence Synthetic polypeptide
QIQLVQSGPELRKPGETVKISCKGSGYTFTHYGINWVKQTPRKDLKWMGWINTHTGEAYYADDFKGRFAFSLETSANTAYLQINNLNNGDMGTYFCTRSHRFGLDYWGQGTSVTVSSASTKGPSVFPLAPASTKGPSVFPLAPQVQLQQPGAELVRPGASVKLSCKASGYTFTSYWMNWVKQRPEQGLEWIGRIDPYDSETHYNQKFKDKAILTVDKSSSTAFVQLTSLTSEDSAVYYCVSDGYWGAGTTVTVSS

SEQ ID NO: 158 - protein, 249 aa, Artificial Sequence
  Description of Artificial Sequence Synthetic polypeptide
DNVLTQSPPSLAVSLGQRATISCKANWPVDYNGDSYLNWYQQKPGQPPKFLIYAASNLESGIPARFSGSGSGTDFNLNIHPVEEEDAATYYCQQSNEDPFTFGSGTKLEIKRTVAAPSVFIFPPTVAAPSVFIFPPDVVMTQTPLTLSVTTGQPASISCKSSQSLLDSDGKTYLNWLFQRPGESPKLLIYVVSKLESGVPDRFTGSGSGTDFTLKISRVEAEDLGVYYCLQATHFPWTFGGGTKLEIKR

SEQ ID NO: 159 - protein, 237 aa, Artificial Sequence; identical to SEQ ID NO: 149
  Description of Artificial Sequence Synthetic polypeptide

SEQ ID NO: 160 - protein, 238 aa, Artificial Sequence
  Description of Artificial Sequence Synthetic polypeptide
DVVMTQTPLTLSVTTGQPASISCKSSQSLLDSDGKTYLNWLFQRPGESPKLLIYVTDILESGVPDRFTGSGSGTDFTLKISRVEAEDLGVYYCLQATHFPWTFGGGTKLEIKRTVAAPSVFIFPPDVVMTQTPLTLSVTTGQPASISCKSSQSLLDSDGKTYLNWLFQRPGESPKLLIYVTDILESGVPDRFTGSGSGTDFTLKISRVEAEDLGVYYCLQATHFPWTFGGGTKLEIKR

SEQ ID NO: 161 - protein, 251 aa, Artificial Sequence
  Description of Artificial Sequence Synthetic polypeptide
EVQLQQSGPDLVKPGASVRISCKASGYTFTDYNLHWVKQSHGKSLEWIGYIYPYNGITGYNQKFKSKATLTVDSSSNTAYMDLRSLTSEDSAVYFCARDAYDYDYLTDWGQGTLVTVSAASTKGPSVFPLAPEVQLQQSGPDLVKPGASVRISCKASGYTFTDYNLHWVKQSHGKSLEWIGYIYPYNGITGYNQKFKSKATLTVDSSSNTAYMDLRSLTSEDSAVYFCARDAYDYDYLTDWGQGTLVTVSA

SEQ ID NO: 162 - protein, 228 aa, Artificial Sequence
  Description of Artificial Sequence Synthetic polypeptide
DILLTQSPVILSVSPGERVSFSCRTSKNVGTNIHWYQQRTNGSPRLLIKYASERLPGIPSRFSGSGSGTDFTLSINSVESEDIADYYCQQSNNWPYTFGGGTKLEIKRTVAAPSVFIFPPDILLTQSPVILSVSPGERVSFSCRTSKNVGTNIHWYQQRTNGSPRLLIKYASERLPGIPSRFSGSGSGTDFTLSINSVESEDIADYYCQQSNNWPYTFGGGTKLEIKR

SEQ ID NO: 163 - protein, 119 aa, Artificial Sequence [sequence omitted]

SEQ ID NO: 164 - protein, 107 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINR

SEQ ID NO: 165 - protein, 119 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 166 - protein, 108 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 167 - protein, 125 aa, Artificial Sequence [sequence omitted]

SEQ ID NO: 168 - protein, 108 aa, Artificial Sequence
  Description of Artificial Sequence Synthetic polypeptide
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKR

SEQ ID NO: 169 - protein, 121 aa, Artificial Sequence [sequence omitted]

SEQ ID NO: 170 - protein, 107 aa, Artificial Sequence
  Description of Artificial Sequence Synthetic polypeptide
QIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 171 - protein, 120 aa, Artificial Sequence [sequence omitted]

SEQ ID NO: 172 - protein, 108 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 173 - DNA, 70 bp, Artificial Sequence
  Description of Artificial Sequence Synthetic primer
tacaggactttgtgtcagaaggatatcaggagctgcgacggtgcgattgatctcaagcttagtgccacta

SEQ ID NO: 174 - DNA, 70 bp, Artificial Sequence
  Description of Artificial Sequence Synthetic primer
tagtggcactaagcttgagatcaatcgcaccgtcgcagctcctgatatccttctgacacaaagtcctgta

SEQ ID NO: 175 - DNA, 35 bp, Artificial Sequence
  Description of Artificial Sequence Synthetic primer
gtcgaggtcgggggatccggccttgccggcctcga

SEQ ID NO: 176 - DNA, 40 bp, Artificial Sequence
  Description of Artificial Sequence Synthetic primer
cgcgtgcccgcccagctgctgggcctgctgctgctgtggt

SEQ ID NO: 177 - DNA, 35 bp, Artificial Sequence; identical to SEQ ID NO: 175
  Description of Artificial Sequence Synthetic primer

SEQ ID NO: 178 - DNA, 45 bp, Artificial Sequence
  Description of Artificial Sequence Synthetic primer
accatggagtttgggctgagctggctttttcttgtcgcgatttta

SEQ ID NO: 179 - DNA, 70 bp, Artificial Sequence
  Description of Artificial Sequence Synthetic primer
ggcccgctctgtttgagctgaacctgaggccctttcgtggaggcggaggacacggtcagagtggtaccct

SEQ ID NO: 180 - DNA, 70 bp, Artificial Sequence
  Description of Artificial Sequence Synthetic primer
agggtaccactctgaccgtgtcctccgcctccacgaaagggcctcaggttcagctcaaacagagcgggcc

SEQ ID NO: 181 - DNA, 70 bp, Artificial Sequence
  Description of Artificial Sequence Synthetic primer
tagtggcactaagcttgagatcaatcgcaccgtcgcagctcctgatatccagatgactcagtttcccagc

SEQ ID NO: 182 - DNA, 70 bp, Artificial Sequence
  Description of Artificial Sequence Synthetic primer
gctgggaaactgagtcatctggatatcaggagctgcgacggtgcgattgatctcaagcttagtgccacta

SEQ ID NO: 183 - DNA, 72 bp, Artificial Sequence
  Description of Artificial Sequence Synthetic primer
gccgcccccggactcaaggagctgcacctcaggccctttcgtggaggcggaggacacggtcagagtggtacc

SEQ ID NO: 184 - DNA, 72 bp, Artificial Sequence
  Description of Artificial Sequence Synthetic primer
ggtaccactctgaccgtgtcctccgcctccacgaaagggcctgaggtgcagctccttgagtccgggggcggc

SEQ ID NO: 185 - protein, 375 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPEVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSS

SEQ ID NO: 186 - protein, 333 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPDIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKR

SEQ ID NO: 187 - protein, 375 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPEVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 188 - protein, 333 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 189 - DNA, 70 bp, Artificial Sequence
  Description of Artificial Sequence Synthetic primer
ctccaccagctggacctcaggccctttcgtggaggcggaggacacggtcagagtggtaccctgcccccag

SEQ ID NO: 190 - DNA, 70 bp, Artificial Sequence
  Description of Artificial Sequence Synthetic primer
ctgggggcagggtaccactctgaccgtgtcctccgcctccacgaaagggcctgaggtccagctggtggag

SEQ ID NO: 191 - DNA, 70 bp, Artificial Sequence
  Description of Artificial Sequence Synthetic primer
tggatggactttgcgtcatctgtatatcaggagctgcgacggtgcgattgatctcaagcttagtgccact

SEQ ID NO: 192 - DNA, 70 bp, Artificial Sequence
  Description of Artificial Sequence Synthetic primer
agtggcactaagcttgagatcaatcgcaccgtcgcagctcctgatatacagatgacgcaaagtccatcca

SEQ ID NO: 193 - protein, 370 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 194 - protein, 333 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 195 - protein, 370 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 196 - protein, 333 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 197 - DNA, 70 bp, Artificial Sequence
  Description of Artificial Sequence Synthetic primer
agcgggagactgggagagcacgatttgaggagctgcgacggtgcgattgatctcaagcttagtgccacta

SEQ ID NO: 198 - DNA, 70 bp, Artificial Sequence
  Description of Artificial Sequence Synthetic primer
tagtggcactaagcttgagatcaatcgcaccgtcgcagctcctcaaatcgtgctctcccagtctcccgct

SEQ ID NO: 199 - DNA, 70 bp, Artificial Sequence
  Description of Artificial Sequence Synthetic primer
gccccaggttgctgcagctgaacctgaggccctttcgtggaggcggaggacacggtcagagtggtaccct

SEQ ID NO: 200 - DNA, 70 bp, Artificial Sequence
  Description of Artificial Sequence Synthetic primer
agggtaccactctgaccgtgtcctccgcctccacgaaagggcctcaggttcagctgcagcaacctggggc

SEQ ID NO: 201 - protein, 371 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 202 - protein, 332 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPQIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 203 - protein, 371 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 204 - protein, 332 aa, Artificial Sequence
  Description of Artificial Sequence Synthetic polypeptide
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEINRTVAAPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPQIVLSQSPAILSPSPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 205 - protein, 5 aa, Artificial Sequence
  Description of Artificial Sequence Synthetic peptide
GGGGS